(19) United States
(12) Patent Application Publication
(10) Pub. No.: US 2024/0415015 A1
(43) Pub. Date: Dec. 12, 2024

(54) MIXTURE INCLUDING 5- AND/OR 6-MEMBERED RING FUSED STRUCTURE, COMPOSITION, ORGANIC ELECTROLUMINESCENT DEVICE AND ELECTRONIC DEVICE THEREOF

(71) Applicant: BEIJING SUMMER SPROUT TECHNOLOGY CO., LTD., Beijing (CN)

(72) Inventors: Le WANG, Beijing (CN); Qiang WANG, Beijing (CN); Ziyan ZHANG, Beijing (CN); Gaoqi ZHANG, Beijing (CN); Chi Yuen Raymond KWONG, Beijing (CN); Chuanjun XIA, Beijing (CN)

(21) Appl. No.: 18/677,029

(22) Filed: May 29, 2024

(30) Foreign Application Priority Data
May 30, 2023 (CN) 202310625861.8
Apr. 19, 2024 (CN) 202410478123.X

(51) Int. Cl. H10K 85/60; C07D 405/04; C07D 405/10; C07D 487/04; C09K 11/06; H10K 50/12; H10K 101/00

(52) U.S. Cl. CPC H10K 85/6572; C07D 405/04; C07D 405/10; C07D 487/04; C09K 11/06; H10K 85/615; H10K 85/626; H10K 85/654; H10K 85/6574; C09K 2211/1018; H10K 50/12; H10K 2101/90

(57) ABSTRACT

Provided are a mixture comprising a 5- and/or 6-membered ring fused structure, a composition, an organic electroluminescent device and an electronic device thereof. The mixture, the composition, the organic electroluminescent device and the electronic device thereof comprise at least three compounds, wherein one of the compounds comprises a fused structure formed by fusing at least four 5- and/or 6-membered rings and the fused structure is not triphenylene. The composition and the mixture can be used as host materials in organic electroluminescent devices to obtain electroluminescent devices with excellent comprehensive performance and in particular, to achieve a significant improvement in lifetime while maintaining high-level device efficiency. In particular, the mixture comprising at least three specific compounds has high evaporation stability and can be used as a single evaporation source in the preparation process of OLED devices, thereby simplifying the production process and reducing the production cost.

### CROSS-REFERENCE TO RELATED APPLICATION(S)

This application claims priority to Chinese Patent Application No. 202310625861.8 filed on May 30, 2023, and Chinese Patent Application No. 202410478123.X filed on Apr. 19, 2024, the disclosure of which are incorporated herein by reference in their entireties.

### TECHNICAL FIELD

The present disclosure relates to a mixture comprising three or more compounds, a composition, an organic electroluminescent device and an electronic device comprising the organic electroluminescent device and, in particular, to a mixture that comprises three or more compounds, one of which comprises a structure formed by fusing at least four 5- and/or 6-membered rings, a composition, an organic electroluminescent device and an electronic device thereof.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which comprises an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modern organic light-emitting diodes (OLEDs). State-of-the-art OLEDs may comprise multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may comprise one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

WO2018217067A1 discloses an organic electroluminescent device whose light-emitting layer includes a first host having a structure of Formula 1

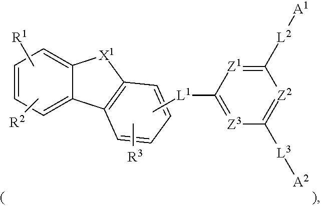

a second host formed by structures of Formula 2

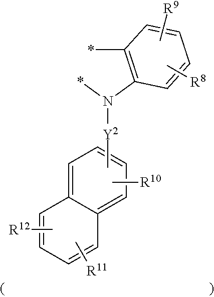

### and Formula 3

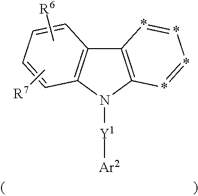

and a phosphorescent dopant with a maximum emission wavelength of 550 nm to 750 nm. This application focuses on an organic electroluminescent device including specific dual host materials and the technical effects thereof, does not disclose at least three compounds having different structures to be included, wherein one of the compounds includes a fused structure formed by fusing at least four 5- and/or 6-membered rings and the fused structure is not triphenylene, and does not disclose an electroluminescent device that includes a composition or mixture having three or more components and the technical effects thereof.

To obtain a device with better comprehensive performance, one manner that may be adopted is to prepare a light-emitting layer by using multiple source materials, and thus, it is particularly important to select a combination of materials that match each other better. Currently, most compound compositions or mixtures disclosed in the existing art are those including a two-component material. Research on compositions including three or more compounds or mixtures having high evaporation stability is still to be further explored, in particular compositions and mixtures that include a fused structure formed by fusing at least four 5- and/or 6-membered rings and in which the fused structure is not triphenylene and electroluminescent devices thereof. Therefore, it is a technical problem to be solved urgently by the persons skilled in the art to focus on the combination of different materials and develop a new combination of materials that match each other better in performance or a mixture having high evaporation stability to be applied to an organic electroluminescent device to obtain better device performance.

### SUMMARY

The present disclosure aims to provide a mixture, a composition and an organic electroluminescent device comprising three or more compounds to solve at least part of the above problems. The new mixture provided by the present disclosure comprises at least three hole or electron transport-type compounds that are different from each other in structure, wherein at least one hole transport-type material comprises a fused structure (which is not triphenylene) formed by fusing at least four 5- and/or 6-membered rings. The new compound composition provided by the present disclosure also comprises at least three compounds, wherein a first compound has a structure of Formula 1 with an indolocarbazole backbone (which also comprises a fused structure formed by fusing at least four 5- and 6-membered rings), a second compound has a structure of Formula 2 with a triazine backbone, a third compound has a structure of Formula 1 or Formula 2, and the first compound, the second compound and the third compound are different from each other in structure. The new compound composition and mixture comprising three or more components disclosed by the present disclosure can be used as host materials in organic electroluminescent devices to obtain electroluminescent devices with excellent comprehensive performance and in particular, to achieve a significant improvement in lifetime while maintaining high-level device efficiency. In particular, the mixture comprising three or more specific compounds in the present disclosure has high evaporation stability and can be used as a single evaporation source in the preparation process of OLED devices, thereby simplifying the production process and reducing the production cost.

According to an embodiment of the present disclosure, a mixture is disclosed. The mixture at least comprises a first compound, a second compound and a third compound;
wherein the first compound is a hole transport-type compound; the second compound is an electron transport-type compound; the third compound is selected from a hole transport-type compound or an electron transport-type compound;
the hole transport-type compound comprises a fused structure formed by fusing at least four 5- and/or 6-membered rings, wherein the fused structure is not triphenylene;
the first compound, the second compound and the third compound are different from each other in chemical structure;
the mixture comprises a pre-mix formed by pre-mixing the first compound, the second compound and the third compound, the mass ratio of at least one compound among the first compound, the second compound and the third compound in the mixture is C₀, and when the mixture is evaporated at a vacuum degree of 10⁻⁶ torr or below and a rate of 0.01-5 A/s, the mixture is evaporated on a surface positioned at a certain distance from the mixture to be evaporated to form n films with a certain thickness, wherein the mass ratio of the compound in an n^{th} film is Cₙ, and n is an integer greater than or equal to 1;
wherein the absolute value |Cₘ−C₀| of the difference between the mass ratio Cₘ of the compound in any one of the n evaporated films and C₀ is less than or equal to 2%, wherein m is an integer selected from 1 to n.

According to another embodiment of the present disclosure, a compound composition is further provided. The compound composition comprises a first compound represented by Formula 1, a second compound represented by Formula 2 and a third compound represented by Formula 1 or Formula 2, wherein the first compound, the second compound and the third compound are different from each other in structure;

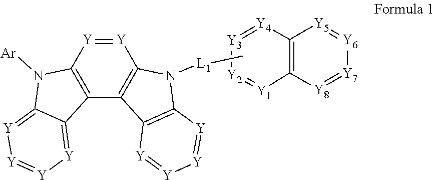

wherein
Ar is selected from substituted or unsubstituted aryl having 6 to 30 carbon atoms, substituted or unsubstituted heteroaryl having 3 to 30 carbon atoms or a combination thereof;
L₁ is selected from a single bond, substituted or unsubstituted arylene having 6 to 30 carbon atoms, substituted or unsubstituted heteroarylene having 3 to 30 carbon atoms or a combination thereof;
Y is, at each occurrence identically or differently, selected from CR_{y} or N;
Y₁ to Y₄ are, at each occurrence identically or differently, selected from C, CR_{y} or N, and at least one of Y₁ to Y₄ is selected from C and joined to L₁;
Y₅ to Y₈ are, at each occurrence identically or differently, selected from CR_{y} or N;
adjacent substituents R_{y} can be optionally joined to form a ring;

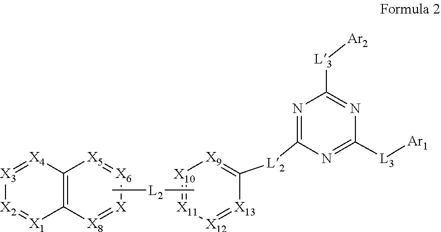

wherein
X₁ to X₄ are, at each occurrence identically or differently, selected from CRₓ or N;
X₅ to X₁₃ are, at each occurrence identically or differently, selected from C, CRₓ or N, at least one of X₅ to X₈ is selected from C and joined to L₂, and at least one of X₉ to X₁₃ is selected from C and joined to L₂;
L₂, L′₂, L₃ and L′₃ are, at each occurrence identically or differently, selected from a single bond, substituted or unsubstituted arylene having 6 to 30 carbon atoms, substituted or unsubstituted heteroarylene having 3 to 30 carbon atoms or a combination thereof;
Ar₁ and Ar₂ are, at each occurrence identically or differently, selected from substituted or unsubstituted aryl having 6 to 30 carbon atoms, substituted or unsubstituted heteroaryl having 3 to 30 carbon atoms or a combination thereof;
adjacent substituents Rₓ can be optionally joined to form a ring;
Rₓ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

According to another embodiment of the present disclosure, an electroluminescent device is further disclosed. The electroluminescent device comprises a first electrode, a second electrode and an organic layer disposed between the first electrode and the second electrode, wherein the organic layer at least comprises the mixture or the composition compound described in the preceding embodiments.

According to an embodiment of the present disclosure, a method for preparing an electroluminescent device is further disclosed, wherein the electroluminescent device comprises the mixture described in the preceding embodiments. The preparation method comprises the following steps:
step 1: providing a substrate, and setting a first electrode on the substrate;
step 2: per-mixing the first compound, the second compound and the third compound to form the mixture described in the preceding embodiments, loading the mixture into a container of a high-vacuum deposition tool, and when the mixture in the container is evaporated at a rate of 0.01-5 Å/s using the high-vacuum deposition tool with a vacuum degree of 10⁻⁶ torr or below, evaporating the mixture on a surface positioned at a certain distance from the mixture to be evaporated to form an organic layer; wherein a mass ratio of at least one compound among the first compound, the second compound and the third compound in the mixture is C₀, and a mass ratio of the at least one compound in the organic layer to a total mass of the first compound, the second compound and the third compound is C′; wherein |C′−C₀|≤2%; and
step 3: depositing a second electrode on the organic layer.

According to an embodiment of the present disclosure, a method for preparing an electroluminescent device is further disclosed, wherein the electroluminescent device comprises the compound composition described in the preceding embodiments. The preparation method comprises the following steps:
step 1: providing a substrate, and setting a first electrode on the substrate;
step 2: loading a first compound, a second compound and a third compound into different containers of a high-vacuum deposition tool, respectively, and when the compounds in the containers are evaporated simultaneously at a rate of 0.01-5 Å/s using the high-vacuum deposition tool with a vacuum degree of 10⁻⁶ torr or below, evaporating the first compound, the second compound and the third compound on a surface positioned at a certain distance from the compounds to be evaporated such that the three compounds are co-deposited to form an organic layer; and
step 3: depositing a second electrode on the organic layer.

According to another embodiment of the present disclosure, an electronic device is further disclosed. The electronic device comprises an electroluminescent device, wherein the electroluminescent device and the preparation method thereof are shown in the preceding embodiments.

The new mixture provided by the present disclosure comprises at least three hole or electron transport-type compounds that are different from each other in structure, wherein at least one hole transport-type material comprises a fused structure (which is not triphenylene) formed by fusing at least four 5- and/or 6-membered rings. The new compound composition provided by the present disclosure also comprises at least three compounds, wherein a first compound has a structure of Formula 1 with an indolocarbazole backbone, a second compound has a structure of Formula 2 with a triazine backbone, the third compound has a structure of Formula 1 or Formula 2, and the first compound, the second compound and the third compound are different from each other in structure. The new compound composition and mixture comprising three or more components disclosed by the present disclosure can be used as host materials in organic electroluminescent devices to obtain electroluminescent devices with excellent comprehensive performance and in particular, to achieve a significant improvement in lifetime while maintaining high-level device efficiency. In particular, the mixture in the present disclosure comprises a fused structure (which is not triphenylene) formed by fusing at least four 5- and/or 6-membered rings as its component, has high evaporation stability, and can be used as a single evaporation source in the preparation process of OLED devices, thereby simplifying the production process and reducing the production cost.

### BRIEF DESCRIPTION OF DRAWINGS

FIG. 1 is a schematic diagram of an organic light-emitting device that may include a mixture or a compound composition disclosed herein.

FIG. 2 is a schematic diagram of another organic light-emitting device that may include a mixture or a compound composition disclosed herein.

### DETAILED DESCRIPTION

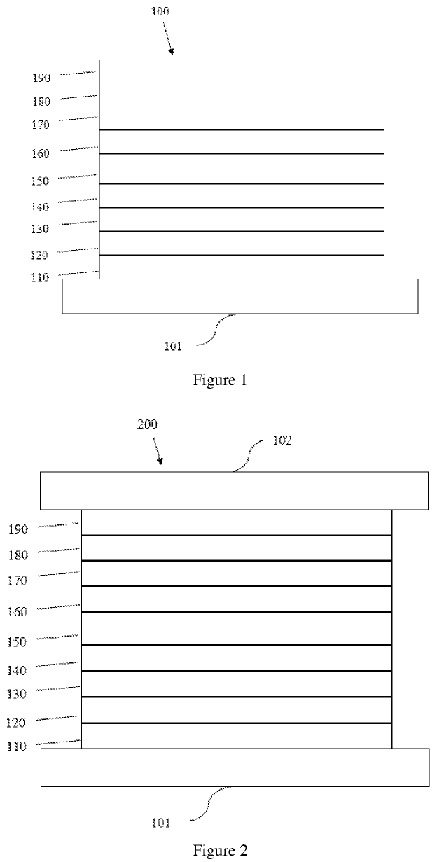

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emissive materials to achieve desired emission spectrum.

In one embodiment, an OLED may be described as having an “organic layer” disposed between a cathode and an anode. This organic layer may include a single layer or multiple layers.

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔE_{S-T}). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small ΔE_{S-T}. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, a n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butyldimethylgermanylmethyl, triethylgermanylmethyl, triethylgermanylethyl, triisopropylgermanylmethyl, triisopropylgermanylethyl, trimethylsilylmethyl, trimethylsilylethyl, trimethylsilylisopropyl, triisopropylsilylmethyl and triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups or heterocycle (heterocyclyl)—as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, wherein at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with at least one aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl—as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclyl, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclyl, arylalkyl, alkoxy, aryloxy, alkenyl, alkynyl, aryl, heteroaryl, alkylsilyl, arylsilyl, alkylgermanyl, arylgermanyl, amino, acyl, carbonyl, a carboxylic acid group, an ester group, sulfinyl, sulfonyl, and phosphino may be substituted with one or more groups selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, unsubstituted heterocyclyl having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl group having 6 to 20 carbon atoms, unsubstituted alkylgermanyl group having 3 to 20 carbon atoms, unsubstituted arylgermanyl group having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen may also be replaced by their other stable isotopes. The replacement by other stable isotopes in the compounds may be preferred due to its enhancements of device efficiency and stability.

In the compounds mentioned in the present disclosure, multiple substitutions refer to a range that includes di-substitutions, up to the maximum available substitutions. When substitution in the compounds mentioned in the present disclosure represents multiple substitutions (including di-, tri-, and tetra-substitutions etc.), that means the substituent may exist at a plurality of available substitution positions on its linking structure, the substituents present at a plurality of available substitution positions may have the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fusedcyclic, and etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

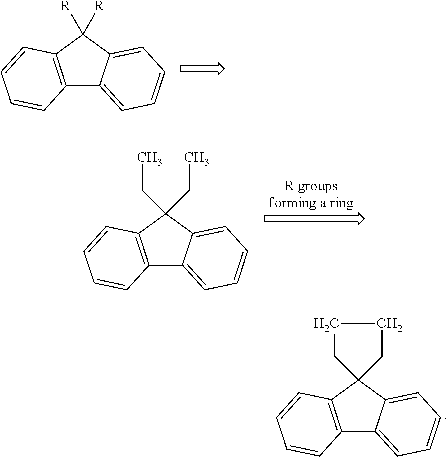

The expression that adjacent substituents can be optionally joined to form a ring is also intended to mean that two substituents bonded to carbon atoms which are directly bonded to each other are joined to each other via a chemical bond to form a ring, which can be exemplified by the following formula:

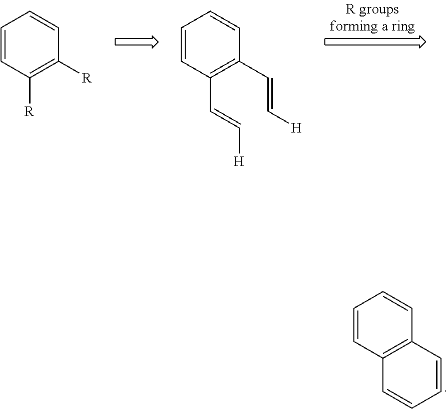

The expression that adjacent substituents can be optionally joined to form a ring is also intended to mean that two substituents bonded to further distant carbon atoms are joined to each other via a chemical bond to form a ring, which can be exemplified by the following formula:

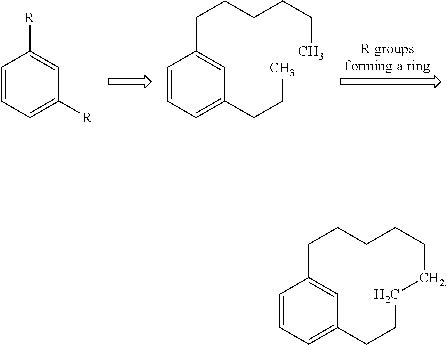

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

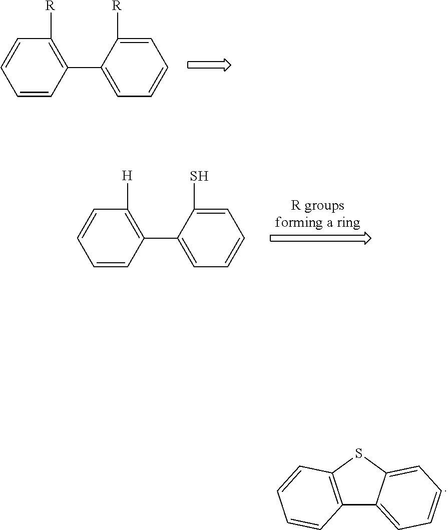

According to an embodiment of the present disclosure, a mixture is further disclosed. The mixture at least comprises a first compound, a second compound and a third compound;
wherein the first compound is a hole transport-type compound; the second compound is an electron transport-type compound; the third compound is selected from a hole transport-type compound or an electron transport-type compound;
the hole transport-type compound comprises a fused structure formed by fusing at least four 5- and/or 6-membered rings, wherein the fused structure is not triphenylene;
the first compound, the second compound and the third compound are different from each other in chemical structure;
the mixture comprises a pre-mix formed by pre-mixing the first compound, the second compound and the third compound, the mass ratio of at least one compound among the first compound, the second compound and the third compound in the mixture is C₀, and when the mixture is evaporated at a vacuum degree of 10⁻⁶ torr or below and a rate of 0.01-5 Å/s, the mixture is evaporated on a surface positioned at a certain distance from the mixture to be evaporated to form n films with a certain thickness, wherein the mass ratio of the at least one compound in an n^{th} film is Cₙ, and n is an integer greater than or equal to 1;
wherein the absolute value |Cₘ−C₀| of the difference between the mass ratio Cₘ of the at least one compound in any one of the n evaporated films and C₀ is less than or equal to 2%, wherein m is an integer selected from 1 to n.

The “fused structure formed by fusing at least four 5- and/or 6-membered rings” herein refers to a chemical structure (group) formed by fusing four or more 5- and/or 6-membered rings, including, but not limited to, an indolocarbazole structure, an azaindolocarbazole structure, a benzofurocarbazole structure, a benzothienocarbazole structure and a benzoselenophenocarbazole structure, or refers to a polycyclic fused structure fused by indolocarbazole.

The “hole transport-type compound” herein refers to a compound whose hole transport capability is greater than its electron transport capability and which can be used as a hole transport material in an electroluminescent device, including, but not limited to, a hole transport compound or a bipolar compound.

The “electron transport-type compound” herein refers to a compound whose electron transport capability is greater than its hole transport capability, that is, the electronic transport capability of the compound is greater than its hole transport capability, including, but not limited to, an electron transport compound or a bipolar compound.

The hole transport capability and the electron transport capability, for example, may be determined by the persons skilled in the art according to the carrier mobility of the material; a material whose electron mobility is greater than its hole mobility is a material with a higher electron transport capability, and on the contrary, a material whose hole mobility greater than its electron mobility is a material with a higher hole transport capability.

According to another embodiment of the present disclosure, the hole transport-type compound comprises at least one heteroatom, and the heteroatom is selected from the group consisting of: a nitrogen atom, an oxygen atom, a sulfur atom, a selenium atom, a phosphorus atom, a silicon atom, a germanium atom, a boron atom, and combinations thereof.

According to another embodiment of the present disclosure, the hole transport-type compound comprises at least one heteroatom, and the heteroatom is selected from the group consisting of: a nitrogen atom, an oxygen atom, a sulfur atom, a selenium atom, and combinations thereof.

According to another embodiment of the present disclosure, the hole transport-type compound comprises at least one heteroatom, and the heteroatom is selected from the group consisting of: a nitrogen atom, an oxygen atom, a sulfur atom, and combinations thereof.

According to another embodiment of the present disclosure, the hole transport-type compound comprises at least one heteroatom, and the heteroatom is selected from a nitrogen atom.

According to another embodiment of the present disclosure, when the third compound is selected from a hole transport-type compound, in evaporation characteristics, the third compound is heavier than the second compound, and the first compound is lighter than the second compound.

According to another embodiment of the present disclosure, when the third compound is selected from an electron transport-type compound, in evaporation characteristics, the second compound is heavier than the first compound, and the third compound is lighter than the first compound.

Herein, any two compounds are pre-mixed to form a two-component mixture. When the two-component mixture is evaporated at a vacuum degree of about 10⁻⁶ torr or below and a rate of 0.01-5 Å/s, the two-component mixture is evaporated on a surface positioned at a certain distance from the mixture to be evaporated to form n films with a certain thickness. As the number of films increases, when the mass ratio of one compound overall tends to increase gradually, the compound is considered as “heavy” in evaporation characteristics. On the contrary, when the mass ratio of one compound overall tends to decrease gradually, the compound is considered as “light” in evaporation characteristics. As shown by the data in Table 5 below, the mass ratio of Compound A-3 overall tends to increase gradually in the films (from 47.079% in the first film to 49.755% in the sixth film) as the number of films increases, then, it is considered that Compound A-3 is heavier than Compound B-1.

According to an embodiment of the present disclosure, the mass ratio of at least one compound among the first compound, the second compound and the third compound in the mixture is C₀, and when the mixture is evaporated at a vacuum degree of 10⁻⁶ torr or below and a rate of 0.01-5 Å/s, the mixture is evaporated on a surface positioned at a certain distance from the mixture to be evaporated to form n films with a certain thickness, wherein the mass ratio of the compound in the n^{th} film is Cₙ; n is an integer greater than or equal to 1; the absolute value |Cₘ−C₀| of the difference between the mass ratio Cₘ of the compound in any one of the n evaporated films and C₀ is less than or equal to 1.5%; m is an integer selected from 1 to n.

According to an embodiment of the present disclosure, the mass ratio of at least one compound among the first compound, the second compound and the third compound in the mixture is C₀, and when the mixture is evaporated at a vacuum degree of 10⁻⁶ torr or below and a rate of 0.01-5 Å/s, the mixture is evaporated on a surface positioned at a certain distance from the mixture to be evaporated to form n films with a certain thickness, wherein the mass ratio of the compound in the n^{th} film is Cₙ; n is an integer greater than or equal to 1; the absolute value |Cₘ−C₀| of the difference between the mass ratio Cₘ of the compound in any one of the n evaporated films and C₀ is less than or equal to 1.0%; m is an integer selected from 1 to n.

According to an embodiment of the present disclosure, the mass ratio of at least one compound among the first compound, the second compound and the third compound in the mixture is C₀, and when the mixture is evaporated at a vacuum degree of 10⁻⁶ torr or below and a rate of 0.01-5 Å/s, the mixture is evaporated on a surface positioned at a certain distance from the mixture to be evaporated to form n films with a certain thickness, wherein the mass ratio of the compound in the n^{th} film is Cₙ; n is an integer greater than or equal to 1; the absolute value |Cₘ−C₀| of the difference between the mass ratio Cₘ of the compound in any one of the n evaporated films and C₀ is less than or equal to 0.5%; m is an integer selected from 1 to n.

According to an embodiment of the present disclosure, the evaporation temperature of the first compound, the second compound and the third compound are 120° C.-390° C.

According to an embodiment of the present disclosure, the evaporation temperature of the first compound, the second compound and the third compound are 140° C.-370° C.

According to an embodiment of the present disclosure, the evaporation temperature of the first compound, the second compound and the third compound are 160° C.-360° C.

According to an embodiment of the present disclosure, the evaporation temperature of the first compound, the second compound and the third compound are 200° C.-350° C.

According to an embodiment of the present disclosure, when the first compound, the second compound and the third compound are evaporated at a vacuum degree of 10⁻⁶ torr or below and a rate of 0.01-5 Å/s, the absolute value of the evaporation temperature difference between any two of the first compound, the second compound and the third compound is less than 30° C.

According to an embodiment of the present disclosure, when the first compound, the second compound and the third compound are evaporated at a vacuum degree of 10⁻⁶ torr or below and a rate of 0.01-5 Å/s, the absolute value of the evaporation temperature difference between any two of the first compound, the second compound and the third compound is less than 20° C.

According to an embodiment of the present disclosure, when the first compound, the second compound and the third compound are evaporated at a vacuum degree of 10⁻⁶ torr or below and a rate of 0.01-5 Å/s, the absolute value of the evaporation temperature difference between any two of the first compound, the second compound and the third compound is less than 10° C.

According to an embodiment of the present disclosure, the hole transport-type compound has a HOMO energy level of −3.5 eV to −6.0 eV.

According to an embodiment of the present disclosure, the hole transport-type compound has a HOMO energy level of −4.0 eV to −5.6 eV.

According to an embodiment of the present disclosure, the electron transport-type compound has a LUMO energy level of −1.9 eV to −3.5 eV.

According to an embodiment of the present disclosure, the electron transport-type compound has a LUMO energy level of −2.0 eV to −3.0 eV.

According to an embodiment of the present disclosure, in the mixture, the triplet energy level T₁ of at least one compound among the first compound, the second compound and the third compound is less than 2.65 eV.

According to an embodiment of the present disclosure, in the mixture, the triplet energy level T₁ of at least one compound among the first compound, the second compound and the third compound is less than is less than 2.60 eV.

According to an embodiment of the present disclosure, the hole transport-type compound and/or the electron transport-type compound include at least one chemical group selected from the group consisting of: indolocarbazole, benzene, pyridine, pyrimidine, carbazole, azacarbazole, dibenzothiophene, azadibenzothiophe, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene, oxazole, thiazole, and combinations thereof.

In this embodiment, substituents or fused ring derivatives of groups such as “indolocarbazole, benzene, pyridine, pyrimidine, carbazole, azacarbazole, dibenzothiophene, azadibenzothiophe, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene, oxazole and thiazole” are also the “chemical group” described in this embodiment, including, but not limited to, “substituted indolocarbazole”, “substituted benzene”, “substituted pyridine” or “benzoindolocarbazole (a fused ring derivative of indolocarbazole)”.

According to an embodiment of the present disclosure, the hole transport-type compound comprises an indolo[2,3-C]carbazolyl group.

According to an embodiment of the present disclosure, the electron transport-type compound comprises a triazinyl group.

According to an embodiment of the present disclosure, in the mixture, the first compound has a structure represented by Formula 1′:

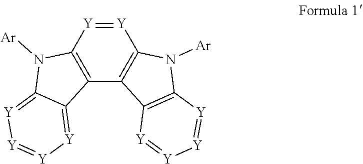

wherein
Ar is selected from substituted or unsubstituted aryl having 6 to 30 carbon atoms, substituted or unsubstituted heteroaryl having 3 to 30 carbon atoms or a combination thereof;
Y is, at each occurrence identically or differently, selected from CR_{y} or N;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{y} can be optionally joined to form a ring.

In this embodiment, the expression that adjacent substituents R_{y} can be optionally joined to form a ring is intended to mean that adjacent two substituents R_{y} can be joined to form a ring. For example, in Formula 1′, one or more of groups of adjacent substituents R_{y} in the group Y can be optionally joined to form a ring. Obviously, it is possible that none of these adjacent substituents are joined to form a ring.

According to an embodiment of the present disclosure, in the mixture, the first compound has a structure represented by Formula 1.

According to an embodiment of the present disclosure, in the mixture, the first compound has a structure represented by Formula 1-1.

According to an embodiment of the present disclosure, in the mixture, the first compound is selected from the group consisting of Compound A-1 to Compound A-263, wherein for the specific structures of Compound A-1 to Compound A-263, reference is made to claim 12.

According to an embodiment of the present disclosure, in the mixture, hydrogen in Compound A-1 to Compound A-263 can be partially or fully substituted with deuterium.

According to an embodiment of the present disclosure, in the mixture, the second compound has a structure represented by Formula 2′:

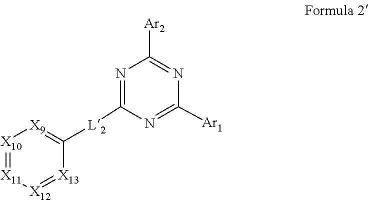

wherein
X₉ to X₁₃ are, at each occurrence identically or differently, selected from CRₓ or N;
L′₂ is selected from a single bond, substituted or unsubstituted arylene having 6 to 30 carbon atoms, substituted or unsubstituted heteroarylene having 3 to 30 carbon atoms or a combination thereof;
Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Ar₁ and Ar₂ are selected from substituted or unsubstituted aryl having 6 to 30 carbon atoms, substituted or unsubstituted heteroaryl having 3 to 30 carbon atoms or a combination thereof;
adjacent substituents Rₓ can be optionally joined to form a ring.

In this embodiment, the expression that adjacent substituents Rₓ can be optionally joined to form a ring is intended to mean that adjacent two substituents Rₓ can be joined to form a ring. For example, one or more of groups of adjacent two substituents Rₓ in X₉ to X₁₃, including, but not limited to, adjacent two substituents Rₓ in X₉ and X₁₀, adjacent two substituents Rₓ in X₁₀ and X₁₁, and adjacent two substituents Rₓ in X₉ and X₁₁, can be optionally joined to form a ring. Obviously, it is possible that none of these adjacent substituents are joined to form a ring.

According to an embodiment of the present disclosure, at least one of Ar₁ or Ar₂ in Formula 2′ is selected from a group containing substituted or unsubstituted dibenzofuryl, substituted or unsubstituted dibenzothienyl, substituted or unsubstituted carbazolyl, substituted or unsubstituted silafluorenyl, substituted or unsubstituted fluorenyl, substituted or unsubstituted naphthyl, substituted or unsubstituted chrysenyl or a combination thereof.

According to an embodiment of the present disclosure, in the mixture, the second compound has a structure represented by Formula 2.

According to an embodiment of the present disclosure, in the mixture, the second compound is selected from the group consisting of Compound B-1 to Compound B-223, wherein for the specific structures of Compound B-1 to Compound B-223, reference is made to claim 13.

According to an embodiment of the present disclosure, hydrogen in Compound B-1 to Compound B-223 can be partially or fully substituted with deuterium.

According to an embodiment of the present disclosure, in the mixture, the third compound and the first compound or the third compound and the second compound are isomers of each other or isotopologues of each other.

Herein, the “isotopologue” includes, but is not limited to, two cases: when the compound formed by substituting hydrogen in the structure of Compound X with deuterium is referred to as Compound X′, Compound X′ and Compound X are isotopologues of each other and Compound X′ and Compound Z (an isomer of Compound X) are also isotopologues of each other. For example, Compound A-3

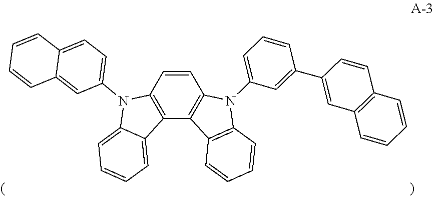 [SMILES: c1cc(-c2ccc3ccccc3c2)cc(-n2c3ccccc3c3c4c5ccccc5n(-c5ccc6ccccc6c5)c4ccc32)c1]

### and Compound A-79

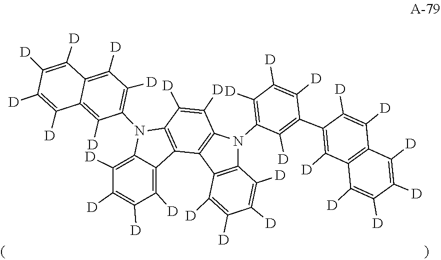 [SMILES: [2H]c1c([2H])c(-c2c([2H])c([2H])c3c([2H])c([2H])c([2H])c([2H])c3c2[2H])c([2H])c(-n2c3c([2H])c([2H])c([2H])c([2H])c3c3c4c5c([2H])c([2H])c([2H])c([2H])c5n(-c5c([2H])c([2H])c6c([2H])c([2H])c([2H])c([2H])c6c5[2H])c4c([2H])c([2H])c32)c1[2H]]

are isotopologues of each other, and Compound A-3

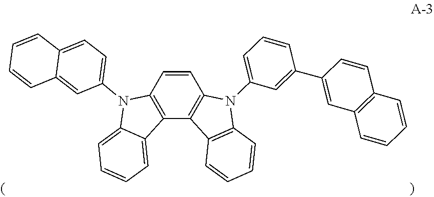 [SMILES: c1cc(-c2ccc3ccccc3c2)cc(-n2c3ccccc3c3c4c5ccccc5n(-c5ccc6ccccc6c5)c4ccc32)c1]

### and Compound A-78

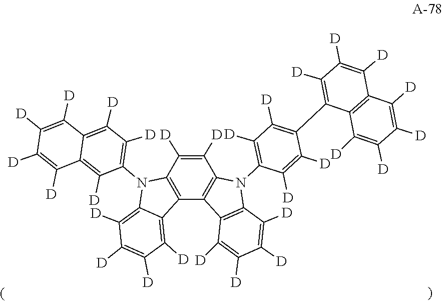 [SMILES: [2H]c1c([2H])c(-n2c3c([2H])c([2H])c([2H])c([2H])c3c3c4c5c([2H])c([2H])c([2H])c([2H])c5n(-c5c([2H])c([2H])c6c([2H])c([2H])c([2H])c([2H])c6c5[2H])c4c([2H])c([2H])c32)c([2H])c([2H])c1-c1c([2H])c([2H])c([2H])c2c([2H])c([2H])c([2H])c([2H])c12]

an isomer of Compound A-79) are also isotopologues of each other.

According to an embodiment of the present disclosure, the third compound has a structure represented by Formula 1′ or Formula 2′.

According to an embodiment of the present disclosure, the third compound has a structure represented by Formula 1 or Formula 2.

According to an embodiment of the present disclosure, the third compound has a structure represented by Formula 1-1 or Formula 2.

According to an embodiment of the present disclosure, the third compound has a structure represented by Formula 1′, and the third compound and the first compound are isomers of each other or isotopologues of each other.

According to an embodiment of the present disclosure, the third compound has a structure represented by Formula 2′, and the third compound and the second compound are isomers of each other or isotopologues of each other.

Herein, the “certain distance” and “certain thickness” described in the process of evaporating the mixture to form films may be adaptively adjusted by the persons skilled in the art according to actual requirements to achieve the purpose of evaporation.

Exemplarily, without limitation, the certain distance may be 10-100 cm, 30- to 80 cm or 35-60 cm. Exemplarily, without limitation, the certain thickness may be 100-10000 Å, 200-8000 Å or 400-5000 Å.

According to an embodiment of the present disclosure, the mass ratio among the first compound, the second compound and the third compound in the mixture is 0.1-99.9:0.1-99.9:0.1-99.9.

According to an embodiment of the present disclosure, the mass ratio among the first compound, the second compound and the third compound in the mixture is 1-99:1-99:1-99.

According to an embodiment of the present disclosure, the mass ratio among the first compound, the second compound and the third compound in the mixture is 10-90:10-90:10-90.

According to an embodiment of the present disclosure, a compound composition is provided. The compound composition comprises a first compound represented by Formula 1, a second compound represented by Formula 2 and a third compound represented by Formula 1 or Formula 2, wherein the first compound, the second compound and the third compound are different from each other in structure;

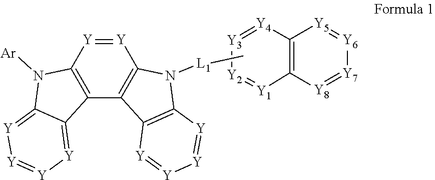

wherein
Ar is selected from substituted or unsubstituted aryl having 6 to 30 carbon atoms, substituted or unsubstituted heteroaryl having 3 to 30 carbon atoms or a combination thereof;
L₁ is selected from a single bond, substituted or unsubstituted arylene having 6 to 30 carbon atoms, substituted or unsubstituted heteroarylene having 3 to 30 carbon atoms or a combination thereof;
Y is, at each occurrence identically or differently, selected from CR_{y} or N;
Y₁ to Y₄ are, at each occurrence identically or differently, selected from C, CR_{y} or N, and at least one of Y₁ to Y₄ is selected from C and joined to L₁;
Y₅ to Y₈ are, at each occurrence identically or differently, selected from CR_{y} or N;
adjacent substituents R_{y} can be optionally joined to form a ring;

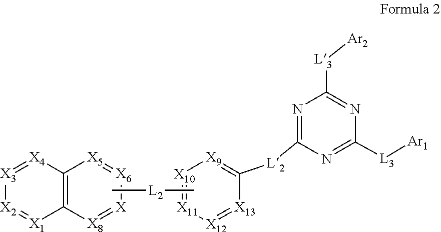

wherein
X₁ to X₄ are, at each occurrence identically or differently, selected from CRₓ or N;
X₈ to X₁₃ are, at each occurrence identically or differently, selected from C, CRₓ or N, at least one of X₅ to X₈ is selected from C and joined to L₂, and at least one of X₉ to X₁₃ is selected from C and joined to L₂;
L₂, L′₂, L₃ and L′₃ are, at each occurrence identically or differently, selected from a single bond, substituted or unsubstituted arylene having 6 to 30 carbon atoms, substituted or unsubstituted heteroarylene having 3 to 30 carbon atoms or a combination thereof;
Ar₁ and Ar₂ are, at each occurrence identically or differently, selected from substituted or unsubstituted aryl having 6 to 30 carbon atoms, substituted or unsubstituted heteroaryl having 3 to 30 carbon atoms or a combination thereof;
adjacent substituents Rₓ can be optionally joined to form a ring;
Rₓ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

In this embodiment, the expression that adjacent substituents R_{y} can be optionally joined to form a ring is intended to mean that adjacent two substituents R_{y} can be joined to form a ring. For example, in Formula 1, one or more of groups of adjacent substituents R_{y} in the group Y and adjacent two substituents R_{y} in Y₁ to Y₈ can be optionally joined to form a ring. Obviously, it is possible that none of these adjacent substituents are joined to form a ring.

In this embodiment, the expression that adjacent substituents Rₓ can be optionally joined to form a ring is intended to mean that adjacent two substituents Rₓ can be joined to form a ring. For example, one or more of groups of adjacent two substituents Rₓ in X₁ to X₁₃, including, but not limited to, adjacent two substituents Rₓ in X₁ and X₂, adjacent two substituents Rₓ in X₁ and X₃, adjacent two substituents Rₓ in X₇ and X₈, adjacent two substituents Rₓ in X₇ and X₁₁, and adjacent two substituents Rₓ in X₆ and X₁₀, can be optionally joined to form a ring. Obviously, it is possible that none of these adjacent substituents are joined to form a ring.

According to an embodiment of the present disclosure, in the compound composition, the first compound has a structure represented by Formula 1-1:

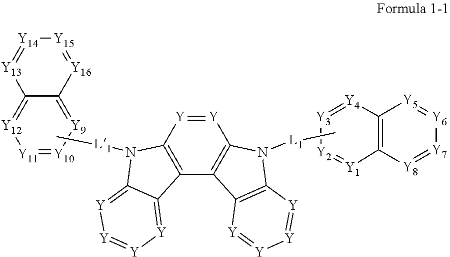

wherein
L₁ and L′₁ are, at each occurrence identically or differently, selected from a single bond, substituted or unsubstituted arylene having 6 to 30 carbon atoms, substituted or unsubstituted heteroarylene having 3 to 30 carbon atoms or a combination thereof;
Y is, at each occurrence identically or differently, selected from CR_{y} or N;
Y₁ to Y₄ and Y₉ to Y₁₂ are, at each occurrence identically or differently, selected from C, CR_{y} or N, at least one of Y₁ to Y₄ is selected from C and joined to L₁, and at least one of Y₉ to Y₁₂ is selected from C and joined to L′₁;
Y₅ to Y₈ and Y₁₃ to Y₁₆ are, at each occurrence identically or differently, selected from CR_{y} or N;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{y} can be optionally joined to form a ring.

In this embodiment, the expression that adjacent substituents R_{y} can be optionally joined to form a ring is intended to mean that adjacent two substituents R_{y} can be joined to form a ring. For example, in Formula 1-1, one or more of groups of adjacent substituents R_{y} in the group Y, adjacent two substituents R_{y} in Y₁ to Y₈ and adjacent two substituents R_{y} in Y₉ to Y₁₆ can be optionally joined to form a ring. Obviously, it is possible that none of these adjacent substituents are joined to form a ring.

According to an embodiment of the present disclosure, L₁ and L′₁ are, at each occurrence identically or differently, selected from a single bond, substituted or unsubstituted arylene having 6 to 20 carbon atoms, substituted or unsubstituted heteroarylene having 3 to 20 carbon atoms or a combination thereof.

According to an embodiment of the present disclosure, L₁ and L′₁ are, at each occurrence identically or differently, selected from a single bond or substituted or unsubstituted arylene having 6 to 20 carbon atoms.

According to an embodiment of the present disclosure, L₁ and L′₁ are, at each occurrence identically or differently, selected from a single bond or substituted or unsubstituted arylene having 6 to 12 carbon atoms.

According to an embodiment of the present disclosure, at least one of L₁ or L′₁ is selected from a single bond.

According to an embodiment of the present disclosure, L₁ and L′₁ are, at each occurrence identically or differently, selected from a single bond, substituted or unsubstituted phenylene, substituted or unsubstituted naphthylene, substituted or unsubstituted biphenylylene, substituted or unsubstituted phenanthrylene, substituted or unsubstituted triphenylenylene, substituted or unsubstituted 9,9-dimethylfluorenylene, substituted or unsubstituted terphenylene, substituted or unsubstituted chrysenylene or a combination thereof.

According to an embodiment of the present disclosure, Y₁ to Y₄ and Y₉ to Y₁₂ are, at each occurrence identically or differently, selected from C or CR_{y}, at least one of Y₁ to Y₄ is selected from C and joined to L₁, and at least one of Y₉ to Y₁₂ is selected from C and joined to L′1.

According to an embodiment of the present disclosure, Y₁ to Y₄ and Y₉ to Y₁₂ are, at each occurrence identically or differently, selected from C or CR_{y}, Y₁ or Y₂ is selected from C and joined to L₁, and Y₁₀ or Y₁₁ is selected from C and joined to L′₁.

According to an embodiment of the present disclosure, at least one Y is selected from N.

According to an embodiment of the present disclosure, Y is selected from CR_{y}.

According to an embodiment of the present disclosure, at least one R_{y} is selected from deuterium.

According to an embodiment of the present disclosure, Y is selected from CR_{y}, R_{y} is selected from deuterium.

According to an embodiment of the present disclosure, R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, and combinations thereof.

According to an embodiment of the present disclosure, R_{y} is, at each occurrence identically or differently, selected from the group consisting of: hydrogen, deuterium, halogen, phenyl, pyridyl, vinyl, adamantyl, methyl, ethyl, isopropyl, cyclopropyl, naphthyl, biphenyl, phenanthryl, triphenylenyl, tert-butyl, trifluoromethyl, 9,9-dimethylfluorenyl, terphenyl, dibenzothienyl, dibenzofuryl, benzothiazolyl, benzoxazolyl, phenanthroxazolyl, phenanthrothiazolyl, carbazolyl, chrysenyl, and combinations thereof.

According to an embodiment of the present disclosure, in the compound composition, the first compound is selected from the group consisting of Compound A-1 to Compound A-199, wherein for the specific structures of Compound A-1 to Compound A-199, reference is made to claim 12.

According to an embodiment of the present disclosure, hydrogen in Compound A-1 to Compound A-199 can be partially or fully substituted with deuterium.

According to an embodiment of the present disclosure, L₂, L′₂, L₃ and L′₃ are, at each occurrence identically or differently, selected from a single bond, substituted or unsubstituted arylene having 6 to 20 carbon atoms, substituted or unsubstituted heteroarylene having 3 to 20 carbon atoms or a combination thereof.

According to an embodiment of the present disclosure, L₂, L′₂, L₃ and L′₃ are, at each occurrence identically or differently, selected from a single bond or substituted or unsubstituted arylene having 6 to 20 carbon atoms.

According to an embodiment of the present disclosure, L₂, L′₂, L₃ and L′₃ are, at each occurrence identically or differently, selected from a single bond or substituted or unsubstituted arylene having 6 to 12 carbon atoms.

According to an embodiment of the present disclosure, at least one of L₂ or L′₂ is selected from a single bond.

According to an embodiment of the present disclosure, at least one of L₃ or L′₃ is selected from a single bond.

According to an embodiment of the present disclosure, L₂ and L′₂ are selected from a single bond, and L₃ and L′₃ are, at each occurrence identically or differently, selected from a single bond or substituted or unsubstituted arylene having 6 to 12 carbon atoms.

According to an embodiment of the present disclosure, L₂, L′₂, L₃ and L′₃ are, at each occurrence identically or differently, selected from a single bond, substituted or unsubstituted phenylene, substituted or unsubstituted naphthylene, substituted or unsubstituted biphenylylene, substituted or unsubstituted phenanthrylene, substituted or unsubstituted triphenylenylene, substituted or unsubstituted 9,9-dimethylfluorenylene, substituted or unsubstituted terphenylene, substituted or unsubstituted chrysenylene or a combination thereof.

According to an embodiment of the present disclosure, X₁ to X₄ are, at each occurrence identically or differently, selected from CRₓ, X₅ to X₁₃ are, at each occurrence identically or differently, selected from C or CRₓ, X₆ or X₇ is selected from C and joined to L₂, and X₁₁ is selected from C and joined to L₂.

According to an embodiment of the present disclosure, Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, and combinations thereof.

According to an embodiment of the present disclosure, at least one Rₓ is selected from deuterium.

According to an embodiment of the present disclosure, Rₓ is, at each occurrence identically or differently, selected from the group consisting of: hydrogen, deuterium, halogen, phenyl, pyridyl, vinyl, adamantyl, methyl, ethyl, isopropyl, cyclopropyl, naphthyl, biphenyl, phenanthryl, triphenylenyl, tert-butyl, trifluoromethyl, 9,9-dimethylfluorenyl, terphenyl, dibenzothienyl, dibenzofuryl, benzothiazolyl, benzoxazolyl, phenanthroxazolyl, phenanthrothiazolyl, carbazolyl, chrysenyl, and combinations thereof.

According to an embodiment of the present disclosure, Ar₁ and Ar₂ have, at each occurrence identically or differently, a structure represented by any one of Formula Ar-1 to Formula Ar-6 or a combination thereof:

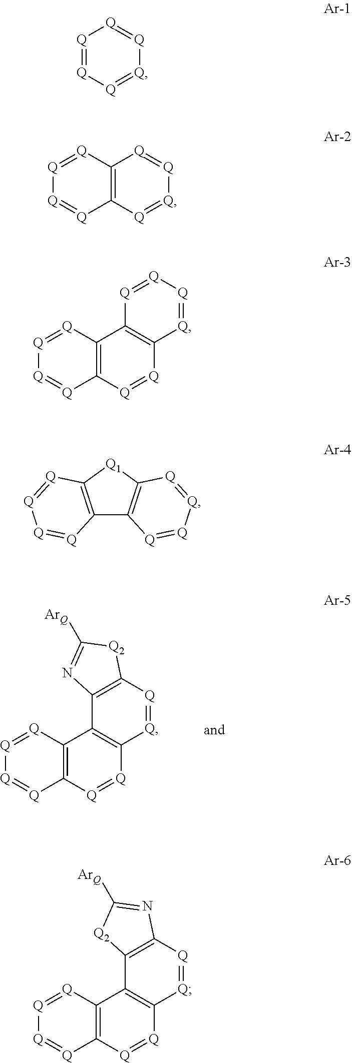

wherein
Ar_{Q} is, at each occurrence identically or differently, selected from substituted or unsubstituted aryl having 6 to 30 carbon atoms, substituted or unsubstituted heteroaryl having 3 to 30 carbon atoms or a combination thereof;
Q is, at each occurrence identically or differently, selected from C, CR_{Q} or N, and at least one Q is selected from C and joined to L₃ or L′₃;
Q₁ is selected from O, S, Se, NR_{Q} or CR_{Q}R_{Q};
Q₂ is selected from O, S or Se;
R_{Q} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{Q} can be optionally joined to form a ring.

In this embodiment, the expression that adjacent substituents R_{Q} can be optionally joined to form a ring is intended to mean that adjacent two substituents R_{Q} can be joined to form a ring. For example, one or more of groups of adjacent substituents R_{Q} in the group Q in Formula Ar-1 to Formula Ar-3 and Formula Ar-5 to Formula Ar-6, adjacent two substituents R_{Q} in the group Q in Formula Ar-4, and adjacent two substituents R_{Q} in the group Q and the group Q₁ in Formula Ar-4 can be optionally joined to form a ring. Obviously, it is possible that none of these adjacent substituents are joined to form a ring.

According to an embodiment of the present disclosure, Ar₁ and Ar₂ have, at each occurrence identically or differently, a structure represented by any one of Formula Ar-1, Formula Ar-2 or Formula Ar-4.

According to an embodiment of the present disclosure, L₃ and L′₃ are, at each occurrence identically or differently, selected from a single bond or substituted or unsubstituted phenylene, and Ar₁ and Ar₂ have, at each occurrence identically or differently, a structure represented by any one of Formula Ar-1 or Formula Ar-4.

According to an embodiment of the present disclosure, -L₃-Ar₁ and -L′₃-Ar₂ have, at each occurrence identically or differently, a structure represented by any one of Formula X-1, Formula X-2 or Formula X-3:

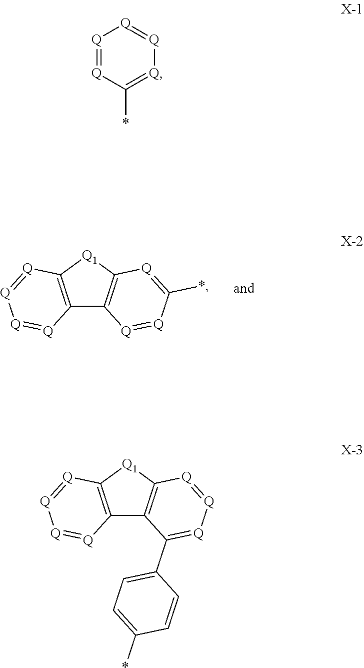

wherein
Q₁ is selected from O, S, Se, NR_{Q} or CR_{Q}R_{Q};
Q is, at each occurrence identically or differently, selected from CR_{Q} or N;
R_{Q} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

In this embodiment, * represents the position of attachment of Formula X-1, Formula X-2 and Formula X-3 to triazinyl in Formula 2.

According to an embodiment of the present disclosure, Q is, at each occurrence identically or differently, selected from CR_{Q}, Q₁ is selected from O, S or CR_{Q}R_{Q}, and Q₂ is selected from O or S.

According to an embodiment of the present disclosure, R_{Q} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, and combinations thereof.

According to an embodiment of the present disclosure, R_{Q} is, at each occurrence identically or differently, selected from the group consisting of: hydrogen, deuterium, halogen, phenyl, pyridyl, vinyl, adamantyl, methyl, ethyl, isopropyl, cyclopropyl, naphthyl, biphenyl, phenanthryl, triphenylenyl, tert-butyl, trifluoromethyl, 9,9-dimethylfluorenyl, terphenyl, dibenzothienyl, dibenzofuryl, benzothiazolyl, benzoxazolyl, phenanthroxazolyl, phenanthrothiazolyl, carbazolyl, chrysenyl, and combinations thereof.

According to an embodiment of the present disclosure, Ar_{Q} is selected from phenyl, naphthyl, biphenyl, pyridyl, phenanthryl, 9,9-dimethylfluorenyl, terphenyl, dibenzothienyl, dibenzofuryl, benzothiazolyl, benzoxazolyl, phenanthroxazolyl, phenanthrothiazolyl, chrysenyl or a combination thereof.

According to an embodiment of the present disclosure, in the compound composition, the second compound is selected from the group consisting of Compound B-1 to Compound B-223, wherein for the specific structures of Compound B-1 to Compound B-223, reference is made to claim 13.

According to an embodiment of the present disclosure, hydrogen in Compound B-1 to Compound B-223 can be partially or fully substituted with deuterium.

According to an embodiment of the present disclosure, at least two compounds among the first compound, the second compound and the third compound are isomers of each other or isotopologues of each other.

According to an embodiment of the present disclosure, the third compound has a structure represented by Formula 1, and the third compound and the first compound are isomers of each other or isotopologues of each other.

According to an embodiment of the present disclosure, the third compound has a structure represented by Formula 2, and the third compound and the second compound are isomers of each other or isotopologues of each other.

According to an embodiment of the present disclosure, hydrogen in the first compound, the second compound and the third compound can be partially or fully substituted with deuterium.

According to an embodiment of the present disclosure, the deuterated rate of at least one compound among the first compound, the second compound and the third compound is 5%-100%.

According to an embodiment of the present disclosure, the deuterated rate of at least one compound among the first compound, the second compound and the third compound is 30%-100%.

According to an embodiment of the present disclosure, the deuterated rate of at least one compound among the first compound, the second compound and the third compound is 50%-90%.

According to an embodiment of the present disclosure, the deuterated rate of at least one compound among the first compound, the second compound and the third compound is 60%-90%.

According to an embodiment of the present disclosure, the “deuterated rate of the compound” refers to a percentage of the number of deuterium in the structure of the first compound, the second compound or the third compound to the total number of hydrogen and deuterium in the compound.

According to an embodiment of the present disclosure, the mass ratio among the first compound, the second compound and the third compound in the composition is 0.1-99.9:0.1-99.9:0.1-99.9.

According to an embodiment of the present disclosure, the mass ratio among the first compound, the second compound and the third compound in the composition is 1-99:1-99:1-99.

According to an embodiment of the present disclosure, the mass ratio among the first compound, the second compound and the third compound in the composition is 10-90:10-90:10-90.

According to an embodiment of the present disclosure, an electroluminescent device is further disclosed. The electroluminescent device comprises a cathode, an anode and an organic layer disposed between the anode and the cathode, wherein the organic layer comprises the mixture described in any one of the preceding embodiments.

According to an embodiment of the present disclosure, an electroluminescent device is further disclosed. The electroluminescent device comprises a cathode, an anode and an organic layer disposed between the anode and the cathode, wherein the organic layer comprises the compound composition described in any one of the preceding embodiments.

According to an embodiment of the present disclosure, in the electroluminescent device, the organic layer is an emissive layer, and the mixture is a host material.

According to an embodiment of the present disclosure, in the electroluminescent device, the organic layer is an emissive layer, and the compound composition is a host material.

According to an embodiment of the present disclosure, in the electroluminescent device, the organic layer is an emissive layer, and the emissive layer further comprises at least one phosphorescent material.

According to an embodiment of the present disclosure, the electroluminescent device emits red light or white light.

According to an embodiment of the present disclosure, in the electroluminescent device, the organic layer is an emissive layer, the emissive layer further comprises at least one phosphorescent material, and the maximum emission wavelength of the phosphorescent material is 580 nm or above.

According to an embodiment of the present disclosure, in the electroluminescent device, the organic layer is an emissive layer, the emissive layer further comprises at least one phosphorescent material, and the maximum emission wavelength of the phosphorescent material is between 600 nm and 900 nm.

According to an embodiment of the present disclosure, the phosphorescent material is a metal complex, and the metal complex has a general formula of M(Lₐ)ₘ(L_{b})ₙ(L_{c})_{q};
wherein M is selected from a metal with a relative atomic mass greater than 40;
Lₐ, L_{b} and L_{c} are a first ligand, a second ligand and a third ligand coordinated to M, respectively; Lₐ, L_{b} and L_{c} can be optionally joined to form a multidentate ligand;
Lₐ, L_{b} and L_{c} may be the same or different; m is 1, 2 or 3; n is 0, 1 or 2; q is 0, 1 or 2; the sum of m, n and q equals an oxidation state of M; when m is greater than or equal to 2, a plurality of Lₐ may be the same or different; when n is 2, two L_{b} may be the same or different; when q is 2, two L_{c} may be the same or different;
Lₐ has a structure represented by Formula 3:

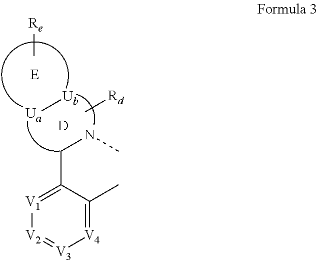

wherein
the ring D is selected from a 5-membered heteroaromatic ring or a 6-membered heteroaromatic ring;
the ring E is selected from a 5-membered unsaturated carbocyclic ring, a benzene ring, a 5-membered heteroaromatic ring or a 6-membered heteroaromatic ring;
the ring D and the ring E are fused via Uₐ and U_{b};
Uₐ and U_{b} are, at each occurrence identically or differently, selected from C or N;
R_{d} and Rₑ represent, at each occurrence identically or differently, mono-substitution, multiple substitutions or no substitution;
V₁ to V₄ are, at each occurrence identically or differently, selected from CRᵥ or N;
R_{d}, Rₑ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{d}, Rₑ and Rᵥ can be optionally joined to form a ring;
L_{b} and L_{c} are, at each occurrence identically or differently, selected from any one of the following structures:

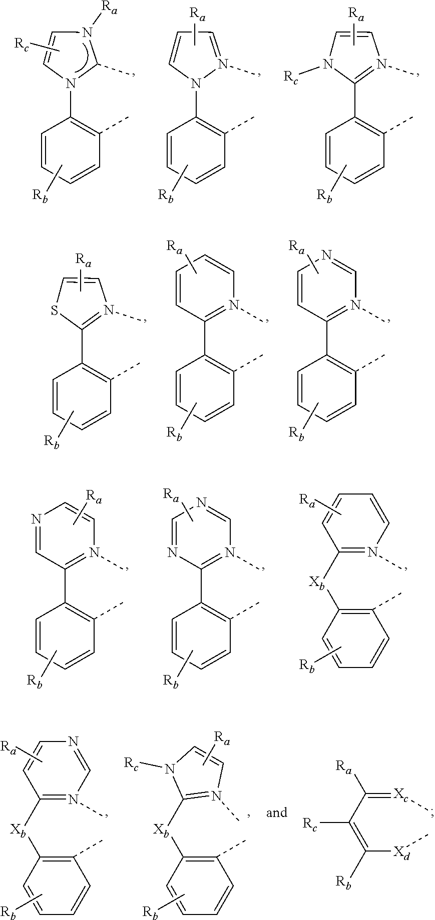

wherein
Rₐ, R_{b} and R_{c} represent, at each occurrence identically or differently, mono-substitution, multiple substitutions or non-substitution;
X_{b} is, at each occurrence identically or differently, selected from the group consisting of: 0, S, Se, NR_{N1} and CR_{C1}R_{C2};
X_{c} and X_{d} are, at each occurrence identically or differently, selected from the group consisting of: O, S, Se, and NR_{N2};
Rₐ, R_{b}, R_{c}, R_{N1}, R_{N2}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
in the structures of the ligands L_{b} and L_{c}, adjacent substituents Rₐ, R_{b}, R_{c}, R_{N1}, R_{N2}, R_{C1} and R_{C2} can be optionally joined to form a ring.

Herein, the expression that adjacent substituents R_{d}, Rₑ and Rᵥ can be optionally joined to form a ring is intended to mean that when substituents R_{d}, Rₑ and Rᵥ exist, any one or more of groups of adjacent substituents, such as adjacent substituents R_{d}, adjacent substituents Rₑ, adjacent substituents Rᵥ, adjacent substituents R_{d} and Rₑ, adjacent substituents R_{d} and Rᵥ, and adjacent substituents Rₑ and Rᵥ, can be joined to form a ring. Obviously, in the presence of substituents R_{d}, Rₑ and Rᵥ, it is possible that none of these adjacent substituents are joined to form a ring.

Herein, the expression that adjacent substituents Rₐ, R_{b}, R_{c}, R_{N1}, R_{N2}, R_{C1} and R_{C2} can be optionally joined to form a ring is intended to mean that any one or more of groups of adjacent substituents, such as two substituents Rₐ, two substituents R_{b}, two substituents R_{c}, substituents Rₐ and R_{b}, substituents Rₐ and R_{c}, substituents R_{b} and R_{c}, substituents Rₐ and R_{N1}, substituents R_{b} and R_{N1}, substituents Rₐ and R_{C1}, substituents Rₐ and R_{C2}, substituents R_{b} and R_{C1}, substituents R_{b} and R_{C2}, substituents Rₐ and R_{N2}, substituents R_{b} and R_{N2}, and substituents R_{C1} and R_{C2}, can be joined to form a ring. For example, adjacent substituents Rₐ and R_{b} in

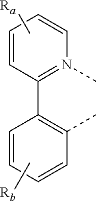

can be optionally joined to form a ring, which can form one or more of the following structures, including, but not limited to:

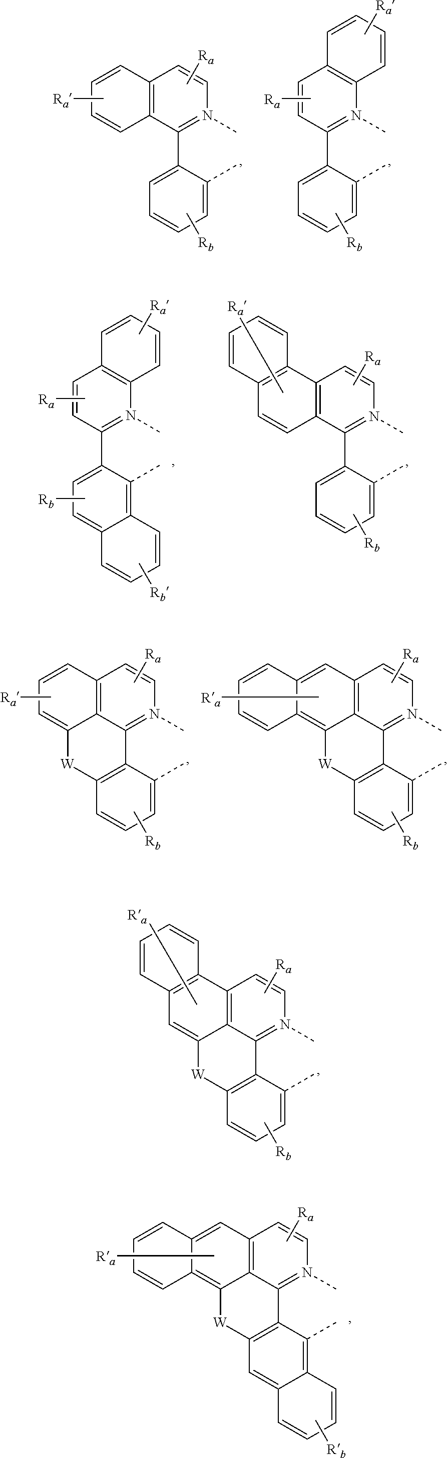

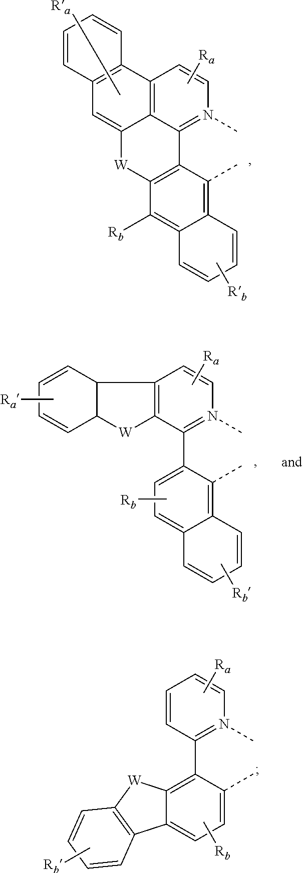

wherein W is selected from O, S, Se, NR′ or CR′R′, and R′, Rₐ′ and R_{b}′ are defined the same as Rₐ. Obviously, it is possible that none of these substituents are joined to form a ring.

According to an embodiment of the present disclosure, in Formula 3, adjacent two substituents Rₑ are joined to form a ring.

According to an embodiment of the present disclosure, in Formula 3, adjacent two substituents Rₑ are joined to form a 5-membered unsaturated carbocyclic ring, a 5-membered heteroaromatic ring or a benzene ring.

According to an embodiment of the present disclosure, in Formula 3, the ring D is a 6-membered heteroaromatic ring, and the ring E is a benzene ring or a 6-membered heteroaromatic ring.

According to an embodiment of the present disclosure, in Formula 3, the ring D is a 6-membered heteroaromatic ring, and the ring E is a 5-membered heteroaromatic ring or a 5-membered unsaturated carbocyclic ring.

According to an embodiment of the present disclosure, in Formula 3, the ring D is a 6-membered heteroaromatic ring, the ring E is a benzene ring or a 6-membered heteroaromatic ring, and adjacent two substituents Rₑ are joined to form a benzene ring or a 6-membered heteroaromatic ring.

According to an embodiment of the present disclosure, in Formula 3, the ring D is a 6-membered heteroaromatic ring, the ring E is a 5-membered heteroaromatic ring or a 5-membered unsaturated carbocyclic ring, and adjacent two substituents Rₑ are joined to form a benzene ring or a 6-membered heteroaromatic ring.

According to an embodiment of the present disclosure, in Formula 3, at least one or two sets of adjacent substituents in R_{d}, Rₑ, Rᵥ are joined to form a ring. For example, two substituents R_{d} are joined to form a ring, two substituents Rₑ are joined to form a ring, two substituents Rᵥ are joined to form a ring, substituents R_{d} and Rₑ are joined to form a ring, substituents R_{d} and Rᵥ are joined to form a ring, substituents Rₑ and Rᵥ are joined to form a ring, two substituents Rₑ are joined to form a ring while two substituents R_{d} are joined to form a ring, two substituents Rᵥ are joined to form a ring while two substituents R_{d} are joined to form a ring, two substituents Rᵥ are joined to form a ring while two substituents Rₑ are joined to form a ring, two substituents Rᵥ are joined to form a ring while substituents Rₑ and Rᵥ are joined to form a ring, or two substituents Rᵥ are joined to form a ring while substituents R_{d} and Rᵥ are joined to form a ring. A similar situation occurs when more groups of adjacent substituents among R_{d}, Rₑ and Rᵥ are joined to form a ring.

According to an embodiment of the present disclosure, in the electroluminescent device, the phosphorescent material is a metal complex, and the metal complex has a general formula of M(Lₐ)ₘ(L_{b})ₙ;
wherein M is selected from a metal with a relative atomic mass greater than 40;
Lₐ and L_{b} are a first ligand and a second ligand coordinated to M, respectively; Lₐ and L_{b} can be optionally joined to form a multidentate ligand;
m is 1, 2 or 3; n is 0, 1 or 2; the sum of m and n equals an oxidation state of M; when m is greater than or equal to 2, a plurality of Lₐ may be the same or different; when n is 2, two L_{b} may be the same or different;
Lₐ has a structure represented by Formula 3:

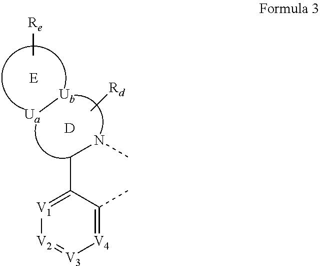

wherein
the ring D is selected from a 5-membered heteroaromatic ring or a 6-membered heteroaromatic ring;
the ring E is selected from a 5-membered unsaturated carbocyclic ring, a benzene ring, a 5-membered heteroaromatic ring or a 6-membered heteroaromatic ring;
the ring D and the ring E are fused via Uₐ and U_{b};
Uₐ and U_{b} are, at each occurrence identically or differently, selected from C or N;
R_{d} and Rₑ represent, at each occurrence identically or differently, mono-substitution, multiple substitutions or no substitution;
V₁ to V₄ are, at each occurrence identically or differently, selected from CRᵥ or N;
R_{d}, R_{c} and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{d}, Rₑ and Rᵥ can be optionally joined to form a ring;
the ligand L_{b} has the following structure:

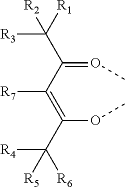

wherein R₁ to R₇ are each independently selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

According to an embodiment of the present disclosure, in the electroluminescent device, the ligand L_{b} has the following structure:

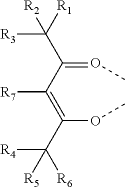

wherein at least one of R₁ to R₃ is selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof; and/or at least one of R₄ to R₆ is selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof.

According to an embodiment of the present disclosure, in the electroluminescent device, the ligand L_{b} has the following structure:

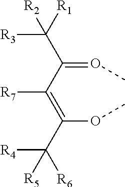

wherein at least two of R₁ to R₃ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof, and/or at least two of R₄ to R₆ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof.

According to an embodiment of the present disclosure, in the electroluminescent device, the ligand L_{b} has the following structure:

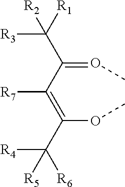

wherein at least two of R₁ to R₃ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof, and/or at least two of R₄ to R₆ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof.

According to an embodiment of the present invention, in the electroluminescent device, the phosphorescent material is an Ir complex, a Pt complex or an Os complex.

According to an embodiment of the present disclosure, in the electroluminescent device, the phosphorescent material is an Ir complex and has a structure represented by any one of Ir(Lₐ)(L_{b})(L_{c}), Ir(Lₐ)₂(L_{b}), Ir(Lₐ)(L_{b})₂, Ir(Lₐ)₂(L_{c}) or Ir(Lₐ)(L_{c})₂.

According to an embodiment of the present disclosure, Lₐ has a structure represented by Formula 3 and comprises at least one structural unit selected from the group consisting of an aromatic ring formed by fusing a 6-membered ring to a 6-membered ring, a heteroaromatic ring formed by fusing a 6-membered ring to a 6-membered ring, an aromatic ring formed by fusing a 6-membered ring to a 5-membered ring, and a heteroaromatic ring formed by fusing a 6-membered ring to a 5-membered ring.

According to an embodiment of the present disclosure, in the electroluminescent device, Lₐ has a structure represented by Formula 3 and comprises at least one structural unit selected from the group consisting of naphthalene, phenanthrene, quinoline, isoquinoline and azaphenanthrene.

According to an embodiment of the present disclosure, in the organic electroluminescent device, the phosphorescent material is an Ir complex and comprises a ligand Lₐ, wherein Lₐ is, at each occurrence identically or differently, selected from any one of the group consisting of the following structures:

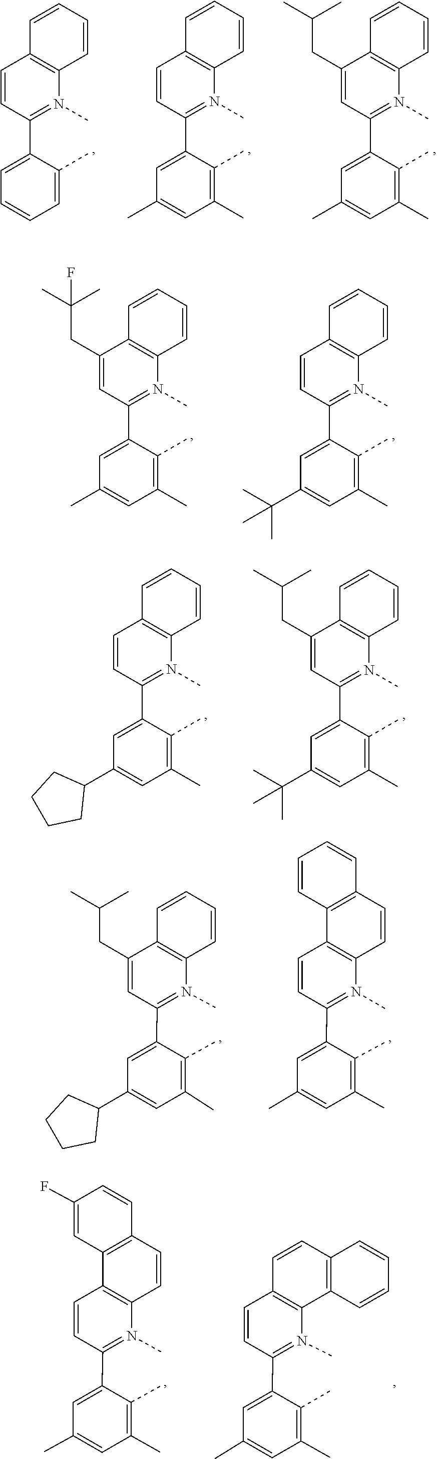

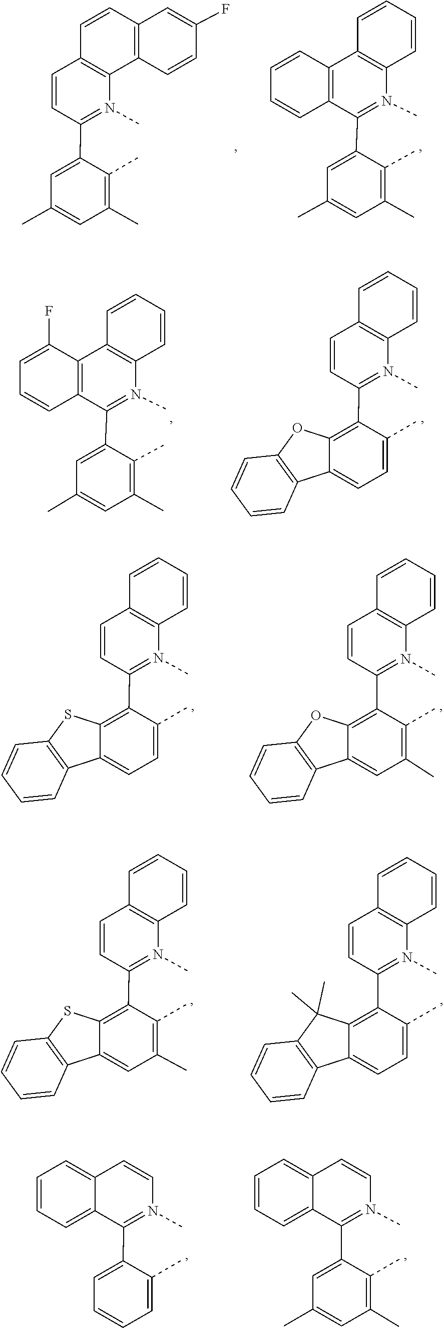

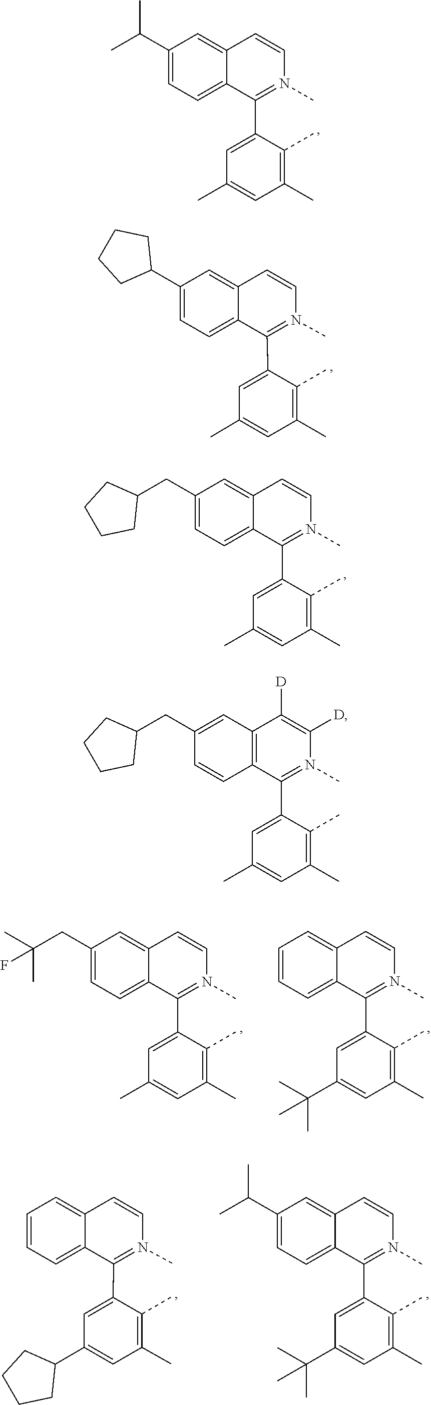

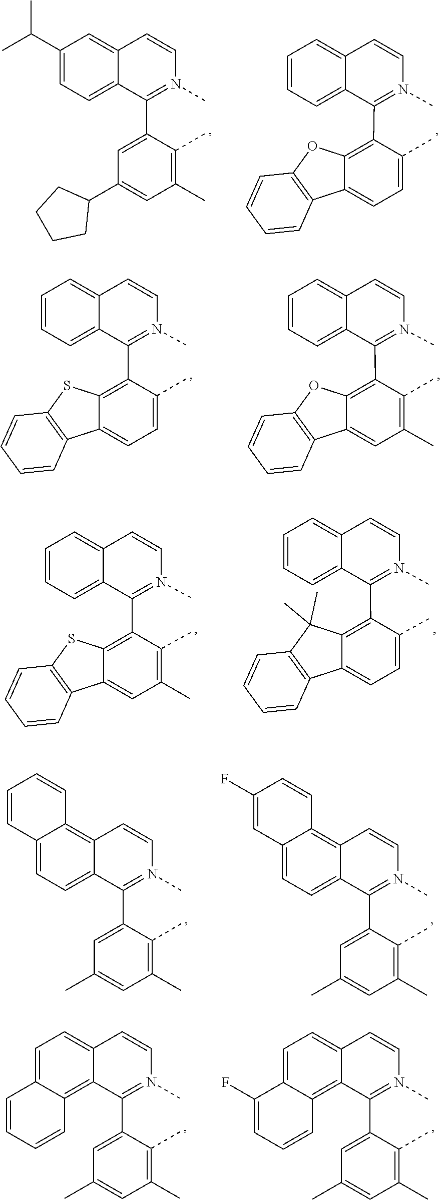

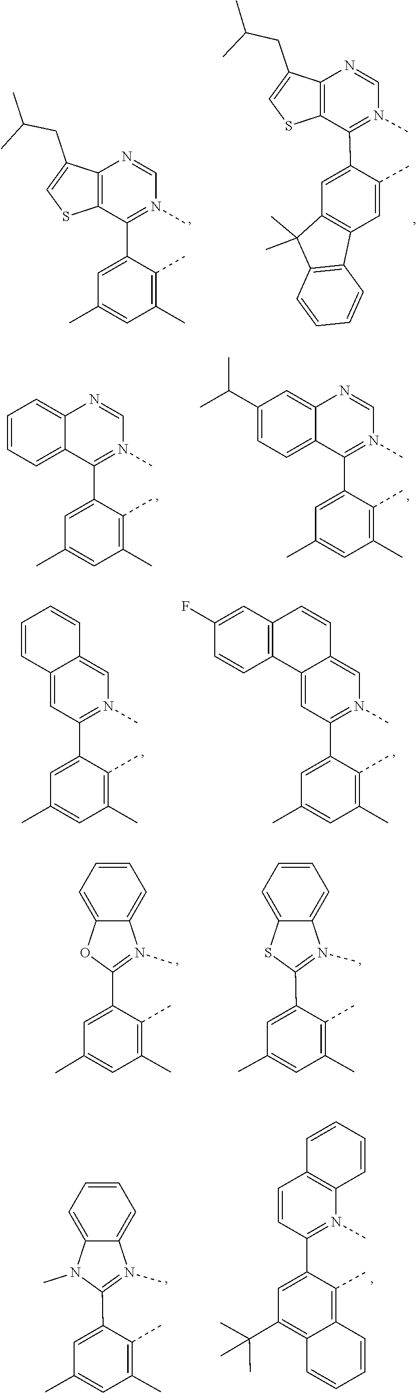

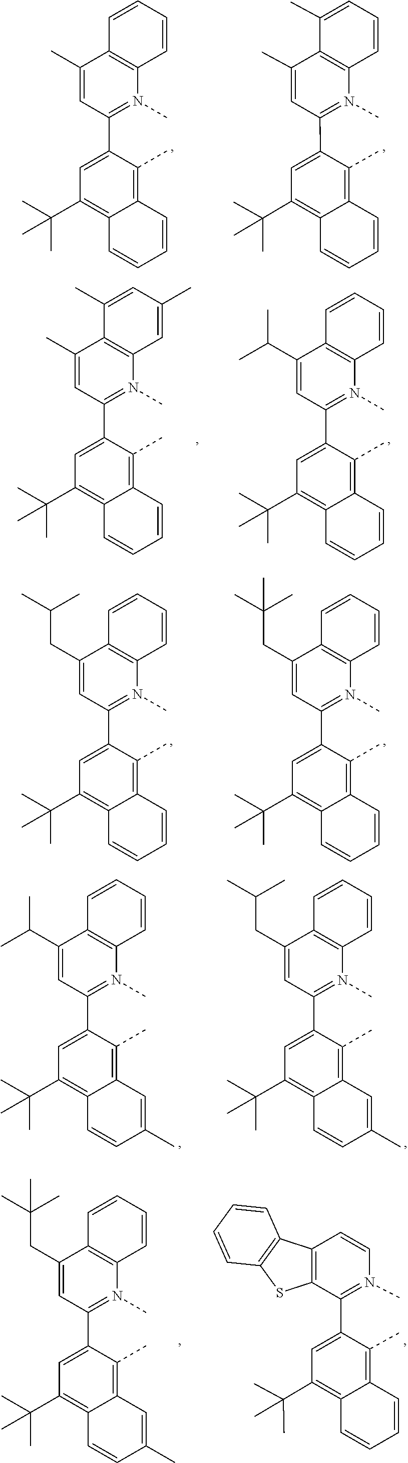

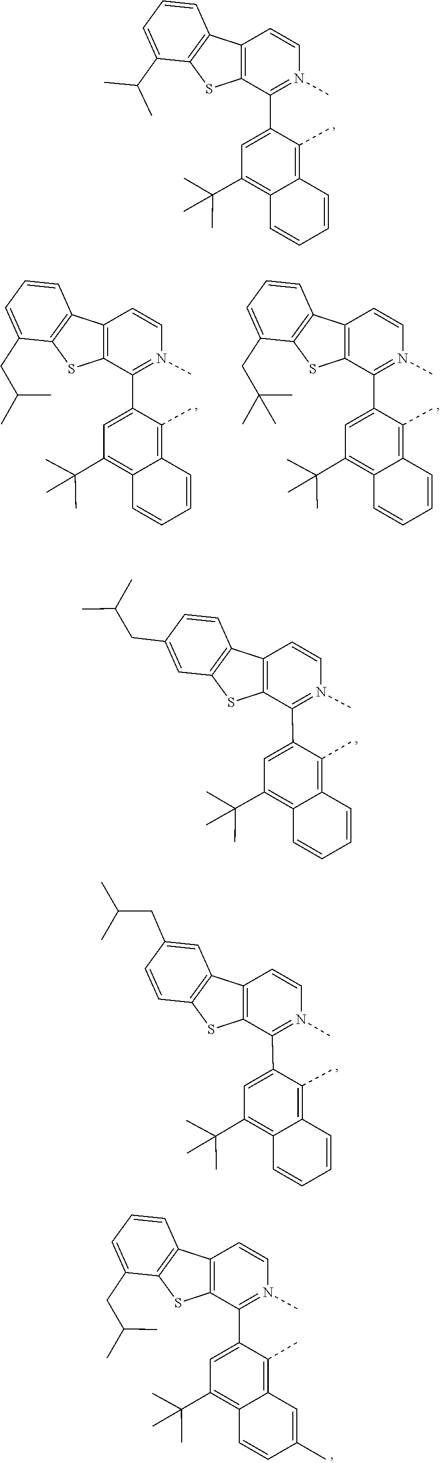

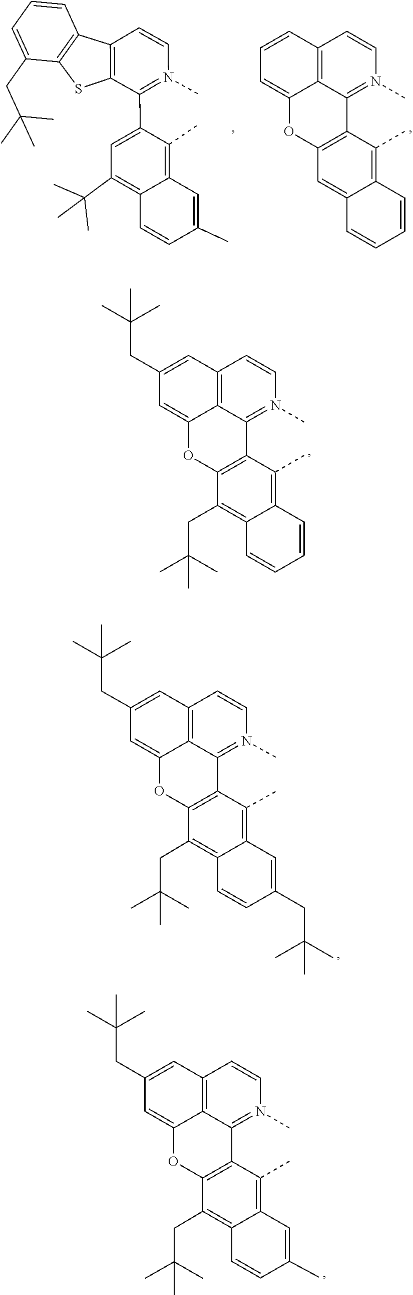

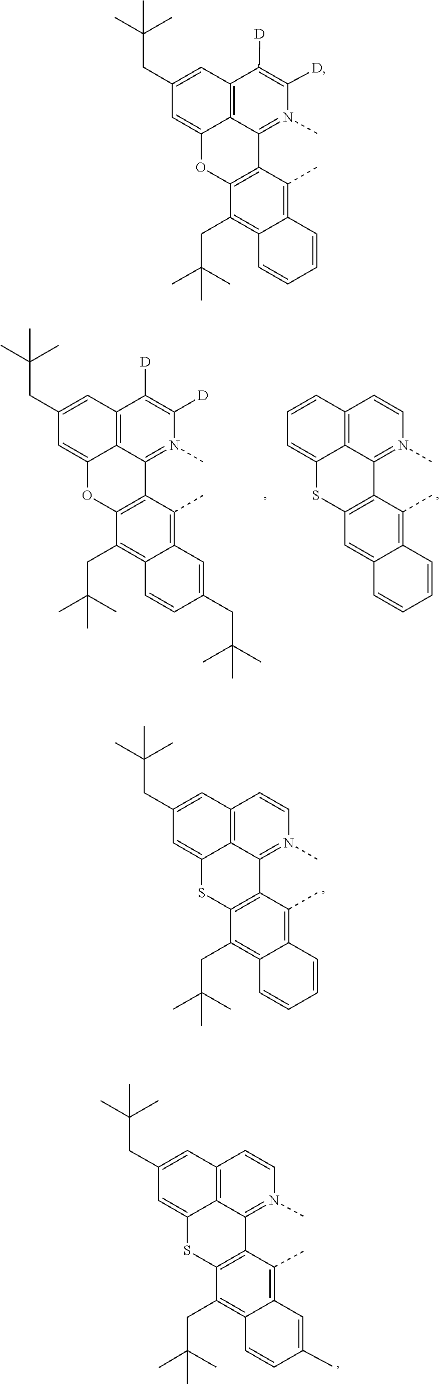

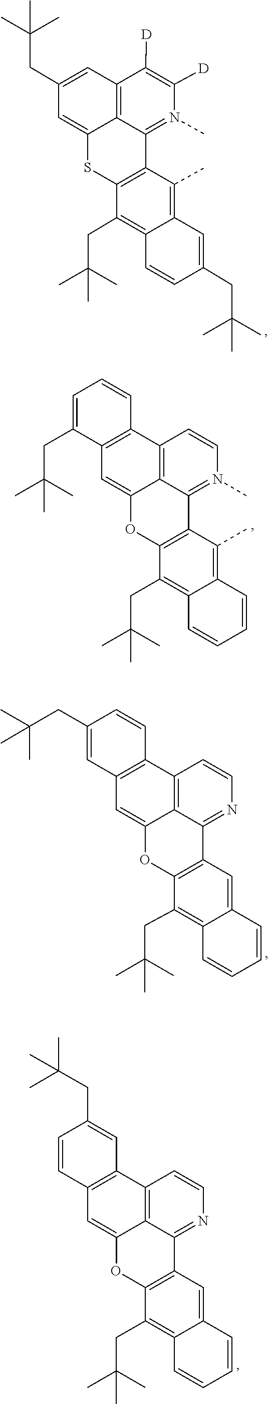

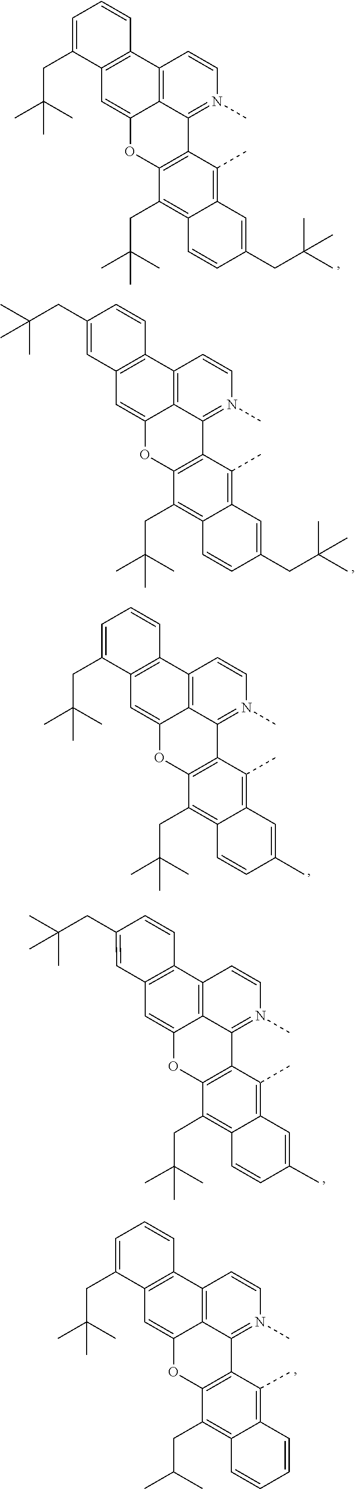

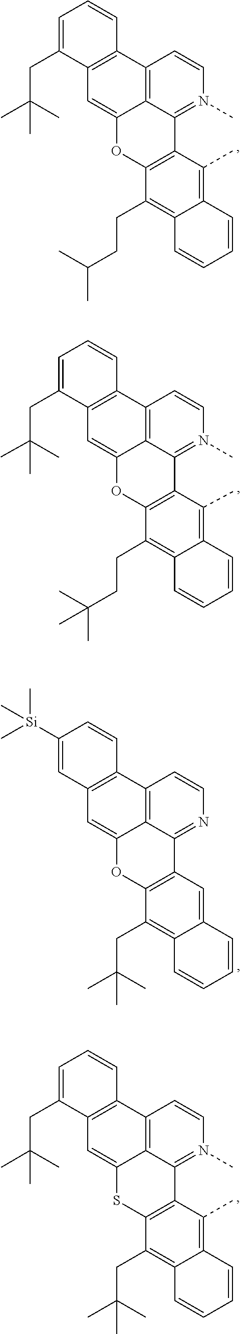

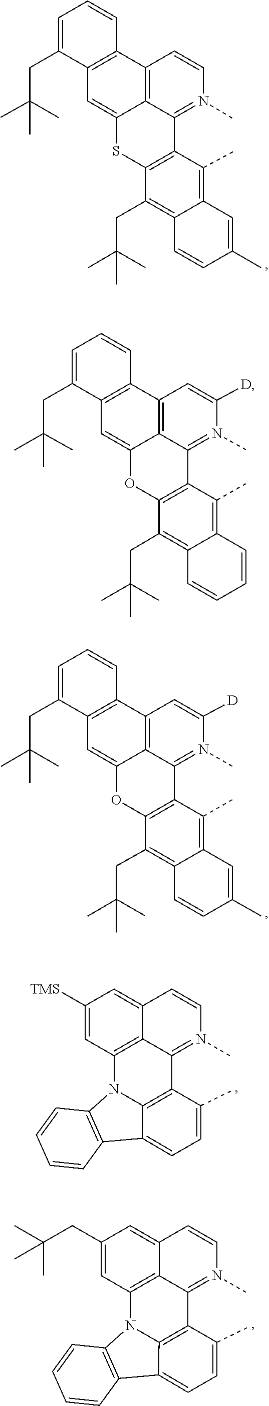

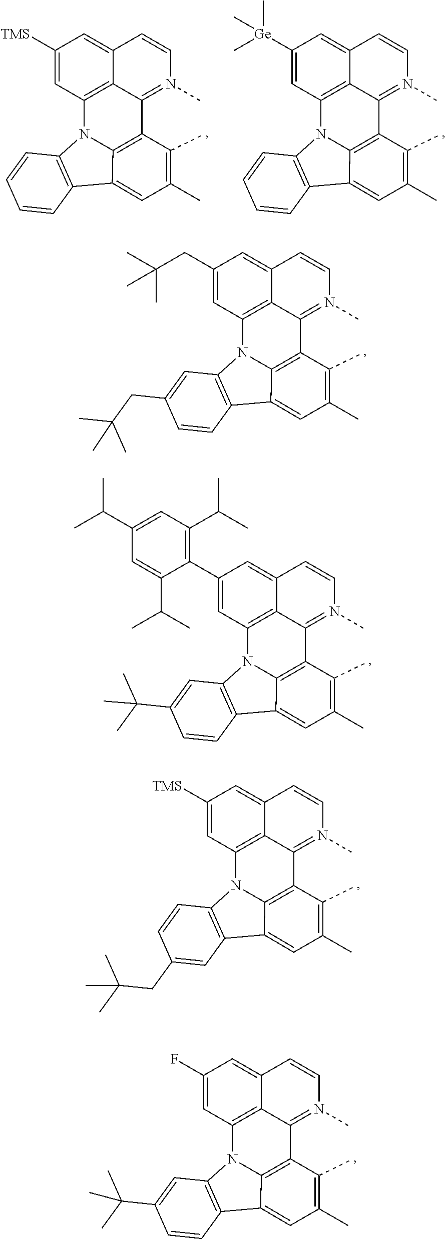

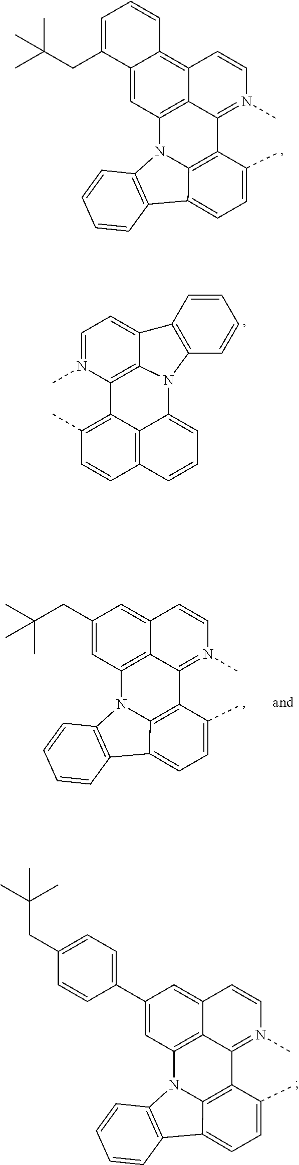

wherein in the above structures, TMS represents trimethylsilyl.

According to an embodiment of the present disclosure, in the organic electroluminescent device, the phosphorescent material is an Ir complex and comprises a ligand L_{b}, wherein L_{b} is, at each occurrence identically or differently, selected from the group consisting of the following structures:

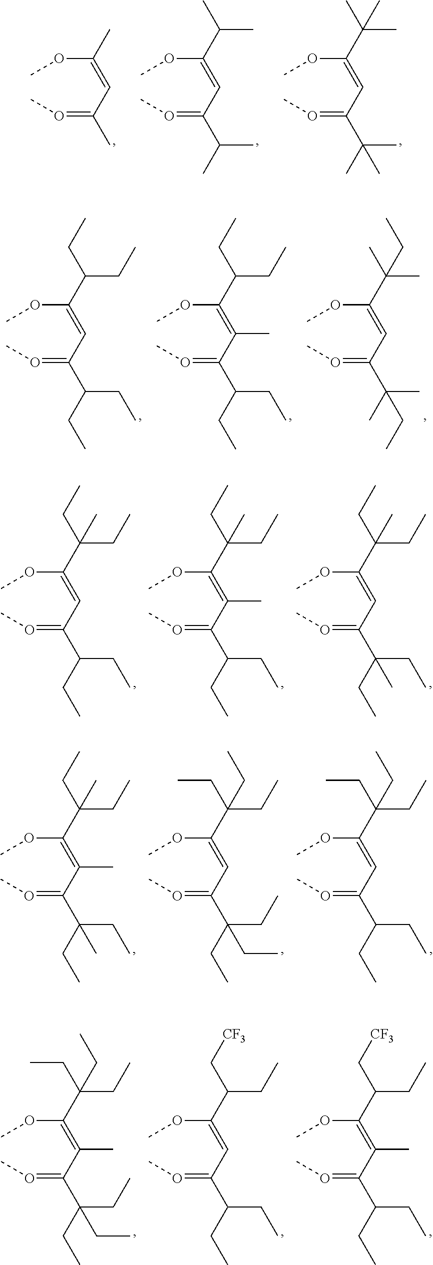

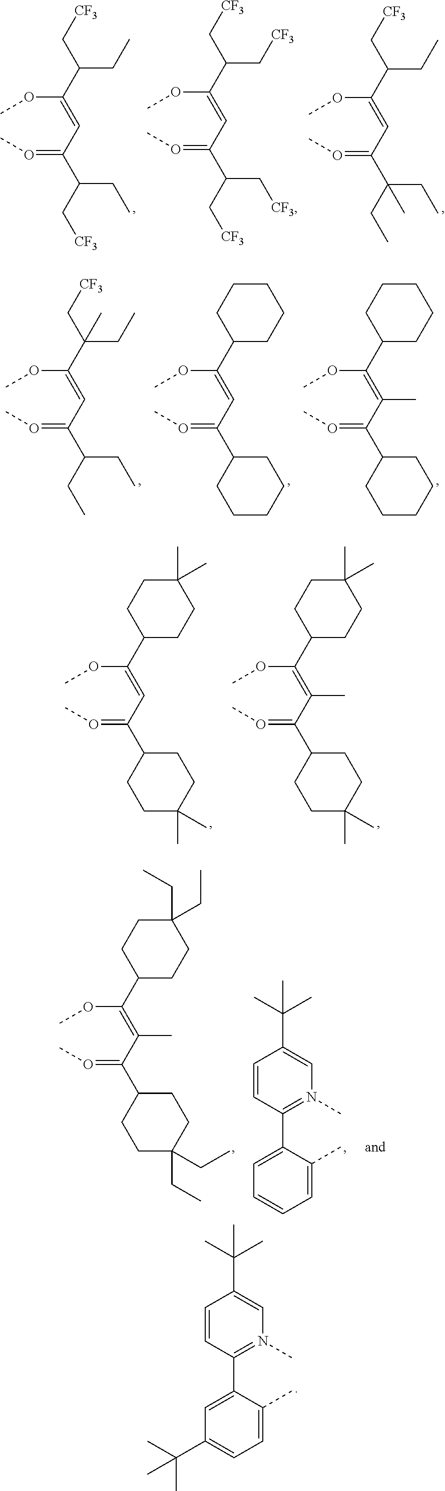

According to an embodiment of the present disclosure, in the organic electroluminescent device, the phosphorescent material is selected from the group consisting of the following structures:

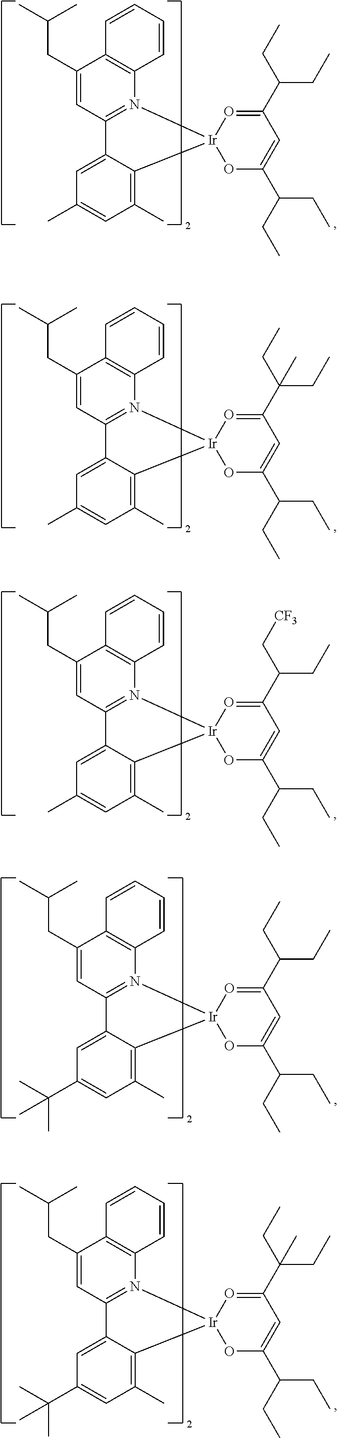

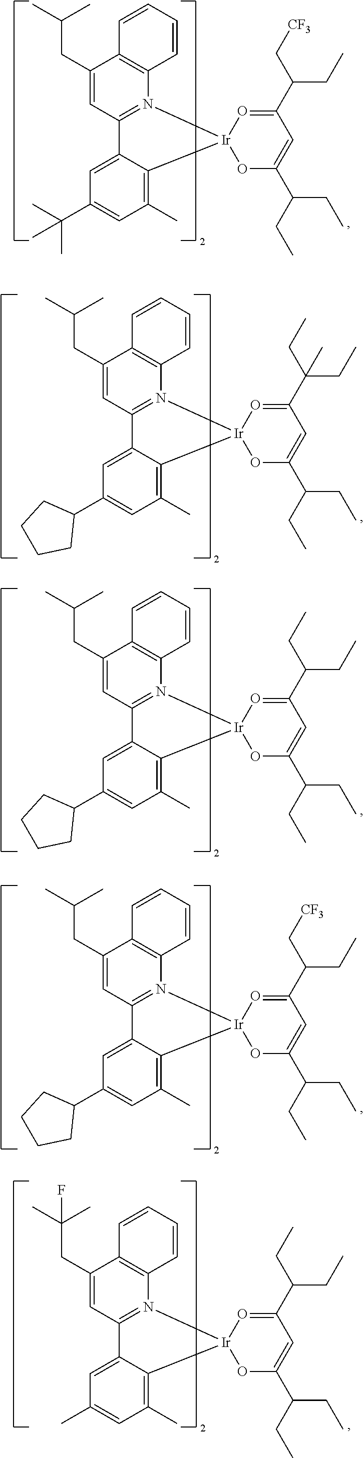

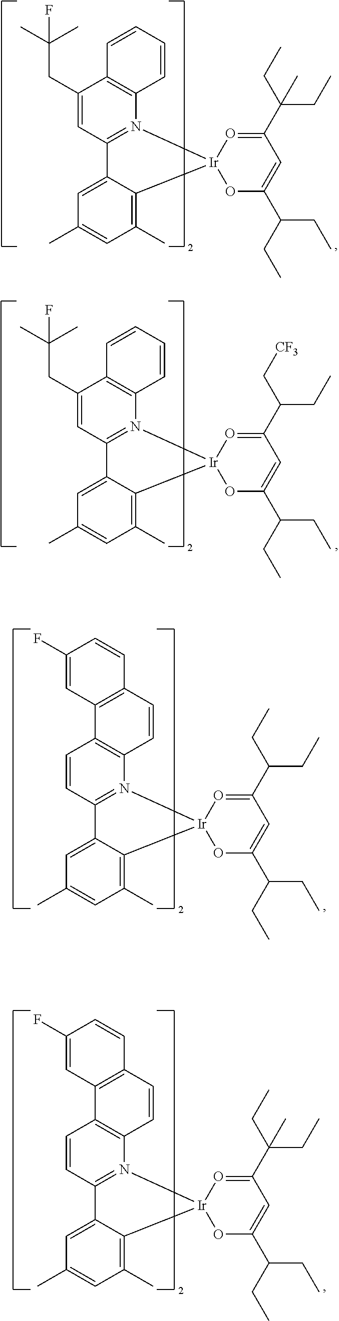

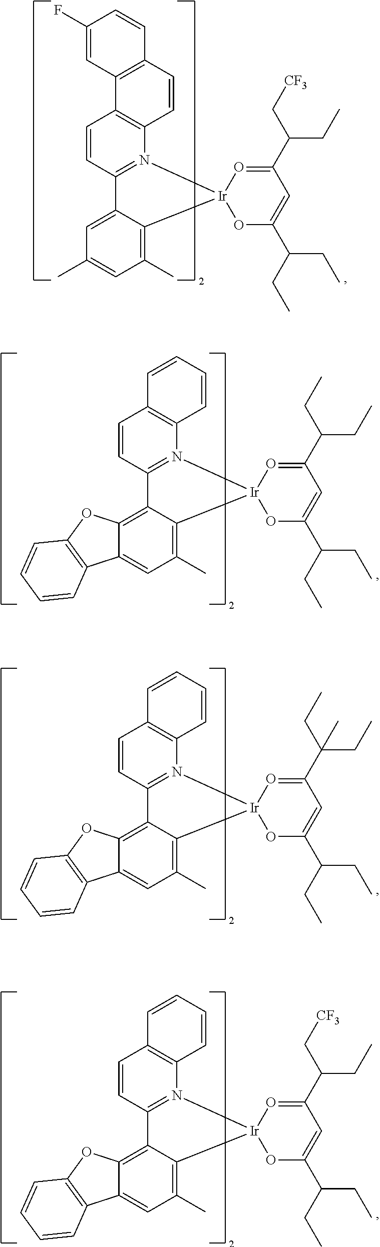

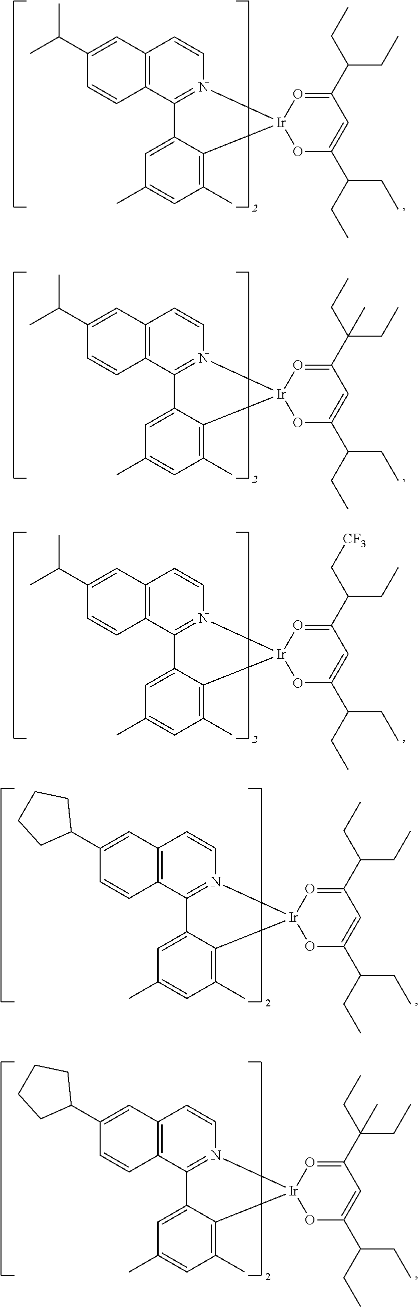

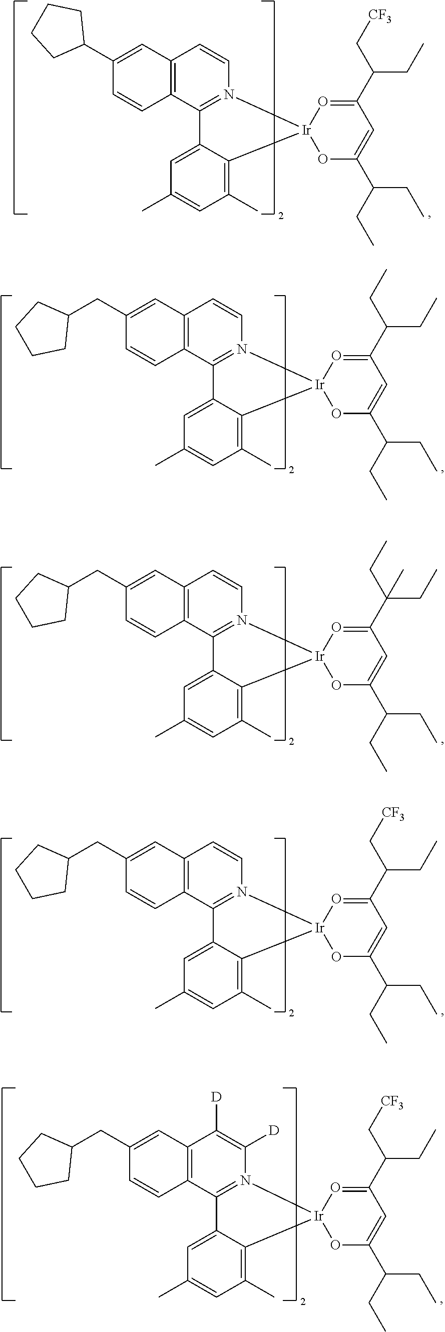

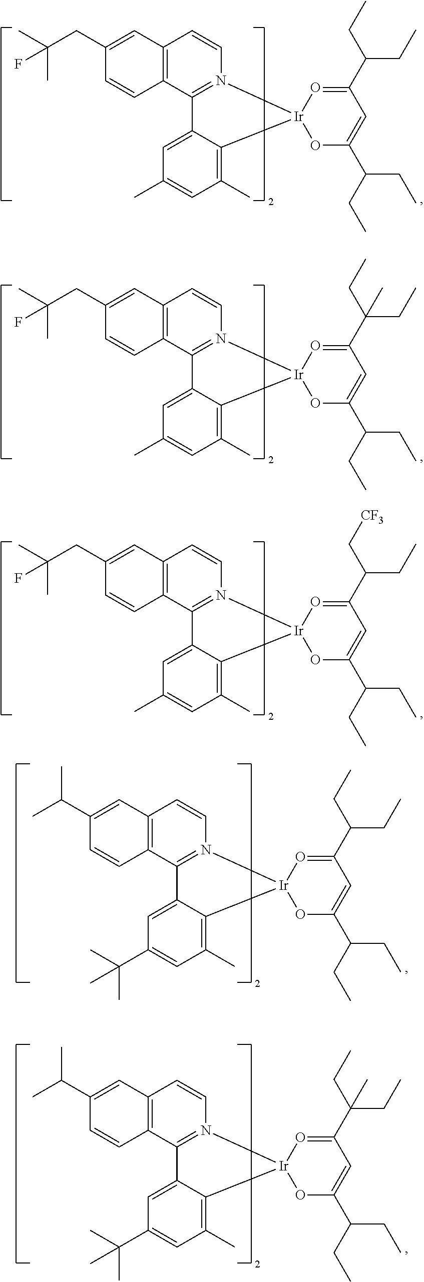

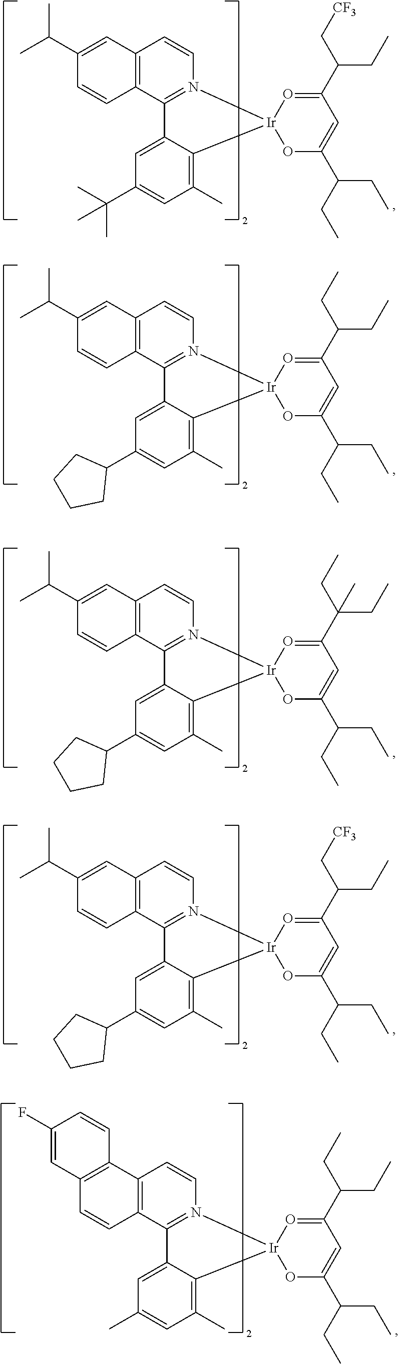

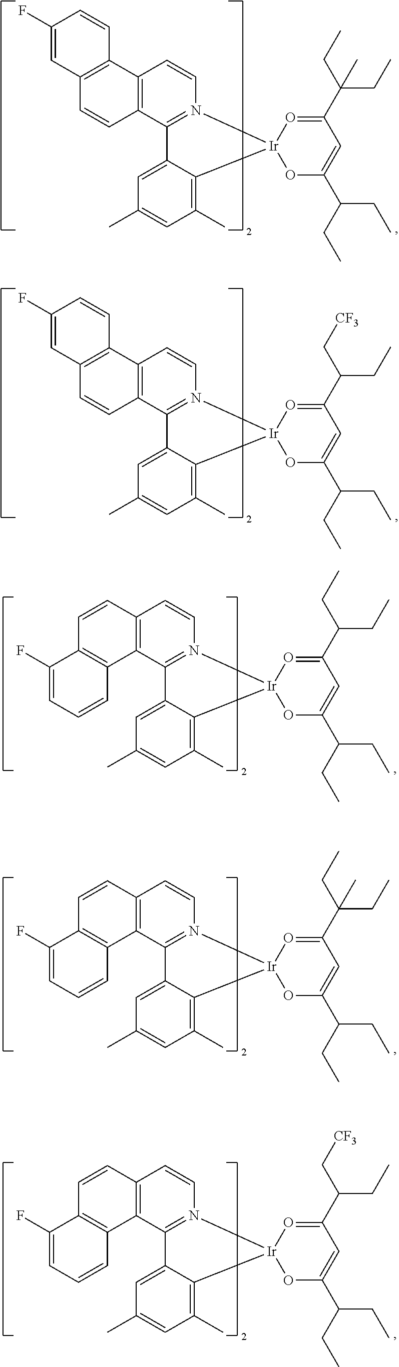

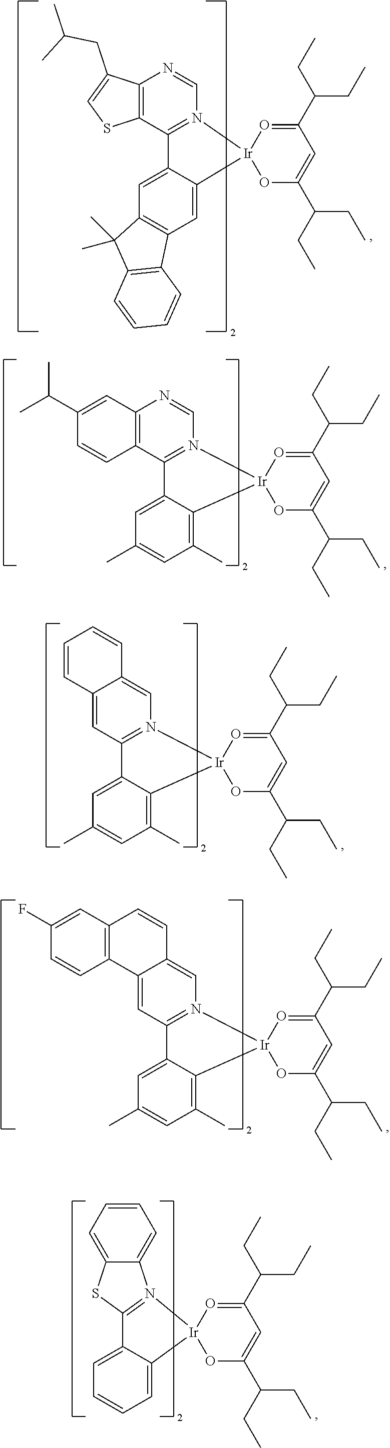

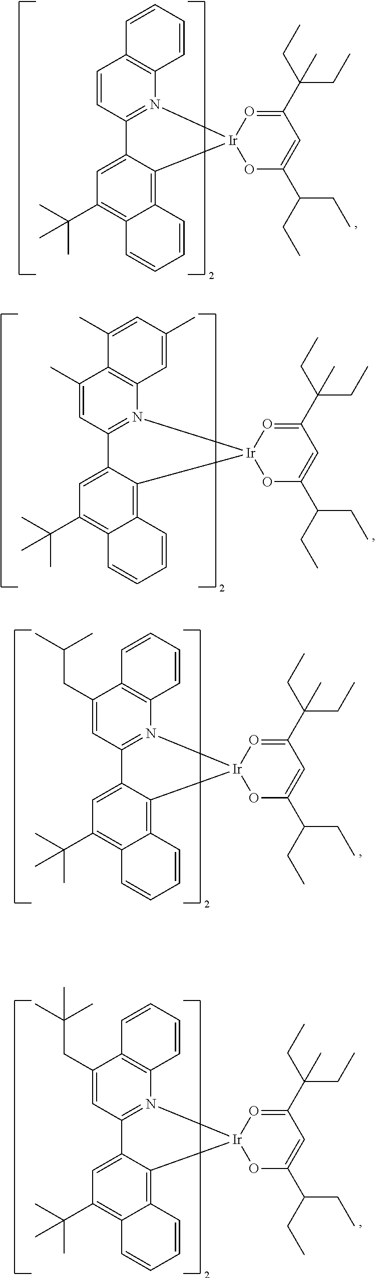

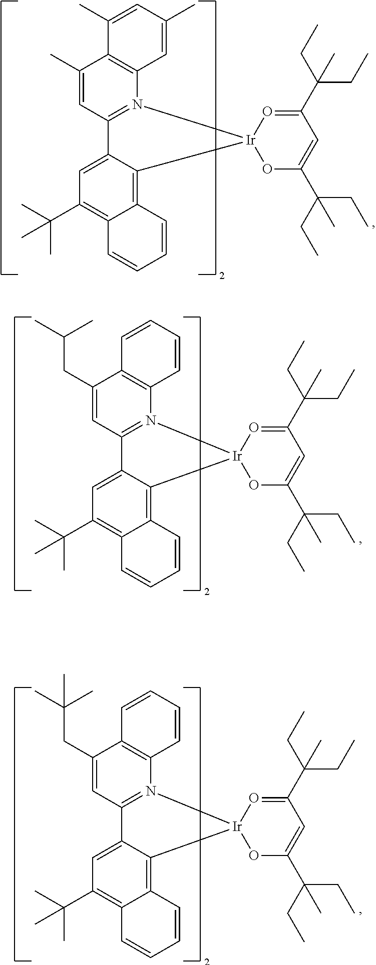

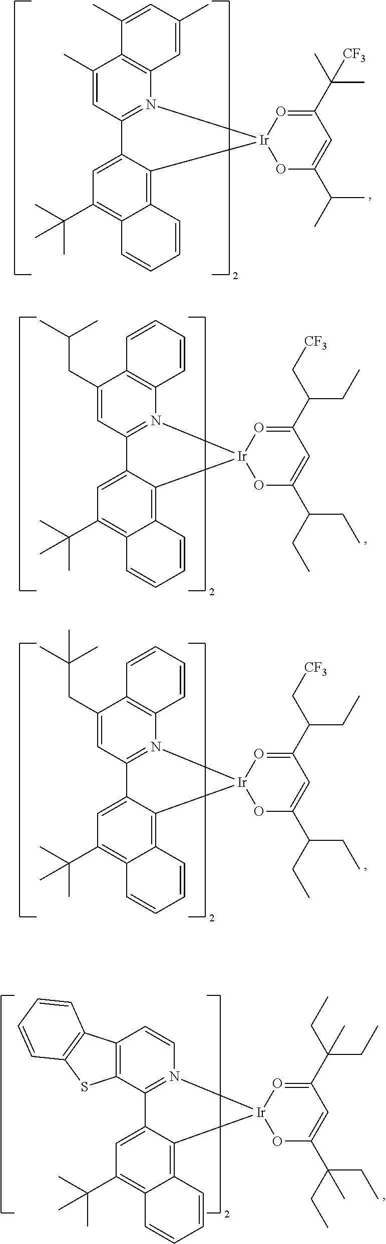

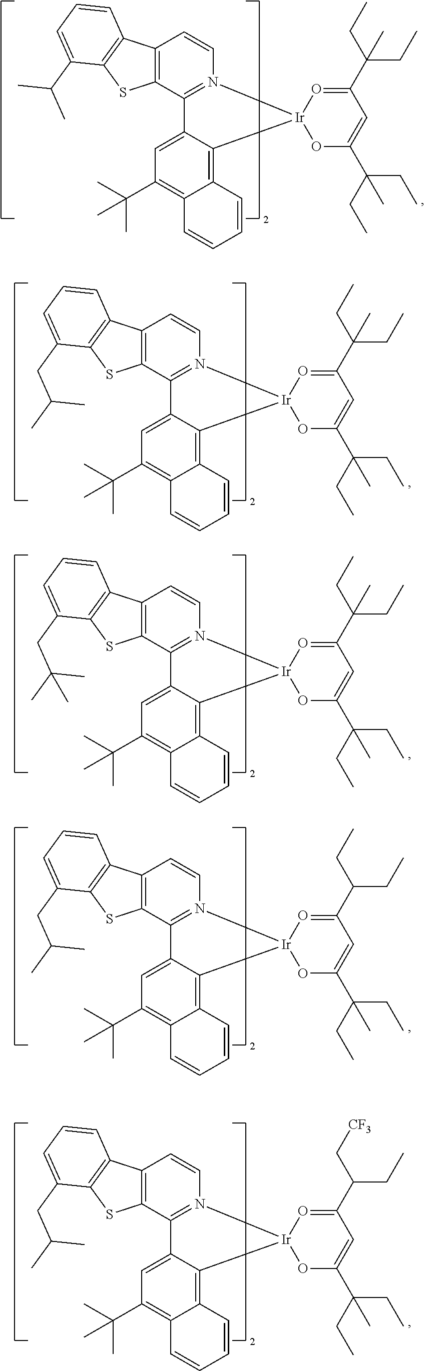

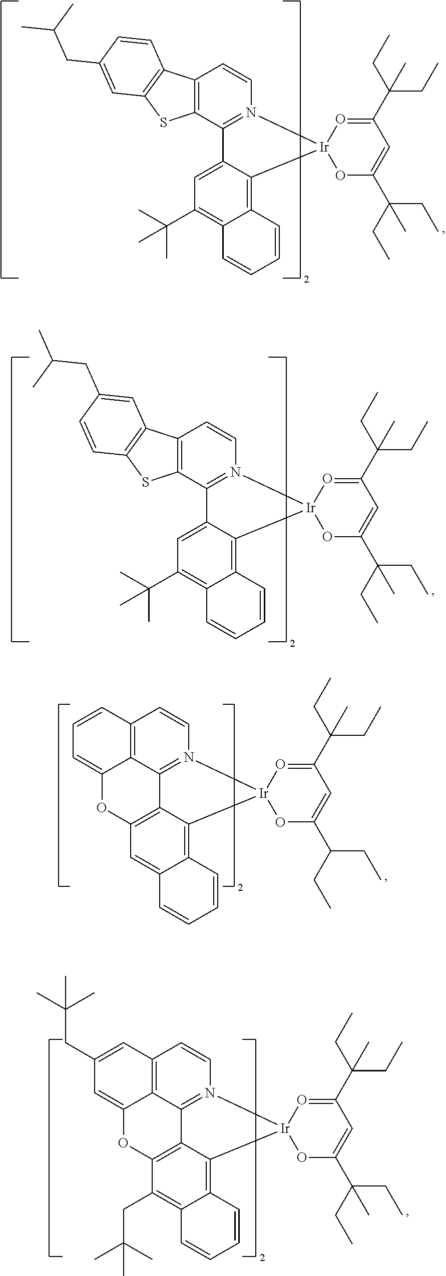

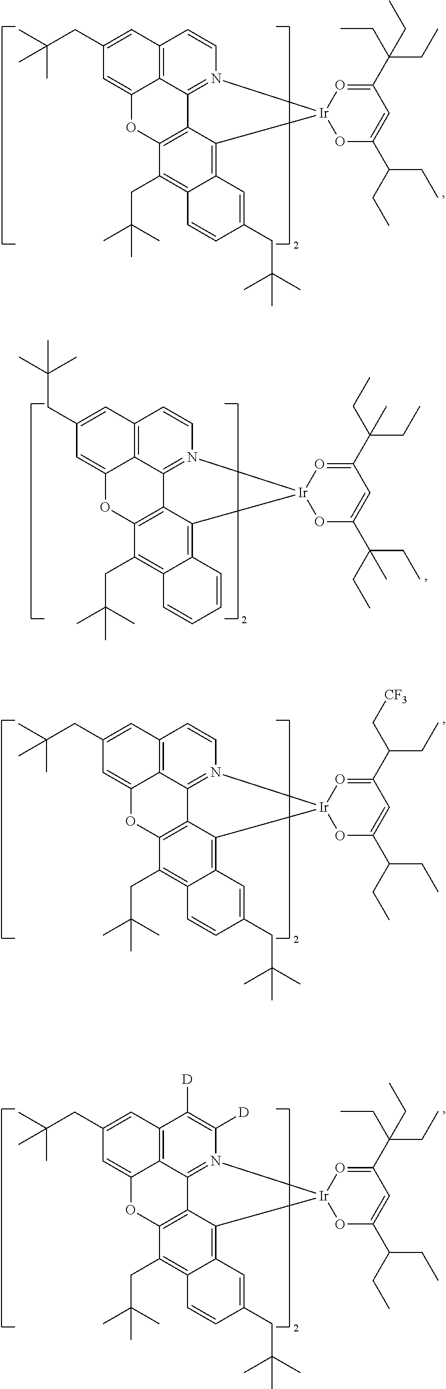

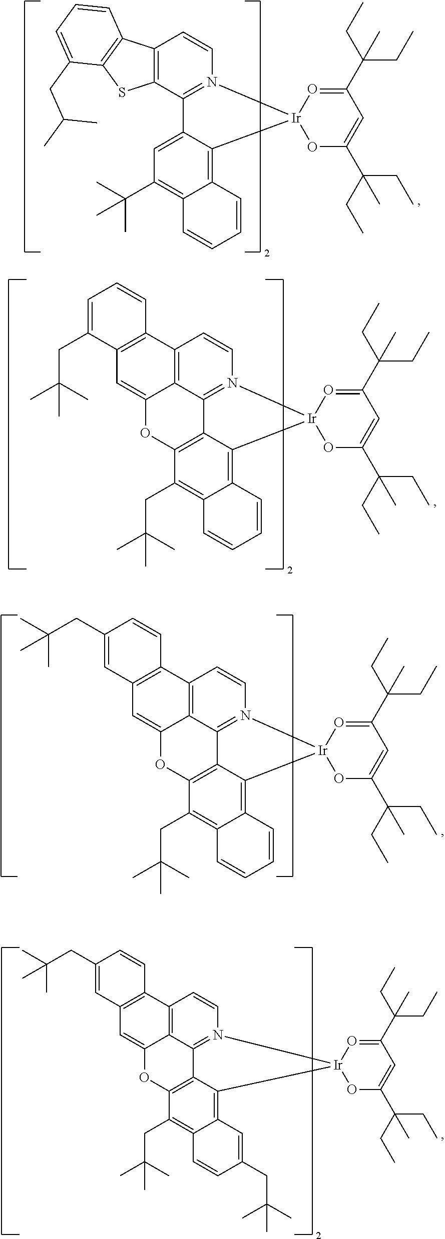

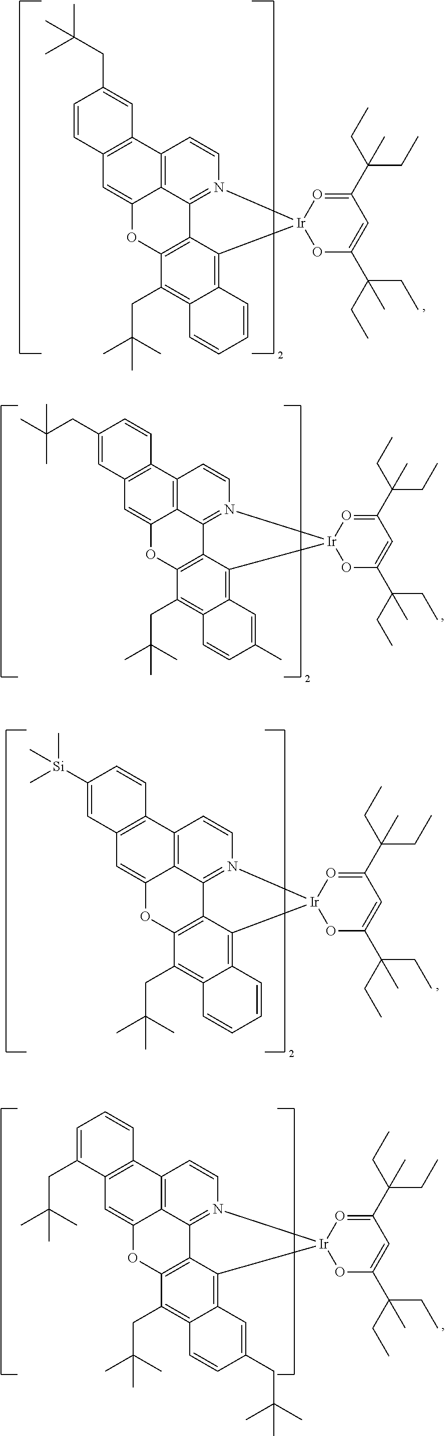

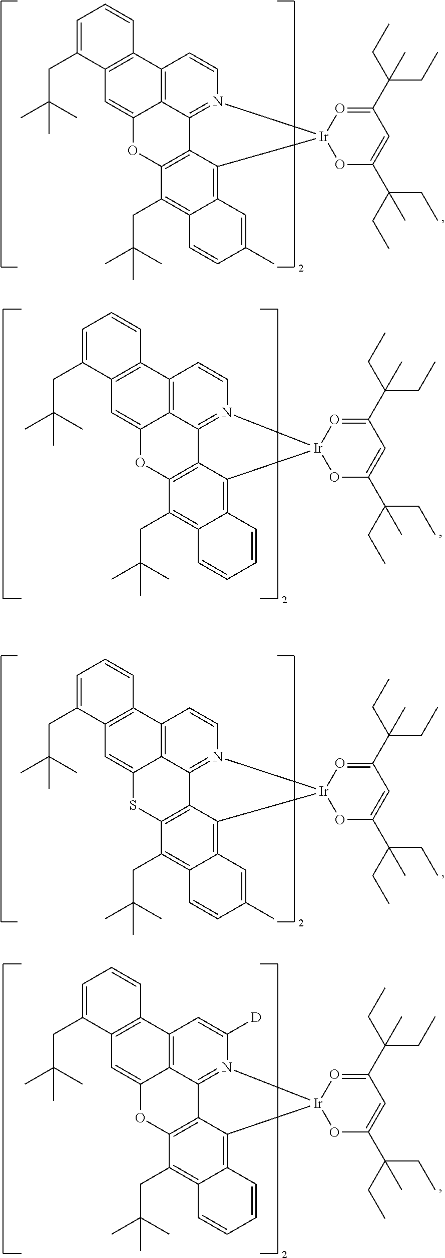

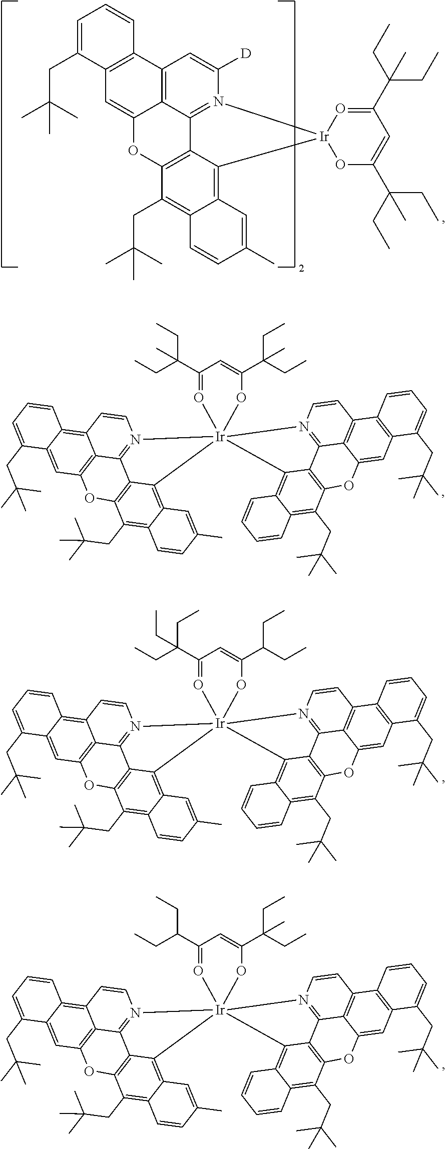

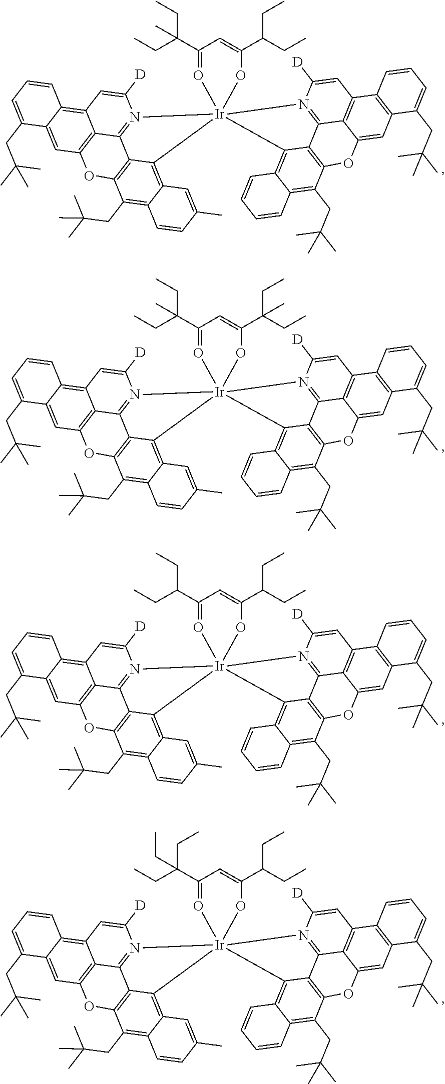

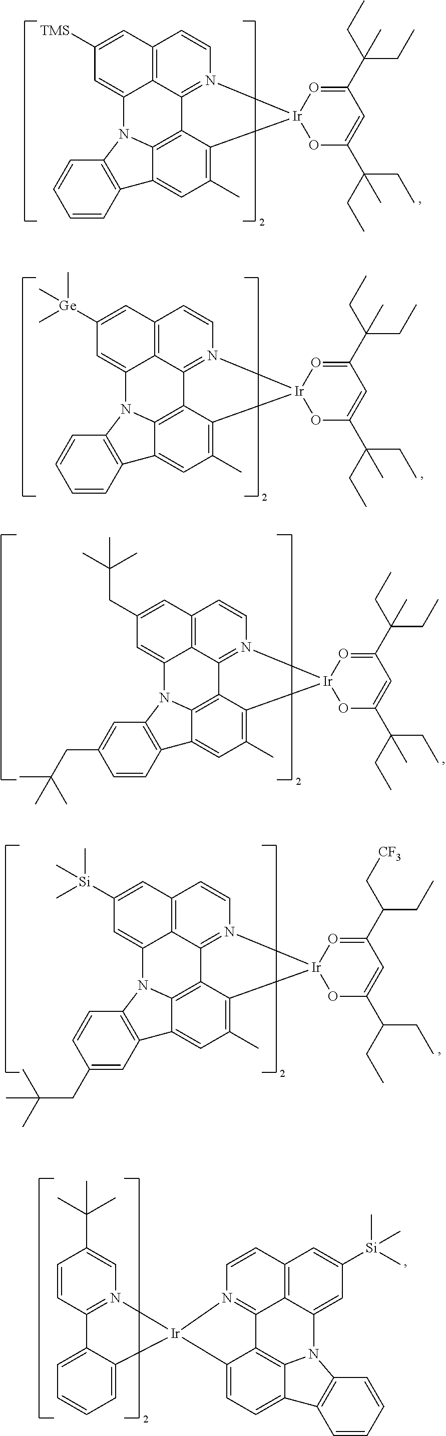

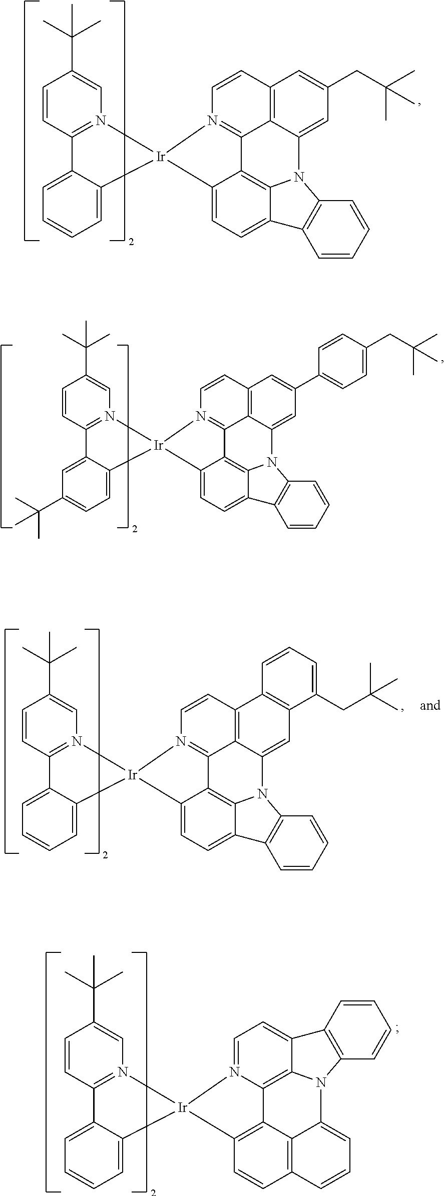

wherein in the above structures, TMS represents trimethylsilyl.

According to an embodiment of the present disclosure, during the preparation of the organic electroluminescent device of the present disclosure, when more than three host materials and an emissive material are co-evaporated to form an emissive layer, the more than three host materials and the emissive material may separately be loaded into different evaporation sources and then co-evaporated to form the emissive layer; or a mixture formed by pre-mixing the more than three host materials may be loaded into one evaporation source and then co-evaporated with the emissive material in another evaporation source to form the emissive layer, wherein the pre-mixing manner can further save the evaporation source. Taking the present disclosure as an example, the first compound having a structure of Formula 1, the second compound having a structure of Formula 2 and the third compound having a structure of Formula 1 or Formula 2 of the present disclosure as well as an emissive material may separately be loaded into different evaporation sources and co-evaporated to form the emissive layer; or a mixture formed by pre-mixing the first compound, the second compound and the third compound may be loaded into one evaporation source and co-evaporated with an emissive material in another evaporation source to form the emissive layer.

According to an embodiment of the present disclosure, a method for preparing an electroluminescent device is further disclosed, wherein the electroluminescent device comprises the mixture described in any one of the preceding embodiments. The preparation method comprises the following steps:
step 1: providing a substrate, and setting a first electrode on the substrate;
step 2: per-mixing the first compound, the second compound and the third compound to form the mixture described in any one of the preceding embodiments, loading the mixture into a container of a high-vacuum deposition tool, and when the mixture in the container is evaporated at a rate of 0.01-5 Å/s using the high-vacuum deposition tool with a vacuum degree of 10⁻⁶ torr or below, evaporating the mixture on a surface positioned at a certain distance from the mixture to be evaporated to form an organic layer; wherein a mass ratio of at least one compound among the first compound, the second compound and the third compound in the mixture is C₀, and a mass ratio of the at least one compound in the organic layer to a total mass of the first compound, the second compound and the third compound is C′; wherein |C′−C₀|≤2%; and
step 3: depositing a second electrode on the organic layer.

According to an embodiment of the present disclosure, a method for preparing an electroluminescent device is further disclosed, wherein the electroluminescent device comprises the compound composition described in any one of the preceding embodiments. The preparation method comprises the following steps:
step 1: providing a substrate, and setting a first electrode on the substrate;
step 2: loading a first compound, a second compound and a third compound into different containers of a high-vacuum deposition tool, respectively, and when the compounds in the containers are evaporated simultaneously at a rate of 0.01-5 Å/s using the high-vacuum deposition tool with a vacuum degree of 10⁻⁶ torr or below, evaporating the first compound, the second compound and the third compound on a surface positioned at a certain distance from the compounds to be evaporated such that the three compounds are co-deposited to form an organic layer; and
step 3: depositing a second electrode on the organic layer.

The device prepared by using the preparation method herein may further comprise another organic layer that may be disposed between the organic layer prepared in step 2 and two electrode layers. The organic layer prepared in step 2 is preferably an emissive layer, and the emissive layer may further comprise another compound and preferably, further comprises a phosphorescence compound.

According to another embodiment of the present disclosure, an electronic device is further disclosed. The electronic device comprises an electroluminescent device, wherein the electroluminescent device and the preparation method thereof are shown in any one of the preceding embodiments.

Combination with Other Materials

The materials described in the present disclosure for a particular layer in an organic light emitting device can be used in combination with various other materials present in the device. The combinations of these materials are described in more detail in U.S. Pat. App. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light emitting device may be used in combination with a variety of other materials present in the device. For example, the compounds disclosed herein may be used in combination with a wide variety of emissive dopants, hosts, transport layers, blocking layers, injection layers, electrodes and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatograph-mass spectrometry produced by SHIMADZU, gas chromatograph-mass spectrometry produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FATAR, life testing system produced by SUZHOU FATAR, and ellipsometer produced by BEIJING ELLITOP, etc.) by methods well known to the persons skilled in the art. As the persons skilled in the art are aware of the above-mentioned equipment use, test methods and other related contents, the inherent data of the sample can be obtained with certainty and without influence, so the above related contents are not further described in this present disclosure.

### Material Synthesis Example

The method for preparing the compound of the present disclosure is not limited herein. Typically, the following compounds are used as examples without limitation, and the synthesis routes and preparation methods thereof are described below.

### Synthesis Example 1: Synthesis of Compound A-3

### Step 1: Synthesis of Compound A-3

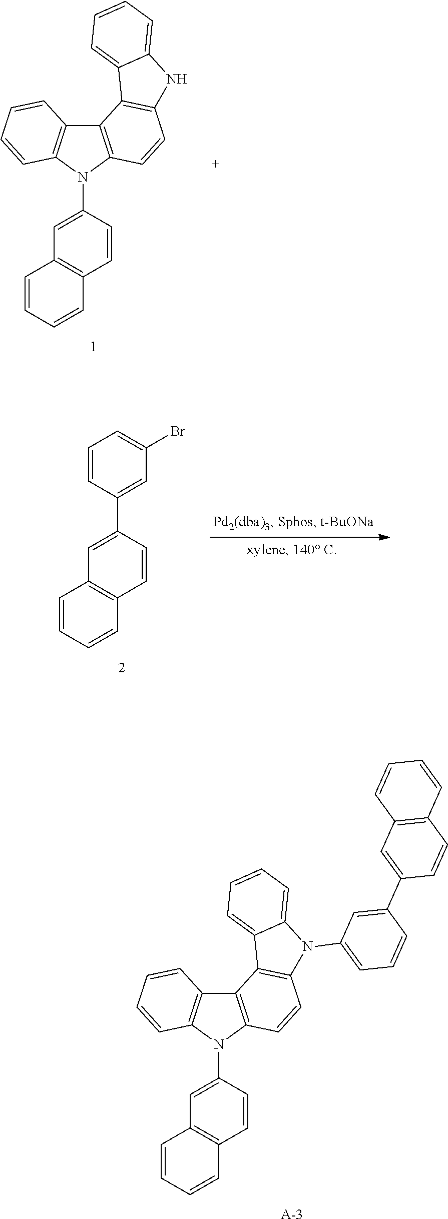 [SMILES: c1ccc2cc(-n3c4ccccc4c4c5c(ccc43)[nH]c3ccccc35)ccc2c1 ; Brc1cccc(-c2ccc3ccccc3c2)c1 ; c1cc(-c2ccc3ccccc3c2)cc(-n2c3ccccc3c3c4c5ccccc5n(-c5ccc6ccccc6c5)c4ccc32)c1 ; "A-3"]

Under nitrogen protection, intermediate 1 (30.0 g, 78.44 mmol), intermediate 2 (24.43 g, 86.28 mmol), tris(dibenzylideneacetone)dipalladium (1.44 g, 1.57 mmol), 2-dicyclohexylphosphino-2′,6′-dimethoxybiphenyl (Sphos, 1.29 g, 3.14 mmol), sodium tert-butoxide (t-BuONa, 15.08 g, 156.88 mmol) and xylene (300 mL) were added to a three-necked flask and reacted at 140° C. for 16 hours. After completion of the reaction, the reaction was cooled to room temperature, the reaction mixture was filtered to give a crude product, and the crude product was extracted with toluene. The organic phase was washed with water and concentrated to give the crude product as a solid, and the crude product was recrystallized in toluene to give Compound A-3 (40 g, with a yield of 87%). The product was confirmed as the target product with a molecular weight of 584.23.

### Synthesis Example 2: Synthesis of Compound A-79

### Step 1: Synthesis of Compound A-79

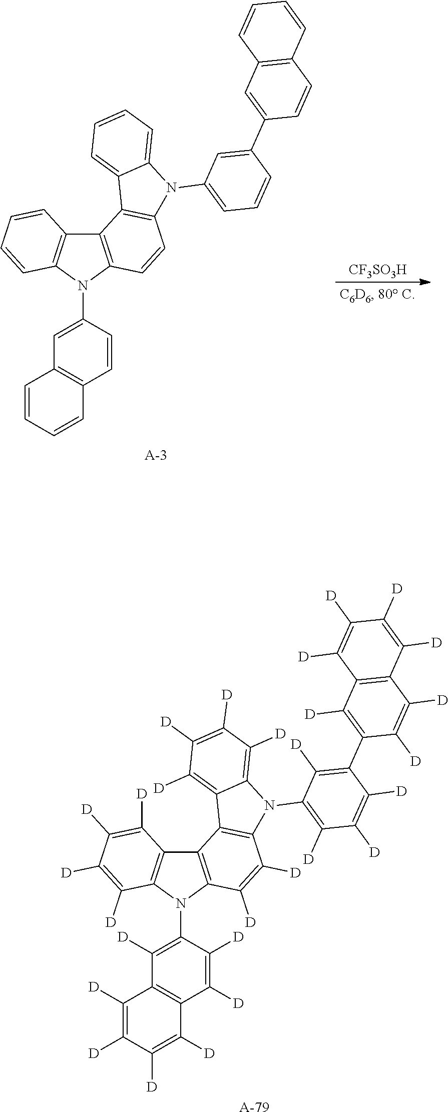 [SMILES: c1cc(-c2ccc3ccccc3c2)cc(-n2c3ccccc3c3c4c5ccccc5n(-c5ccc6ccccc6c5)c4ccc32)c1 ; "CF3SO3H" ; "A-3" ; [2H]c1c([2H])c(-c2c([2H])c([2H])c3c([2H])c([2H])c([2H])c([2H])c3c2[2H])c([2H])c(-n2c3c([2H])c([2H])c([2H])c([2H])c3c3c4c5c([2H])c([2H])c([2H])c([2H])c5n(-c5c([2H])c([2H])c6c([2H])c([2H])c([2H])c([2H])c6c5[2H])c4c([2H])c([2H])c32)c1[2H] ; "A-79"]

Under nitrogen protection, Compound A-3 (20 g, 34.20 mmol), trifluoromethanesulfonic acid (CF₃SO₃H, 5.13 g, 34.20 mmol) and deuterated benzene (C₆D6, 150 mL) were added to a three-necked flask and reacted at 80° C. for 16 hours. After completion of the reaction, the reaction was cooled to room temperature and quenched with deuterium water. After the reaction was carried out for 10 minutes, a potassium phosphate solution was added, and the crude product was extracted with dichloride. The organic phase was washed with water, and the crude product was purified by column chromatography with PE:DCM (4:1) to give Compound A-79 (11 g, with a yield of 52%). The product was confirmed as the target product with a molecular weight of 612.40.

### Synthesis Example 3: Synthesis of Compound A-4

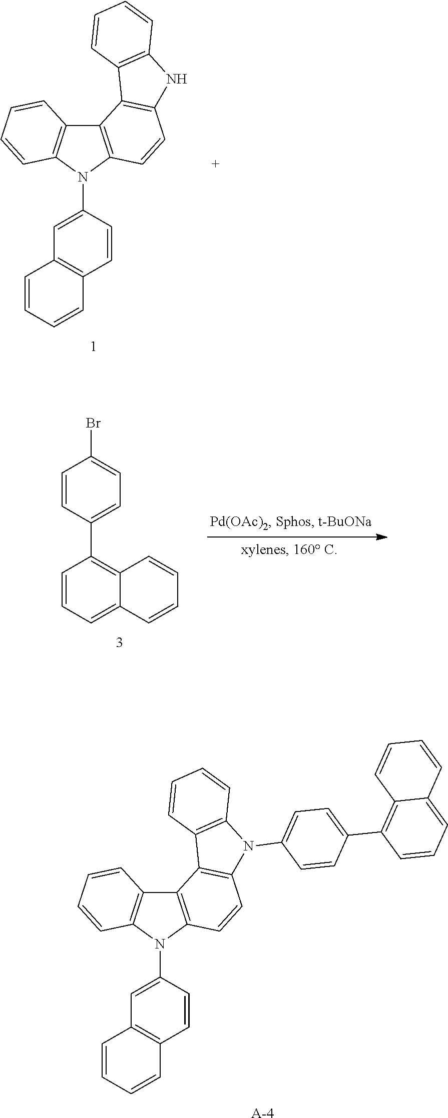 [SMILES: c1ccc2cc(-n3c4ccccc4c4c5c(ccc43)[nH]c3ccccc35)ccc2c1 ; Brc1ccc(-c2cccc3ccccc23)cc1 ; c1ccc2cc(-n3c4ccccc4c4c5c6ccccc6n(-c6ccc(-c7cccc8ccccc78)cc6)c5ccc43)ccc2c1 ; "A-4"]

Under nitrogen protection, intermediate 1 (10.0 g, 26.15 mmol), intermediate 3 (7.4 g, 26.15 mmol), palladium acetate (Pd(OAc)₂, 293.5 mg, 1.31 mmol), 2-dicyclohexylphosphino-2′,6′-dimethoxybiphenyl (Sphos, 1.07 g, 2.61 mmol), sodium tert-butoxide (t-BuONa, 5.03 g, 52.29 mmol) and xylene (100 mL) were added to a three-necked flask and reacted at 160° C. overnight. After completion of the reaction, the reaction was cooled to room temperature and quenched with water, and the reaction mixture was extracted with dichloromethane. The organic phase was washed with water and concentrated to give the crude product as a solid, and the crude product was purified by column chromatography with PE:DCM (4:1) to give Compound A-4 (11.4 g, with a yield of 75%). The product was confirmed as the target product with a molecular weight of 584.23.

### Synthesis Example 4: Synthesis of Compound A-78

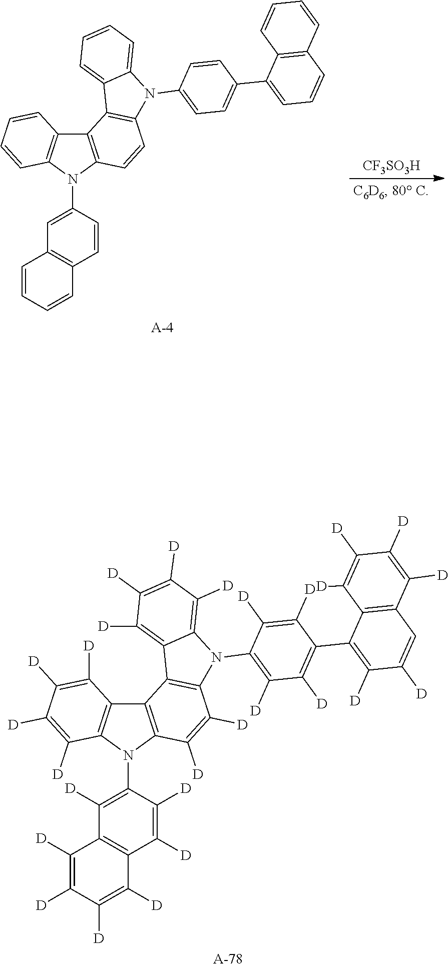

Under nitrogen protection, Compound A-4 (4.4 g, 7.52 mmol), trifluoromethanesulfonic acid (1.13 g, 7.52 mmol) and deuterated benzene (40 mL) were added to a three-necked flask and reacted at 80° C. overnight. After completion of the reaction, the reaction was cooled to room temperature and quenched with deuterium water. After the reaction was carried out for 10 minutes, a potassium phosphate solution was added, and the crude product was extracted with dichloride. The organic phase was washed with water, and the crude product was purified by column chromatography with PE:DCM (4:1) to give Compound A-78 (3.0 g, with a yield of 65%). The product was confirmed as the target product with a molecular weight of 612.40.

### Synthesis Example 5: Synthesis of Compound A-80

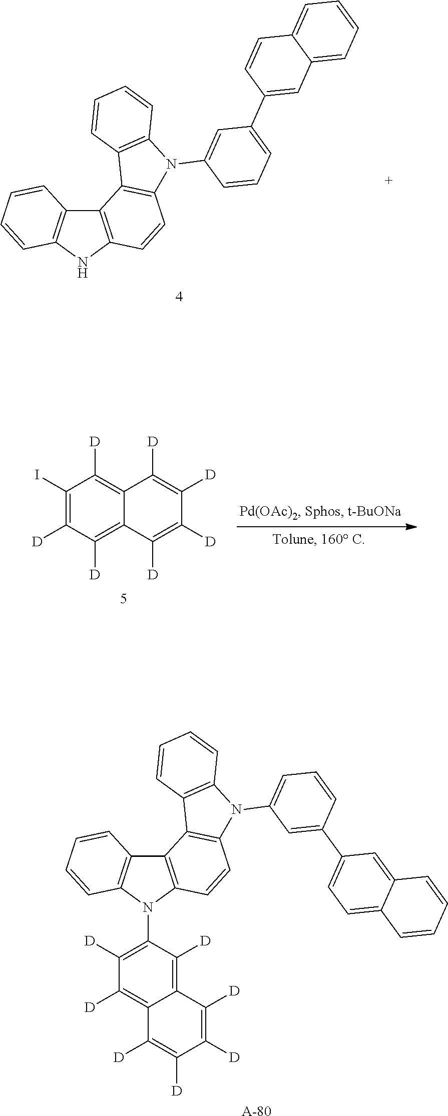 [SMILES: c1cc(-c2ccc3ccccc3c2)cc(-n2c3ccccc3c3c4c(ccc32)[nH]c2ccccc24)c1 ; [2H]c1c([2H])c([2H])c2c([2H])c(I)c([2H])c([2H])c2c1[2H] ; [2H]c1c([2H])c([2H])c2c([2H])c(-n3c4ccccc4c4c5c6ccccc6n(-c6cccc(-c7ccc8ccccc8c7)c6)c5ccc43)c([2H])c([2H])c2c1[2H] ; "A-80"]

Under nitrogen protection, intermediate 4 (2 g, 4.36 mmol), intermediate 5 (1.14 g, 4.36 mmol), palladium acetate (79.88 mg, 0.09 mmol), 2-dicyclohexylphosphino-2′,6′-dimethoxybiphenyl (71.6 mg, 0.17 mmol), sodium tert-butoxide (838.31 mg, 8.72 mmol) and toluene (50 mL) were added to a three-necked flask and reacted at 160° C. for 16 hours. After completion of the reaction, the reaction mixture was extracted with ethyl acetate. The organic phase was washed with water and concentrated to remove solvents, and the crude product was purified by column chromatography with PE:DCM (4:1) to give Compound A-80 (1.8 g, with a yield of 70%). The product was confirmed as the target product with a molecular weight of 591.27.

### Synthesis Example 6: Synthesis of Compound B-1

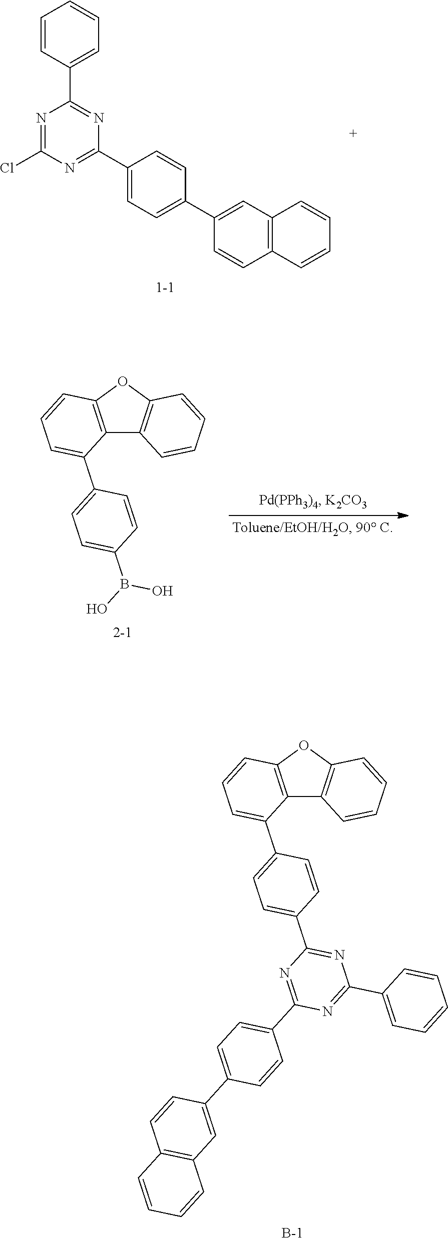

Under nitrogen protection, intermediate 1-1 (8.2 g, 20.8 mmol), intermediate 2-1 (6 g, 20.8 mmol), tetrakis(triphenylphosphine)palladium (721 mg, 0.6 mmol), potassium carbonate (5.7 g, 41.6 mmol) and solvents (toluene/ethanol/water,80/20/20 mL) were added to a three-necked flask and reacted at 90° C. overnight. After completion of the reaction, the reaction was cooled to room temperature, distilled water was added to precipitate a white solid, and the reaction mixture was filtered to give the solid. The solid was recrystallized in toluene to give Compound B-1 (9.3 g, with a yield of 74%). The product was confirmed as the target product with a molecular weight of 601.22.

### Synthesis Example 7: Synthesis of Compound B-2

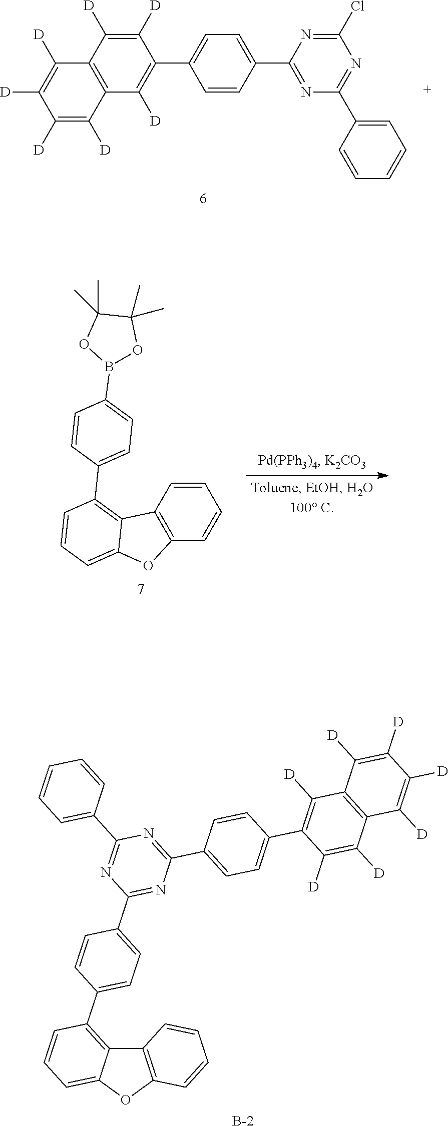

Under nitrogen protection, intermediate 6 (3.00 g, 7.48 mmol), intermediate 7 (2.77 g, 7.48 mmol), tetrakis(triphenylphosphine)palladium (0.17 g, 0.15 mmol), potassium carbonate (2.0 g, 14.97 mmol), toluene (80 mL), ethanol (20 mL) and water (20 mL) were added to a three-necked flask and reacted at 100° C. for 16 hours. After completion of the reaction, the reaction was cooled to room temperature, and the reaction mixture was filtered to give a solid. The crude product was recrystallized with toluene to give Compound B-2 (3.0 g, with a yield of 65.8%). The product was confirmed as the target product with a molecular weight of 608.26.

### Synthesis Example 8: Synthesis of Compound B-222

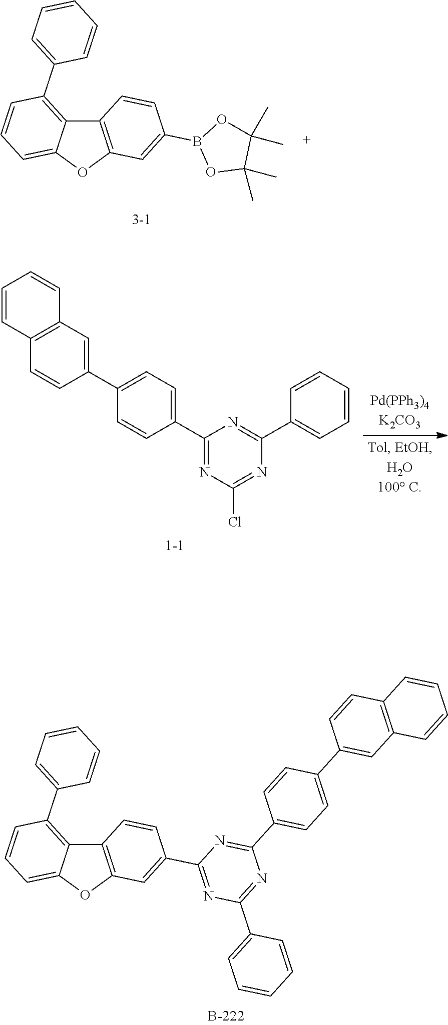 [SMILES: CC1(C)OB(c2ccc3c(c2)oc2cccc(-c4ccccc4)c23)OC1(C)C ; Clc1nc(-c2ccccc2)nc(-c2ccc(-c3ccc4ccccc4c3)cc2)n1 ; c1ccc(-c2nc(-c3ccc(-c4ccc5ccccc5c4)cc3)nc(-c3ccc4c(c3)oc3cccc(-c5ccccc5)c34)n2)cc1]

Under nitrogen protection, intermediate 3-1 (10 g, 27.0 mmol), intermediate 1-1 (10.6 g, 27.0 mmol), tetrakis(triphenylphosphine)palladium (1,500 mg, 1.3 mmol), potassium carbonate (7.5 g, 54.0 mmol), toluene (300 mL), ethanol (50 mL) and water (50 mL) were added to a three-necked flask and reacted at 100° C. for 16 hours. After completion of the reaction, a larger amount of solid was precipitated, and the reaction mixture was filtered to give the solid. The solid was washed three times with water and ethanol separately. The crude product was recrystallized with toluene to give Compound B-222 (9.8 g, with a yield of 61%). The product was confirmed as the target product with a molecular weight of 601.22.

The persons skilled in the art will appreciate that the above preparation methods are merely exemplary. The persons skilled in the art can obtain other compound structures of the present disclosure via modifications of the preparation methods.

Measurement of the triplet energy level (T₁) of the compound: Herein, the triplet energy level (T₁) of the compound was measured at an ultra-low temperature using properties of long-lived triplet excitons. Specifically, a compound to be measured was dissolved in a 2-methyltetrahydrofuran solvent to prepare a solution having a concentration of 10⁻⁵ M. The solution was loaded into a quartz tube, placed into a Dewar flask and cooled to 77 K. The solution of the compound to be measured was irradiated with a light source of 350 nm to measure a phosphorescence spectrum. The spectrum was measured using a spectrophotometer F98 produced by SHANGHAI LENGGUANG TECHNOLOGY CO., LTD. In the phosphorescence spectrum, the longitudinal axis represented a phosphorescence intensity, and the horizontal axis represented a wavelength. The minimum value λ₁ (nm) of a peak on the shorter wavelength side of the phosphorescence spectrum was taken, and the wavelength value was substituted into the following conversion formula F₁ to calculate the triplet energy of the compound to be measured. The conversion formula F₁ is as follows: T₁ (eV)=1240/λ₁. The results of the triplet energy levels of part of the compounds herein measured in accordance with the above method are recorded in Table 11.

TABLE 11
Triplet energy levels (T₁) of part of the compounds

| Compound ID | λ₁ (nm) | T₁ (eV) |
|---|---|---|
| Compound A-1 | 480 | 2.58 |
| Compound A-3 | 496 | 2.50 |
| Compound A-4 | 494 | 2.51 |
| Compound A-78 | 492 | 2.52 |
| Compound A-79 | 493 | 2.51 |
| Compound A-86 | 492 | 2.52 |
| Compound A-107 | 496 | 2.50 |
| Compound B-1 | 516 | 2.40 |
| Compound B-222 | 515 | 2.41 |

Herein, the values of highest occupied molecular orbital (HOMO) energy levels and lowest unoccupied molecular orbital (LUMO) energy levels of all the compounds were measured by cyclic voltammetry (CV). The measurement was conducted using an electrochemical workstation CorrTest CS120 produced by WUHAN CORRTEST INSTRUMENTS CORP., LTD and using a three-electrode working system where a platinum disk electrode served as a working electrode, an Ag/AgNO₃ electrode served as a reference electrode, and a platinum wire electrode served as an auxiliary electrode. The measurement was conducted at 25° C. With anhydrous DCM as a solvent and 0.1 mol/L tetrabutylammonium hexafluorophosphate as a supporting electrolyte, the compound to be measured was prepared into a solution of 10⁻³ mol/L, and nitrogen was introduced into the solution for 10 minutes for oxygen removal before the measurement. The parameters of the instrument were set as follows: a scan rate of 100 mV/s, a potential interval of 0.5 mV, and a measurement window of 1 V to −0.5 V. Herein, all “HOMO energy levels” and “LUMO energy levels” were expressed as negative values, and the smaller the numerical value (i.e., the larger the absolute value), the deeper the energy level. “/” represented Not Measured.

TABLE 12
HOMO\LUMO energy levels of part of the materials

| Compound | HOMO [eV] | LUMO [eV] |
|---|---|---|
| A-1 | −5.329 | / |
| A-3 | −5.332 | −2.118 |
| A-4 | −5.330 | / |
| A-78 | −5.326 | / |
| A-79 | −5.323 | / |
| A-86 | −5.337 | / |
| A-107 | −5.318 | / |
| B-1 | / | −2.829 |
| B-2 | / | −2.853 |
| B-222 | / | −2.807 |

### Device Example

### Device Example 1

First, a glass substrate having an indium tin oxide (ITO) anode having a thickness of 120 nm was cleaned and then treated with UV ozone and oxygen plasma. After the treatment, the substrate was dried in a nitrogen-filled glovebox to remove moisture, loaded on a substrate support and transferred into a vacuum chamber. Organic layers specified below were sequentially evaporated by vacuum thermal evaporation on the ITO anode at a rate of 0.01 to 5 Angstroms per second (A/s) and a vacuum degree of about 10⁻⁶ torr. Compound HT and Compound HI were co-evaporated at a mass ratio of 97:3 as a hole injection layer (HIL) with a thickness of 100 Å. Compound HT was evaporated as a hole transport layer (HTL) with a thickness of 400 Å. Compound EB was evaporated as an electron blocking layer (EBL) with a thickness of 50 Å. Then, Compound A-78 of the present disclosure serving as a first host, Compound A-79 of the present disclosure serving as a third host, Compound B-1 of the present disclosure serving as a second host and Compound RD serving as a dopant were loaded into four different evaporation sources and co-evaporated at a mass ratio of 9.8:39.2:49:2 as an emissive layer (EML) with a thickness 400 Å. Compound HB was evaporated as a hole blocking layer (HBL) with a thickness of 50 Å. On the hole blocking layer, Compound ET and 8-hydroxyquinolinolato-lithium (Liq) were co-evaporated at a mass ratio of 40:60 as an electron transport layer (ETL) with a thickness of 350 Å. Finally, 8-hydroxyquinolinolato-lithium (Liq) with a thickness of 10 Å was evaporated as an electron injection layer (EIL), and Al with a thickness of 1200 Å was evaporated as a cathode. The device was transferred back to the glovebox and encapsulated with a glass lid such that the device was completed.

### Device Example 2

The implementation in Device Example 2 was the same as the implementation in Device Example 1, except that Compound A-78 was substituted with Compound A-3 as the first host in the emissive layer (EML), wherein the mass ratio of Compound A-3:Compound A-79:Compound B-1:Compound RD was 24.5:24.5:49:2.

### Device Example 3

The implementation in Device Example 3 was the same as the implementation in Device Example 2, except that Compound A-79 was substituted with Compound A-1 as the third host in the emissive layer (EML).

### Device Example 4

The implementation in Device Example 4 was the same as the implementation in Device Example 1, except that Compound A-78 and Compound A-79 were substituted with Compound A-1 and Compound B-222 as the first host and the third host in the emissive layer (EML), respectively, wherein the mass ratio of Compound A-1:Compound B-222:Compound B-1:Compound RD was 49:24.5:24.5:2.

### Device Comparative Example 1

The implementation in Device Comparative Example 1 was the same as the implementation in Device Example 1, except that Compound A-78 and Compound A-79 were substituted with Compound A-3 as the host in the emissive layer (EML), wherein the mass ratio of Compound A-3:Compound B-1:Compound RD was 49:49:2.

### Device Comparative Example 2

The implementation in Device Comparative Example 2 was the same as the implementation in Device Example 1, except that Compound A-78, Compound A-79 and Compound B-1 were substituted with Compound A-1 as the host in the emissive layer (EML), wherein the mass ratio of Compound A-1:Compound RD was 98:2.

Detailed structures and thicknesses of layers of the devices are shown in the following table. A layer using more than one material was obtained by doping different compounds at their mass ratio as recorded.

TABLE 1
Device structures in Device Examples 1-4 and Device Comparative Examples 1-2

| Device | | | | | | |
|---|---|---|---|---|---|---|
| ID | HIL | HTL | EBL | EML | HBL | ETL |
| Example 1 | Compound HT:Compound HI | Compound HT | Compound EB | Compound | Compound | Compound |
| | (97:3) (100 Å) | (400 Å) | (50 Å) | A-78:Compound | HB | ET:Liq |
| | | | | A-79:Compound | (50 Å) | (40:60) |
| | | | | B-1:Compound RD | | (350 Å) |
| | | | | (9.8:39.2:49:2) | | |
| | | | | (400 Å) | | |
| Example 2 | Compound HT:Compound HI | Compound HT | Compound EB | Compound | Compound | Compound |
| | (97:3) (100 Å) | (400 Å) | (50 Å) | A-3:Compound | HB | ET:Liq |
| | | | | A-79:Compound | (50 Å) | (40:60) |
| | | | | B-1:Compound RD | | (350 Å) |
| | | | | (24.5:24.5:49:2) | | |
| | | | | (400 Å) | | |
| Example 3 | Compound HT:Compound HI | Compound HT | Compound EB | Compound | Compound | Compound |
| | (97:3) (100 Å) | (400 Å) | (50 Å) | A-3:Compound | HB | ET:Liq |
| | | | | A-1:Compound | (50 Å) | (40:60) |
| | | | | B-1:Compound RD | | (350 Å) |
| | | | | (24.5:24.5:49:2) | | |
| | | | | (400 Å) | | |
| Example 4 | Compound HT:Compound HI | Compound HT | Compound EB | Compound | Compound | Compound |
| | (97:3) (100 Å) | (400 Å) | (50 Å) | A-1:Compound | HB | ET:Liq |
| | | | | B-222:Compound | (50 Å) | (40:60) |
| | | | | B-1:Compound RD | | (350 Å) |
| | | | | (49:24.5:24.5:2) | | |
| | | | | (400 Å) | | |
| Comparative | Compound HT:Compound HI | Compound HT | Compound EB | Compound | Compound | Compound |
| Example 1 | (97:3) (100 Å) | (400 Å) | (50 Å) | A-3:Compound | HB | ET:Liq |
| | | | | B-1:Compound RD | (50 Å) | (40:60) |
| | | | | (49:49:2) (400 Å) | | (350 Å) |
| Comparative | Compound HT:Compound HI | Compound HT | Compound EB | Compound | Compound | Compound |
| Example 2 | (97:3) (100 Å) | (400 Å) | (50 Å) | A-1:Compound RD | HB | ET:Liq |
| | | | | (98:2) (400 Å) | (50 Å) | (40:60) |
| | | | | | | (350 Å) |

The structures of the compounds used in the devices are shown below:

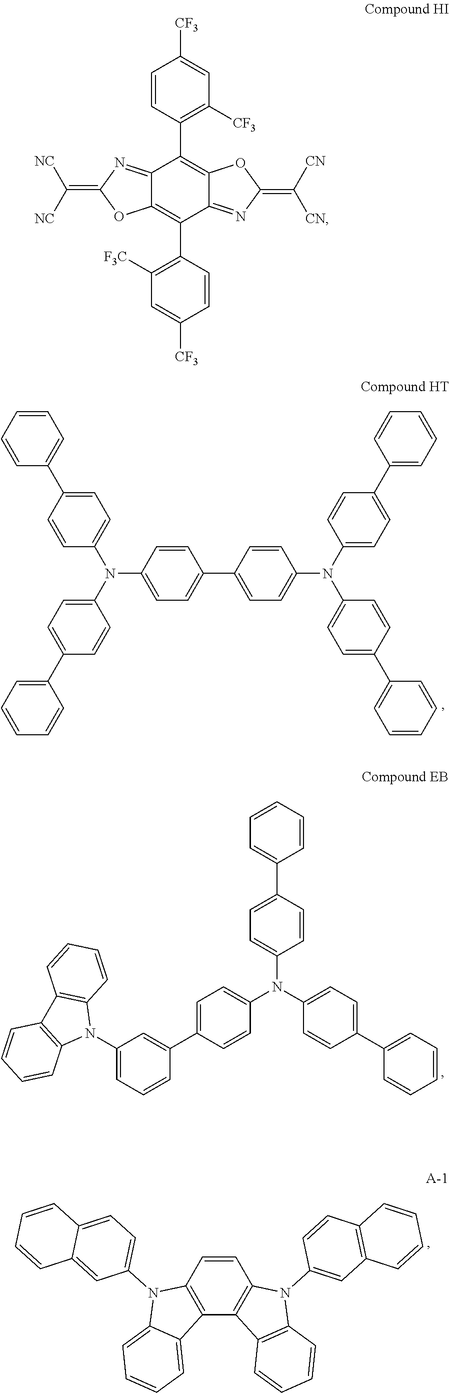 [SMILES: N#CC(C#N)=c1nc2c(-c3ccc(C(F)(F)F)cc3C(F)(F)F)c3oc(=C(C#N)C#N)nc3c(-c3ccc(C(F)(F)F)cc3C(F)(F)F)c2o1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2ccc(N(c3ccc(-c4ccccc4)cc3)c3ccc(-c4cccc(-n5c6ccccc6c6ccccc65)c4)cc3)cc2)cc1 ; c1ccc2cc(-n3c4ccccc4c4c5c6ccccc6n(-c6ccc7ccccc7c6)c5ccc43)ccc2c1]

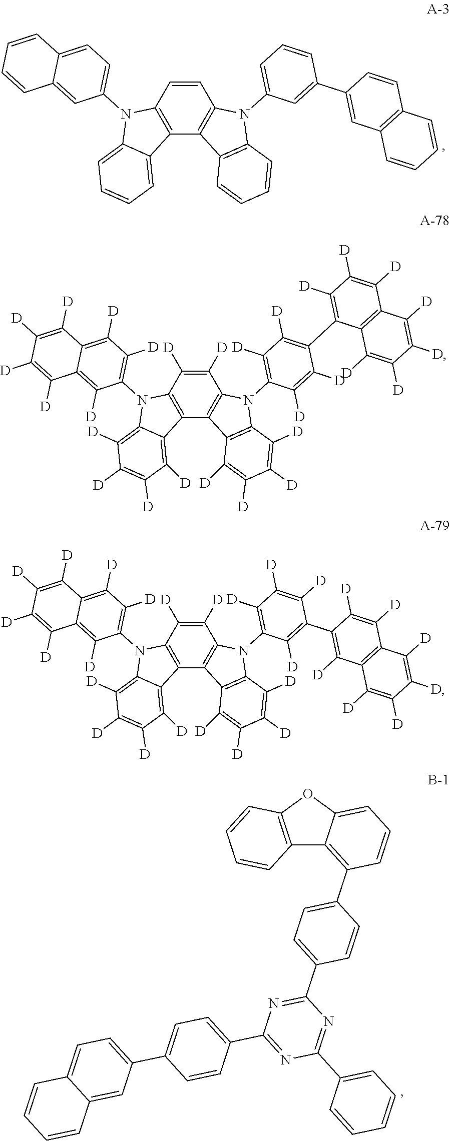

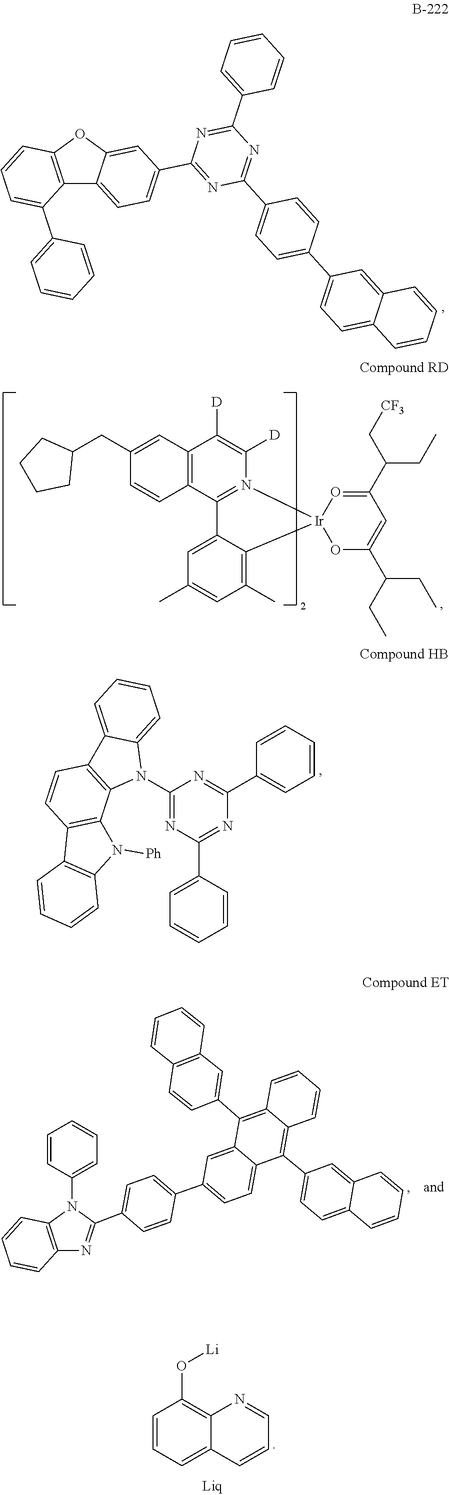

The maximum emission wavelength (λₘₐₓ), current efficiency (CE) and external quantum efficiency (EQE) of the device examples and device comparative examples measured at a constant current of 15 mA/cm² as well as the device lifetime (LT97) measured at a constant current of 80 mA/cm² were shown in Table 2, wherein the device lifetime (LT97) refers to the time for the device to decay to 97% of its initial brightness.

TABLE 2
Device data in Device Examples 1 to 4
and Device Comparative Examples 1 to 2

| Device ID | λₘₐₓ (nm) | CE [cd/A] | EQE [%] | LT97 (h) |
|---|---|---|---|---|
| Example 1 | 618 | 25.1 | 25.6 | 97 |
| Example 2 | 620 | 24.9 | 25.4 | 51 |
| Example 3 | 618 | 25.1 | 25.6 | 52 |
| Example 4 | 620 | 24.5 | 25.1 | 110 |
| Comparative Example 1 | 619 | 24.8 | 25.2 | 45 |
| Comparative Example 2 | 618 | 20.6 | 19.9 | 3.8 |

### Discussion

As can be seen from the device structures in Table 1, the difference between Examples 1 to 4 and Comparative Example 1 is that the compound composition disclosed in the present disclosure was specifically selected as the host material in the examples, wherein in Examples 1 to 3, the first compound having the structure of Formula 1 was selected as the first host material, the second compound having the structure of Formula 2 was selected as the second host material, and the third compound having the structure of Formula 1 was selected as the third host material. Compared to Device Comparative Example 1 using dual host materials, on the basis that the maximum emission wavelengths of Examples 1, 2 and 3 remained basically the same as the maximum emission wavelength of Device Comparative Example 1, the lifetimes of Examples 1, 2 and 3 were significantly improved by 115%, 13.3% and 15.5%, respectively, and in addition, the CEs and EQEs of Examples 1, 2 and 3 showed further improvements relative to high levels of CE and EQE of Comparative Example 1. Compared to Comparative Example 2 using a single host material, Device Examples 1 to 3 where a compound composition comprising a three-component material with a specific structure of the present disclosure was used as the host material in the emissive layer achieved significant improvements in both efficiency and lifetime.

In Example 4, the third compound having the structure of Formula 2 was specifically selected as the third host material. As can be seen from the data in Table 2, compared to Comparative Example 1 using two host materials, Example 4 achieved an unexpected and significant improvement of 144% in lifetime while maintaining high levels of CE and EQE. As can be seen from the above, the organic electroluminescent device of the present disclosure using a compound composition comprising the first compound that has a structure of Formula 1 of indolocarbazole, the second compound that has a structure of Formula 2 of triazine and the third compound that has a structure of Formula 1 or Formula 2 but has a different structure from the structure of the first or second compound as the host material had excellent comprehensive device performance and in particular, achieved a significant improvement in lifetime.

In particular, as surprisingly found from the data in Table 2, when at least two of the selected first, second and third compounds were isomers of each other (as in Device Examples 1 and 4), the organic electroluminescent device of the present disclosure could achieve more excellent device performance and achieved a breakthrough improvement in device lifetime of 115% to 144% even compared to Comparative Example 1 with relative good performance.

In conclusion, when the compound composition of the present disclosure comprising the first compound having a structure of Formula 1, the second compound having a structure of Formula 2 and the third compound having a structure of Formula 1 or Formula 2 (the three compounds have different structures) is applied to the organic electroluminescent device as the host material, the organic electroluminescent device achieves excellent comprehensive performance, in particular, achieves an improvement in lifetime, and has broad application prospects.

As can be seen from the above device data, when the emissive layer is prepared using multiple (usually more than three) raw materials, the light-emitting device is enabled to obtain better comprehensive performance. However, if the device using multiple raw materials is prepared by using a normal preparation method, that is, each component serves as a separate evaporation source (such as the preparation of the emissive layer in above Device Examples 1 to 4), the preparation process and the cost will be increased. The desired approach is to pre-mix the multi-component material to form a pre-mix with high evaporation stability and then use the pre-mix as a single evaporation source to reduce the complexity of the vacuum deposition process.

However, the stability of the components in the film formed by evaporation of the mixture has a large impact on device performance. Therefore, a pre-mixture capable of being used as a single evaporation source must be stable during co-evaporation, that is, the proportion deviation of the compositions of the film formed by evaporation of the mixture is as small as possible during the vacuum deposition process. However, when two compounds are mixed, it is difficult to achieve a stable co-evaporation mixture since the possible interaction between the two compounds has an effect on the evaporation film-forming stability of the two compounds.

The inventors of the present disclosure further provide, as a result of their research, a new mixture comprising at least three hole or electron transport-type compounds that are different from each other in structure, wherein at least one hole transport-type material comprises a fused structure which is formed by fusing at least four 5- and/or 6-membered rings and which is not triphenylene. The mixture of the present disclosure can achieve stable single-source co-evaporation to ensure stable device performance. In particular, for the mixture provided herein comprising more than three different hole and electron transport-type compounds, the evaporation film-forming stability of the mixture can be improved by selecting hole and electron transport-type compounds having specific “heavy” and “light” evaporation property relationships. The stability of the mixture can be demonstrated by compositional analysis of the film prepared with a single co-evaporation source comprising the three-component pre-mix, as specifically shown below:

### Mixture Example

Mixture Example 1: Compounds A-79, A-78 and B-1 were pre-melted and mixed at a mass ratio of A-79:A-78:B-1=4:1:5 to form a mixture MX1 of the present disclosure, the mixture MX1 was then milled to a powder and loaded into an evaporation source, the distance from the evaporation source to the glass substrate was set to be 35-60 cm, and the mixture MX1 was evaporated at a rate of 2 Å/s and a vacuum degree of about 10⁻⁶ torr and deposited onto the glass substrate. After a film with a thickness of 400 Å was deposited, the depositing and cooling of the evaporation source kept going with the substrate continuously replaced to form multiple films having a thickness of 400 Å. The compositions of the films were analyzed by HPLC, and the results were collected in Table 3.

Table 3 records and presents the compositions (%) of the films deposited sequentially from the pre-mix MX1, analyzed by HPLC. The HPLC analysis conditions used are: analytical column used for testing: ODS column; eluent: acetonitrile; detection wavelength: 272 nm.

TABLE 3
Composition proportion of the mixture MX1 in the films

| Film | A-79 + A-78 (%) | B-1 (%) |
|---|---|---|
| Film 1 | 49.197 | 50.803 |
| Film 2 | 49.138 | 50.862 |
| Film 3 | 49.125 | 50.875 |
| Film 4 | 49.171 | 50.829 |
| Film 5 | 49.096 | 50.904 |
| Film 6 | 49.129 | 50.871 |

Mixture Example 2: Compounds A-3, A-4 and B-1 were pre-melted and mixed at a mass ratio of A-3:A-4:B-1=3.2:0.8:6 to form a mixture MX2 of the present disclosure, the pre-mixed mixture MX2 was then milled to a powder and loaded into an evaporation source, the distance from the evaporation source to the glass substrate was set to be 35-60 cm, and the mixture MX2 was evaporated at a rate of 2 Å/s and a vacuum degree of about 10⁻⁶ torr and deposited onto the glass substrate. After a film with a thickness of 400 Å was deposited, the depositing and cooling of the source kept going with the substrate continuously replaced to form multiple films having a thickness of 400 Å. The compositions of the films were analyzed by HPLC, and the results were collected in Table 4.

Table 4 records and presents the compositions (%) of the films deposited sequentially from the pre-mix MX2, analyzed by HPLC. The HPLC analysis conditions used are: analytical column used for testing: ODS column; eluent: acetonitrile; detection wavelength: 272 nm.

TABLE 4
Composition proportion of the mixture MX2 in the films

| Film | A-3 + A-4 (%) | B-1 (%) |
|---|---|---|
| Film 1 | 40.791 | 59.209 |
| Film 2 | 40.737 | 59.263 |
| Film 3 | 40.783 | 59.217 |
| Film 4 | 40.734 | 59.266 |
| Film 5 | 40.791 | 59.209 |
| Film 6 | 40.831 | 59.169 |

Mixture Example 3: Compounds A-86, A-107 and B-1 were pre-melted and mixed at a mass ratio of A-86:A-107:B-1=4:1:5 to form a mixture MX3 of the present disclosure, the pre-mixed mixture MX3 was then milled to a powder and loaded into an evaporation source, the distance from the evaporation source to the glass substrate was set to be 35-60 cm, and the mixture MX3 was evaporated at a rate of 2 Å/s and a vacuum degree of about 10⁻⁶ torr and deposited onto the glass substrate. After a film with a thickness of 400 Å was deposited, the depositing and cooling of the source kept going with the substrate continuously replaced to form multiple films having a thickness of 400 Å. The compositions of the films were analyzed by HPLC, and the results were collected in Table 14.

Table 14 records and presents the compositions (%) of the films deposited sequentially from the pre-mix MX3, analyzed by HPLC. The HPLC analysis conditions used are: analytical column used for testing: ODS column; eluent: acetonitrile; detection wavelength: 272 nm.

TABLE 14
Composition proportion of the mixture MX3 in the films

| Film | A-86 + A-107 (%) | B-1 (%) |
|---|---|---|
| Film 1 | 50.737 | 49.263 |
| Film 2 | 50.780 | 49.220 |
| Film 3 | 50.825 | 49.175 |
| Film 4 | 50.800 | 49.200 |
| Film 5 | 50.803 | 49.197 |
| Film 6 | 50.803 | 49.197 |

Mixture Comparative Example 1: Compounds A-3 and B-1 were pre-melted and mixed at a mass ratio of A-3:B-1=5:5 to form a comparative mixture C-MX1, the pre-mixed mixture was then milled to a powder and loaded into an evaporation source, the distance from the evaporation source to the glass substrate was set to be 35-60 cm, and the mixture C-MX1 was co-evaporated at a rate of 2 Å/s and a vacuum degree of about 10⁻⁶ torr and deposited onto the glass substrate. After a film with a thickness of 400 Å was deposited, the depositing and cooling of the source kept going with the substrate continuously replaced to form multiple films having a thickness of 400 Å. The compositions of the films were analyzed by HPLC, and the results were collected in Table 5.

Table 5 records and presents the compositions (%) of the films deposited sequentially from the two-component pre-mix C-MX1, analyzed by HPLC. The HPLC analysis conditions used are: analytical column used for testing: ODS column; eluent: acetonitrile; detection wavelength: 272 nm.

TABLE 5
Composition proportion of the comparative
mixture C-MX1 in the films

| Film | A-3 (%) | B-1 (%) |
|---|---|---|
| Film 1 | 47.079 | 52.921 |
| Film 2 | 48.622 | 51.378 |
| Film 3 | 49.039 | 50.961 |
| Film 4 | 49.228 | 50.772 |
| Film 5 | 49.406 | 50.594 |
| Film 6 | 49.755 | 50.245 |

Mixture Comparative Example 2: Compounds A-79 and B-1 were pre-melted and mixed at a mass ratio of A-79:B-1=5:5 to form a comparative mixture C-MX2, the pre-mixed mixture was then milled to a powder and loaded into an evaporation source, the distance from the evaporation source to the glass substrate was set to be 35-60 cm, and the mixture was co-evaporated at a rate of 2 Å/s and a vacuum degree of about 10⁻⁶ torr and deposited onto the glass substrate. After a film with a thickness of 400 Å was deposited, the depositing and cooling of the source kept going with the substrate continuously replaced to form multiple films having a thickness of 400 Å. The compositions of the films were analyzed by HPLC, and the results were collected in Table 6.

Table 6 records and presents the compositions (%) of the films deposited sequentially from the two-component pre-mix C-MX2, analyzed by HPLC. The HPLC analysis conditions used are: analytical column used for testing: ODS column; eluent: acetonitrile; detection wavelength: 272 nm.

TABLE 6
Composition proportion of the comparative
mixture C-MX2 in the films

| Film | A-79 (%) | B-1 (%) |
|---|---|---|
| Film 1 | 48.575 | 51.425 |
| Film 2 | 48.981 | 51.019 |
| Film 3 | 49.698 | 50.302 |
| Film 4 | 50.329 | 49.671 |
| Film 5 | 51.749 | 48.251 |
| Film 6 | 52.177 | 47.823 |

Mixture Comparative Example 3: Compounds A-4 and B-1 were pre-melted and mixed at a mass ratio of A-4:B-1=6:4 to form a comparative mixture C-MX3, the pre-mixed mixture C-MX3 was then milled to a powder and loaded into an evaporation source, the distance from the evaporation source to the glass substrate was set to be 35-60 cm, and the mixture was co-evaporated at a rate of 2 Å/s and a vacuum degree of about 10⁻⁶ torr and deposited onto the glass substrate. After a film with a thickness of 400 Å was deposited, the depositing and cooling of the source kept going with the substrate continuously replaced to form multiple films having a thickness of 400 Å. The compositions of the films were analyzed by HPLC, and the results were collected in Table 7.

Table 7 records and presents the compositions (%) of the films deposited sequentially from the two-component pre-mix C-MX3, analyzed by HPLC. The HPLC analysis conditions used are: analytical column used for testing: ODS column; eluent: acetonitrile; detection wavelength: 272 nm.

TABLE 7
Composition proportion of the mixture C-MX3 in the films

| Film | A-4 (%) | B-1 (%) |
|---|---|---|
| Film 1 | 55.989 | 44.011 |
| Film 2 | 55.745 | 44.255 |
| Film 3 | 55.555 | 44.445 |
| Film 4 | 55.401 | 44.599 |
| Film 5 | 55.363 | 44.637 |
| Film 6 | 55.146 | 44.854 |

Mixture Comparative Example 4: Compounds A-78 and B-1 were pre-melted and mixed at a mass ratio of A-78:B-1=6:4 to form a comparative mixture C-MX4, the pre-mixed mixture was then milled to a powder and loaded into an evaporation source, the distance from the evaporation source to the glass substrate was set to be 35-60 cm, and the mixture was co-evaporated at a rate of 2 Å/s and a vacuum degree of about 10⁻⁶ torr and deposited onto the glass substrate. After a film with a thickness of 400 Å was deposited, the depositing and cooling of the source kept going with the substrate continuously replaced to form multiple films having a thickness of 400 Å. The compositions of the films were analyzed by HPLC, and the results were collected in Table 8.

In Table 8, the data are from the HPLC compositions of the films deposited sequentially from the two-component pre-mix. The HPLC analysis conditions used are: an ODS column was used with acetonitrile as the eluent, and the detection wavelength was 272 nm.

TABLE 8
Composition proportion of the mixture C-MX4 in the films

| Film | A-78 | B-1 |
|---|---|---|
| Film 1 | 57.823 | 42.177 |
| Film 2 | 56.220 | 43.780 |
| Film 3 | 55.883 | 44.117 |
| Film 4 | 55.789 | 44.211 |
| Film 5 | 55.894 | 44.106 |
| Film 6 | 55.811 | 44.189 |

Discussion: As can be seen from the data in Tables 3 to 8 and Table 14, in the mixture of the present disclosure comprising three or more different hole and electron transport-type compounds, a hole transport-type compound having a structure formed by fusing at least four 5- and/or 6-membered rings was specifically selected and cooperated with two or more of the other compounds, thereby achieving the desired stable single-source co-evaporation. In particular, the inventors were also surprised to find that the mixture of the present disclosure could be formed by selecting and mixing hole and electron transport-type compounds having specific heavy and light evaporation property relationships, thereby further improving the evaporation stability of the mixture.

Specifically, as can be seen from Tables 5 to 8, when a two-component comparative mixture was evaporated to form films, the proportions of components in each film generally changed regularly. For example, according to the data of the films in Table 5, the mass ratio of A-3 in the films 1 to 6 gradually increased, that is, A-3 was heavier than B-1; similarly, A-79 was heavier than B-1, and A-4 and A-78 were both lighter than B-1.

The stability of the mass ratio of a certain component in a film was calculated according to the data in Tables 3 to 8 and Table 14, wherein Cₘ denotes the mass ratio of the compound in an m^{th} film, C₀ denotes the mass ratio of the compound in the pre-mix, |Cₘ−C₀| denotes the absolute value of the difference between the mass ratio Cₘ of the compound in any of n films and C₀, m is an integer selected from 1 to n, and |Cₘ−C₀|ₘₐₓ denotes the maximum value of the absolute value of the difference between the mass ratio Cₘ of the compound in any one of the n films and C₀. Taking B-1 as an example, the stability data are as follows:

TABLE 13
Film stability data

| Mixture ID | \|Cₘ − C₀\|ₘₐₓ |
|---|---|
| C-MX1 | 2.921% |
| C-MX2 | 2.177% |
| C-MX3 | 4.854% |
| C-MX4 | 4.211% |
| MX1 | 0.904% |
| MX2 | 0.831% |
| MX3 | 0.825% |

Taking MX1 as an example, the calculation of the above data is as follows: as can be seen from Mixture Example 1, C₀ of Compound B-1 was 50%, n=6, and m was selected from 1, 2, 3, 4, 5 and 6; |C₁−C₀|=0.803%, |C₂−C₀|=0.862%, |C₃−C₀|=0.875%, |C₄−C₀|=0.829%, |C₅−C₀|=0.904%, and |C₆−C₀|=0.871%; as can be determined from the above data, |Cₘ−C₀|ₘₐₓ=0.904%.

In Mixture Example 1, the mixture MX1 of the present disclosure was prepared by selecting and pre-mixing the electron transport-type compound B-1, the hole transport-type compound A-79 which was heavier than B-1 and the hole transport-type compound A-78 which was lighter than B-1. As can be seen from the data in Table 3, the proportions of components in the films prepared by the mixture MX1 were almost unchanged, and the absolute value |Cₙ−C₀| of the difference between the mass ratio of B-1 in any of the films and the mass ratio of B-1 in the pre-mix was less than or equal to 2%. Similarly, as can be seen from the data in Table 4, the proportions of the components in the films prepared by the mixture MX2 of the present disclosure fluctuated slightly. The above data proves that the mixture of the present disclosure has higher stability when evaporated as a single evaporation source, and when applied to OLED devices, can simplify the production process and reduce the production cost.

In addition, the mixture of the present disclosure, when used to prepare devices, can simplify the process and provide excellent comprehensive device performance. In order to support the above viewpoints of the inventors, the following device examples of OLED devices prepared from the pre-mix of the present disclosure are provided.

### Device Example 2-1

First, a glass substrate having an indium tin oxide (ITO) anode having a thickness of 120 nm was cleaned and then treated with UV ozone and oxygen plasma. After the treatment, the substrate was dried in a nitrogen-filled glovebox to remove moisture, loaded on a substrate support and transferred into a vacuum chamber. Organic layers specified below were sequentially evaporated by vacuum thermal evaporation on the ITO anode at a rate of 0.01 to 5 Å/s and a vacuum degree of about 10⁻⁶ torr. Compound HT and Compound HI were co-evaporated as a hole injection layer (HIL) with a thickness of 100 Å. Compound HT was evaporated as a hole transport layer (HTL) with a thickness of 400 Å. Compound EB was evaporated as an electron blocking layer (EBL) with a thickness of 50 Å. Then, the mixture of the present disclosure which served as the host and which was formed by pre-mixing Compound A-78, Compound A-79 and Compound B-1 (the mass ratio among Compound A-78, Compound A-79 and Compound B-1 in the mixture was 9.8:39.2:49) was loaded into one evaporation source and co-evaporated with Compound RD-1 serving as a dopant in another evaporation source as an emissive layer (EML) with a thickness 400 Å. Compound HB was evaporated as a hole blocking layer (HBL) with a thickness of 50 Å. On the hole blocking layer, Compound ET and 8-hydroxyquinolinolato-lithium (Liq) were co-evaporate as an electron transport layer (ETL) with a thickness of 350 Å. Finally, 8-hydroxyquinolinolato-lithium (Liq) with a thickness of 10 Å was evaporated as an electron injection layer (EIL), and Al with a thickness of 1200 Å was evaporated as a cathode. The device was transferred back to the glovebox and encapsulated with a glass lid such that the device was completed.

### Device Example 2-2

The implementation in Device Example 2-2 was the same as the implementation in Device Example 2-1, except that Compound RD-1 was substituted with Compound RD-2 as the dopant in the emissive layer (EML).

### Device Example 2-3

The implementation in Device Example 2-3 was the same as the implementation in Device Example 2-1, except that Compound RD-1 was substituted with Compound RD-3 as the dopant in the emissive layer (EML).

### Device Example 2-4

The implementation in Device Example 2-4 was the same as the implementation in Device Example 2-1, except that Compound RD-1 was substituted with Compound RD-4 as the dopant in the emissive layer (EML).

### Device Example 2-5

The implementation in Device Example 2-5 was the same as the implementation in Device Example 2-4, except that the mixture of the present disclosure formed by pre-mixing Compound A-3, Compound A-4 and Compound B-1 was substituted for the pre-mix of Compound A-78, Compound A-79 and Compound B-1 as the host in the emissive layer (EML), loaded into one evaporation source and co-evaporated with Compound RD-4 serving as a dopant in another evaporation source as an emissive layer (EML), wherein the mass ratio among Compound A-3, Compound A-4 and Compound B-1 in the pre-mixed mixture was 9.8:39.2:49.

### Device Example 2-6

The implementation in Device Example 2-6 was the same as the implementation in Device Example 2-1, except that Compound RD-1 was substituted with Compound RD-5 as the dopant in the emissive layer (EML).

### Device Example 2-7

The implementation in Device Example 2-7 was the same as the implementation in Device Example 2-6, except that the mixture of the present disclosure formed by pre-mixing Compound A-86, Compound A-107 and Compound B-1 was substituted for the pre-mix of Compound A-78, Compound A-79 and Compound B-1 as the host in the emissive layer (EML), loaded into one evaporation source and co-evaporated with Compound RD-5 serving as a dopant in another evaporation source as an emissive layer (EML), wherein the mass ratio among Compound A-107, Compound A-86 and Compound B-1 in the pre-mixed mixture was 9.8:39.2:49.

Detailed structures and thicknesses of layers of the devices are shown in the following table. A layer using more than one material was obtained by doping different compounds at their mass ratio as recorded.

TABLE 9
Device structures in Device Examples 2-1 to 2-7

| Device | | | | | | |
|---|---|---|---|---|---|---|
| ID | HIL | HTL | EBL | EML | HBL | ETL |
| Example | Compound | Compound | Compound | (Compound A-78 + | Compound | Compound |
| 2-1 | HT:Compound | HT | EB | Compound A-79 + | HB | ET:Liq |
| | HI (97:3) | (400 Å) | (50 Å) | Compound | (50 Å) | (40:60) |
| | (100 Å) | | | B-1):Compound RD-1 | | (350 Å) |
| | | | | (98:2) (400 Å) | | |
| Example | Compound | Compound | Compound | (Compound A-78 + | Compound | Compound |
| 2-2 | HT:Compound | HT | EB | Compound A-79 + | HB | ET:Liq |
| | HI (97:3) | (400 Å) | (50 Å) | Compound | (50 Å) | (40:60) |
| | (100 Å) | | | B-1):Compound RD-2 | | (350 Å) |
| | | | | (98:2) (400 Å) | | |
| Example | Compound | Compound | Compound | (Compound | Compound | Compound |
| 2-3 | HT:Compound | HT | EB | A-78+Compound | HB | ET:Liq |
| | HI (97:3) | (400 Å) | (50 Å) | A-79+Compound | (50 Å) | (40:60) |
| | (100 Å) | | | B-1):Compound RD-3 | | (350 Å) |
| | | | | (98:2) (400 Å) | | |
| Example | Compound | Compound | Compound | (Compound A-78 + | Compound | Compound |
| 2-4 | HT:Compound | HT | EB | Compound A-79 + | HB | ET:Liq |
| | HI (97:3) | (400 Å) | (50 Å) | Compound | (50 Å) | (40:60) |
| | (100 Å) | | | B-1):Compound RD-4 | | (350 Å |
| | | | | (98:2) (400 Å) | | |
| Example | Compound | Compound | Compound | (Compound A-3 + | Compound | Compound |
| 2-5 | HT:Compound | HT | EB | Compound A-4 + | HB | ET:Liq |
| | HI (97:3) | (400 Å) | (50 Å) | Compound | (50 Å) | (40:60) |
| | (100 Å) | | | B-1):Compound RD-4 | | (350 Å) |
| | | | | (98:2) (400 Å) | | |
| Example | Compound | Compound | Compound | (Compound A-78 + | Compound | Compound |
| 2-6 | HT:Compound | HT | EB | Compound A-79 + | HB | ET:Liq |
| | HI (97:3) | (400 Å) | (50 Å) | Compound | (50 Å) | (40:60) |
| | (100 Å) | | | B-1):Compound RD-5 | | (350 Å) |
| | | | | (98:2) (400 Å) | | |
| Example | Compound | Compound | Compound | (Compound A-86 + | Compound | Compound |
| 2-7 | HT:Compound | HT | EB | Compound A-107 + | HB | ET:Liq |
| | HI (97:3) | (400 Å) | (50 Å) | Compound | (50 Å) | (40:60) |
| | (100 Å) | | | B-1):Compound RD-5 | | (350 Å) |
| | | | | (98:2) (400 Å) | | |

The new materials used in the devices are as follows:

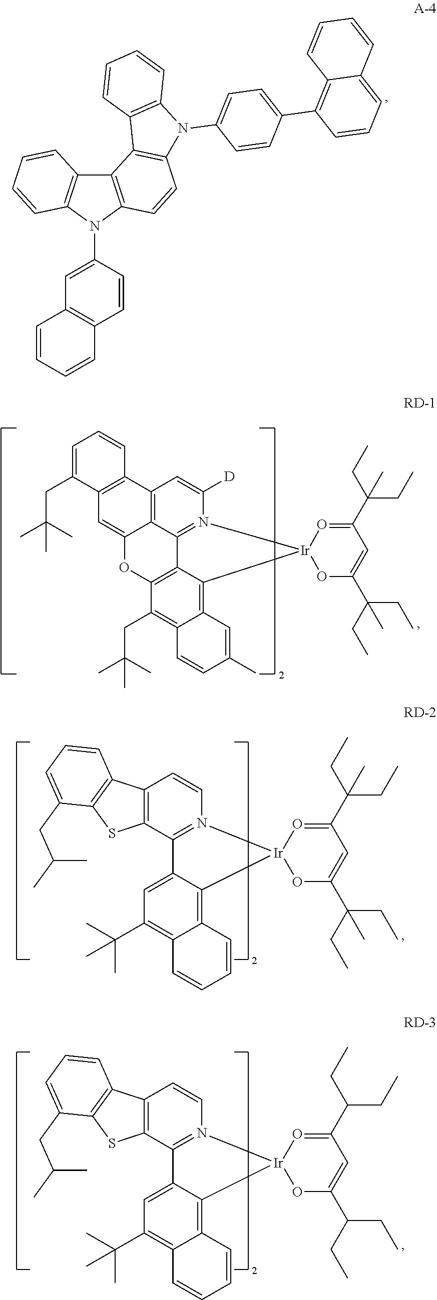

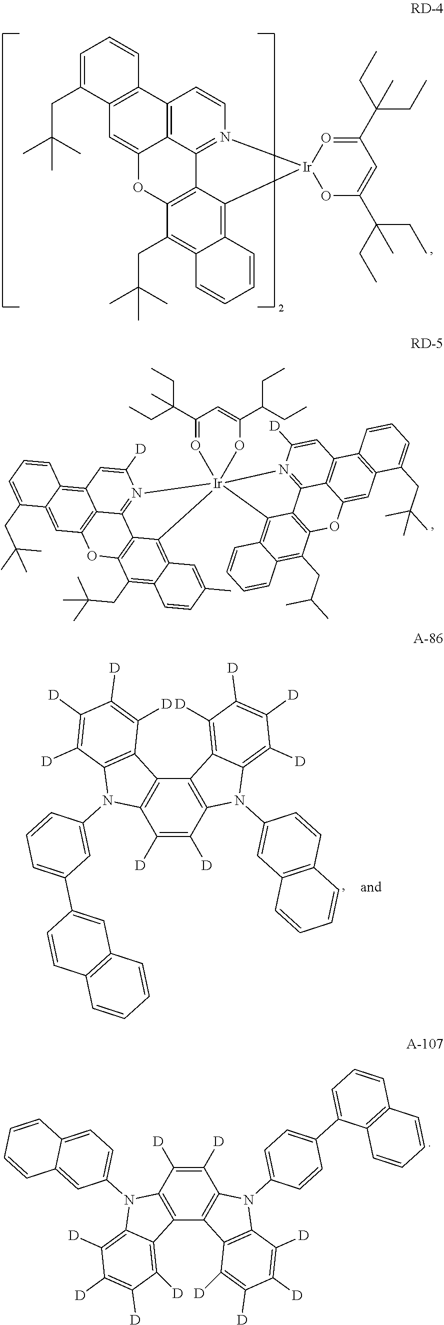

The maximum emission wavelength (λₘₐₓ), current efficiency (CE) and external quantum efficiency (EQE) of the device examples and device comparative examples measured at a constant current of 15 mA/cm² as well as the device lifetime (LT97) measured at a constant current of 80 mA/cm² were shown in Table 10, wherein the device lifetime (LT97) refers to the time for the device to decay to 97% of its initial brightness.

TABLE 10
Device data in Device Examples 2-1 to 2-7

| Device ID | λₘₐₓ (nm) | CE [cd/A] | EQE [%] | LT97 (h) |
|---|---|---|---|---|
| Example 2-1 | 623 | 27.3 | 26.4 | 116 |
| Example 2-2 | 623 | 26.8 | 26.4 | 79 |
| Example 2-3 | 623 | 27.8 | 26.4 | 142 |
| Example 2-4 | 620 | 29.4 | 26.5 | 61 |
| Example 2-5 | 620 | 28.8 | 26.1 | 62 |
| Example 2-6 | 621 | 27.8 | 25.9 | 126 |
| Example 2-7 | 621 | 27.9 | 25.9 | 135 |

The above devices were electroluminescent devices prepared by the emissive layers formed by co-evaporating a mixture as a single evaporation source and an emissive material in another evaporation source. For example, the emissive layer of Device Example 2-1 was formed by co-evaporating a mixture of the present disclosure (formed by pre-mixing Compound A-78, Compound A-79 and Compound B-1) as a single evaporation source and Compound RD-1.

The data in Table 10 show that Device Examples 2-1 to 2-7 all had high external quantum efficiency (EQE), the device lifetime reached a relatively high level, and all of the devices showed excellent comprehensive device performance. As can be seen, when the mixture of the present disclosure was used as a single evaporation source in the preparation process of OLED devices, the production process can be simplified, and the production cost can be reduced; at the same time, the devices in the Device Examples 2-1 to 2-7 in which the mixture of the present disclosure was used as the host material obtained excellent performance with high lifetime and efficiency.

The above data show that for the new mixture provided by the present disclosure comprising three electron and hole transport-type materials with different structures, the mixture is provided with high evaporation stability by selecting a specific hole transport material having a fused structure (not triphenylene) formed by fusing at least four 5- and/or 6-membered rings and, preferably, by using a hole transport material having an indolocarbazole structure. Such new mixtures, when cooperating with different phosphorescent dopant materials, can provide excellent device performance and have broad application prospects.

It is to be understood that various embodiments described herein are merely illustrative and not intended to limit the scope of the present disclosure. Therefore, it is apparent to the persons skilled in the art that the present disclosure as claimed may include variations of specific embodiments and preferred embodiments described herein. Many of the materials and structures described herein may be replaced with other materials and structures without departing from the spirit of the present disclosure. It is to be understood that various theories as to why the present disclosure works are not intended to be limiting.

What is claimed is:

1. A mixture, comprising at least a first compound, a second compound and a third compound;
wherein the first compound is a hole transport-type compound; the second compound is an electron transport-type compound; the third compound is selected from a hole transport-type compound or an electron transport-type compound;
the hole transport-type compound comprises a fused structure formed by fusing at least four 5- and/or 6-membered rings, wherein the fused structure is not triphenylene;
the first compound, the second compound and the third compound are different from each other in chemical structure;
the mixture comprises a pre-mix formed by pre-mixing the first compound, the second compound and the third compound, a mass ratio of at least one compound among the first compound, the second compound and the third compound in the mixture is C₀, and when the mixture is evaporated at a vacuum degree of 10⁻⁶ torr or below and a rate of 0.01-5 Å/s, the mixture is evaporated on a surface positioned at a certain distance from the mixture to be evaporated to form n films with a certain thickness, wherein a mass ratio of the compound in an n^{th} film is Cₙ, and n is an integer greater than or equal to 1;
wherein an absolute value |Cₘ−C₀| of a difference between a mass ratio Cₘ of the compound in any one of the n evaporated films and C₀ is less than or equal to 2%, and m is an integer selected from 1 to n.

2. The mixture of claim 1, wherein the hole transport-type compound comprises at least one heteroatom, and the heteroatom is selected from the group consisting of: a nitrogen atom, an oxygen atom, a sulfur atom, a selenium atom, a phosphorus atom, a silicon atom, a germanium atom, a boron atom, and combinations thereof; preferably, the heteroatom is selected from the group consisting of: a nitrogen atom, an oxygen atom, a sulfur atom, a selenium atom, and combinations thereof; preferably, the heteroatom is selected from the group consisting of: a nitrogen atom, an oxygen atom, a sulfur atom, and combinations thereof; more preferably, the heteroatom is selected from a nitrogen atom.

3. The mixture of claim 1, wherein when the third compound is selected from a hole transport-type compound, in evaporation characteristics, the third compound is heavier than the second compound, and the first compound is lighter than the second compound;
when the third compound is selected from an electron transport-type compound, in evaporation characteristics, the second compound is heavier than the first compound, and the third compound is lighter than the first compound.

4. The mixture of claim 1, wherein the mass ratio of the at least one compound among the first compound, the second compound and the third compound in the mixture is C₀, and when the mixture is evaporated at a vacuum degree of 10⁻⁶ torr or below and a rate of 0.01-5 Å/s, the mixture is evaporated on a surface positioned at a certain distance from the mixture to be evaporated to form n films with a certain thickness, wherein the mass ratio of the compound in the n^{th} film is Cₙ; n is an integer greater than or equal to 1; wherein the absolute value |Cₘ−C₀| of the difference between the mass ratio Cₘ of the compound in any one of the n evaporated films and C₀ is less than or equal to 1.5%; preferably, |Cₘ−C₀| is less than or equal to 1.0%; more preferably, |Cₘ−C₀| is less than or equal to 0.5%.

5. The mixture of claim 1, wherein the evaporation temperature of the first compound, the second compound and the third compound are 120° C.-390° C.; preferably, the evaporation temperature of the first compound, the second compound and the third compound are 140° C.-370° C.; more preferably, the evaporation temperature of the first compound, the second compound and the third compound are 160° C.-360° C.; most preferably, the evaporation temperature of the first compound, the second compound and the third compound are 200° C.-350° C.

6. The mixture of claim 1, wherein when the first compound, the second compound and the third compound are evaporated at a vacuum degree of 10⁻⁶ torr or below and a rate of 0.01-5 Å/s, an absolute value of an evaporation temperature difference between any two of the first compound, the second compound and the third compound is less than 30° C.; preferably, the absolute value of the evaporation temperature difference between any two of the first compound, the second compound and the third compound is less than 20° C.; preferably, the absolute value of the evaporation temperature difference between any two of the first compound, the second compound and the third compound is less than 10° C.

7. The mixture of claim 1, wherein a triplet energy level T₁ of at least one of the first compound, the second compound or the third compound is less than 2.65 eV; preferably, T₁ is less than 2.60 eV.

8. The mixture of claim 1, wherein the hole transport-type compound and/or the electron transport-type compound comprise at least one chemical group selected from the group consisting of: indolocarbazole, benzene, pyridine, pyrimidine, carbazole, azacarbazole, dibenzothiophene, azadibenzothiophe, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene, oxazole, thiazole, and combinations thereof;
preferably, the hole transport-type compound comprises an indolo[2,3-C]carbazolyl group, and the electron transport-type compound comprises a triazinyl group.

9. The mixture of claim 1, wherein the first compound has a structure represented by Formula 1′: 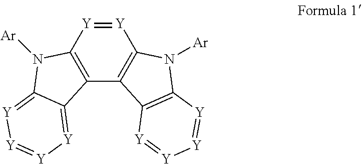
wherein
Ar is selected from substituted or unsubstituted aryl having 6 to 30 carbon atoms, substituted or unsubstituted heteroaryl having 3 to 30 carbon atoms or a combination thereof;
Y is, at each occurrence identically or differently, selected from CR_{y} or N;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{y} can be optionally joined to form a ring

10. The mixture of claim 1, wherein the second compound has a structure represented by Formula 2′: 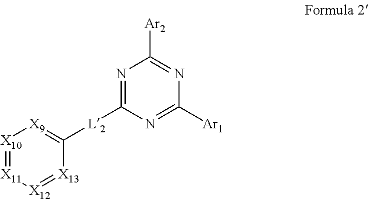
wherein
X₉ to X₁₃ are, at each occurrence identically or differently, selected from CRₓ or N;
L′₂ is selected from a single bond, substituted or unsubstituted arylene having 6 to 30 carbon atoms, substituted or unsubstituted heteroarylene having 3 to 30 carbon atoms or a combination thereof;
Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Ar₁ and Ar₂ are selected from substituted or unsubstituted aryl having 6 to 30 carbon atoms, substituted or unsubstituted heteroaryl having 3 to 30 carbon atoms or a combination thereof;
preferably, at least one of Ar₁ or Ar₂ is selected from a group comprising substituted or unsubstituted dibenzofuryl, substituted or unsubstituted dibenzothienyl, substituted or unsubstituted carbazolyl, substituted or unsubstituted silafluorenyl, substituted or unsubstituted fluorenyl, substituted or unsubstituted naphthyl, substituted or unsubstituted chrysenyl or a combination thereof;
adjacent substituents Rₓ can be optionally joined to form a ring.

11. The mixture of claim 10, wherein the third compound has a structure represented by Formula 1′ or Formula 2′:
preferably, the at least two of the first compound, the second compound or the third compound are isomers of each other or isotopologues of each other; the third compound has a structure represented by Formula 1′, and the third compound and the first compound are isomers of each other or isotopologues of each other; or the third compound has a structure represented by Formula 2′, and the third compound and the second compound are isomers of each other or isotopologues of each other.

12. The mixture of claim 1, wherein the first compound is selected from the group consisting of Compound A-1 to Compound A-263, wherein the specific structures of Compound A-1 to Compound A-263 are as follows: 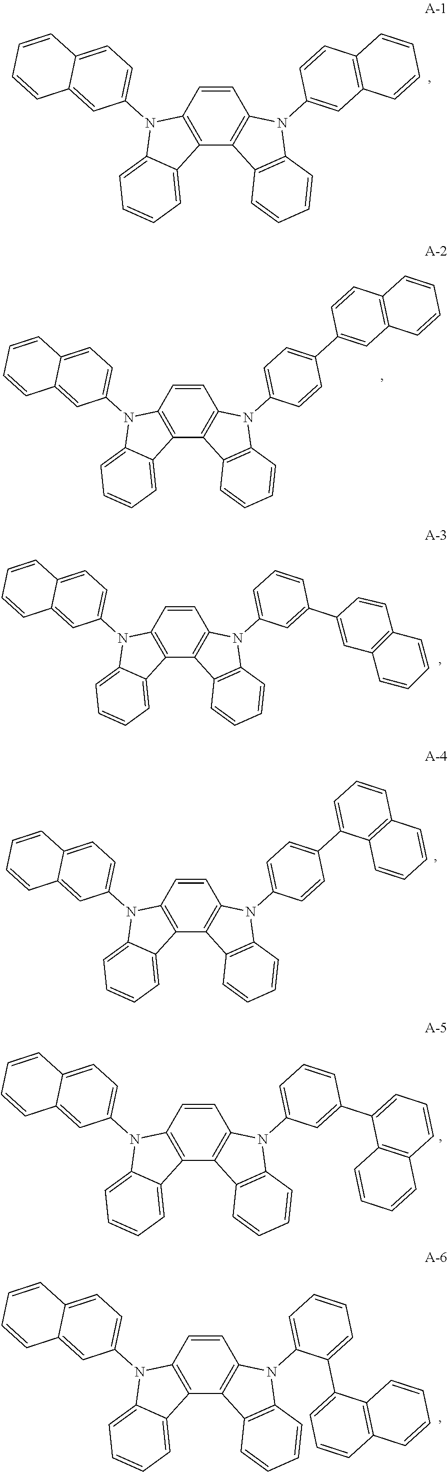 [SMILES: c1ccc2cc(-n3c4ccccc4c4c5c6ccccc6n(-c6ccc7ccccc7c6)c5ccc43)ccc2c1 ; c1ccc2cc(-c3ccc(-n4c5ccccc5c5c6c7ccccc7n(-c7ccc8ccccc8c7)c6ccc54)cc3)ccc2c1 ; c1cc(-c2ccc3ccccc3c2)cc(-n2c3ccccc3c3c4c5ccccc5n(-c5ccc6ccccc6c5)c4ccc32)c1 ; c1ccc2cc(-n3c4ccccc4c4c5c6ccccc6n(-c6ccc(-c7cccc8ccccc78)cc6)c5ccc43)ccc2c1 ; c1cc(-c2cccc3ccccc23)cc(-n2c3ccccc3c3c4c5ccccc5n(-c5ccc6ccccc6c5)c4ccc32)c1 ; c1ccc(-n2c3ccccc3c3c4c5ccccc5n(-c5ccc6ccccc6c5)c4ccc32)c(-c2cccc3ccccc23)c1] 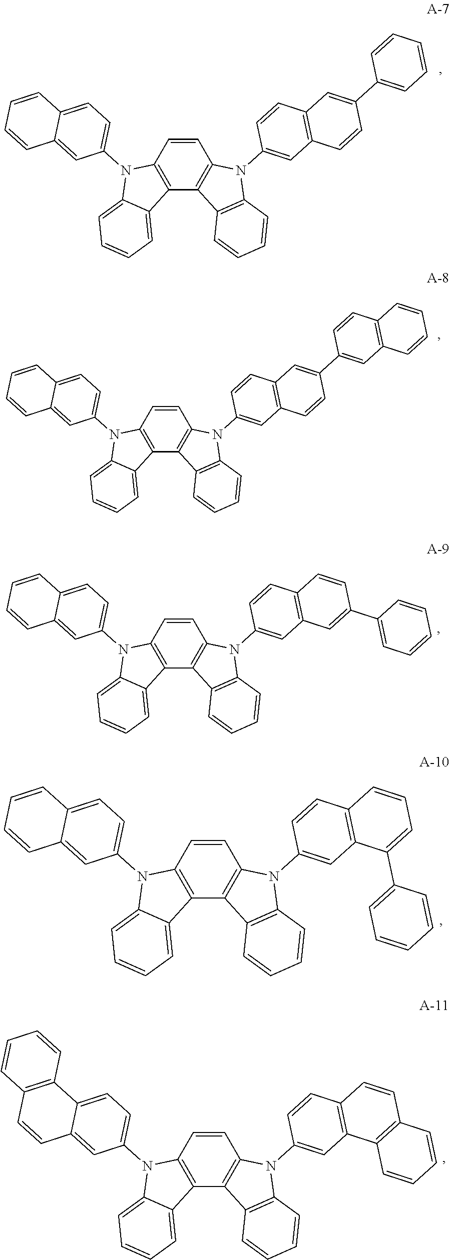 [SMILES: c1ccc(-c2ccc3cc(-n4c5ccccc5c5c6c7ccccc7n(-c7ccc8ccccc8c7)c6ccc54)ccc3c2)cc1 ; c1ccc2cc(-c3ccc4cc(-n5c6ccccc6c6c7c8ccccc8n(-c8ccc9ccccc9c8)c7ccc65)ccc4c3)ccc2c1 ; c1ccc(-c2ccc3ccc(-n4c5ccccc5c5c6c7ccccc7n(-c7ccc8ccccc8c7)c6ccc54)cc3c2)cc1 ; c1ccc(-c2cccc3ccc(-n4c5ccccc5c5c6c7ccccc7n(-c7ccc8ccccc8c7)c6ccc54)cc23)cc1 ; c1ccc2c(c1)ccc1cc(-n3c4ccccc4c4c5c6ccccc6n(-c6ccc7ccc8ccccc8c7c6)c5ccc43)ccc12] 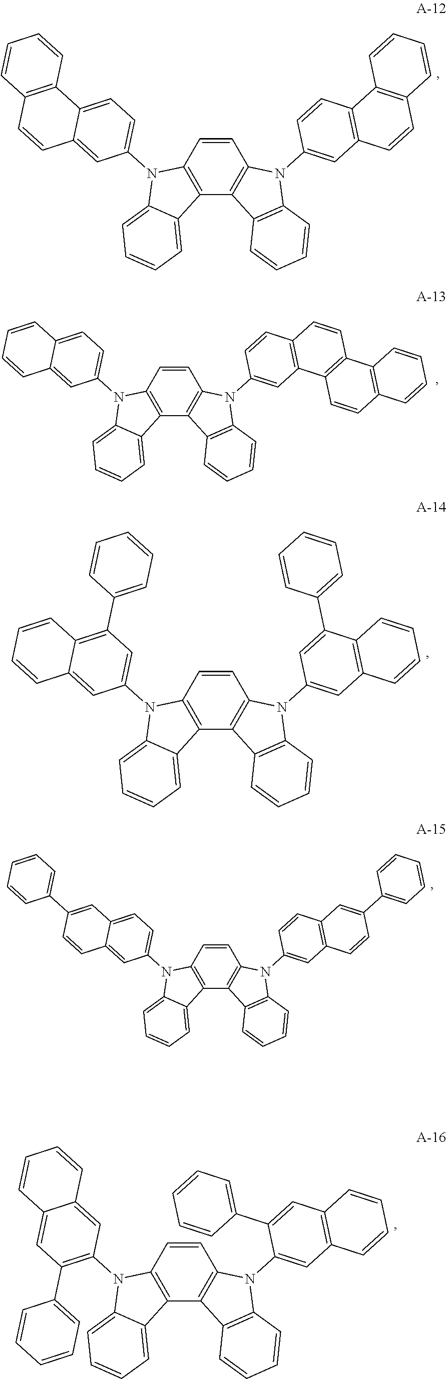 [SMILES: c1ccc2c(c1)ccc1cc(-n3c4ccccc4c4c5c6ccccc6n(-c6ccc7c(ccc8ccccc87)c6)c5ccc43)ccc12 ; c1ccc2cc(-n3c4ccccc4c4c5c6ccccc6n(-c6ccc7ccc8c9ccccc9ccc8c7c6)c5ccc43)ccc2c1 ; c1ccc(-c2cc(-n3c4ccccc4c4c5c6ccccc6n(-c6cc(-c7ccccc7)c7ccccc7c6)c5ccc43)cc3ccccc23)cc1 ; c1ccc(-c2ccc3cc(-n4c5ccccc5c5c6c7ccccc7n(-c7ccc8cc(-c9ccccc9)ccc8c7)c6ccc54)ccc3c2)cc1 ; c1ccc(-c2cc3ccccc3cc2-n2c3ccccc3c3c4c5ccccc5n(-c5cc6ccccc6cc5-c5ccccc5)c4ccc32)cc1] 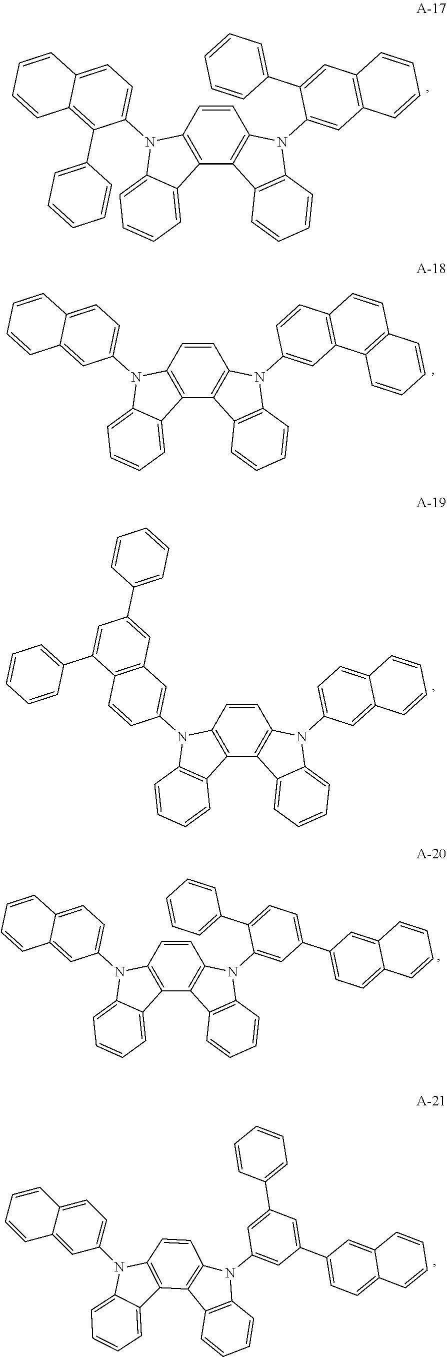 [SMILES: c1ccc(-c2cc3ccccc3cc2-n2c3ccccc3c3c4c5ccccc5n(-c5ccc6ccccc6c5-c5ccccc5)c4ccc32)cc1 ; c1ccc2cc(-n3c4ccccc4c4c5c6ccccc6n(-c6ccc7ccc8ccccc8c7c6)c5ccc43)ccc2c1 ; c1ccc(-c2cc(-c3ccccc3)c3ccc(-n4c5ccccc5c5c6c7ccccc7n(-c7ccc8ccccc8c7)c6ccc54)cc3c2)cc1 ; c1ccc(-c2ccc(-c3ccc4ccccc4c3)cc2-n2c3ccccc3c3c4c5ccccc5n(-c5ccc6ccccc6c5)c4ccc32)cc1 ; c1ccc(-c2cc(-c3ccc4ccccc4c3)cc(-n3c4ccccc4c4c5c6ccccc6n(-c6ccc7ccccc7c6)c5ccc43)c2)cc1] 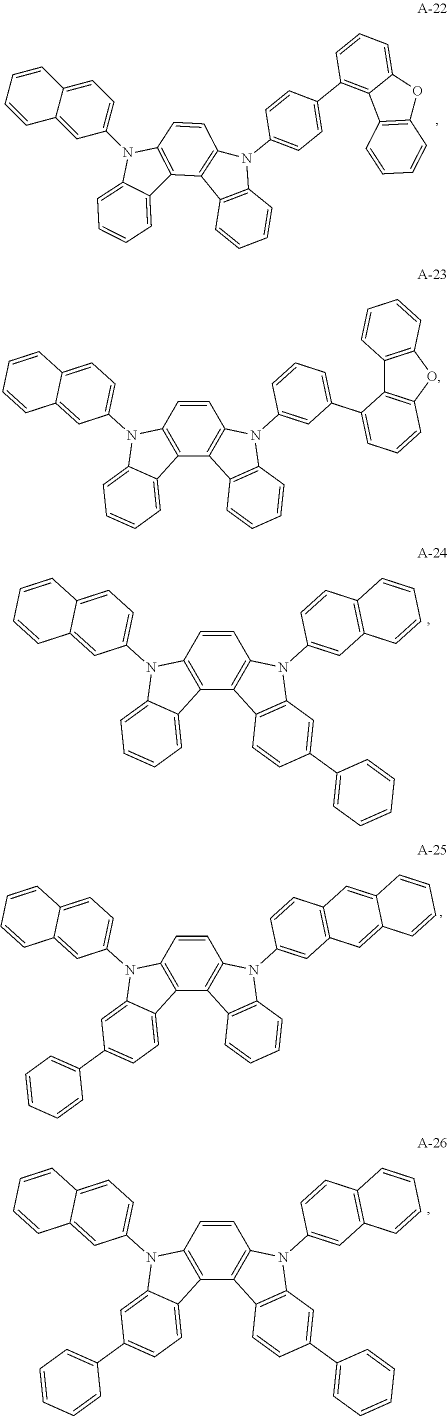 [SMILES: c1ccc2cc(-n3c4ccccc4c4c5c6ccccc6n(-c6ccc(-c7cccc8oc9ccccc9c78)cc6)c5ccc43)ccc2c1 ; c1cc(-c2cccc3oc4ccccc4c23)cc(-n2c3ccccc3c3c4c5ccccc5n(-c5ccc6ccccc6c5)c4ccc32)c1 ; c1ccc(-c2ccc3c4c5c6ccccc6n(-c6ccc7ccccc7c6)c5ccc4n(-c4ccc5ccccc5c4)c3c2)cc1 ; c1ccc(-c2ccc3c4c5c6ccccc6n(-c6ccc7cc8ccccc8cc7c6)c5ccc4n(-c4ccc5ccccc5c4)c3c2)cc1 ; c1ccc(-c2ccc3c4c5c6ccc(-c7ccccc7)cc6n(-c6ccc7ccccc7c6)c5ccc4n(-c4ccc5ccccc5c4)c3c2)cc1] 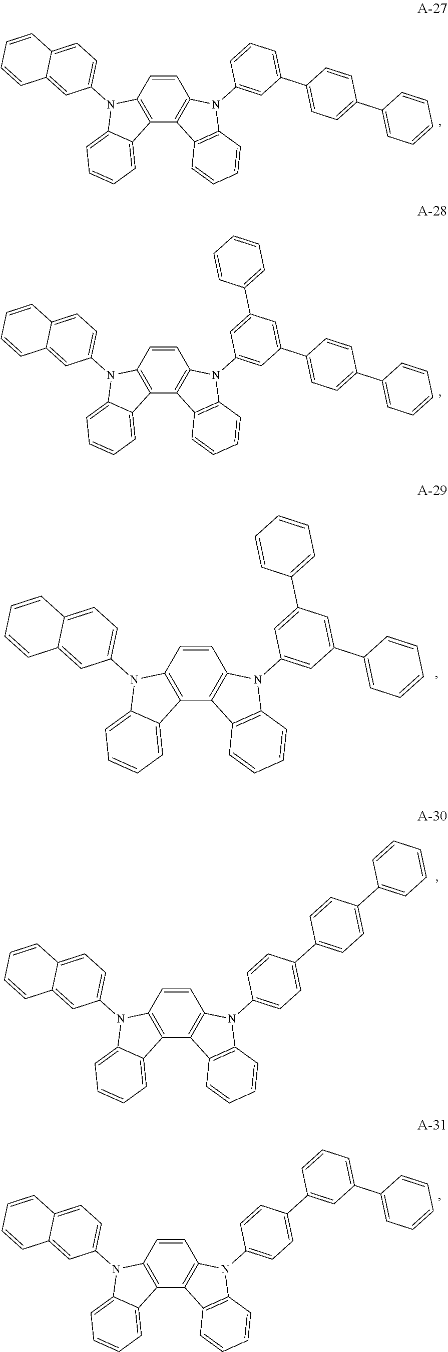 [SMILES: c1ccc(-c2ccc(-c3cccc(-n4c5ccccc5c5c6c7ccccc7n(-c7ccc8ccccc8c7)c6ccc54)c3)cc2)cc1 ; c1ccc(-c2ccc(-c3cc(-c4ccccc4)cc(-n4c5ccccc5c5c6c7ccccc7n(-c7ccc8ccccc8c7)c6ccc54)c3)cc2)cc1 ; c1ccc(-c2cc(-c3ccccc3)cc(-n3c4ccccc4c4c5c6ccccc6n(-c6ccc7ccccc7c6)c5ccc43)c2)cc1 ; c1ccc(-c2ccc(-c3ccc(-n4c5ccccc5c5c6c7ccccc7n(-c7ccc8ccccc8c7)c6ccc54)cc3)cc2)cc1 ; c1ccc(-c2cccc(-c3ccc(-n4c5ccccc5c5c6c7ccccc7n(-c7ccc8ccccc8c7)c6ccc54)cc3)c2)cc1] 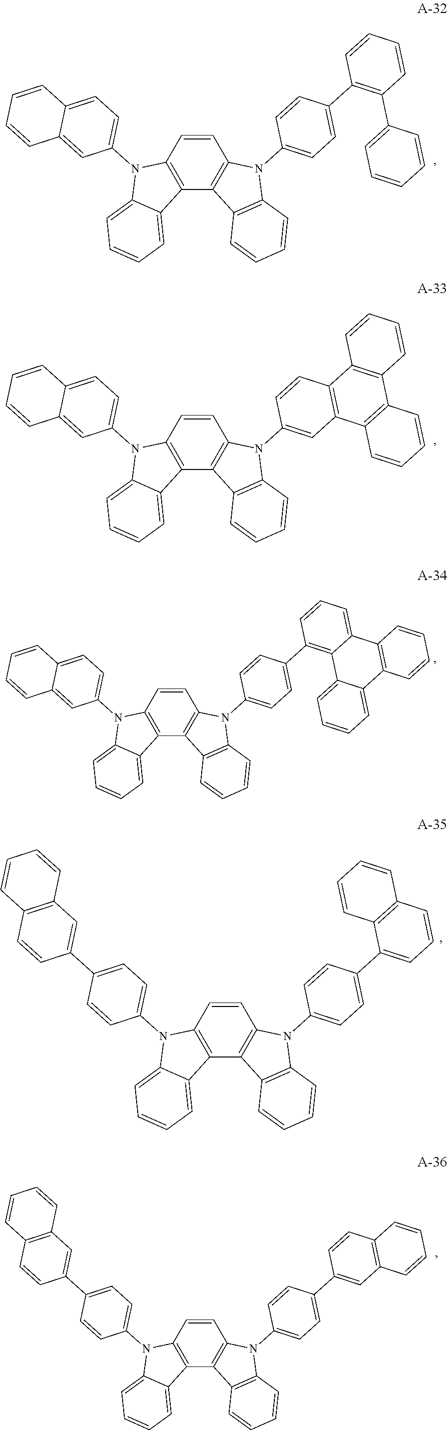 [SMILES: c1ccc(-c2ccccc2-c2ccc(-n3c4ccccc4c4c5c6ccccc6n(-c6ccc7ccccc7c6)c5ccc43)cc2)cc1 ; c1ccc2cc(-n3c4ccccc4c4c5c6ccccc6n(-c6ccc7c8ccccc8c8ccccc8c7c6)c5ccc43)ccc2c1 ; c1ccc2cc(-n3c4ccccc4c4c5c6ccccc6n(-c6ccc(-c7cccc8c9ccccc9c9ccccc9c78)cc6)c5ccc43)ccc2c1 ; c1ccc2cc(-c3ccc(-n4c5ccccc5c5c6c7ccccc7n(-c7ccc(-c8cccc9ccccc89)cc7)c6ccc54)cc3)ccc2c1 ; c1ccc2cc(-c3ccc(-n4c5ccccc5c5c6c7ccccc7n(-c7ccc(-c8ccc9ccccc9c8)cc7)c6ccc54)cc3)ccc2c1] 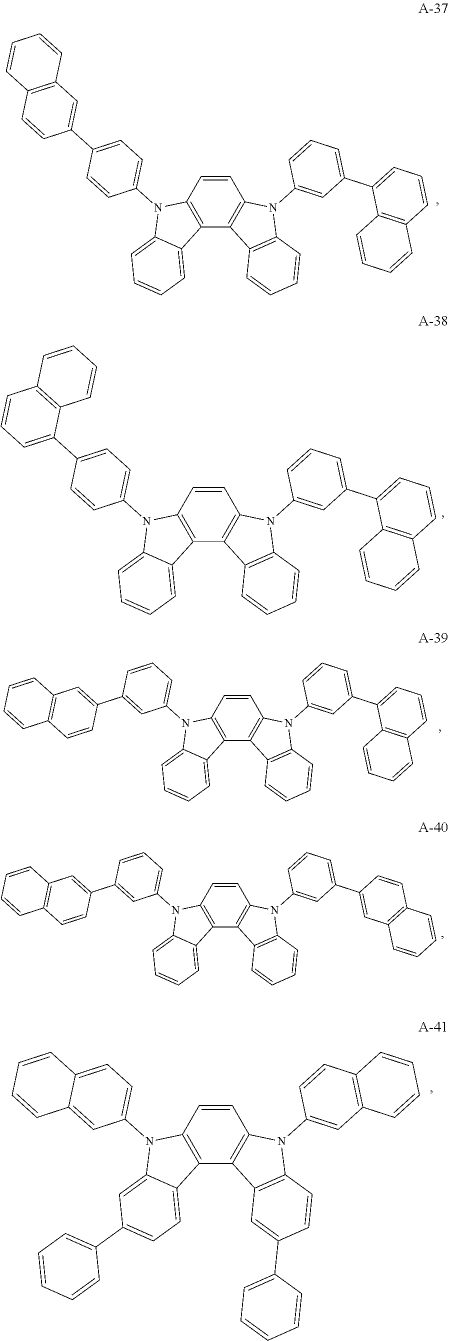 [SMILES: c1cc(-c2cccc3ccccc23)cc(-n2c3ccccc3c3c4c5ccccc5n(-c5ccc(-c6ccc7ccccc7c6)cc5)c4ccc32)c1 ; c1cc(-c2cccc3ccccc23)cc(-n2c3ccccc3c3c4c5ccccc5n(-c5ccc(-c6cccc7ccccc67)cc5)c4ccc32)c1 ; c1cc(-c2ccc3ccccc3c2)cc(-n2c3ccccc3c3c4c5ccccc5n(-c5cccc(-c6cccc7ccccc67)c5)c4ccc32)c1 ; c1cc(-c2ccc3ccccc3c2)cc(-n2c3ccccc3c3c4c5ccccc5n(-c5cccc(-c6ccc7ccccc7c6)c5)c4ccc32)c1 ; c1ccc(-c2ccc3c(c2)c2c4c5ccc(-c6ccccc6)cc5n(-c5ccc6ccccc6c5)c4ccc2n3-c2ccc3ccccc3c2)cc1] 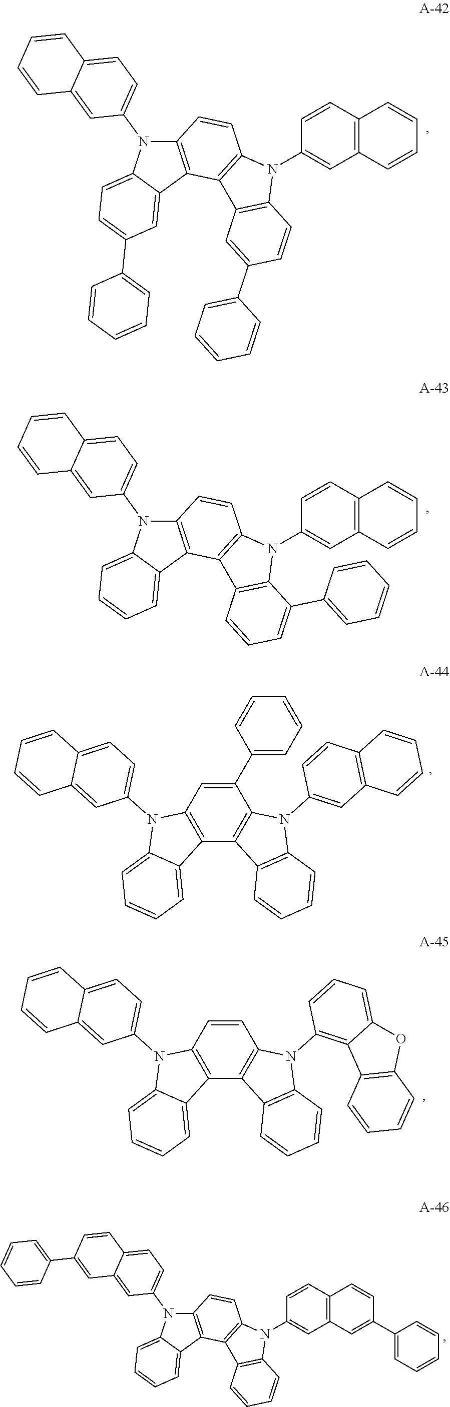 [SMILES: c1ccc(-c2ccc3c(c2)c2c4c5cc(-c6ccccc6)ccc5n(-c5ccc6ccccc6c5)c4ccc2n3-c2ccc3ccccc3c2)cc1 ; c1ccc(-c2cccc3c4c5c6ccccc6n(-c6ccc7ccccc7c6)c5ccc4n(-c4ccc5ccccc5c4)c23)cc1 ; c1ccc(-c2cc3c(c4ccccc4n3-c3ccc4ccccc4c3)c3c4ccccc4n(-c4ccc5ccccc5c4)c23)cc1 ; c1ccc2cc(-n3c4ccccc4c4c5c6ccccc6n(-c6cccc7oc8ccccc8c67)c5ccc43)ccc2c1 ; c1ccc(-c2ccc3ccc(-n4c5ccccc5c5c6c7ccccc7n(-c7ccc8ccc(-c9ccccc9)cc8c7)c6ccc54)cc3c2)cc1] 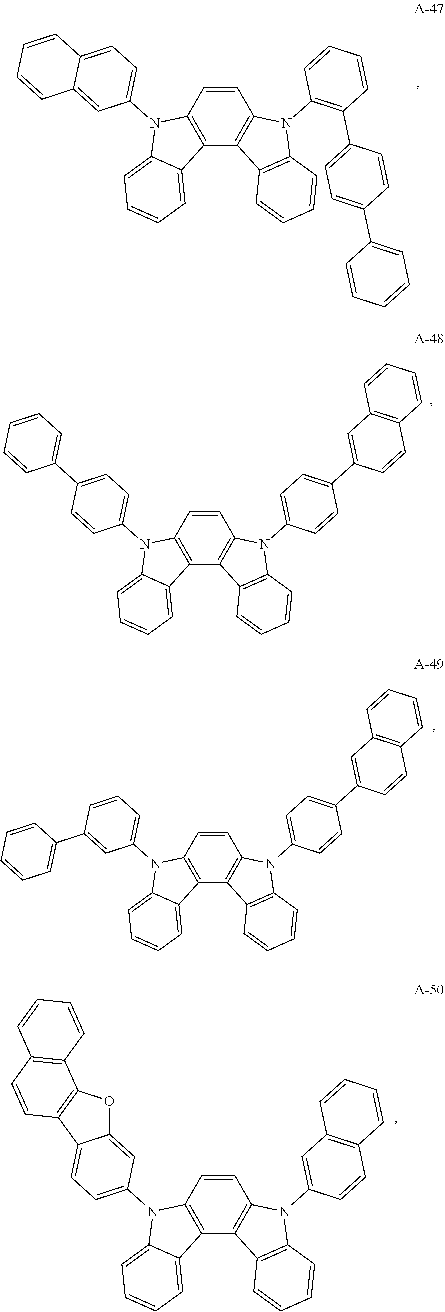 [SMILES: c1ccc(-c2ccc(-c3ccccc3-n3c4ccccc4c4c5c6ccccc6n(-c6ccc7ccccc7c6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2cccc(-n3c4ccccc4c4c5c6ccccc6n(-c6ccc(-c7ccc8ccccc8c7)cc6)c5ccc43)c2)cc1 ; c1ccc2cc(-n3c4ccccc4c4c5c6ccccc6n(-c6ccc7c(c6)oc6c8ccccc8ccc76)c5ccc43)ccc2c1] 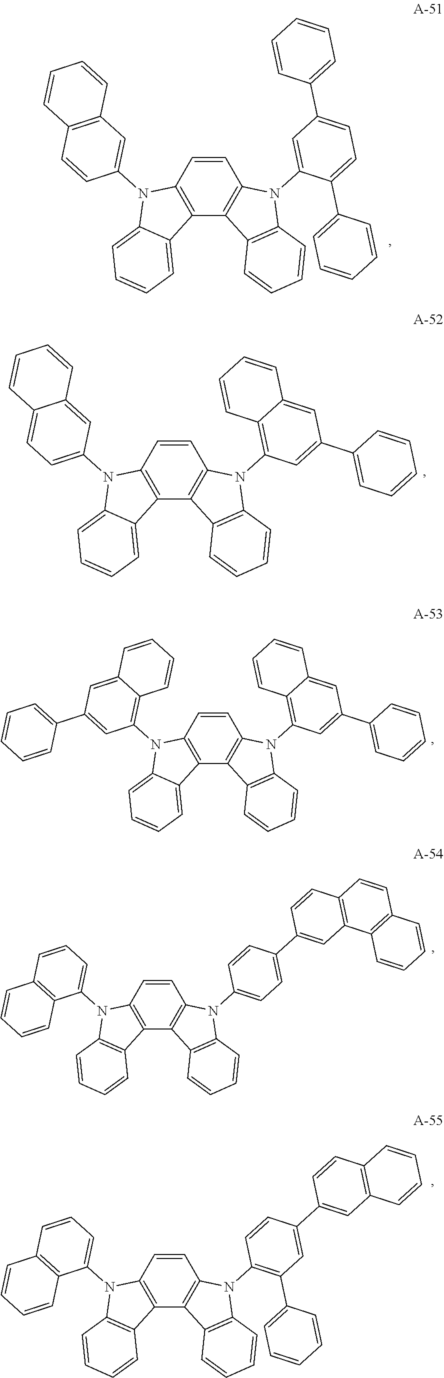 [SMILES: c1ccc(-c2ccc(-c3ccccc3)c(-n3c4ccccc4c4c5c6ccccc6n(-c6ccc7ccccc7c6)c5ccc43)c2)cc1 ; c1ccc(-c2cc(-n3c4ccccc4c4c5c6ccccc6n(-c6ccc7ccccc7c6)c5ccc43)c3ccccc3c2)cc1 ; c1ccc(-c2cc(-n3c4ccccc4c4c5c6ccccc6n(-c6cc(-c7ccccc7)cc7ccccc67)c5ccc43)c3ccccc3c2)cc1 ; c1ccc2c(-n3c4ccccc4c4c5c6ccccc6n(-c6ccc(-c7ccc8ccc9ccccc9c8c7)cc6)c5ccc43)cccc2c1 ; c1ccc(-c2cc(-c3ccc4ccccc4c3)ccc2-n2c3ccccc3c3c4c5ccccc5n(-c5cccc6ccccc56)c4ccc32)cc1] 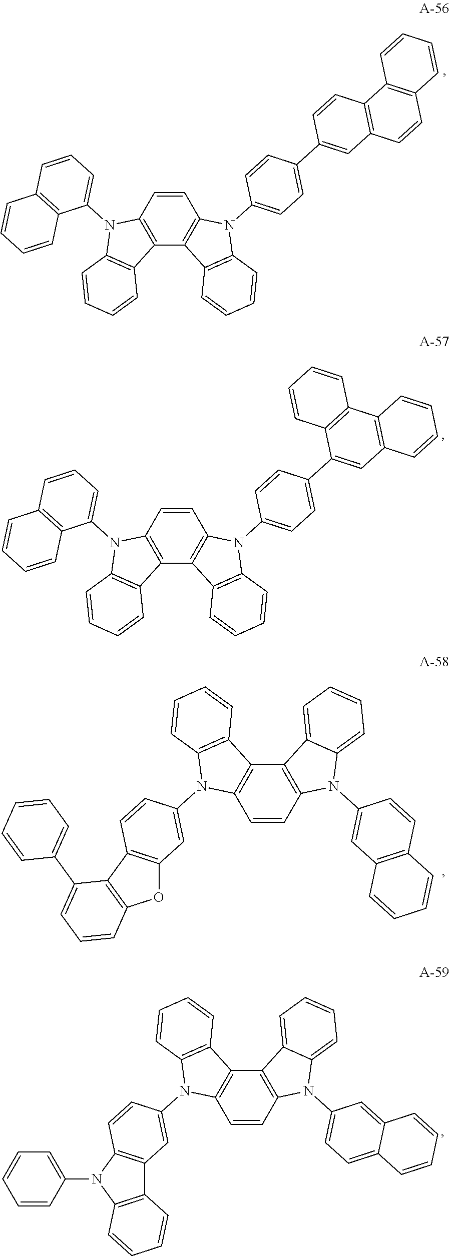 [SMILES: c1ccc2c(-n3c4ccccc4c4c5c6ccccc6n(-c6ccc(-c7ccc8c(ccc9ccccc98)c7)cc6)c5ccc43)cccc2c1 ; c1ccc2c(-n3c4ccccc4c4c5c6ccccc6n(-c6ccc(-c7cc8ccccc8c8ccccc78)cc6)c5ccc43)cccc2c1 ; c1ccc(-c2cccc3oc4cc(-n5c6ccccc6c6c7c8ccccc8n(-c8ccc9ccccc9c8)c7ccc65)ccc4c23)cc1 ; c1ccc(-n2c3ccccc3c3cc(-n4c5ccccc5c5c6c7ccccc7n(-c7ccc8ccccc8c7)c6ccc54)ccc32)cc1] 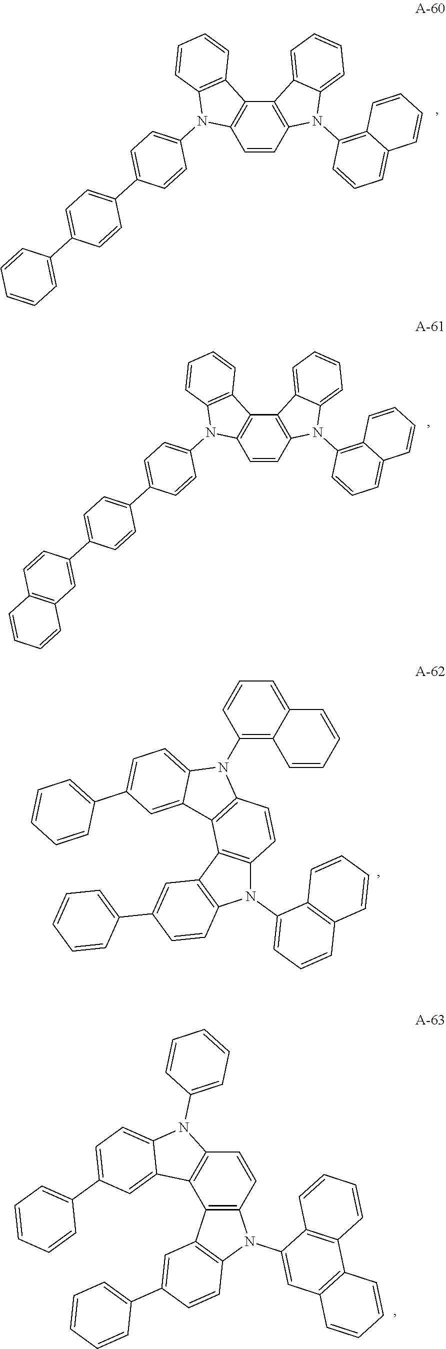 [SMILES: c1ccc(-c2ccc(-c3ccc(-n4c5ccccc5c5c6c7ccccc7n(-c7cccc8ccccc78)c6ccc54)cc3)cc2)cc1 ; c1ccc2cc(-c3ccc(-c4ccc(-n5c6ccccc6c6c7c8ccccc8n(-c8cccc9ccccc89)c7ccc65)cc4)cc3)ccc2c1 ; c1ccc(-c2ccc3c(c2)c2c4c5cc(-c6ccccc6)ccc5n(-c5cccc6ccccc56)c4ccc2n3-c2cccc3ccccc23)cc1 ; c1ccc(-c2ccc3c(c2)c2c4c5cc(-c6ccccc6)ccc5n(-c5cc6ccccc6c6ccccc56)c4ccc2n3-c2ccccc2)cc1] 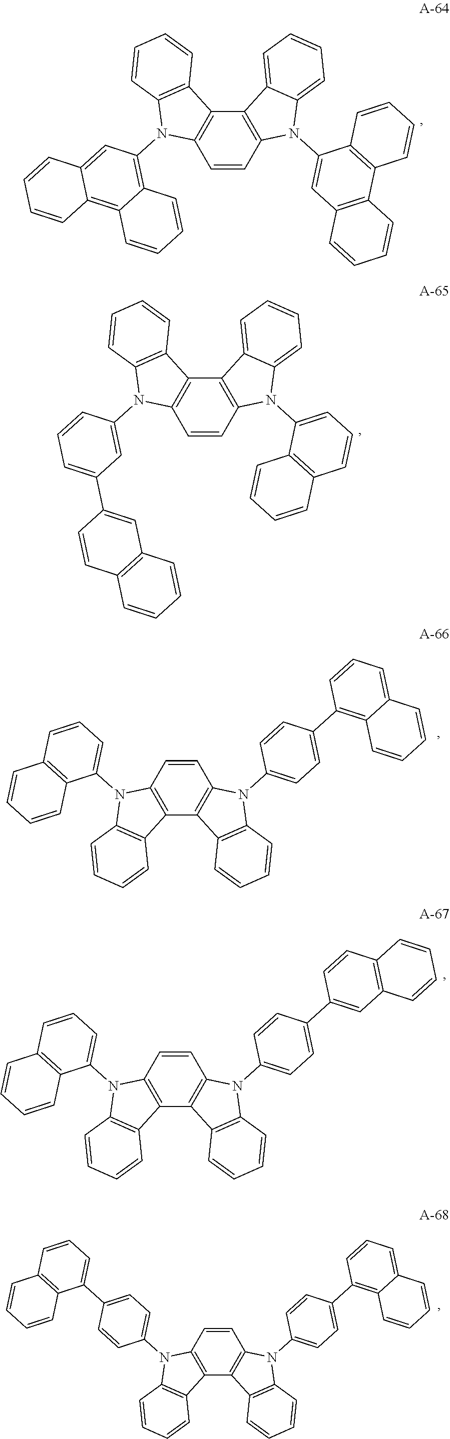 [SMILES: c1ccc2c(c1)cc(-n1c3ccccc3c3c4c5ccccc5n(-c5cc6ccccc6c6ccccc56)c4ccc31)c1ccccc12 ; c1cc(-c2ccc3ccccc3c2)cc(-n2c3ccccc3c3c4c5ccccc5n(-c5cccc6ccccc56)c4ccc32)c1 ; c1ccc2c(-c3ccc(-n4c5ccccc5c5c6c7ccccc7n(-c7cccc8ccccc78)c6ccc54)cc3)cccc2c1 ; c1ccc2cc(-c3ccc(-n4c5ccccc5c5c6c7ccccc7n(-c7cccc8ccccc78)c6ccc54)cc3)ccc2c1 ; c1ccc2c(-c3ccc(-n4c5ccccc5c5c6c7ccccc7n(-c7ccc(-c8cccc9ccccc89)cc7)c6ccc54)cc3)cccc2c1] 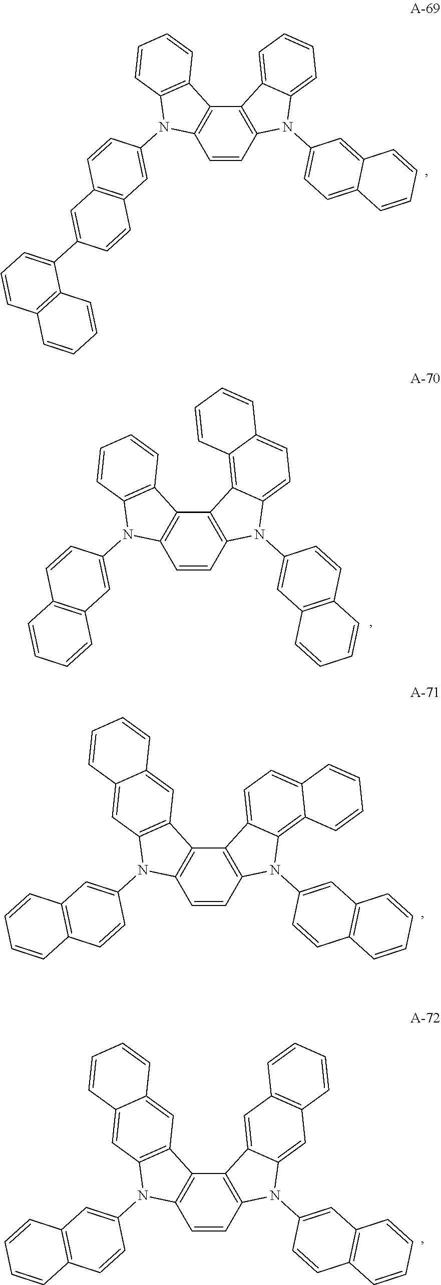 [SMILES: c1ccc2cc(-n3c4ccccc4c4c5c6ccccc6n(-c6ccc7cc(-c8cccc9ccccc89)ccc7c6)c5ccc43)ccc2c1 ; c1ccc2cc(-n3c4ccccc4c4c5c6c7ccccc7ccc6n(-c6ccc7ccccc7c6)c5ccc43)ccc2c1 ; c1ccc2cc(-n3c4cc5ccccc5cc4c4c5c6ccc7ccccc7c6n(-c6ccc7ccccc7c6)c5ccc43)ccc2c1 ; c1ccc2cc(-n3c4cc5ccccc5cc4c4c5c6cc7ccccc7cc6n(-c6ccc7ccccc7c6)c5ccc43)ccc2c1] 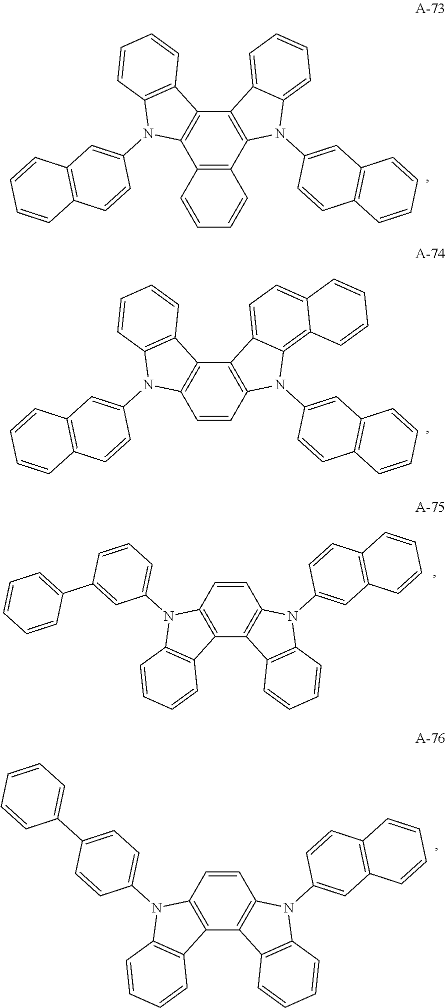 [SMILES: c1ccc2cc(-n3c4ccccc4c4c5c6ccccc6n(-c6ccc7ccccc7c6)c5c5ccccc5c43)ccc2c1 ; c1ccc2cc(-n3c4ccccc4c4c5c6ccc7ccccc7c6n(-c6ccc7ccccc7c6)c5ccc43)ccc2c1 ; c1ccc(-c2cccc(-n3c4ccccc4c4c5c6ccccc6n(-c6ccc7ccccc7c6)c5ccc43)c2)cc1 ; c1ccc(-c2ccc(-n3c4ccccc4c4c5c6ccccc6n(-c6ccc7ccccc7c6)c5ccc43)cc2)cc1] 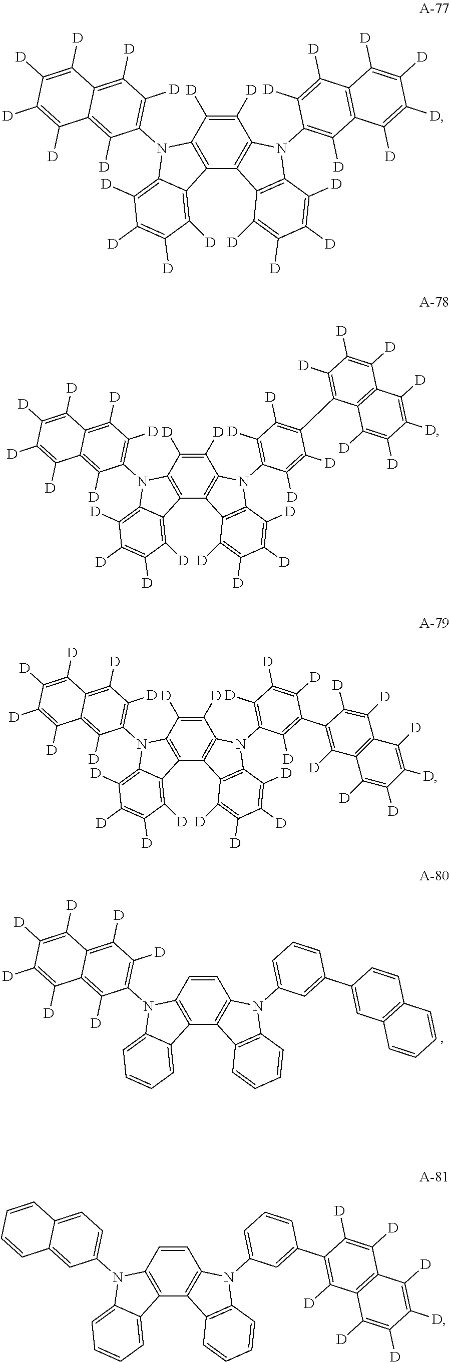 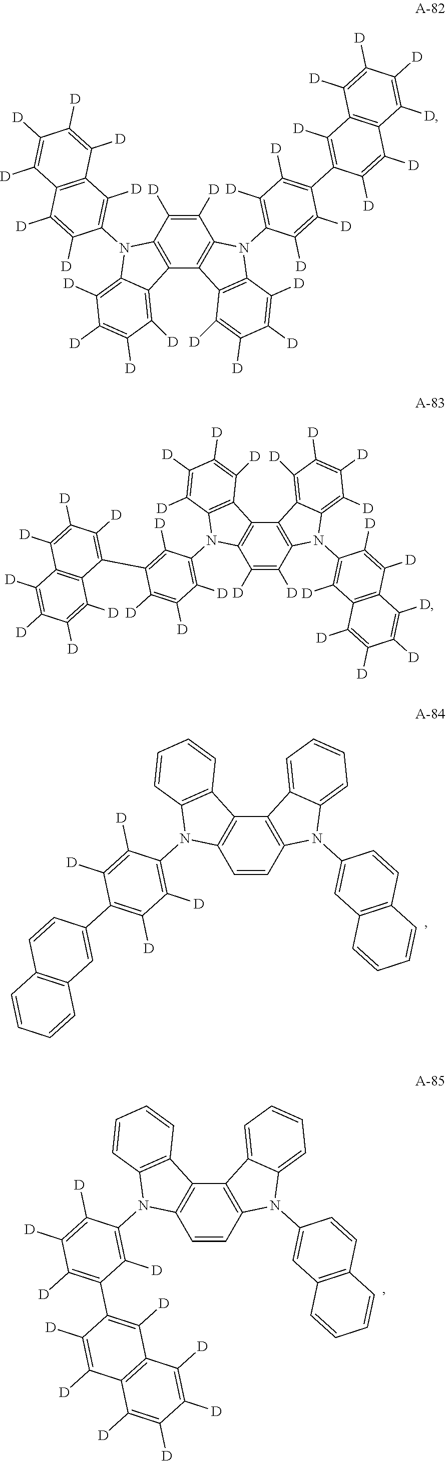 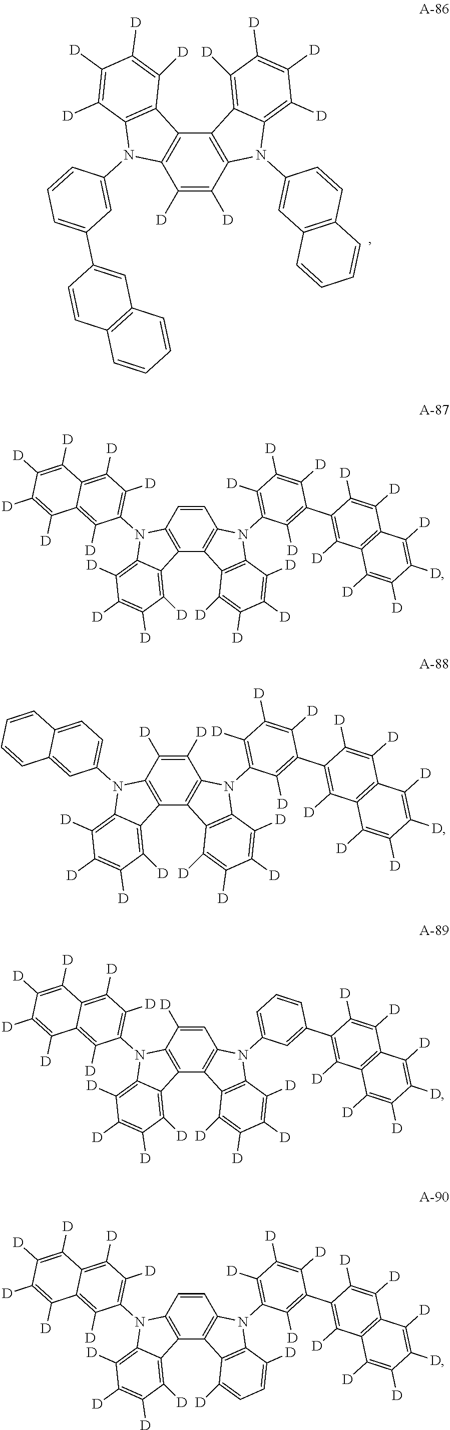 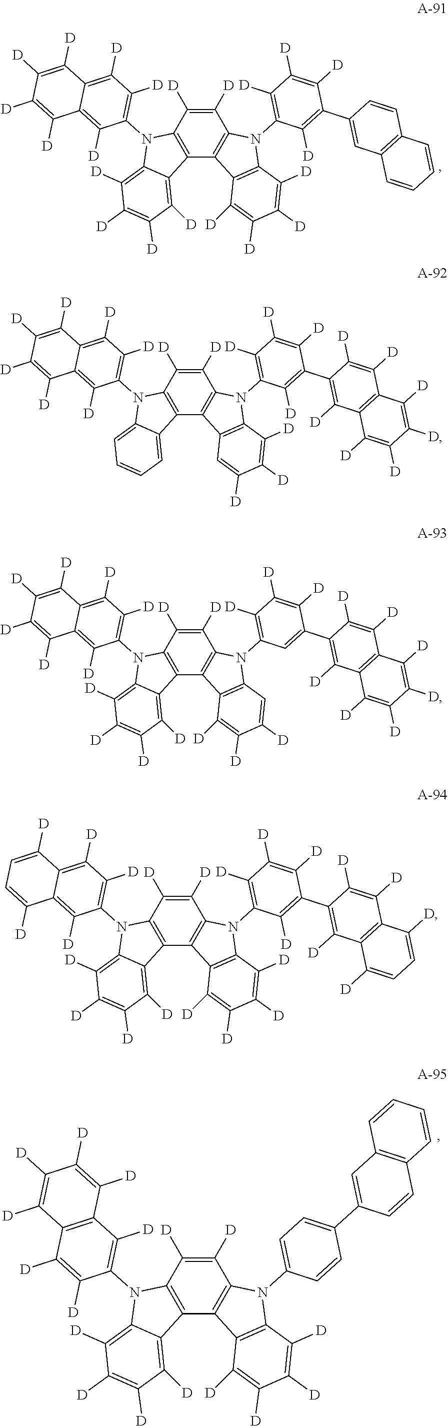 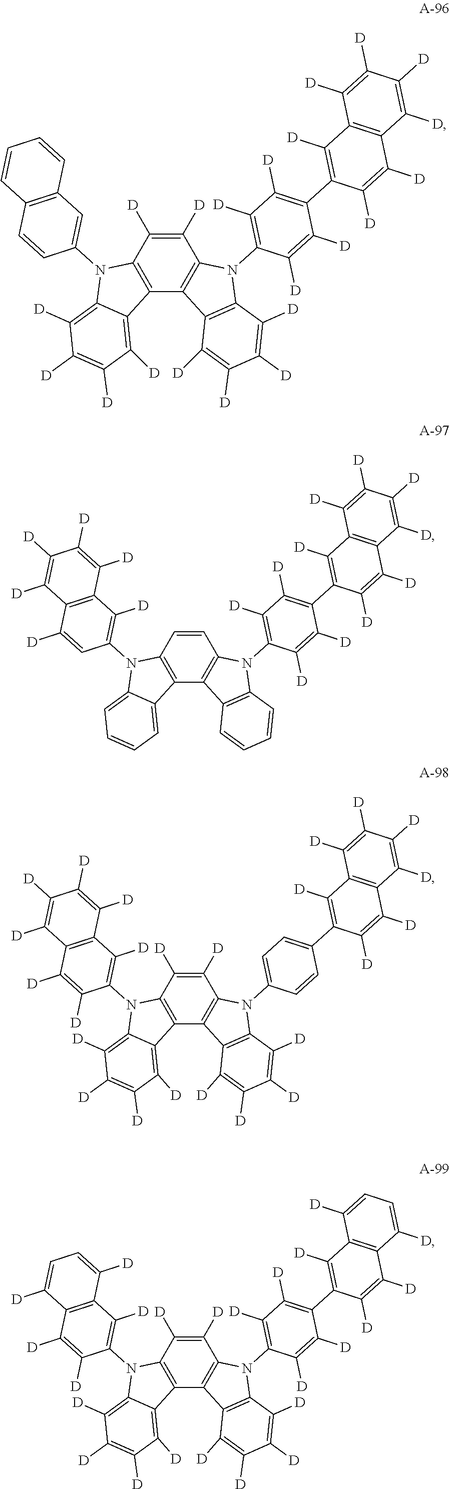 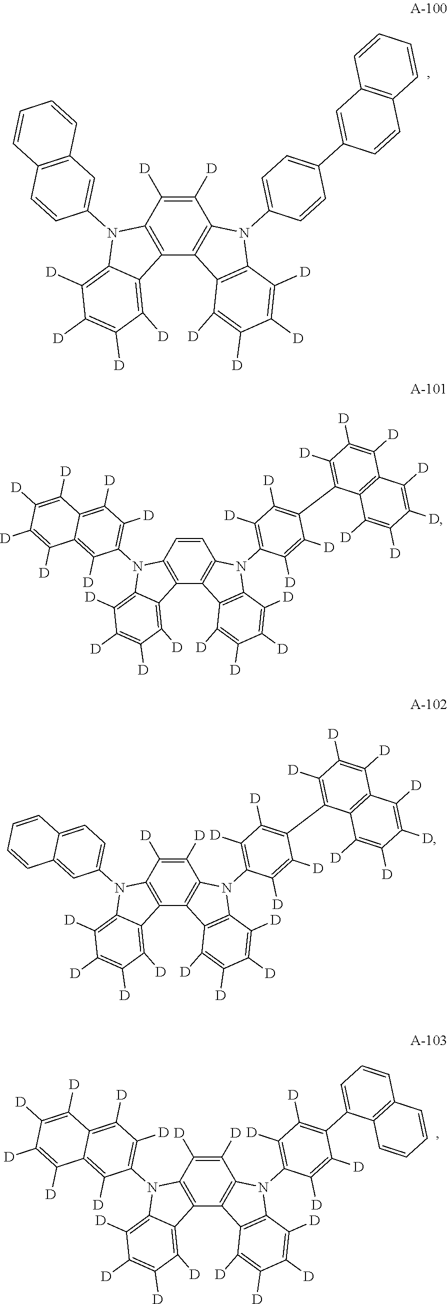 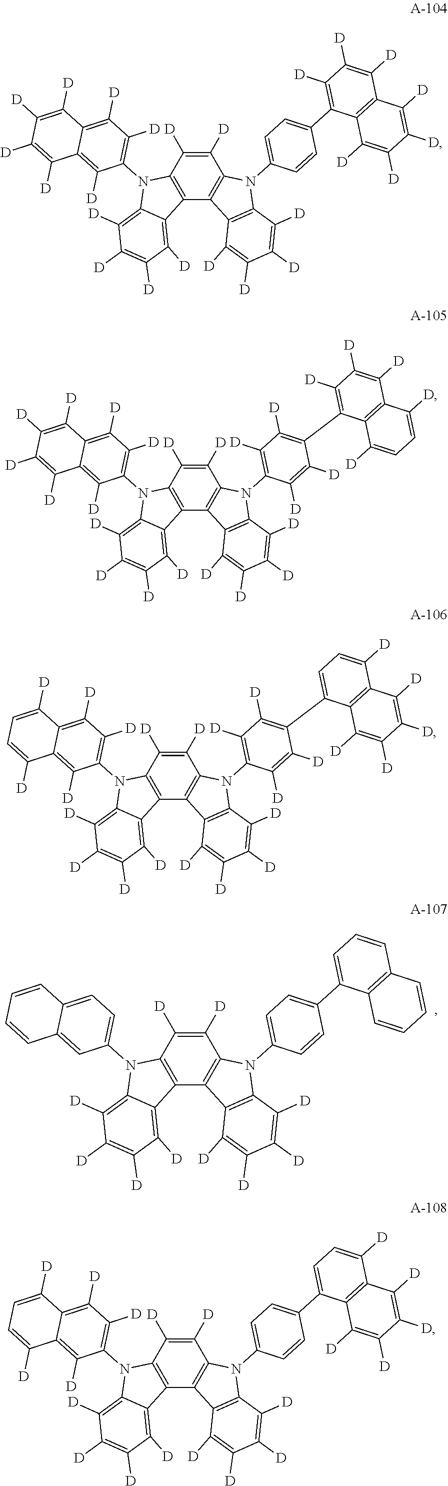 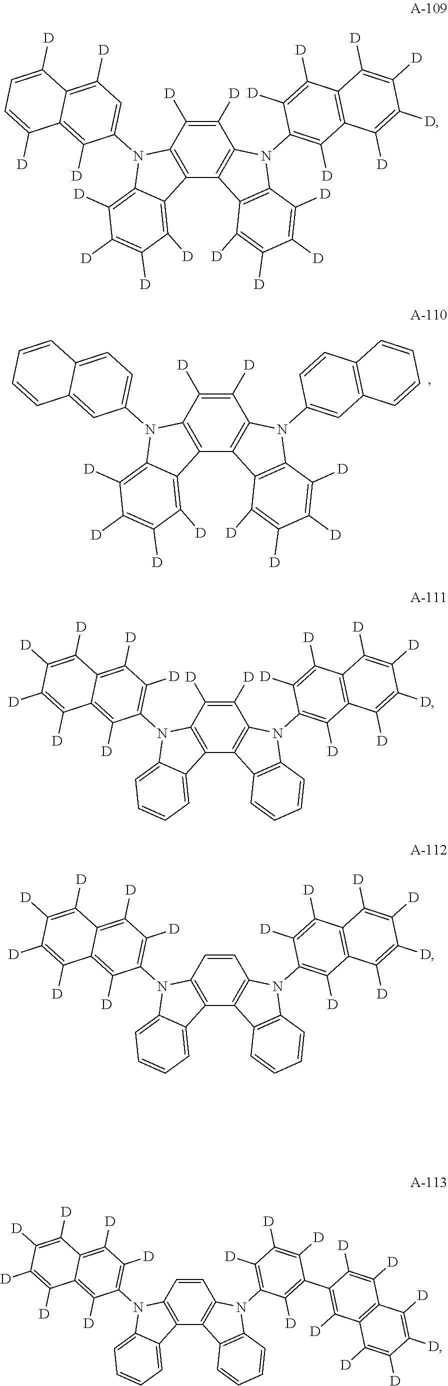 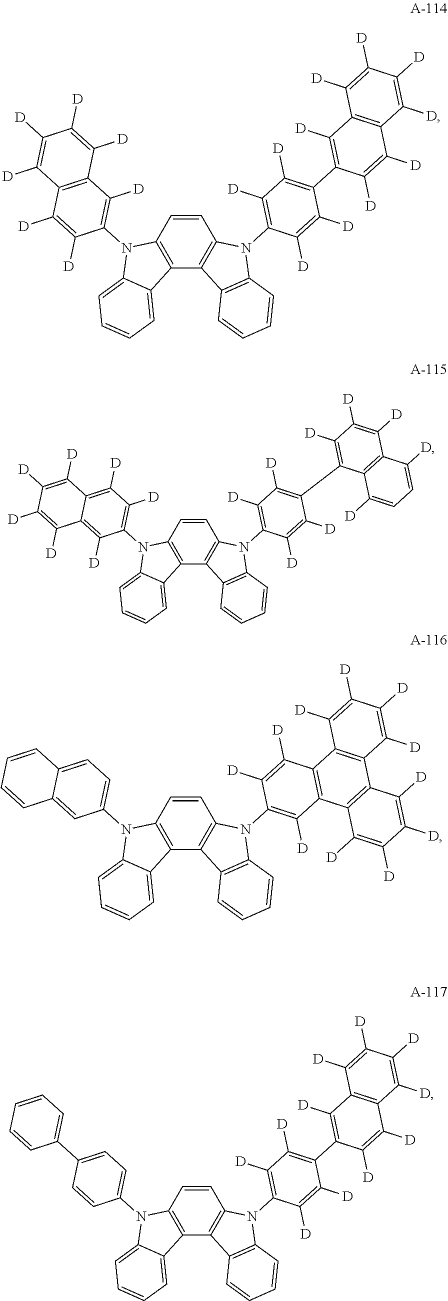 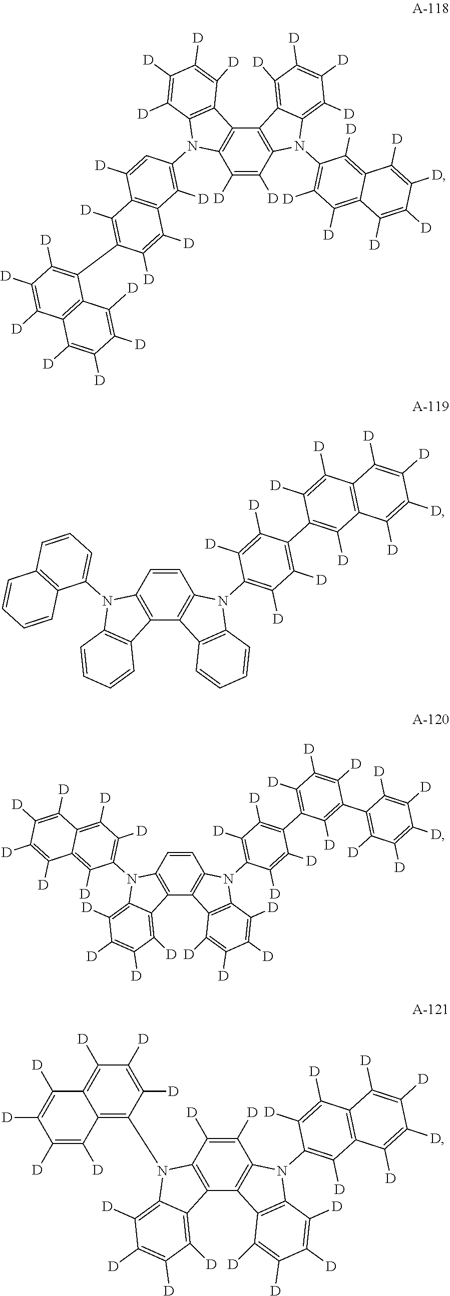 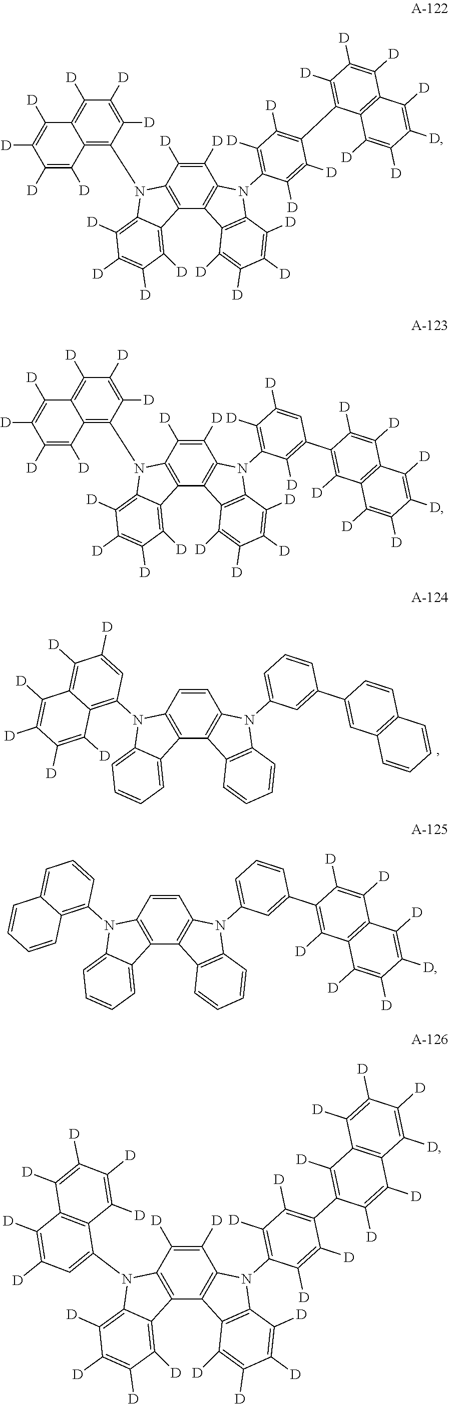 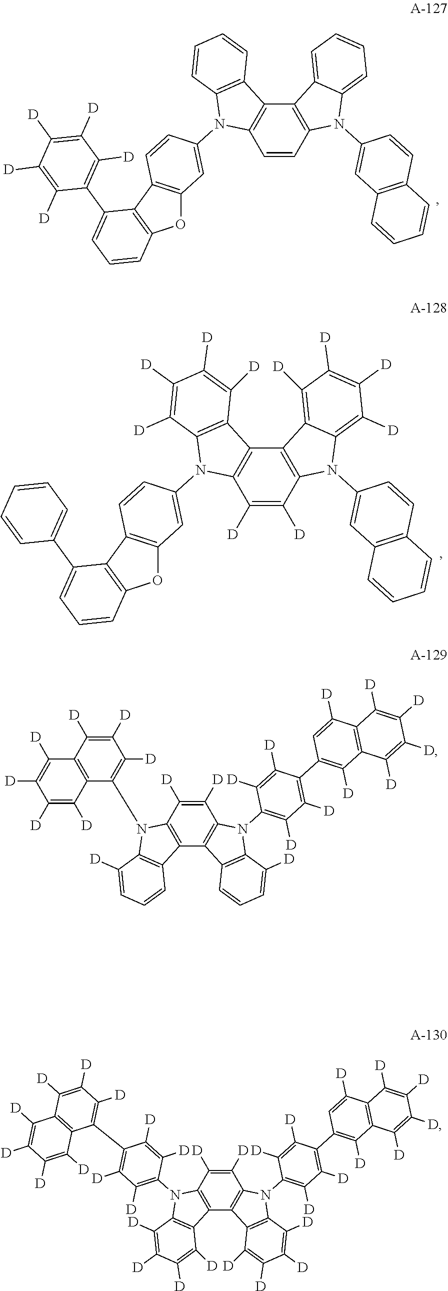 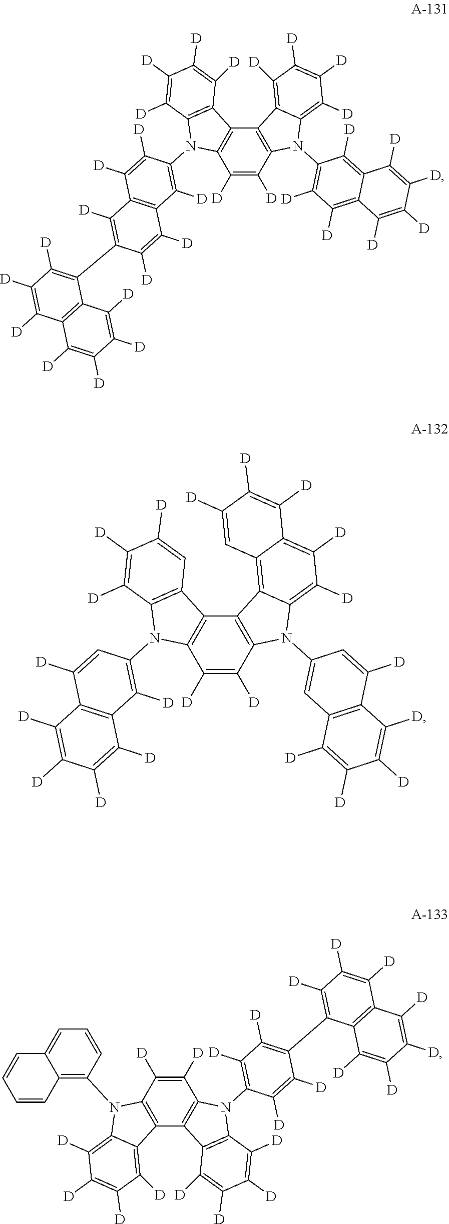 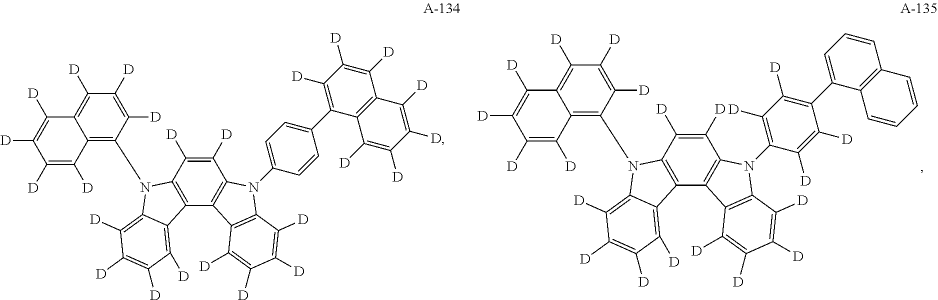 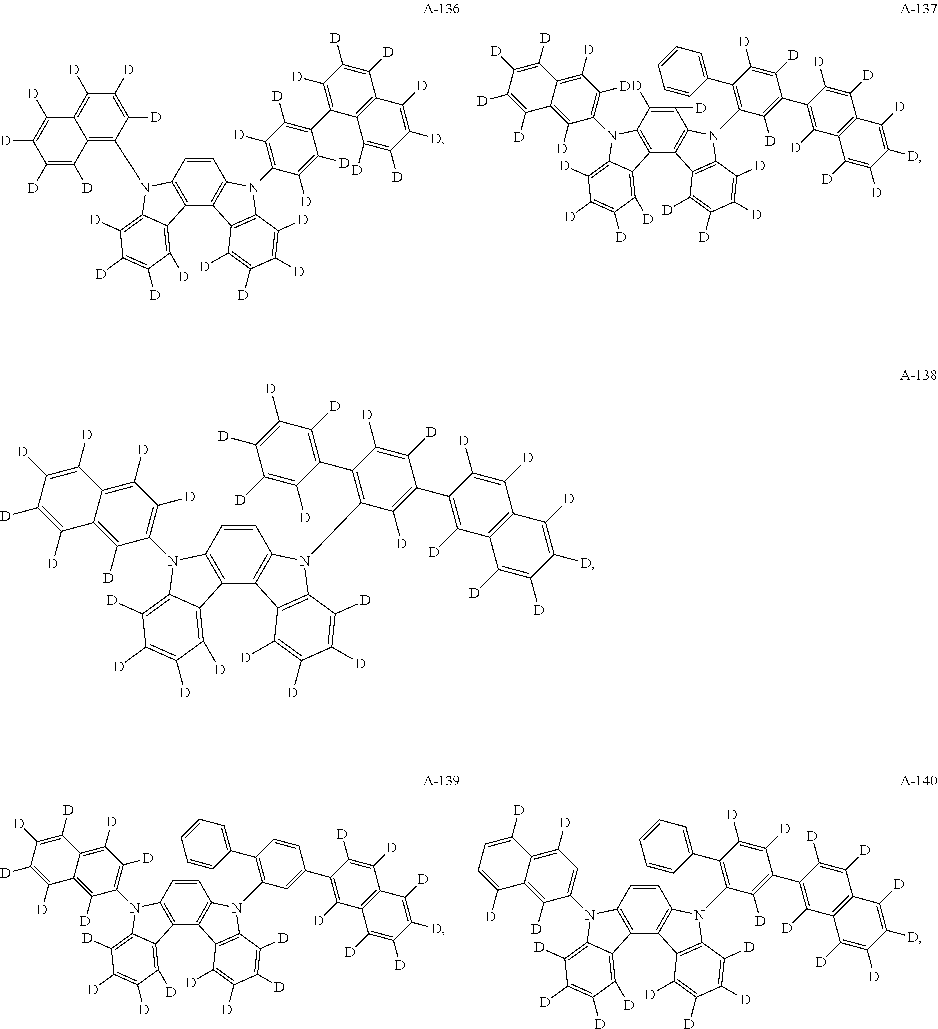 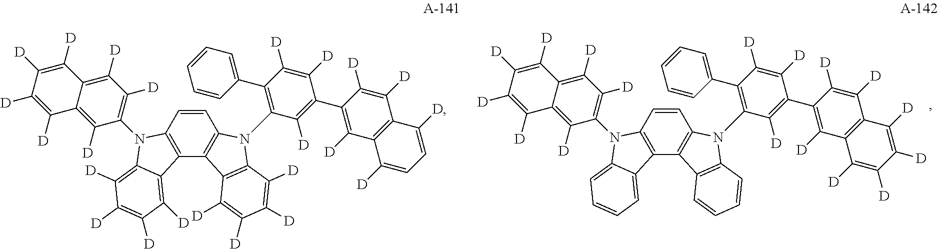 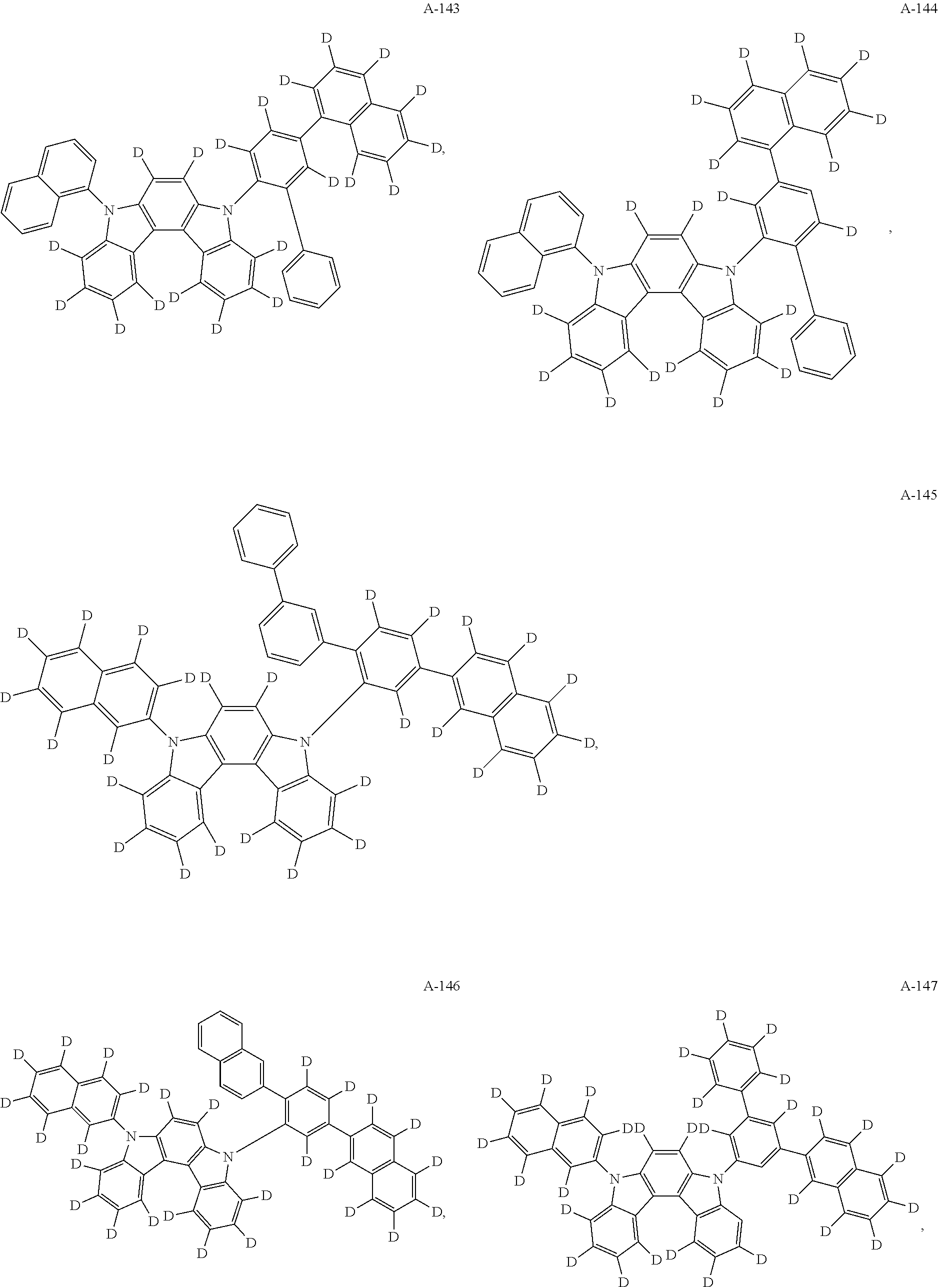 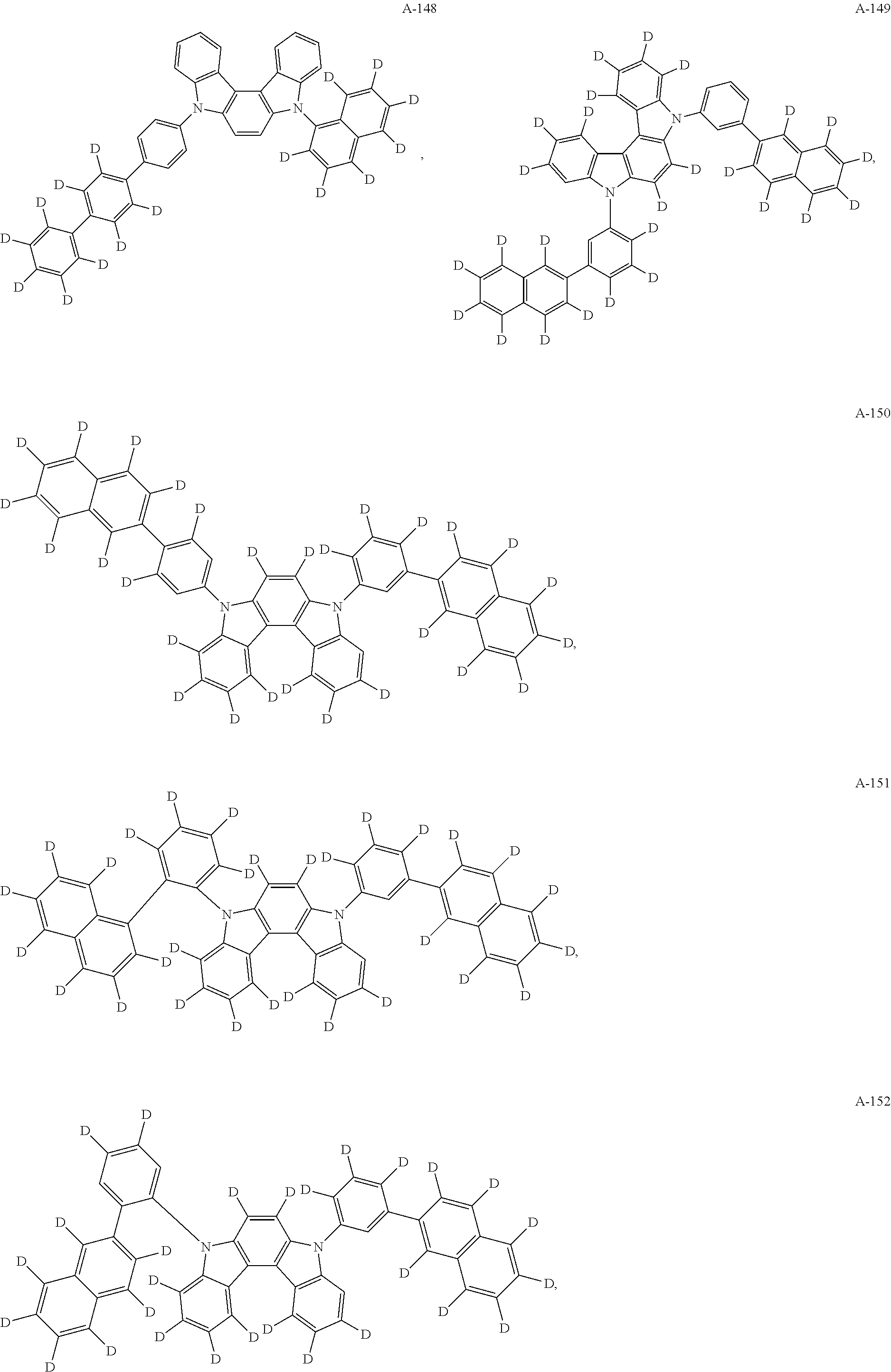 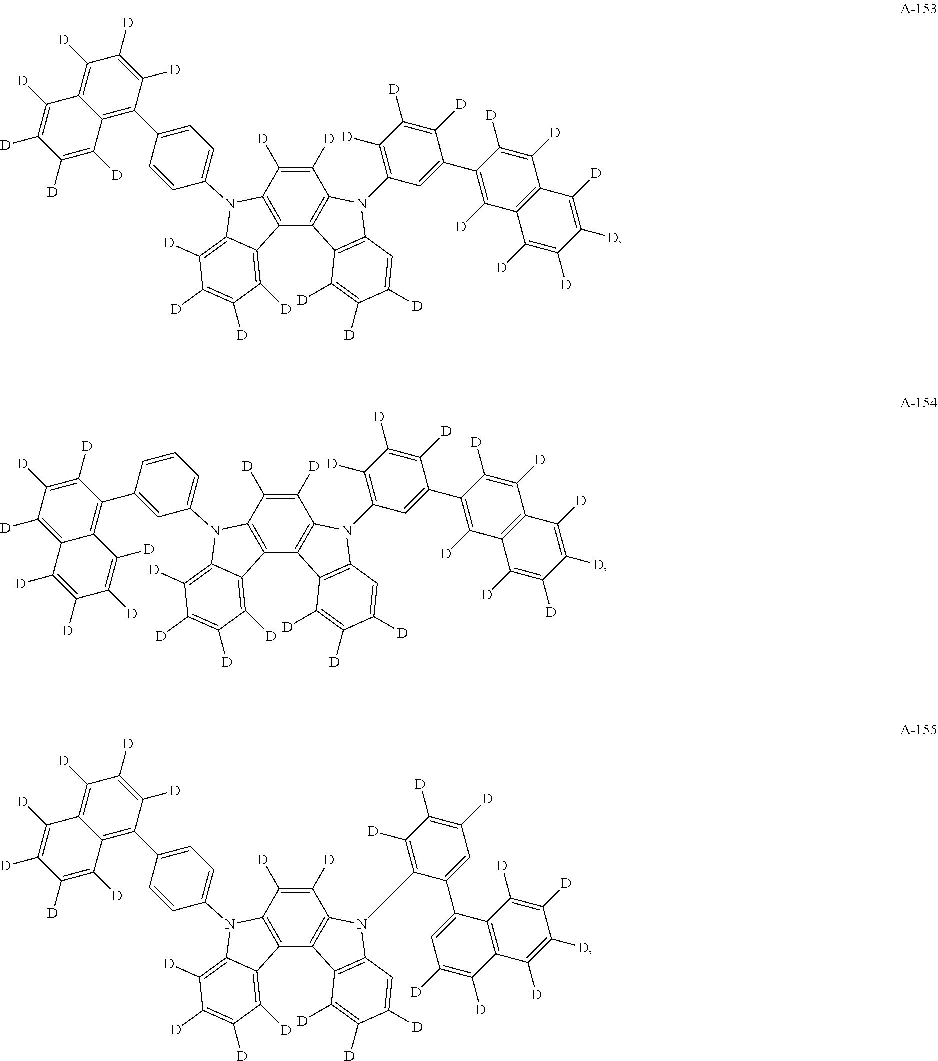 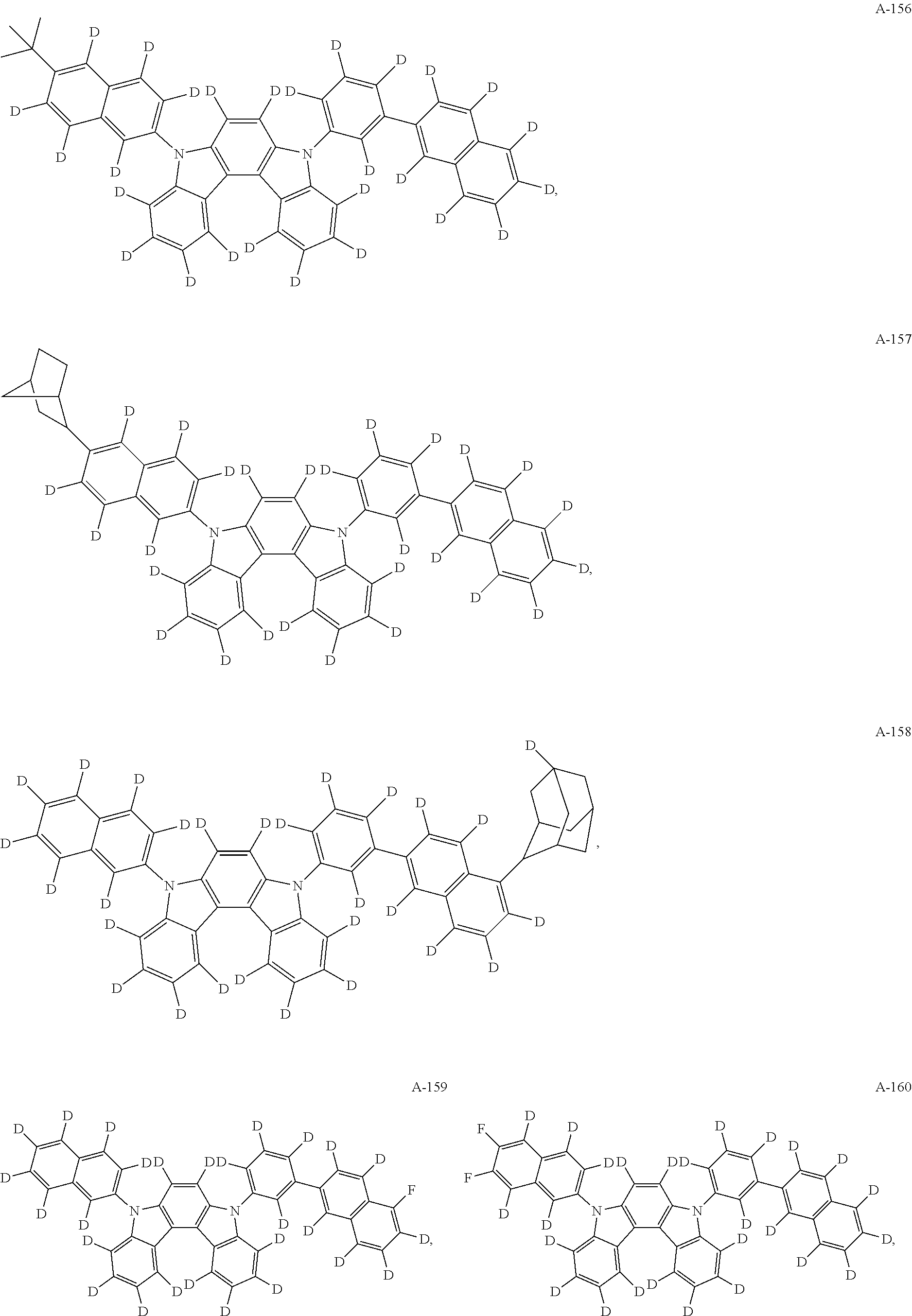 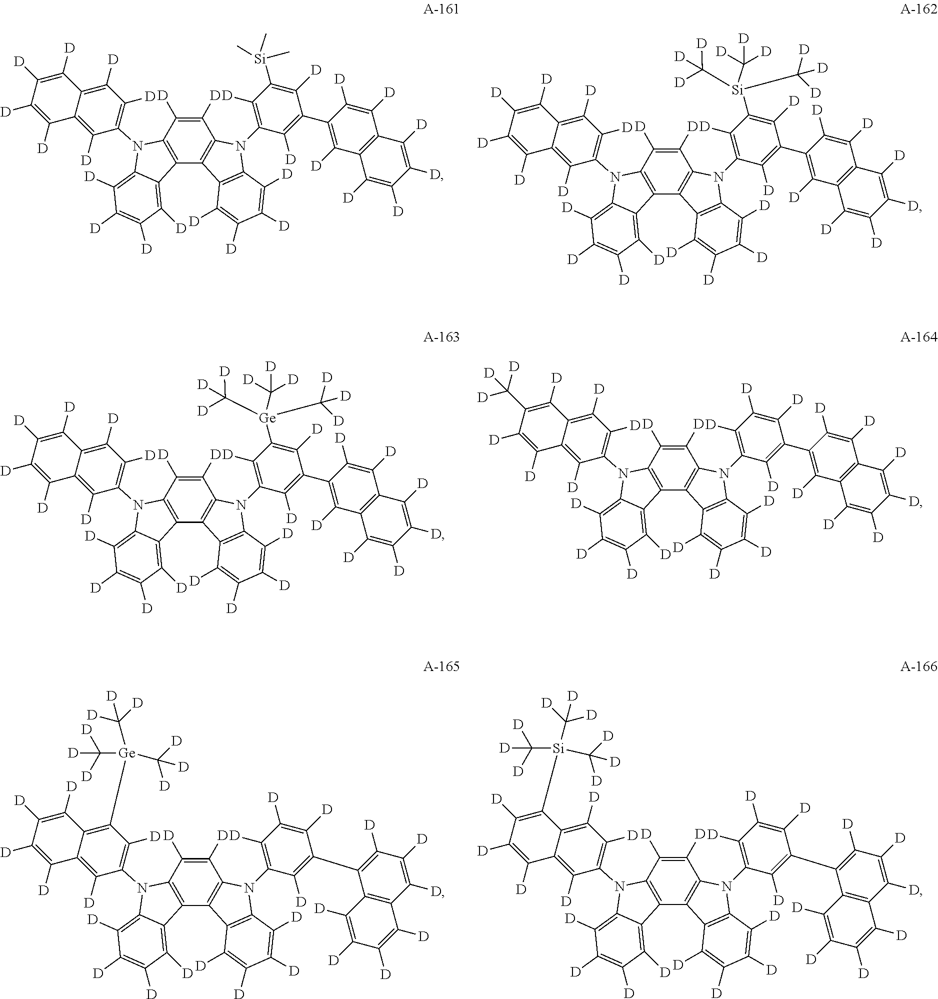 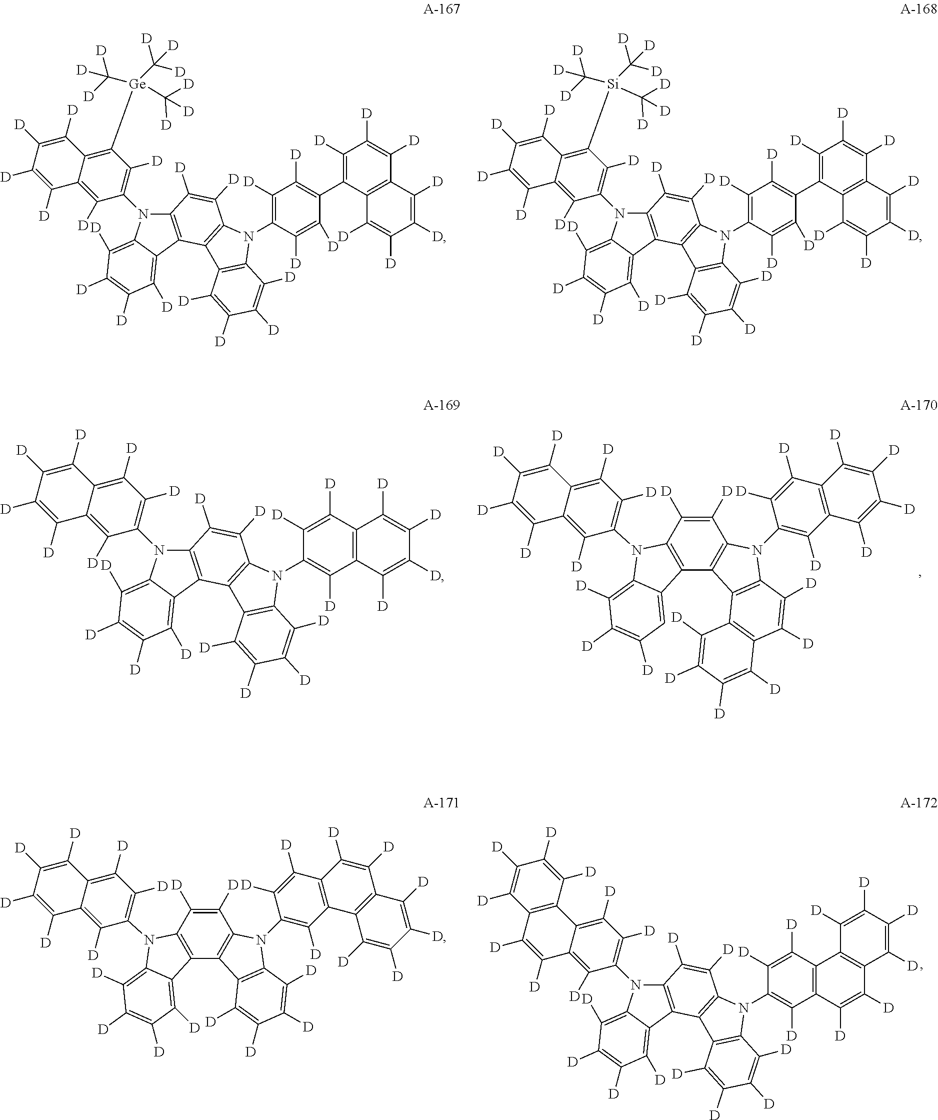 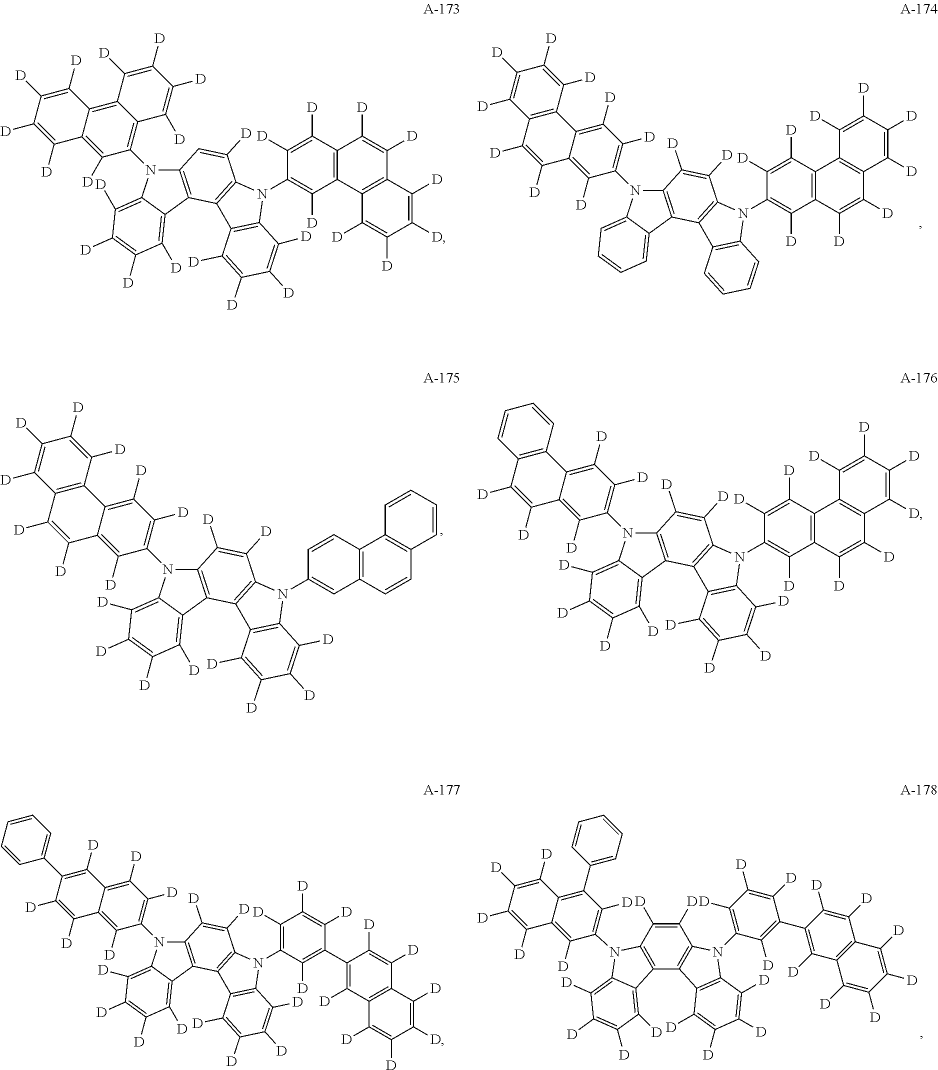 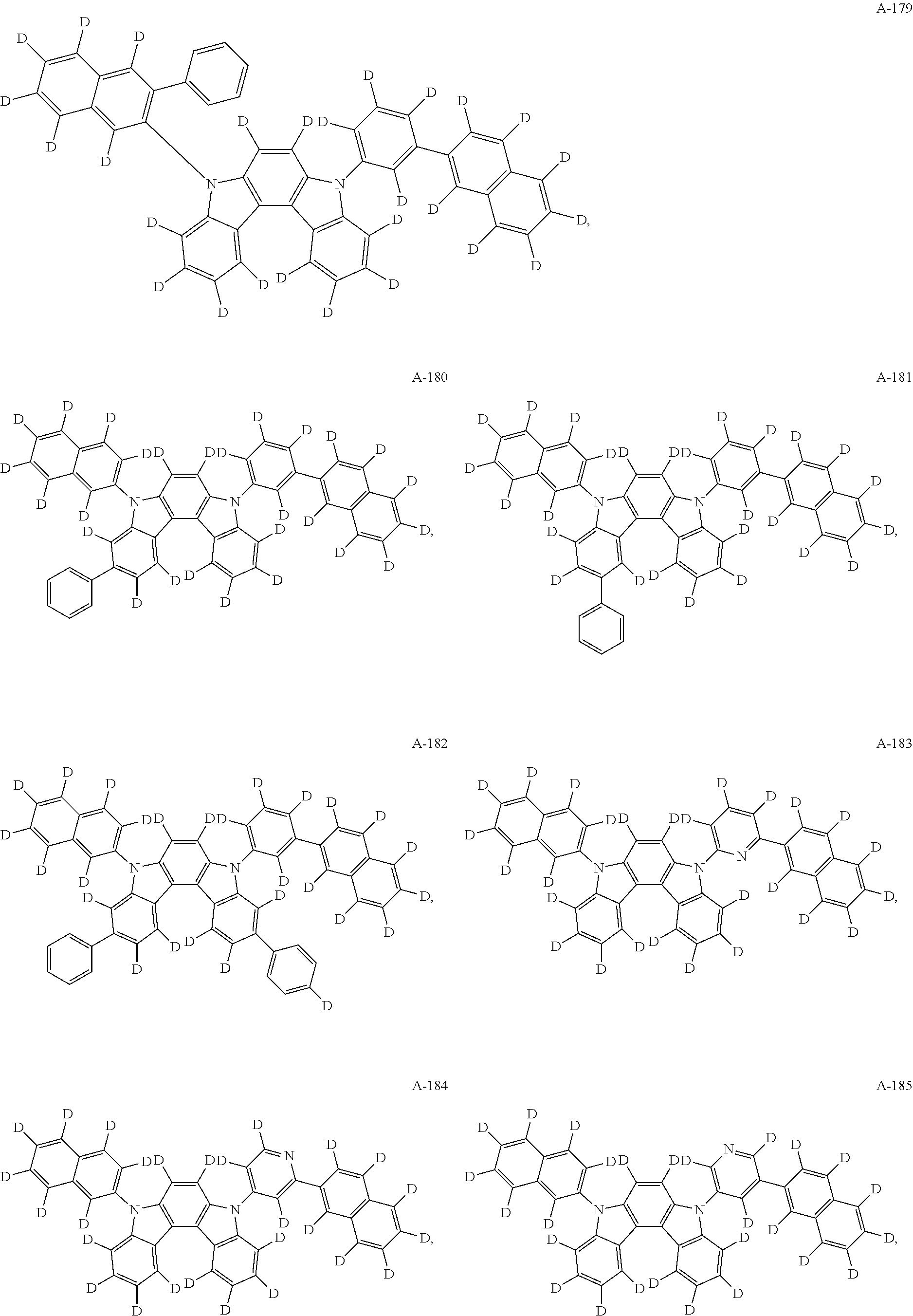 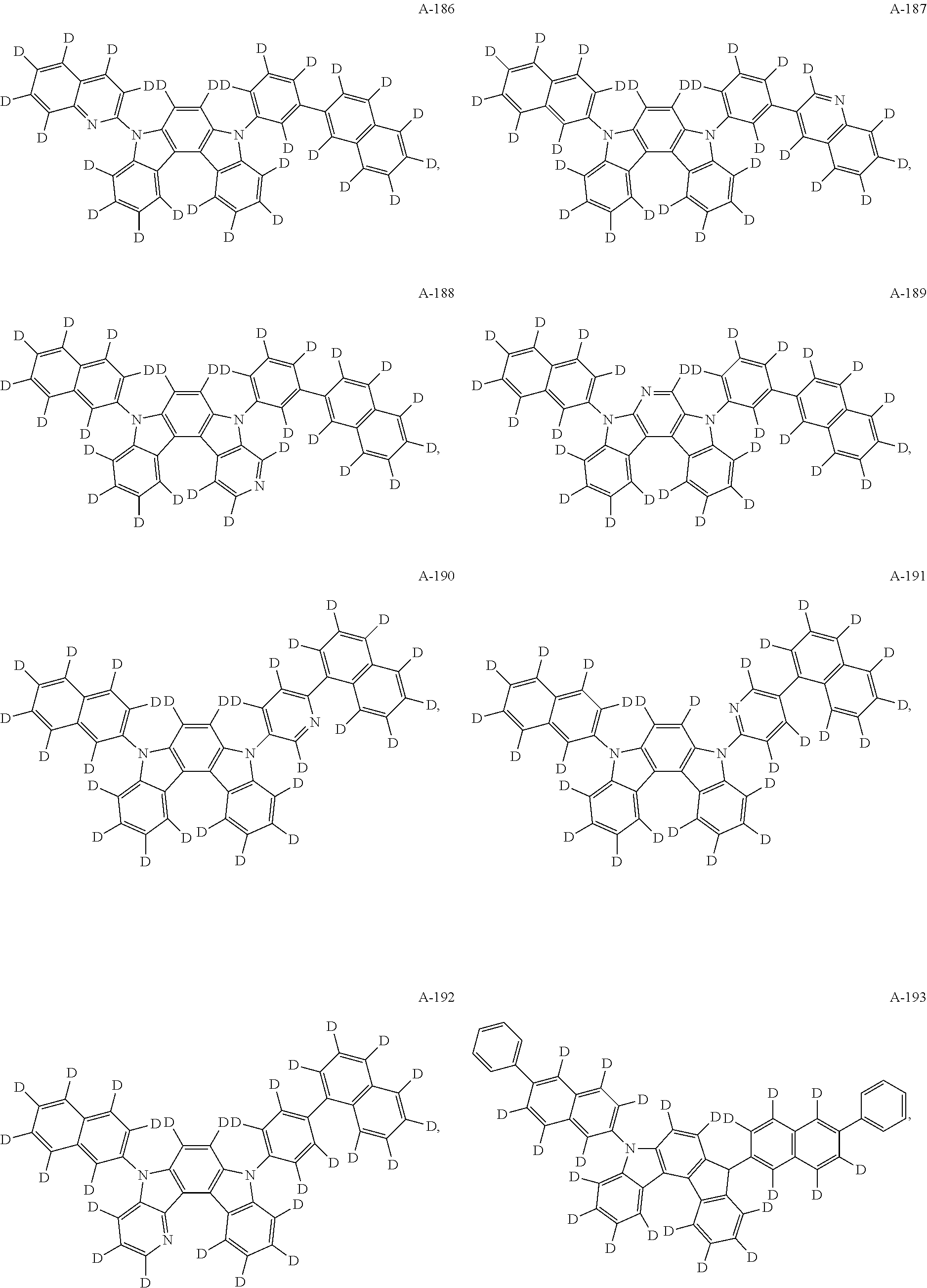 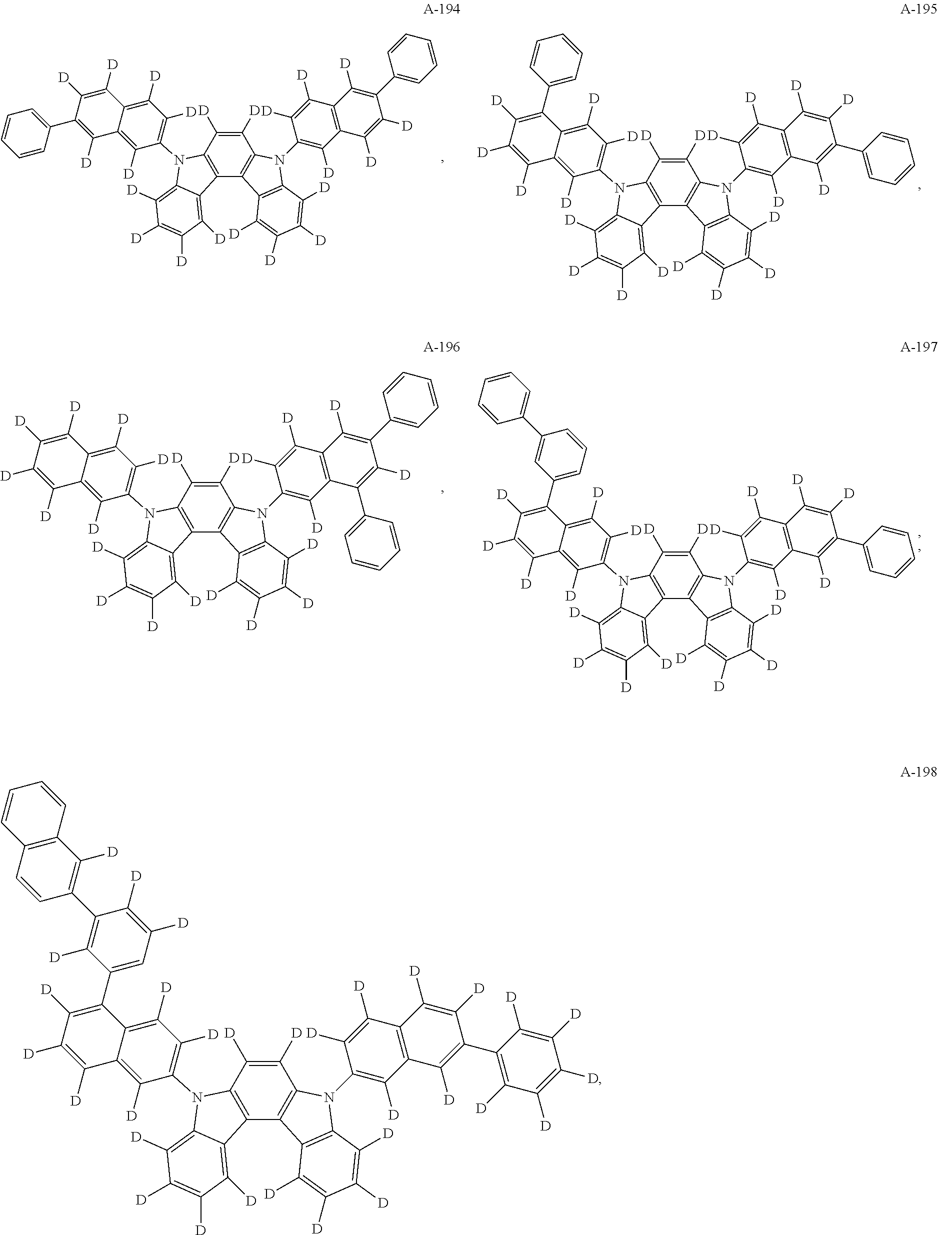 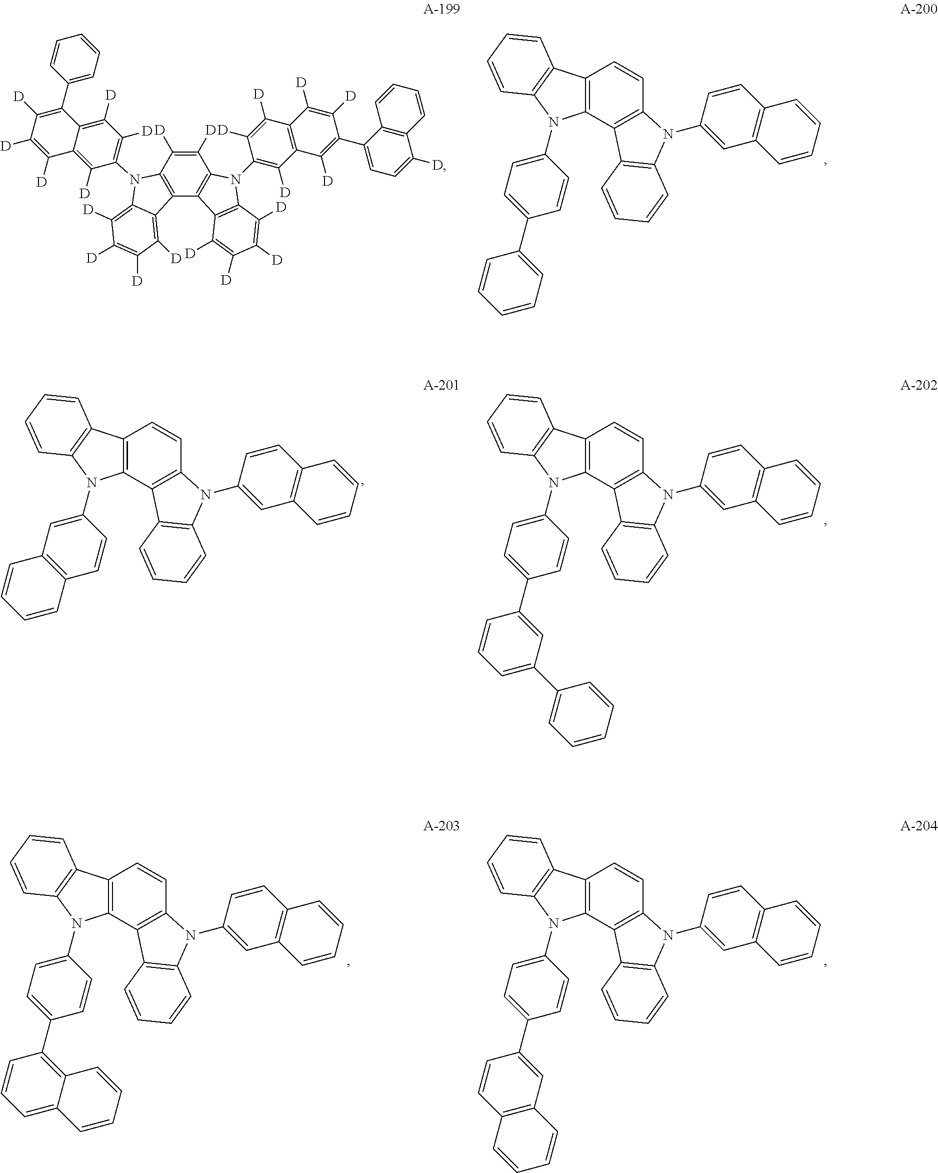 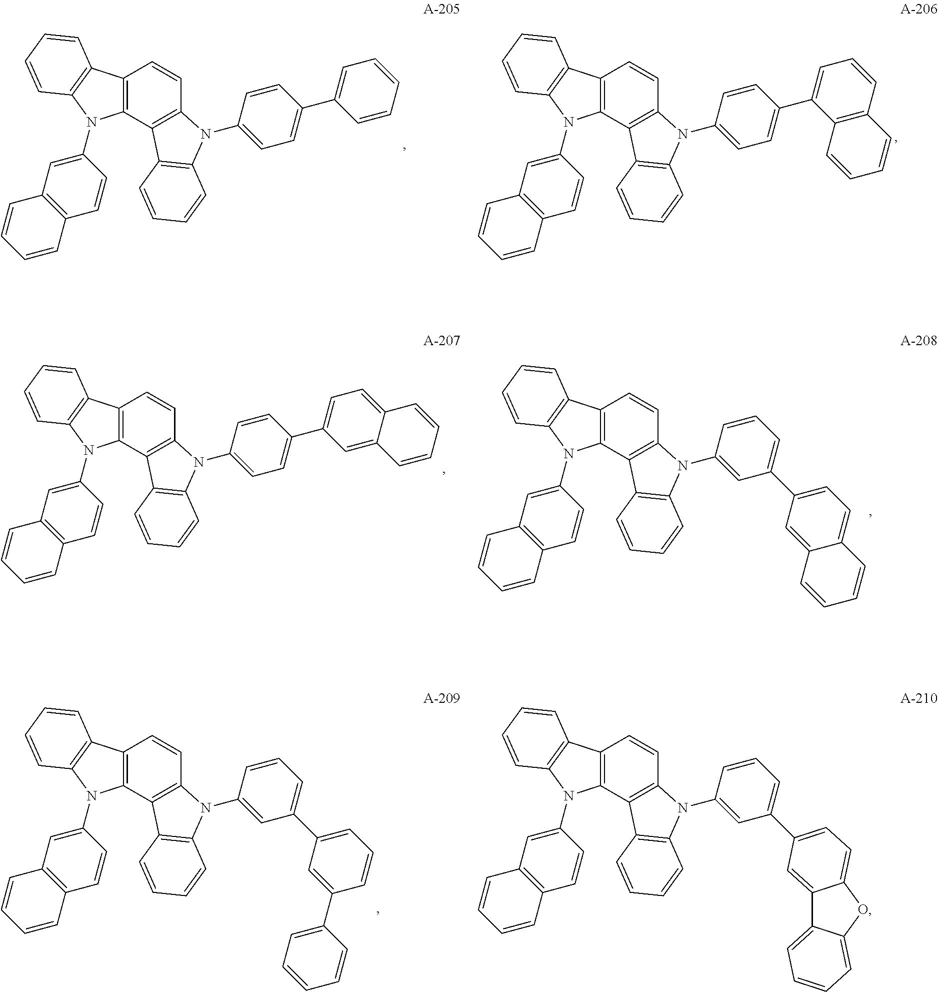 [SMILES: c1ccc(-c2ccc(-n3c4ccccc4c4c3ccc3c5ccccc5n(-c5ccc6ccccc6c5)c34)cc2)cc1 ; c1ccc2cc(-n3c4ccccc4c4ccc5c(c6ccccc6n5-c5ccc(-c6cccc7ccccc67)cc5)c43)ccc2c1 ; c1ccc2cc(-c3ccc(-n4c5ccccc5c5c4ccc4c6ccccc6n(-c6ccc7ccccc7c6)c45)cc3)ccc2c1 ; c1cc(-c2ccc3ccccc3c2)cc(-n2c3ccccc3c3c2ccc2c4ccccc4n(-c4ccc5ccccc5c4)c23)c1 ; c1ccc(-c2cccc(-c3cccc(-n4c5ccccc5c5c4ccc4c6ccccc6n(-c6ccc7ccccc7c6)c45)c3)c2)cc1 ; c1cc(-c2ccc3oc4ccccc4c3c2)cc(-n2c3ccccc3c3c2ccc2c4ccccc4n(-c4ccc5ccccc5c4)c23)c1] 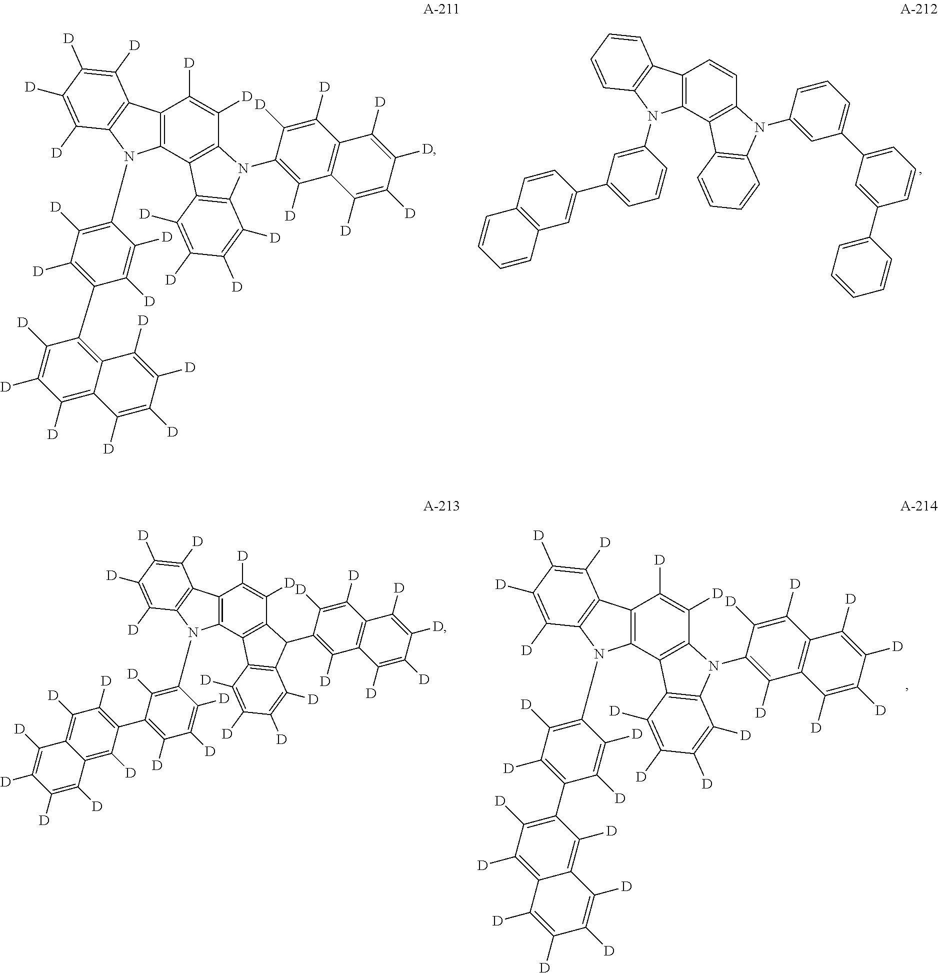 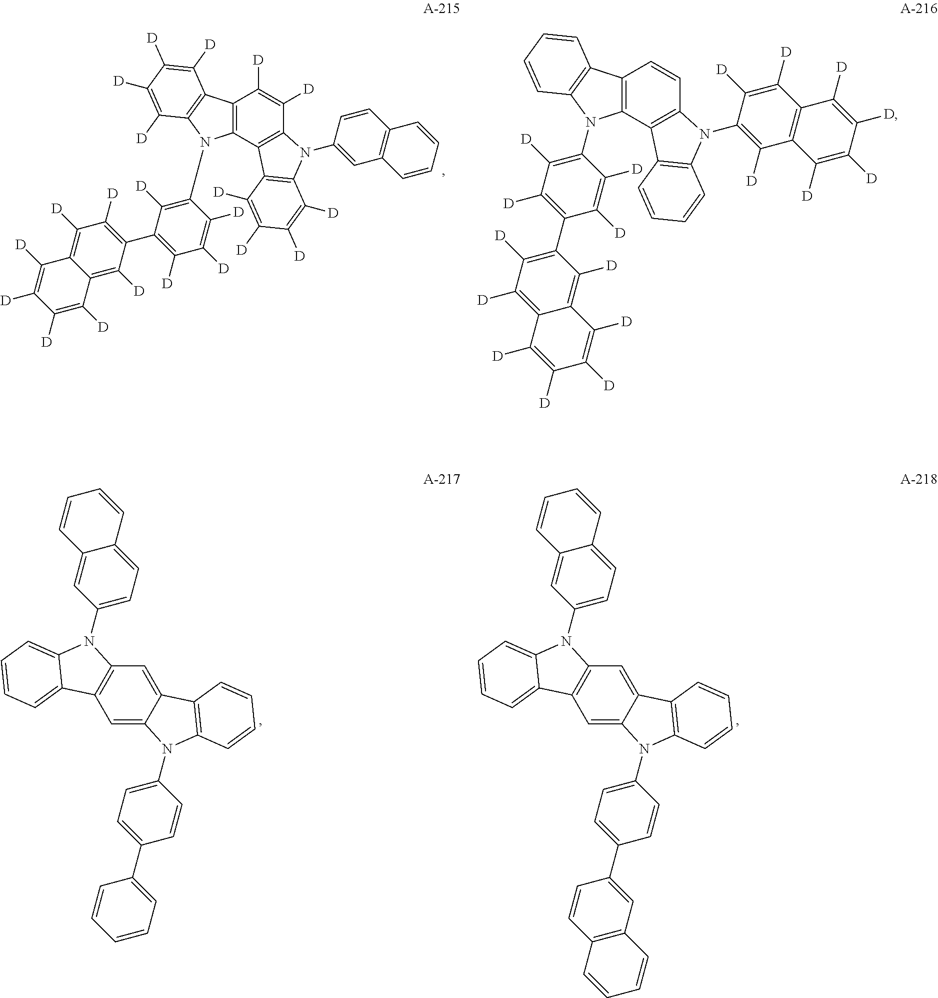 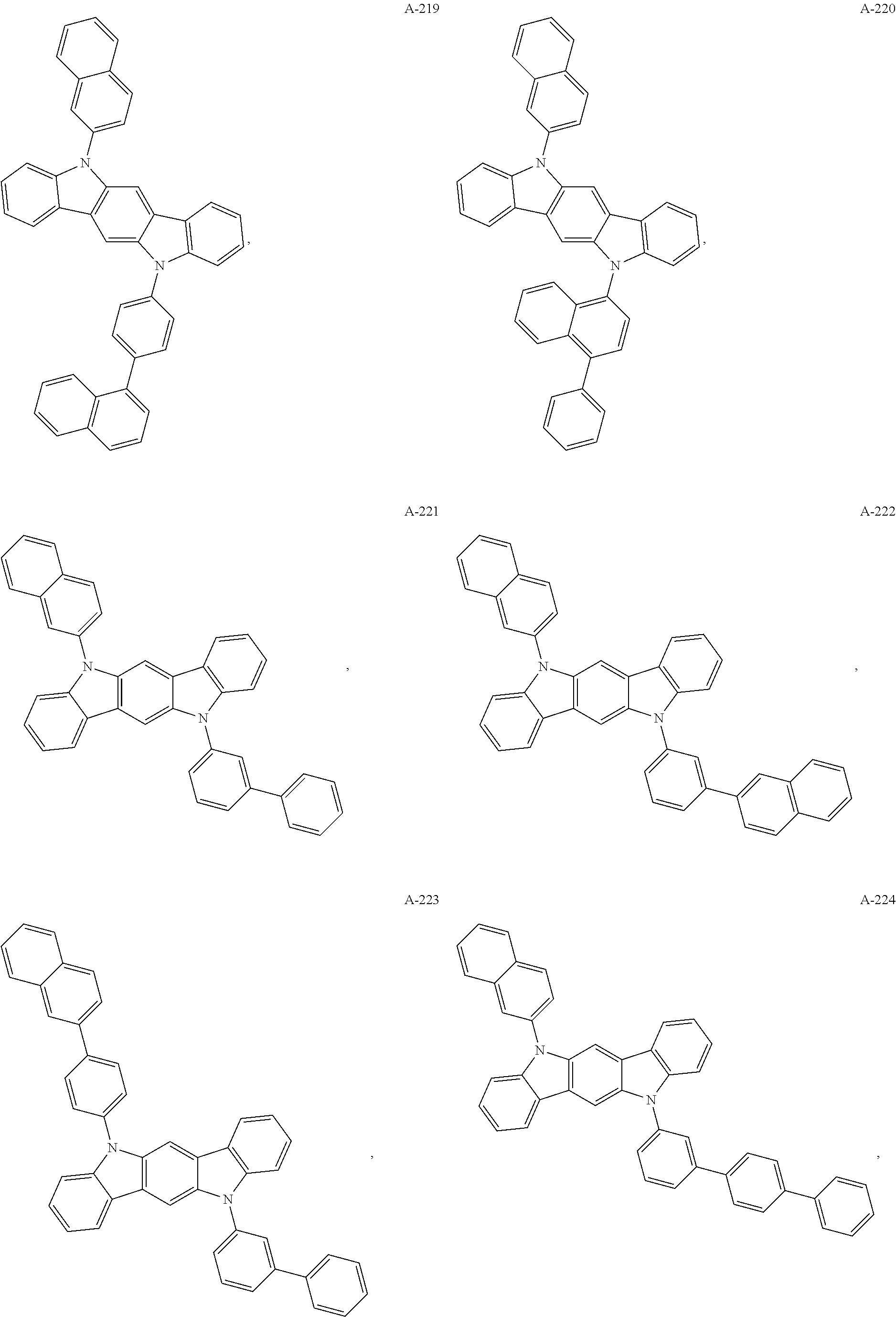 [SMILES: c1ccc2cc(-n3c4ccccc4c4cc5c(cc43)c3ccccc3n5-c3ccc(-c4cccc5ccccc45)cc3)ccc2c1 ; c1ccc(-c2ccc(-n3c4ccccc4c4cc5c(cc43)c3ccccc3n5-c3ccc4ccccc4c3)c3ccccc23)cc1 ; c1ccc(-c2cccc(-n3c4ccccc4c4cc5c(cc43)c3ccccc3n5-c3ccc4ccccc4c3)c2)cc1 ; c1cc(-c2ccc3ccccc3c2)cc(-n2c3ccccc3c3cc4c(cc32)c2ccccc2n4-c2ccc3ccccc3c2)c1 ; c1ccc(-c2cccc(-n3c4ccccc4c4cc5c(cc43)c3ccccc3n5-c3ccc(-c4ccc5ccccc5c4)cc3)c2)cc1 ; c1ccc(-c2ccc(-c3cccc(-n4c5ccccc5c5cc6c(cc54)c4ccccc4n6-c4ccc5ccccc5c4)c3)cc2)cc1] 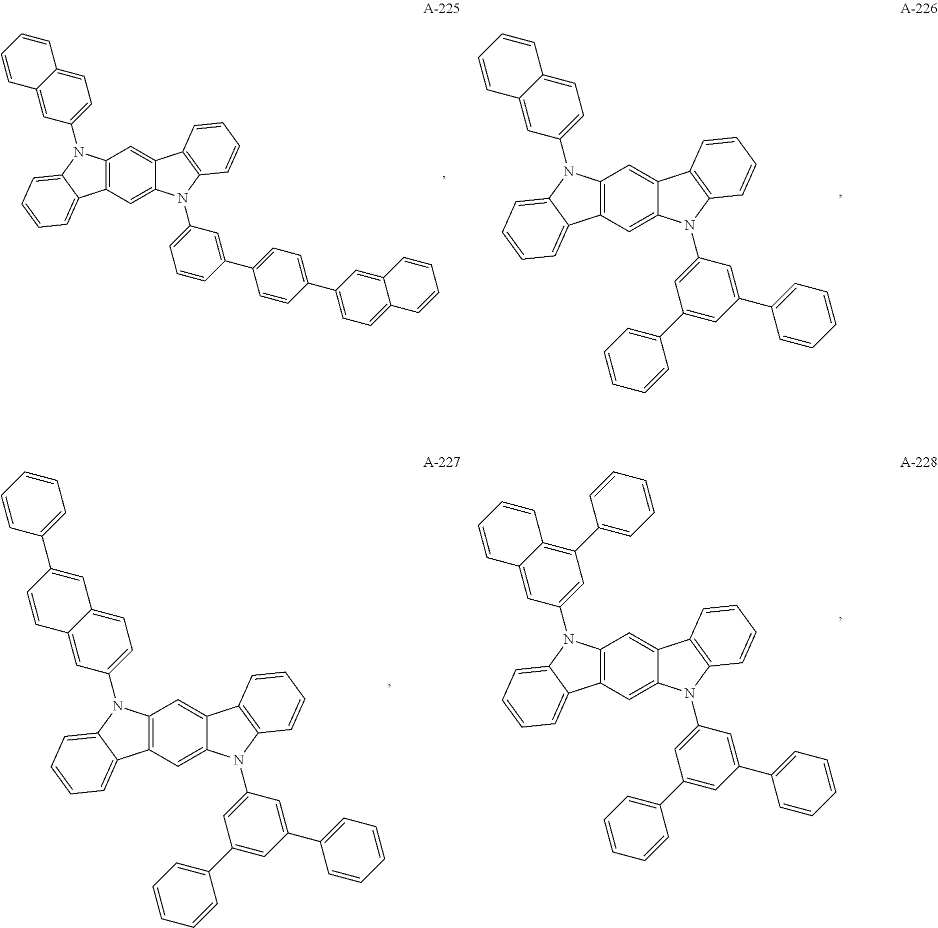 [SMILES: c1cc(-c2ccc(-c3ccc4ccccc4c3)cc2)cc(-n2c3ccccc3c3cc4c(cc32)c2ccccc2n4-c2ccc3ccccc3c2)c1 ; c1ccc(-c2cc(-c3ccccc3)cc(-n3c4ccccc4c4cc5c(cc43)c3ccccc3n5-c3ccc4ccccc4c3)c2)cc1 ; c1ccc(-c2cc(-c3ccccc3)cc(-n3c4ccccc4c4cc5c(cc43)c3ccccc3n5-c3ccc4cc(-c5ccccc5)ccc4c3)c2)cc1 ; c1ccc(-c2cc(-c3ccccc3)cc(-n3c4ccccc4c4cc5c(cc43)c3ccccc3n5-c3cc(-c4ccccc4)c4ccccc4c3)c2)cc1] 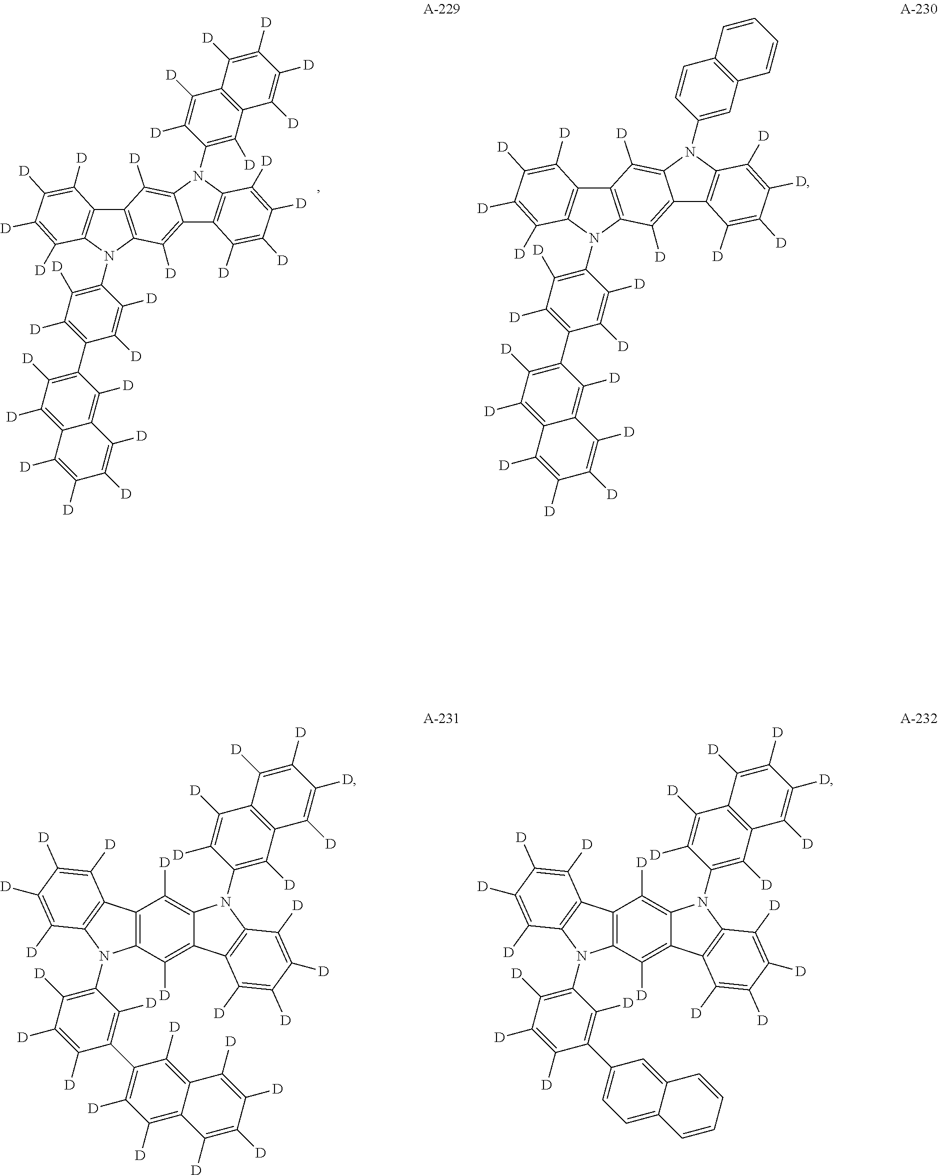 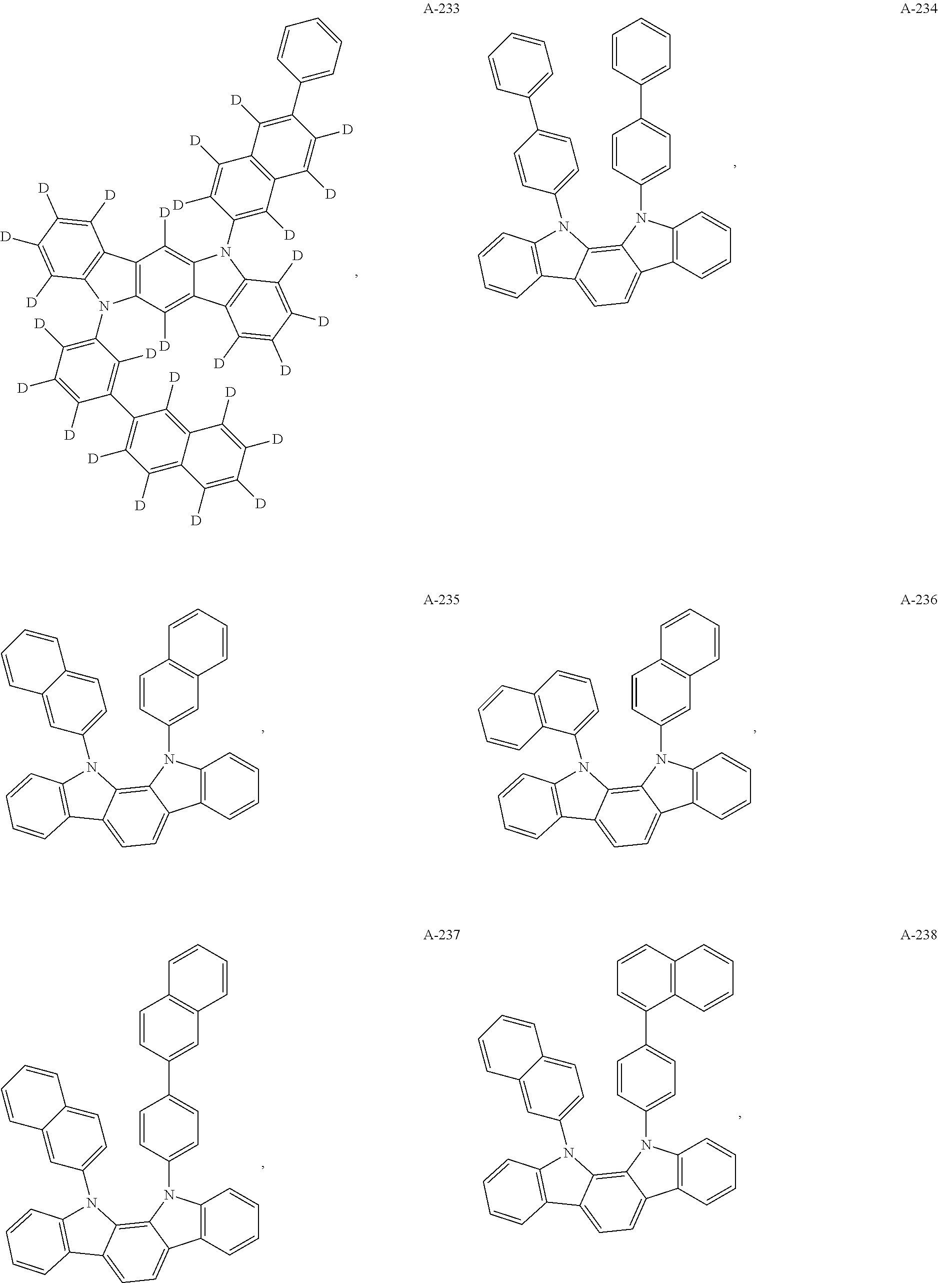 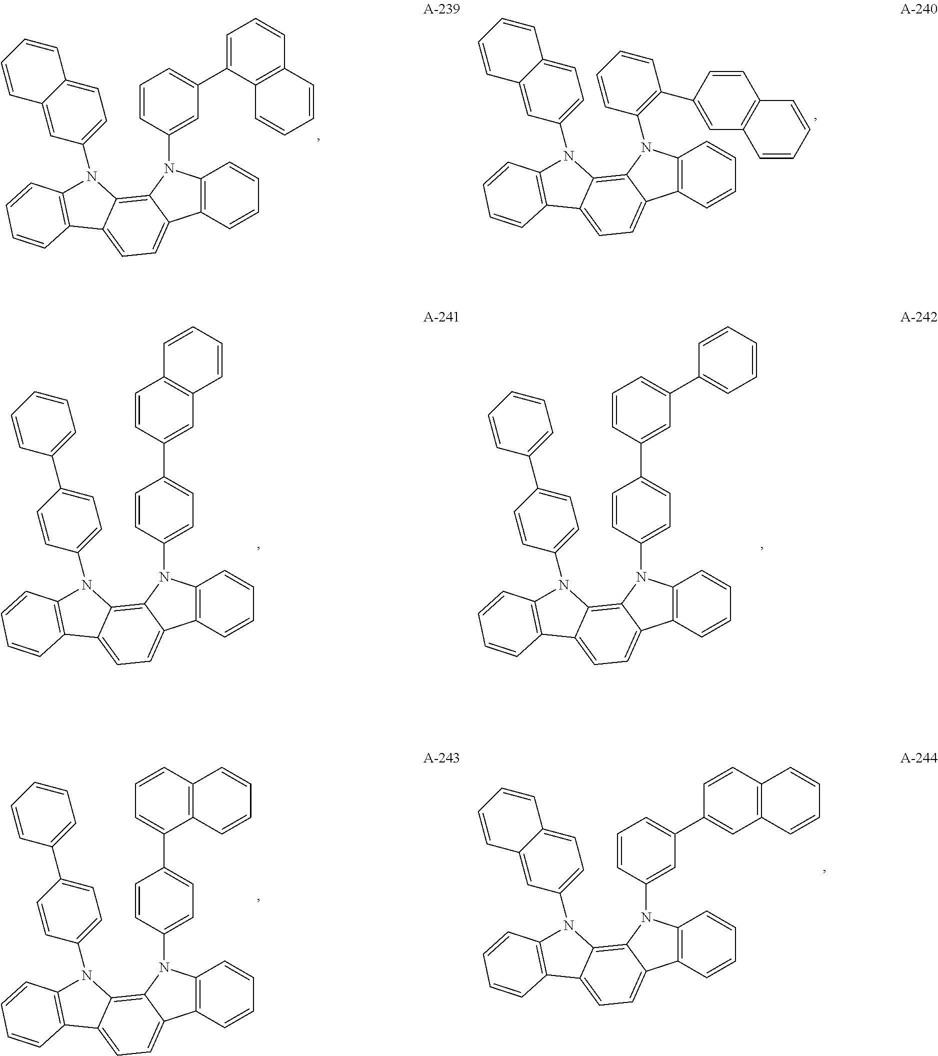 [SMILES: c1cc(-c2cccc3ccccc23)cc(-n2c3ccccc3c3ccc4c5ccccc5n(-c5ccc6ccccc6c5)c4c32)c1 ; c1ccc(-n2c3ccccc3c3ccc4c5ccccc5n(-c5ccc6ccccc6c5)c4c32)c(-c2ccc3ccccc3c2)c1 ; c1ccc(-c2ccc(-n3c4ccccc4c4ccc5c6ccccc6n(-c6ccc(-c7ccc8ccccc8c7)cc6)c5c43)cc2)cc1 ; c1ccc(-c2ccc(-n3c4ccccc4c4ccc5c6ccccc6n(-c6ccc(-c7cccc(-c8ccccc8)c7)cc6)c5c43)cc2)cc1 ; c1ccc(-c2ccc(-n3c4ccccc4c4ccc5c6ccccc6n(-c6ccc(-c7cccc8ccccc78)cc6)c5c43)cc2)cc1 ; c1cc(-c2ccc3ccccc3c2)cc(-n2c3ccccc3c3ccc4c5ccccc5n(-c5ccc6ccccc6c5)c4c32)c1] 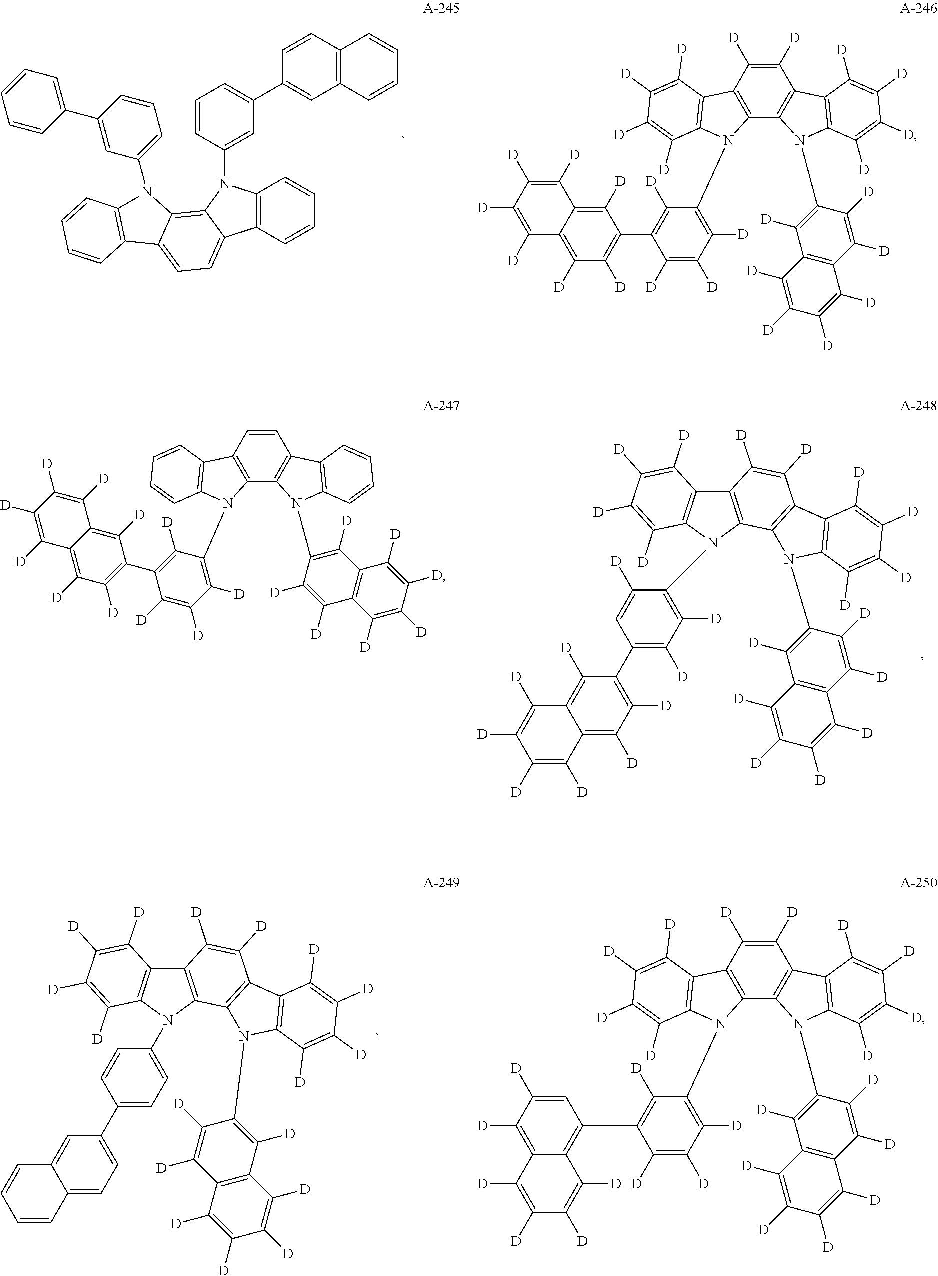 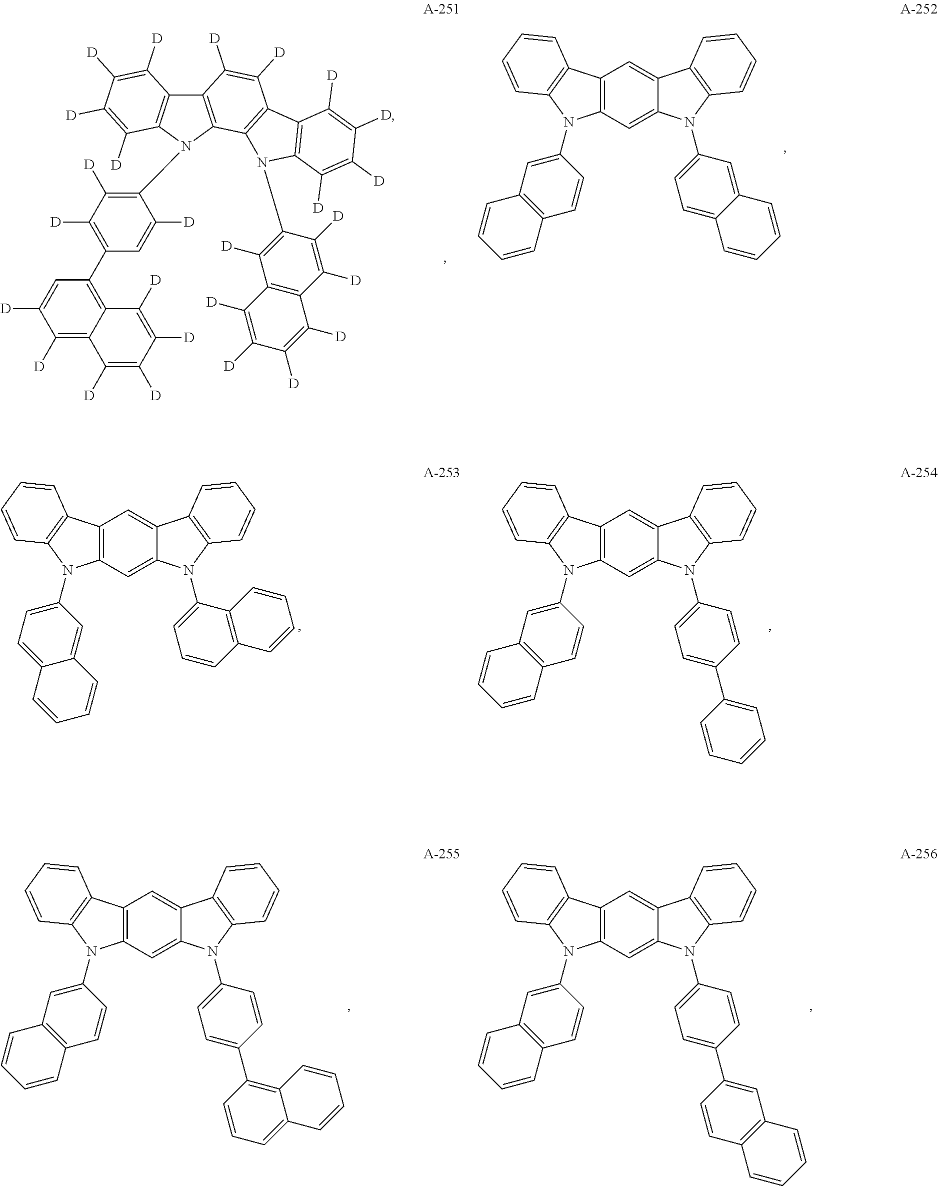 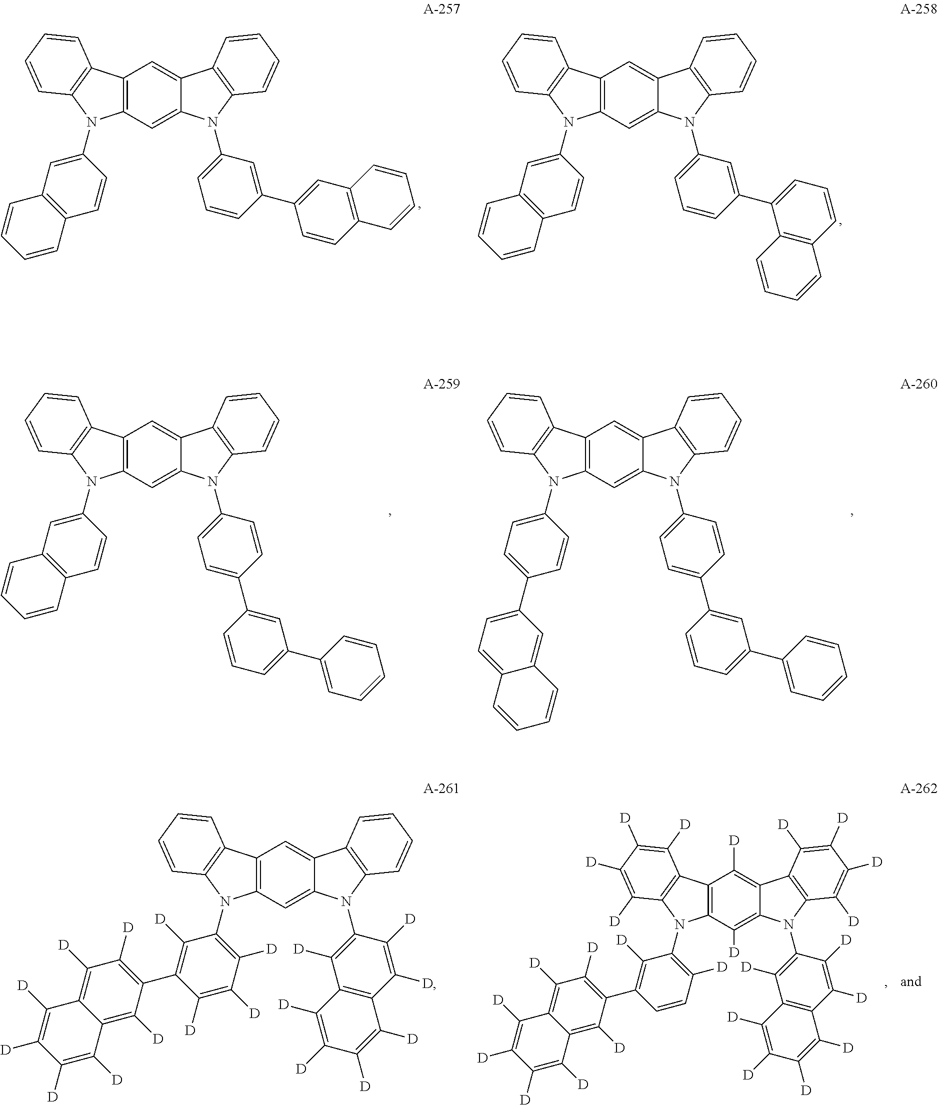 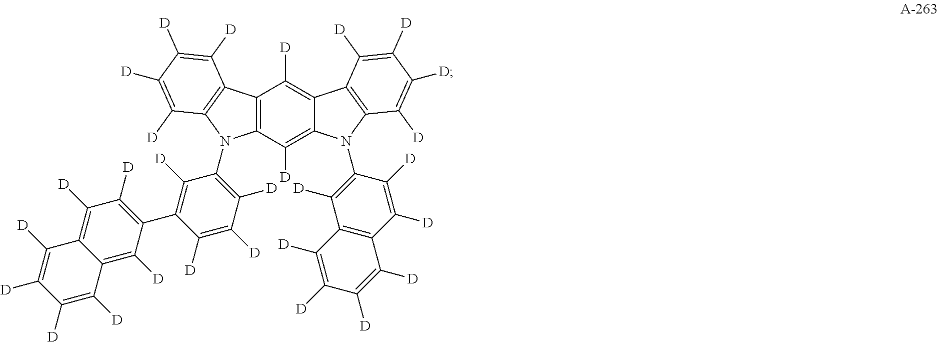
wherein hydrogen in Compound A-1 to Compound A-263 can be partially or fully substituted with deuterium.

13. The mixture of claim 1, wherein the second compound is selected from the group consisting of Compound B-1 to Compound B-223, wherein the specific structures of Compound B-1 to Compound B-223 are as follows: 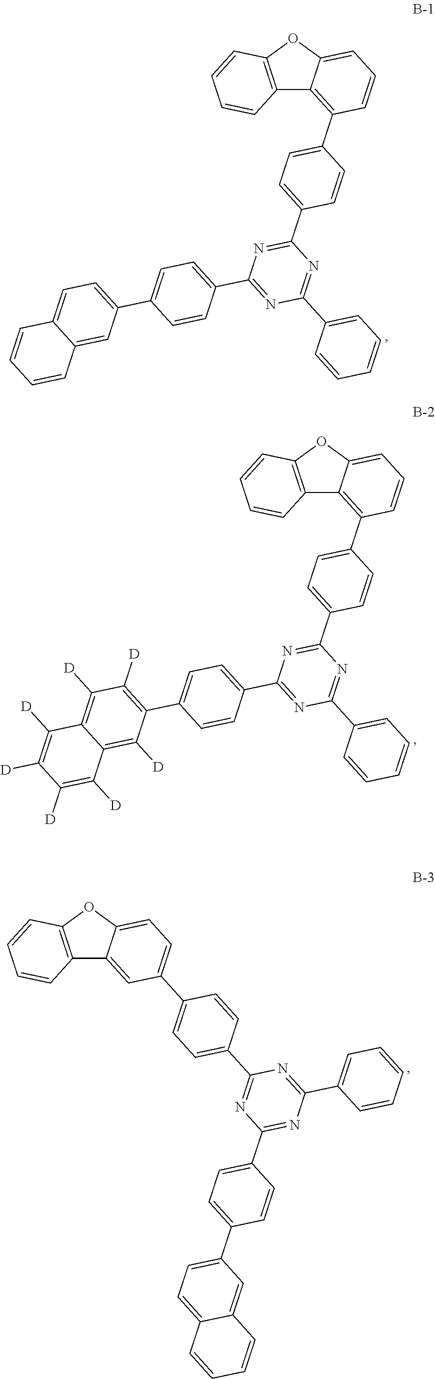 [SMILES: c1ccc(-c2nc(-c3ccc(-c4ccc5ccccc5c4)cc3)nc(-c3ccc(-c4cccc5oc6ccccc6c45)cc3)n2)cc1 ; [2H]c1c([2H])c([2H])c2c([2H])c(-c3ccc(-c4nc(-c5ccccc5)nc(-c5ccc(-c6cccc7oc8ccccc8c67)cc5)n4)cc3)c([2H])c([2H])c2c1[2H] ; c1ccc(-c2nc(-c3ccc(-c4ccc5ccccc5c4)cc3)nc(-c3ccc(-c4ccc5oc6ccccc6c5c4)cc3)n2)cc1] 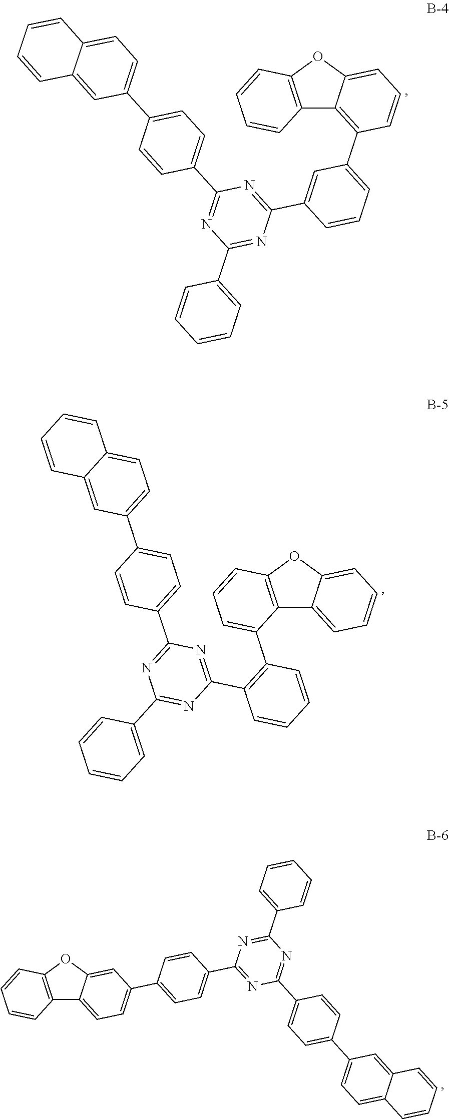 [SMILES: c1ccc(-c2nc(-c3ccc(-c4ccc5ccccc5c4)cc3)nc(-c3cccc(-c4cccc5oc6ccccc6c45)c3)n2)cc1 ; c1ccc(-c2nc(-c3ccc(-c4ccc5ccccc5c4)cc3)nc(-c3ccccc3-c3cccc4oc5ccccc5c34)n2)cc1 ; c1ccc(-c2nc(-c3ccc(-c4ccc5ccccc5c4)cc3)nc(-c3ccc(-c4ccc5c(c4)oc4ccccc45)cc3)n2)cc1] 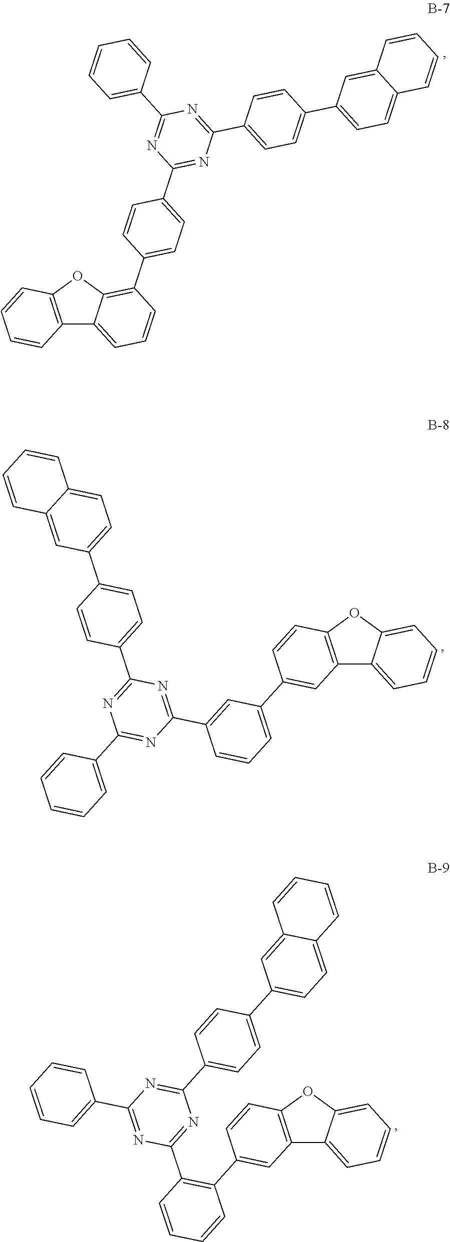 [SMILES: c1ccc(-c2nc(-c3ccc(-c4ccc5ccccc5c4)cc3)nc(-c3ccc(-c4cccc5c4oc4ccccc45)cc3)n2)cc1 ; c1ccc(-c2nc(-c3ccc(-c4ccc5ccccc5c4)cc3)nc(-c3cccc(-c4ccc5oc6ccccc6c5c4)c3)n2)cc1 ; c1ccc(-c2nc(-c3ccc(-c4ccc5ccccc5c4)cc3)nc(-c3ccccc3-c3ccc4oc5ccccc5c4c3)n2)cc1] 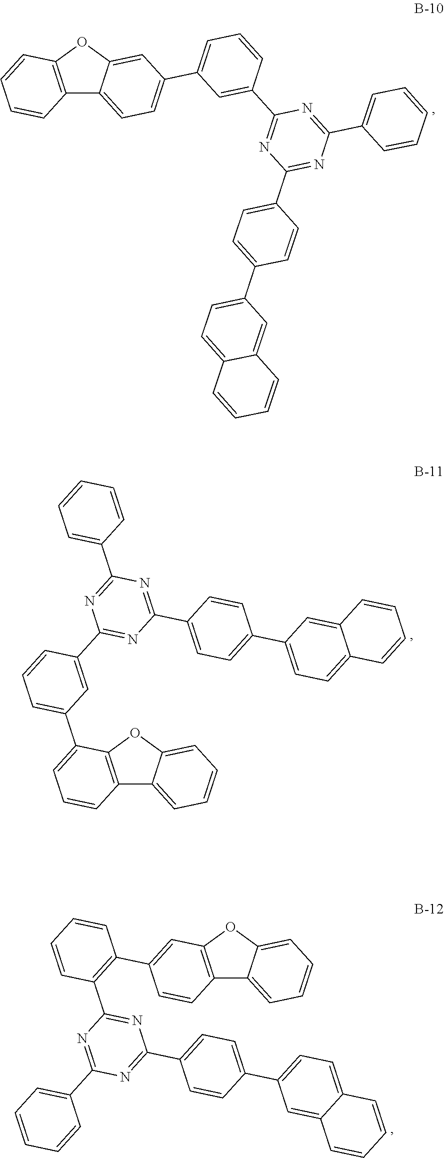 [SMILES: c1ccc(-c2nc(-c3ccc(-c4ccc5ccccc5c4)cc3)nc(-c3cccc(-c4ccc5c(c4)oc4ccccc45)c3)n2)cc1 ; c1ccc(-c2nc(-c3ccc(-c4ccc5ccccc5c4)cc3)nc(-c3cccc(-c4cccc5c4oc4ccccc45)c3)n2)cc1 ; c1ccc(-c2nc(-c3ccc(-c4ccc5ccccc5c4)cc3)nc(-c3ccccc3-c3ccc4c(c3)oc3ccccc34)n2)cc1] 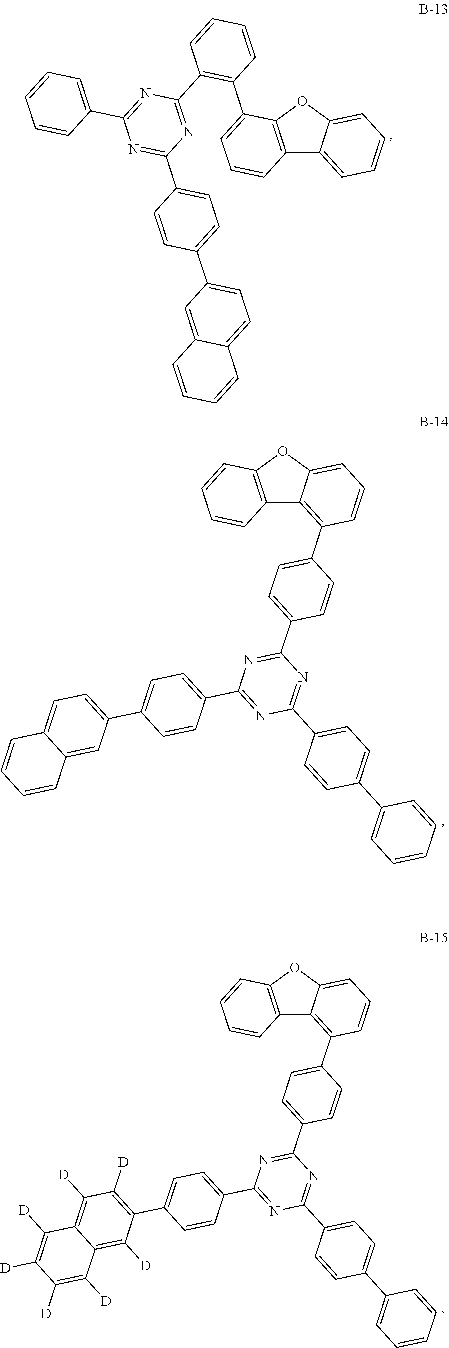 [SMILES: c1ccc(-c2nc(-c3ccc(-c4ccc5ccccc5c4)cc3)nc(-c3ccccc3-c3cccc4c3oc3ccccc34)n2)cc1 ; c1ccc(-c2ccc(-c3nc(-c4ccc(-c5ccc6ccccc6c5)cc4)nc(-c4ccc(-c5cccc6oc7ccccc7c56)cc4)n3)cc2)cc1 ; [2H]c1c([2H])c([2H])c2c([2H])c(-c3ccc(-c4nc(-c5ccc(-c6ccccc6)cc5)nc(-c5ccc(-c6cccc7oc8ccccc8c67)cc5)n4)cc3)c([2H])c([2H])c2c1[2H]] 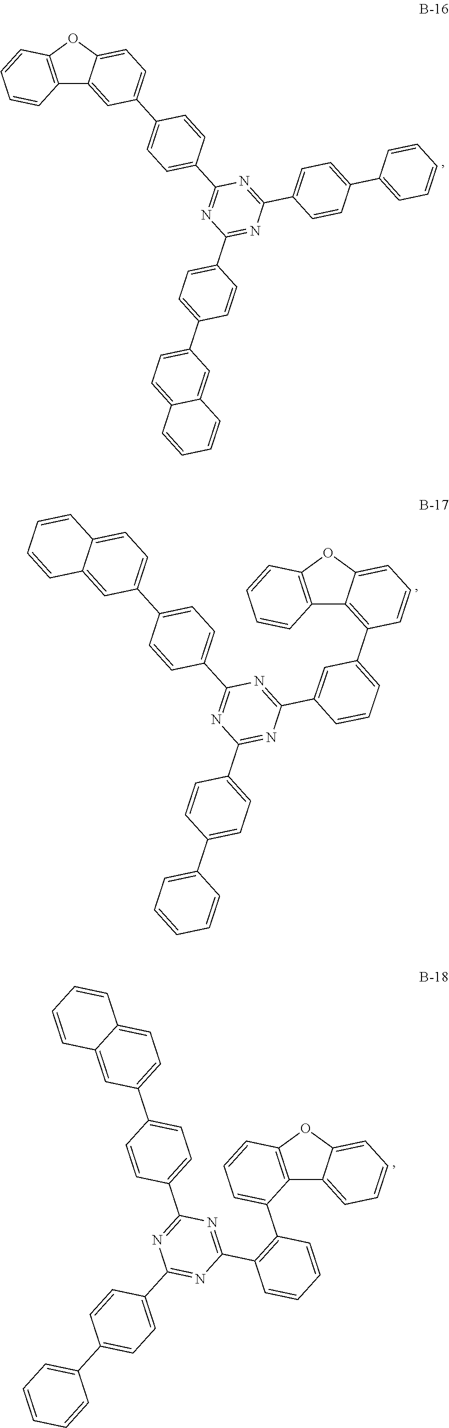 [SMILES: c1ccc(-c2ccc(-c3nc(-c4ccc(-c5ccc6ccccc6c5)cc4)nc(-c4ccc(-c5ccc6oc7ccccc7c6c5)cc4)n3)cc2)cc1 ; c1ccc(-c2ccc(-c3nc(-c4ccc(-c5ccc6ccccc6c5)cc4)nc(-c4cccc(-c5cccc6oc7ccccc7c56)c4)n3)cc2)cc1 ; c1ccc(-c2ccc(-c3nc(-c4ccc(-c5ccc6ccccc6c5)cc4)nc(-c4ccccc4-c4cccc5oc6ccccc6c45)n3)cc2)cc1] 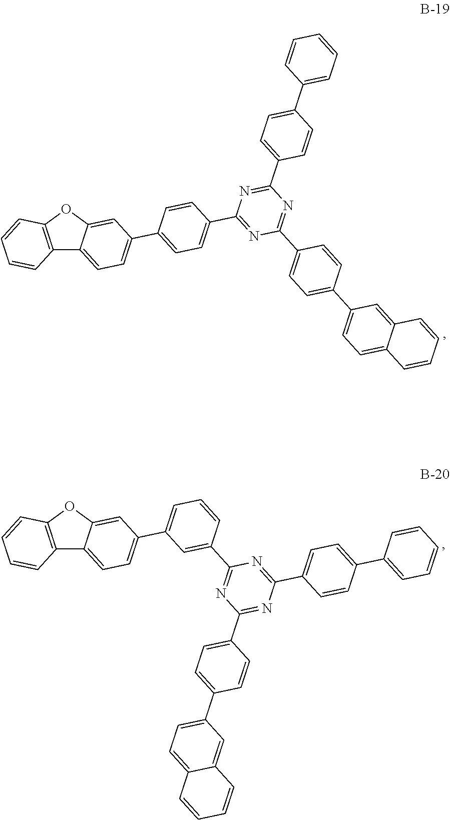 [SMILES: c1ccc(-c2ccc(-c3nc(-c4ccc(-c5ccc6ccccc6c5)cc4)nc(-c4ccc(-c5ccc6c(c5)oc5ccccc56)cc4)n3)cc2)cc1 ; c1ccc(-c2ccc(-c3nc(-c4ccc(-c5ccc6ccccc6c5)cc4)nc(-c4cccc(-c5ccc6c(c5)oc5ccccc56)c4)n3)cc2)cc1] 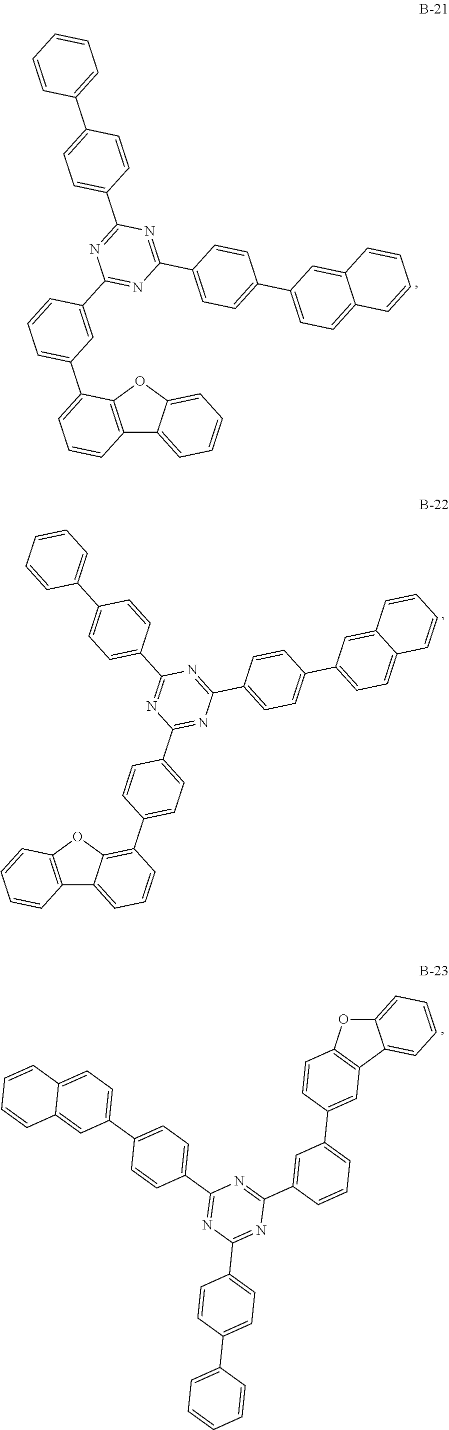 [SMILES: c1ccc(-c2ccc(-c3nc(-c4ccc(-c5ccc6ccccc6c5)cc4)nc(-c4cccc(-c5cccc6c5oc5ccccc56)c4)n3)cc2)cc1 ; c1ccc(-c2ccc(-c3nc(-c4ccc(-c5ccc6ccccc6c5)cc4)nc(-c4ccc(-c5cccc6c5oc5ccccc56)cc4)n3)cc2)cc1 ; c1ccc(-c2ccc(-c3nc(-c4ccc(-c5ccc6ccccc6c5)cc4)nc(-c4cccc(-c5ccc6oc7ccccc7c6c5)c4)n3)cc2)cc1] 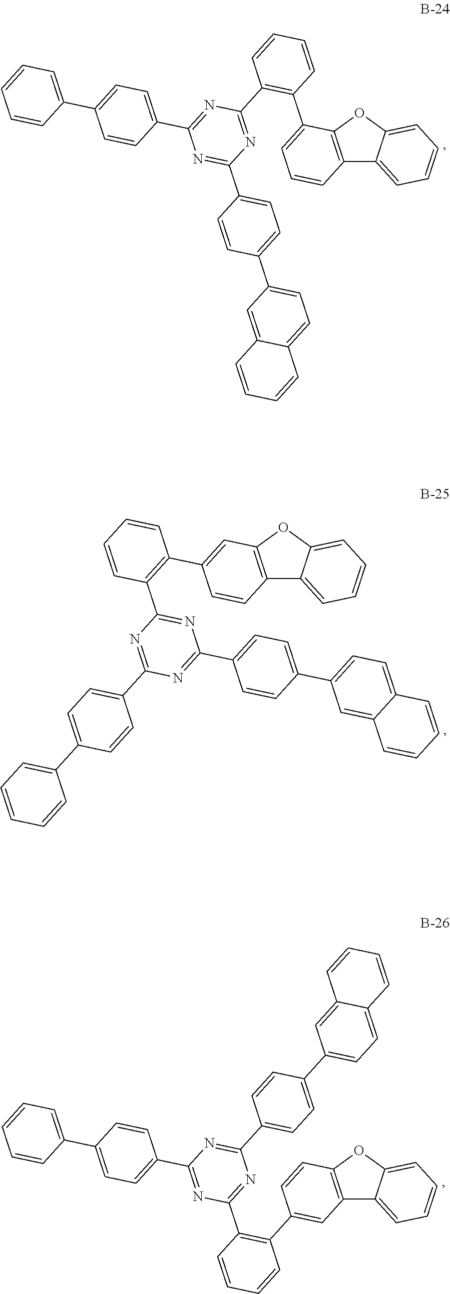 [SMILES: c1ccc(-c2ccc(-c3nc(-c4ccc(-c5ccc6ccccc6c5)cc4)nc(-c4ccccc4-c4cccc5c4oc4ccccc45)n3)cc2)cc1 ; c1ccc(-c2ccc(-c3nc(-c4ccc(-c5ccc6ccccc6c5)cc4)nc(-c4ccccc4-c4ccc5c(c4)oc4ccccc45)n3)cc2)cc1 ; c1ccc(-c2ccc(-c3nc(-c4ccc(-c5ccc6ccccc6c5)cc4)nc(-c4ccccc4-c4ccc5oc6ccccc6c5c4)n3)cc2)cc1] 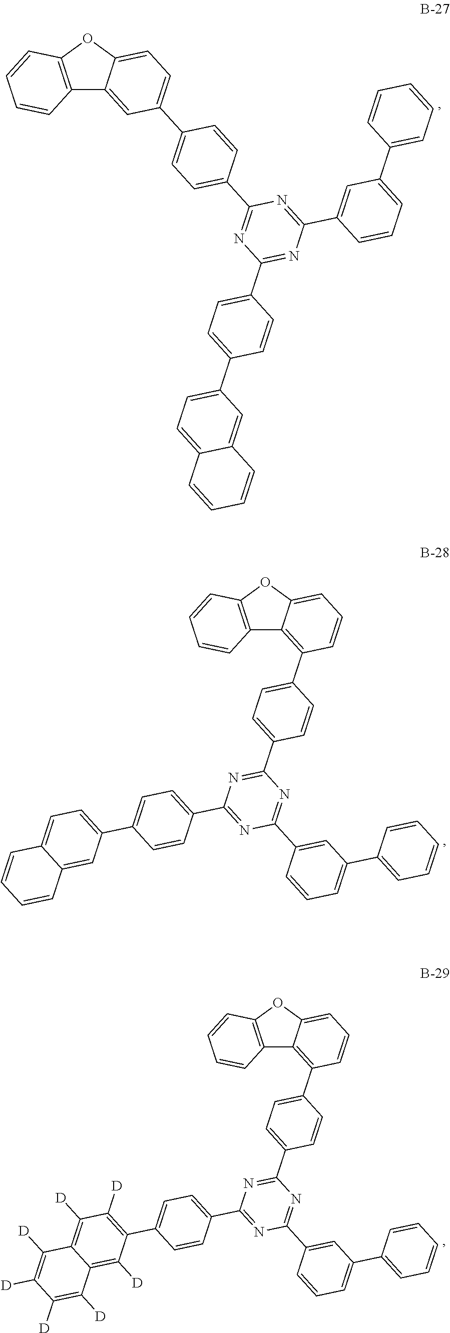 [SMILES: c1ccc(-c2cccc(-c3nc(-c4ccc(-c5ccc6ccccc6c5)cc4)nc(-c4ccc(-c5ccc6oc7ccccc7c6c5)cc4)n3)c2)cc1 ; c1ccc(-c2cccc(-c3nc(-c4ccc(-c5ccc6ccccc6c5)cc4)nc(-c4ccc(-c5cccc6oc7ccccc7c56)cc4)n3)c2)cc1 ; [2H]c1c([2H])c([2H])c2c([2H])c(-c3ccc(-c4nc(-c5ccc(-c6cccc7oc8ccccc8c67)cc5)nc(-c5cccc(-c6ccccc6)c5)n4)cc3)c([2H])c([2H])c2c1[2H]] 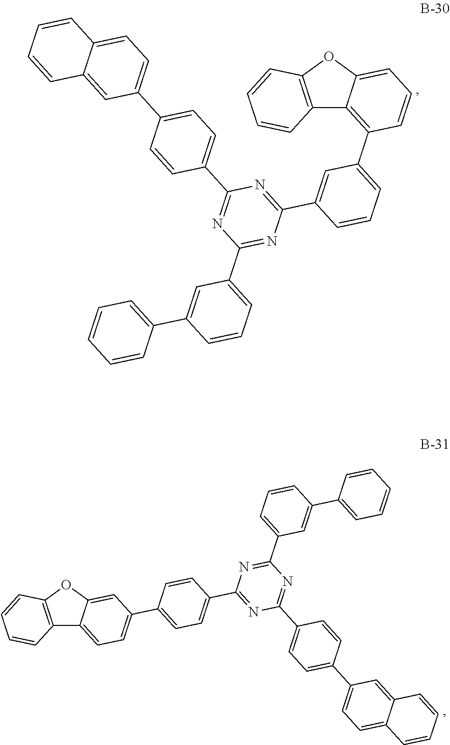 [SMILES: c1ccc(-c2cccc(-c3nc(-c4ccc(-c5ccc6ccccc6c5)cc4)nc(-c4cccc(-c5cccc6oc7ccccc7c56)c4)n3)c2)cc1 ; c1ccc(-c2cccc(-c3nc(-c4ccc(-c5ccc6ccccc6c5)cc4)nc(-c4ccc(-c5ccc6c(c5)oc5ccccc56)cc4)n3)c2)cc1] 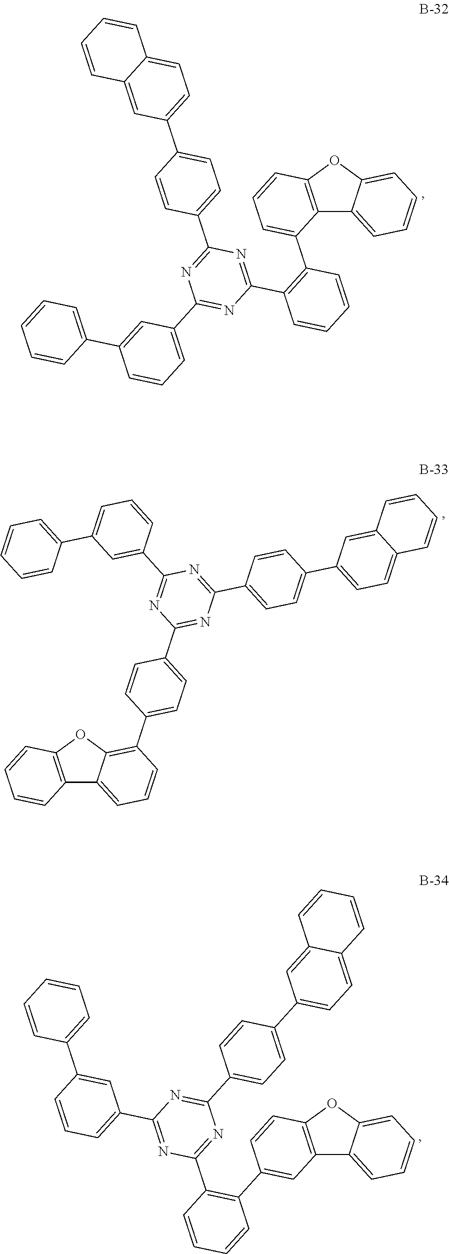 [SMILES: c1ccc(-c2cccc(-c3nc(-c4ccc(-c5ccc6ccccc6c5)cc4)nc(-c4ccccc4-c4cccc5oc6ccccc6c45)n3)c2)cc1 ; c1ccc(-c2cccc(-c3nc(-c4ccc(-c5ccc6ccccc6c5)cc4)nc(-c4ccc(-c5cccc6c5oc5ccccc56)cc4)n3)c2)cc1 ; c1ccc(-c2cccc(-c3nc(-c4ccc(-c5ccc6ccccc6c5)cc4)nc(-c4ccccc4-c4ccc5oc6ccccc6c5c4)n3)c2)cc1] 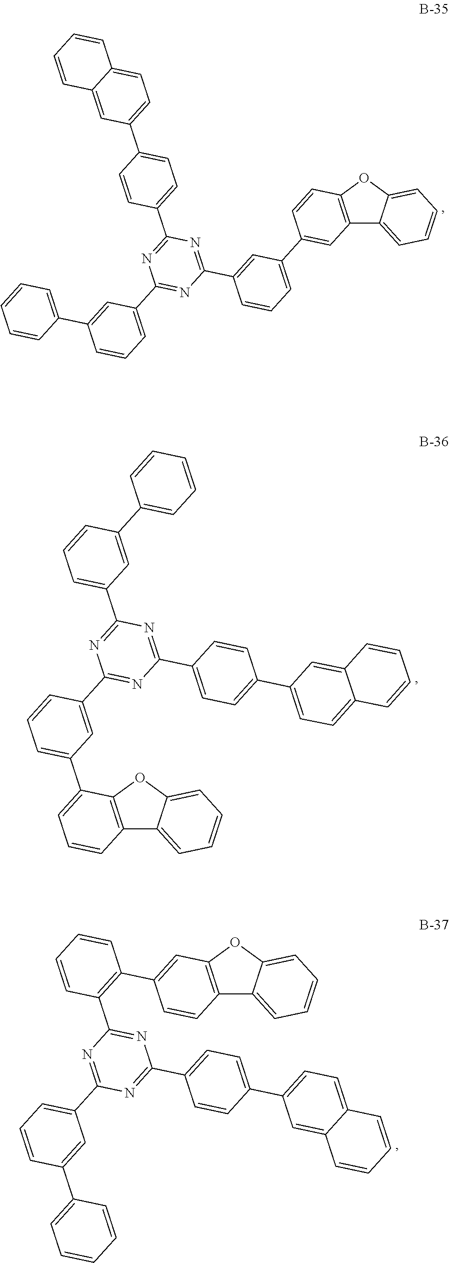 [SMILES: c1ccc(-c2cccc(-c3nc(-c4ccc(-c5ccc6ccccc6c5)cc4)nc(-c4cccc(-c5ccc6oc7ccccc7c6c5)c4)n3)c2)cc1 ; c1ccc(-c2cccc(-c3nc(-c4ccc(-c5ccc6ccccc6c5)cc4)nc(-c4cccc(-c5cccc6c5oc5ccccc56)c4)n3)c2)cc1 ; c1ccc(-c2cccc(-c3nc(-c4ccc(-c5ccc6ccccc6c5)cc4)nc(-c4ccccc4-c4ccc5c(c4)oc4ccccc45)n3)c2)cc1] 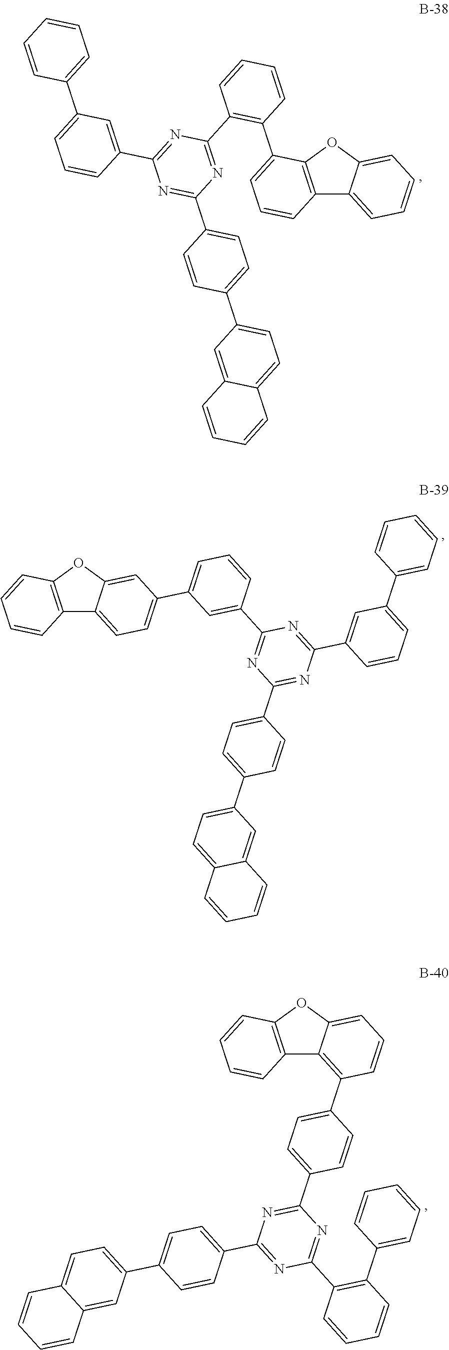 [SMILES: c1ccc(-c2cccc(-c3nc(-c4ccc(-c5ccc6ccccc6c5)cc4)nc(-c4ccccc4-c4cccc5c4oc4ccccc45)n3)c2)cc1 ; c1ccc(-c2cccc(-c3nc(-c4ccc(-c5ccc6ccccc6c5)cc4)nc(-c4cccc(-c5ccc6c(c5)oc5ccccc56)c4)n3)c2)cc1 ; c1ccc(-c2ccccc2-c2nc(-c3ccc(-c4ccc5ccccc5c4)cc3)nc(-c3ccc(-c4cccc5oc6ccccc6c45)cc3)n2)cc1] 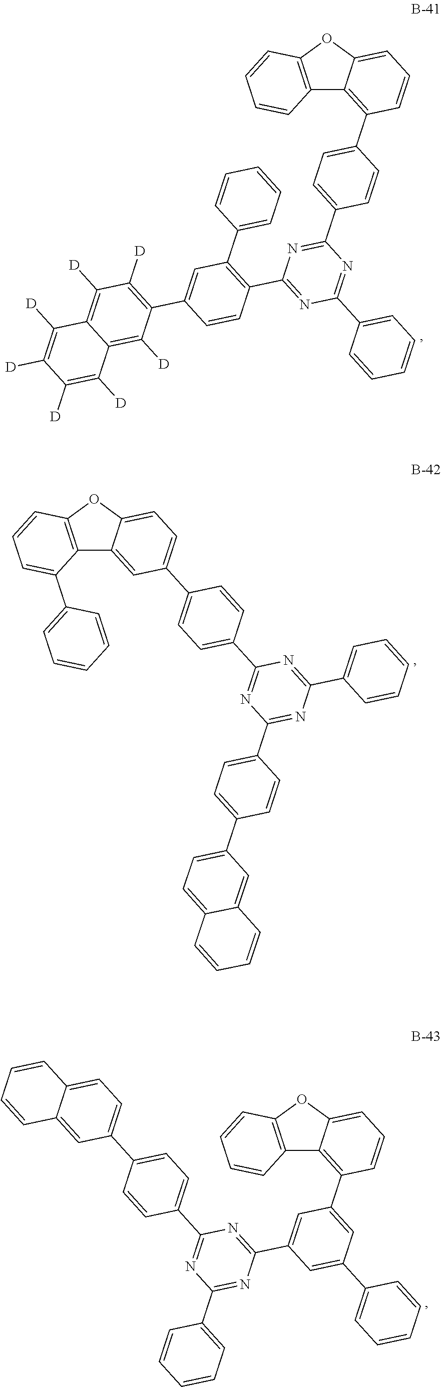 [SMILES: [2H]c1c([2H])c([2H])c2c([2H])c(-c3ccc(-c4nc(-c5ccccc5)nc(-c5ccc(-c6cccc7oc8ccccc8c67)cc5)n4)c(-c4ccccc4)c3)c([2H])c([2H])c2c1[2H] ; c1ccc(-c2nc(-c3ccc(-c4ccc5ccccc5c4)cc3)nc(-c3ccc(-c4ccc5oc6cccc(-c7ccccc7)c6c5c4)cc3)n2)cc1 ; c1ccc(-c2cc(-c3nc(-c4ccccc4)nc(-c4ccc(-c5ccc6ccccc6c5)cc4)n3)cc(-c3cccc4oc5ccccc5c34)c2)cc1] 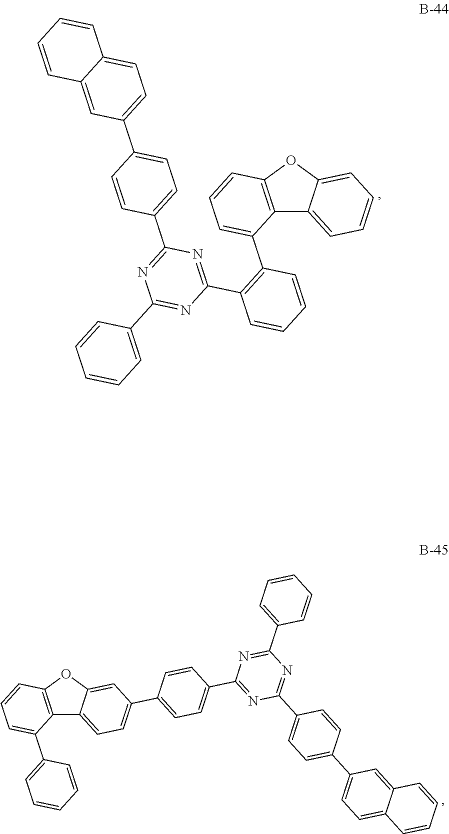 [SMILES: c1ccc(-c2nc(-c3ccc(-c4ccc5ccccc5c4)cc3)nc(-c3ccccc3-c3cccc4oc5ccccc5c34)n2)cc1 ; c1ccc(-c2nc(-c3ccc(-c4ccc5ccccc5c4)cc3)nc(-c3ccc(-c4ccc5c(c4)oc4cccc(-c6ccccc6)c45)cc3)n2)cc1] 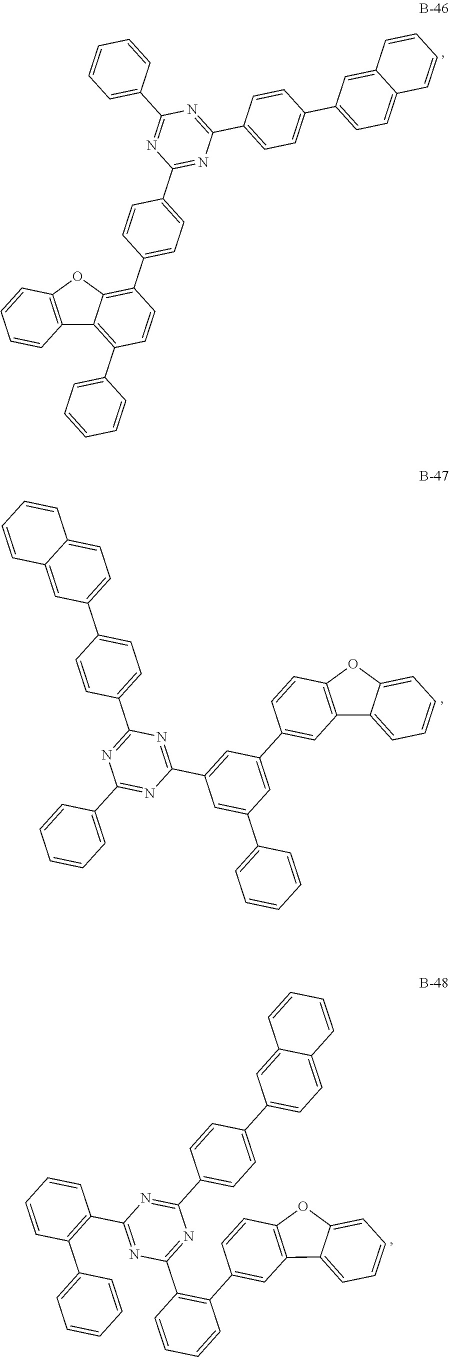 [SMILES: c1ccc(-c2nc(-c3ccc(-c4ccc5ccccc5c4)cc3)nc(-c3ccc(-c4ccc(-c5ccccc5)c5c4oc4ccccc45)cc3)n2)cc1 ; c1ccc(-c2cc(-c3ccc4oc5ccccc5c4c3)cc(-c3nc(-c4ccccc4)nc(-c4ccc(-c5ccc6ccccc6c5)cc4)n3)c2)cc1 ; c1ccc(-c2ccccc2-c2nc(-c3ccc(-c4ccc5ccccc5c4)cc3)nc(-c3ccccc3-c3ccc4oc5ccccc5c4c3)n2)cc1] 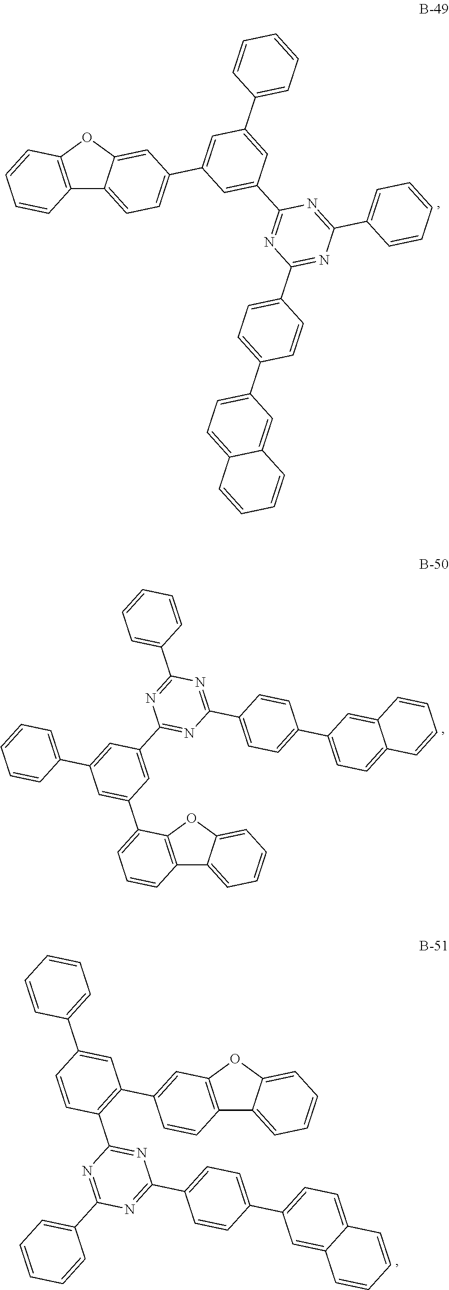 [SMILES: c1ccc(-c2cc(-c3ccc4c(c3)oc3ccccc34)cc(-c3nc(-c4ccccc4)nc(-c4ccc(-c5ccc6ccccc6c5)cc4)n3)c2)cc1 ; c1ccc(-c2cc(-c3nc(-c4ccccc4)nc(-c4ccc(-c5ccc6ccccc6c5)cc4)n3)cc(-c3cccc4c3oc3ccccc34)c2)cc1 ; c1ccc(-c2ccc(-c3nc(-c4ccccc4)nc(-c4ccc(-c5ccc6ccccc6c5)cc4)n3)c(-c3ccc4c(c3)oc3ccccc34)c2)cc1] 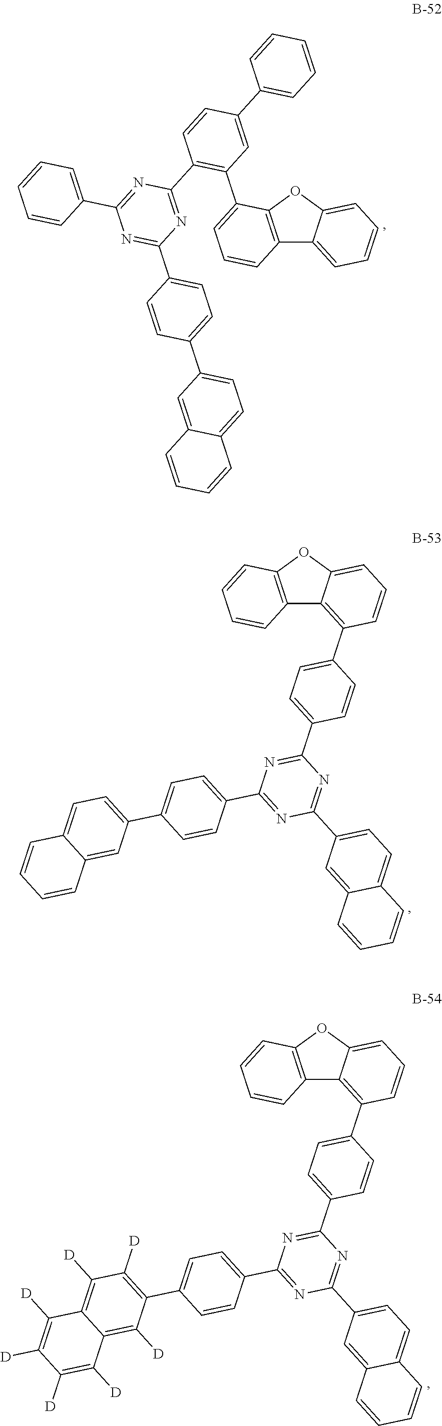 [SMILES: c1ccc(-c2ccc(-c3nc(-c4ccccc4)nc(-c4ccc(-c5ccc6ccccc6c5)cc4)n3)c(-c3cccc4c3oc3ccccc34)c2)cc1 ; c1ccc2cc(-c3ccc(-c4nc(-c5ccc(-c6cccc7oc8ccccc8c67)cc5)nc(-c5ccc6ccccc6c5)n4)cc3)ccc2c1 ; [2H]c1c([2H])c([2H])c2c([2H])c(-c3ccc(-c4nc(-c5ccc(-c6cccc7oc8ccccc8c67)cc5)nc(-c5ccc6ccccc6c5)n4)cc3)c([2H])c([2H])c2c1[2H]] 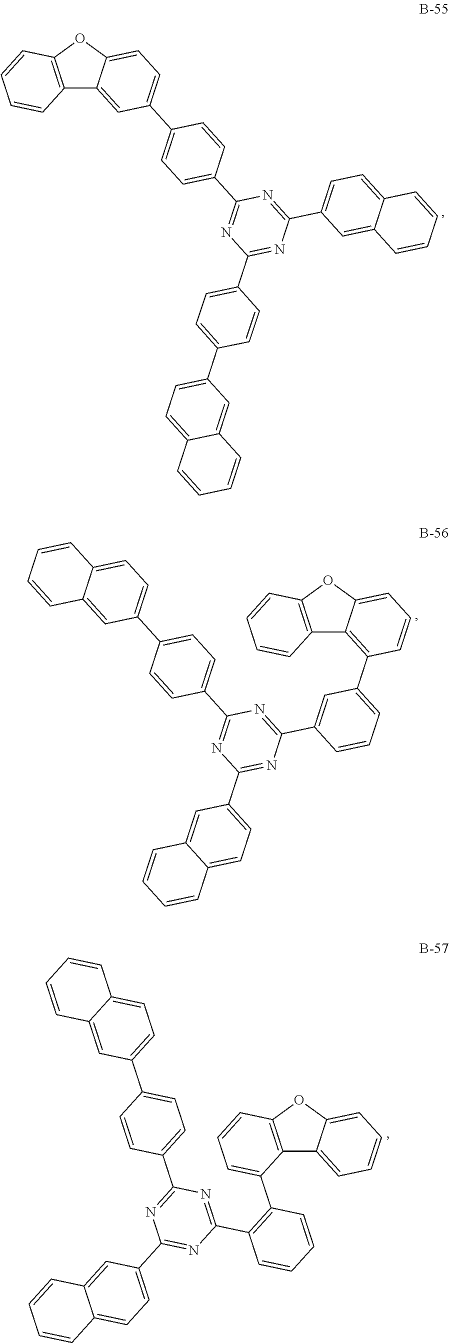 [SMILES: c1ccc2cc(-c3ccc(-c4nc(-c5ccc(-c6ccc7oc8ccccc8c7c6)cc5)nc(-c5ccc6ccccc6c5)n4)cc3)ccc2c1 ; c1cc(-c2nc(-c3ccc(-c4ccc5ccccc5c4)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4ccc5ccccc5c4)cc3)nc(-c3ccc4ccccc4c3)n2)c1] 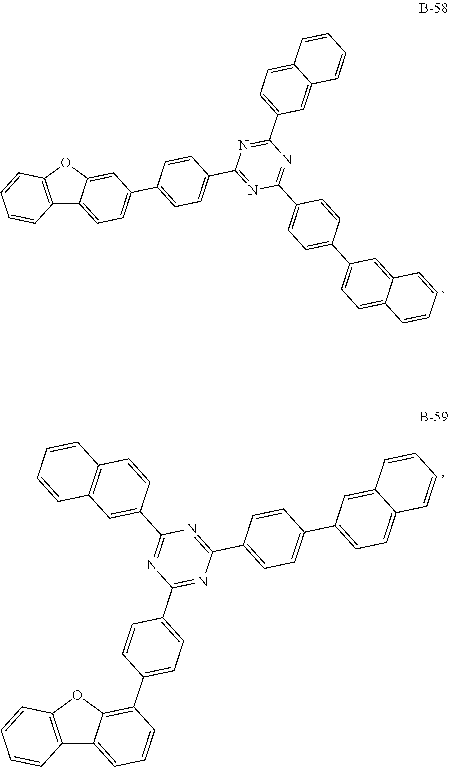 [SMILES: c1ccc2cc(-c3ccc(-c4nc(-c5ccc(-c6ccc7c(c6)oc6ccccc67)cc5)nc(-c5ccc6ccccc6c5)n4)cc3)ccc2c1 ; c1ccc2cc(-c3ccc(-c4nc(-c5ccc(-c6cccc7c6oc6ccccc67)cc5)nc(-c5ccc6ccccc6c5)n4)cc3)ccc2c1] 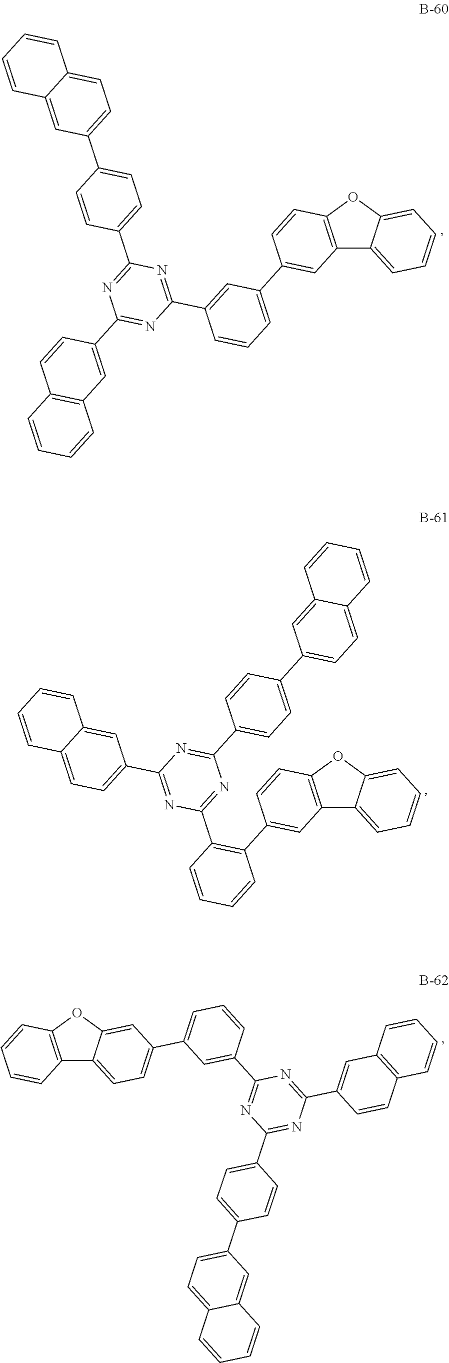 [SMILES: c1cc(-c2ccc3oc4ccccc4c3c2)cc(-c2nc(-c3ccc(-c4ccc5ccccc5c4)cc3)nc(-c3ccc4ccccc4c3)n2)c1 ; c1ccc(-c2nc(-c3ccc(-c4ccc5ccccc5c4)cc3)nc(-c3ccc4ccccc4c3)n2)c(-c2ccc3oc4ccccc4c3c2)c1 ; c1cc(-c2ccc3c(c2)oc2ccccc23)cc(-c2nc(-c3ccc(-c4ccc5ccccc5c4)cc3)nc(-c3ccc4ccccc4c3)n2)c1] 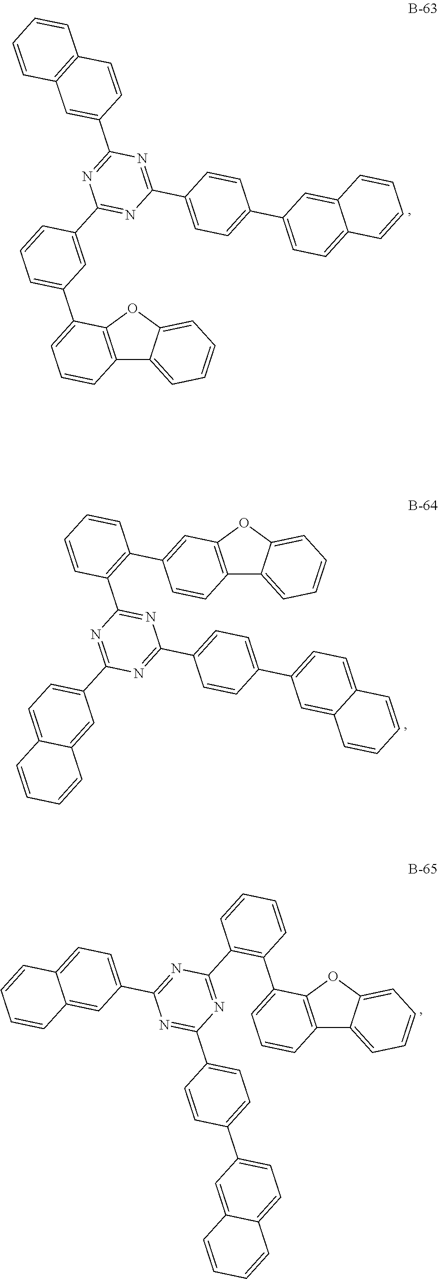 [SMILES: c1cc(-c2nc(-c3ccc(-c4ccc5ccccc5c4)cc3)nc(-c3ccc4ccccc4c3)n2)cc(-c2cccc3c2oc2ccccc23)c1 ; c1ccc(-c2nc(-c3ccc(-c4ccc5ccccc5c4)cc3)nc(-c3ccc4ccccc4c3)n2)c(-c2ccc3c(c2)oc2ccccc23)c1 ; c1ccc(-c2cccc3c2oc2ccccc23)c(-c2nc(-c3ccc(-c4ccc5ccccc5c4)cc3)nc(-c3ccc4ccccc4c3)n2)c1] 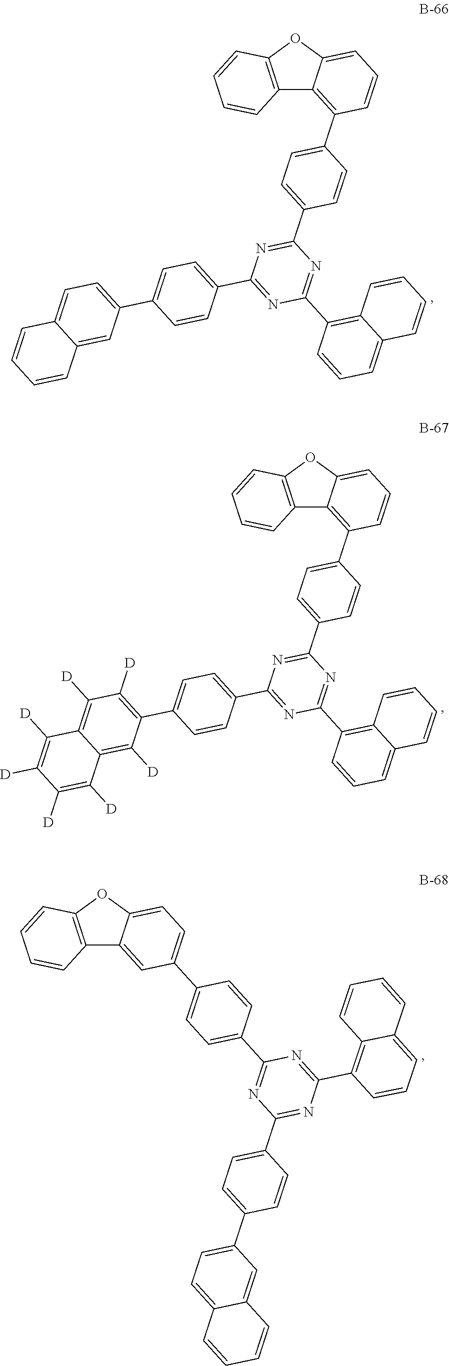 [SMILES: c1ccc2cc(-c3ccc(-c4nc(-c5ccc(-c6cccc7oc8ccccc8c67)cc5)nc(-c5cccc6ccccc56)n4)cc3)ccc2c1 ; [2H]c1c([2H])c([2H])c2c([2H])c(-c3ccc(-c4nc(-c5ccc(-c6cccc7oc8ccccc8c67)cc5)nc(-c5cccc6ccccc56)n4)cc3)c([2H])c([2H])c2c1[2H] ; c1ccc2cc(-c3ccc(-c4nc(-c5ccc(-c6ccc7oc8ccccc8c7c6)cc5)nc(-c5cccc6ccccc56)n4)cc3)ccc2c1] 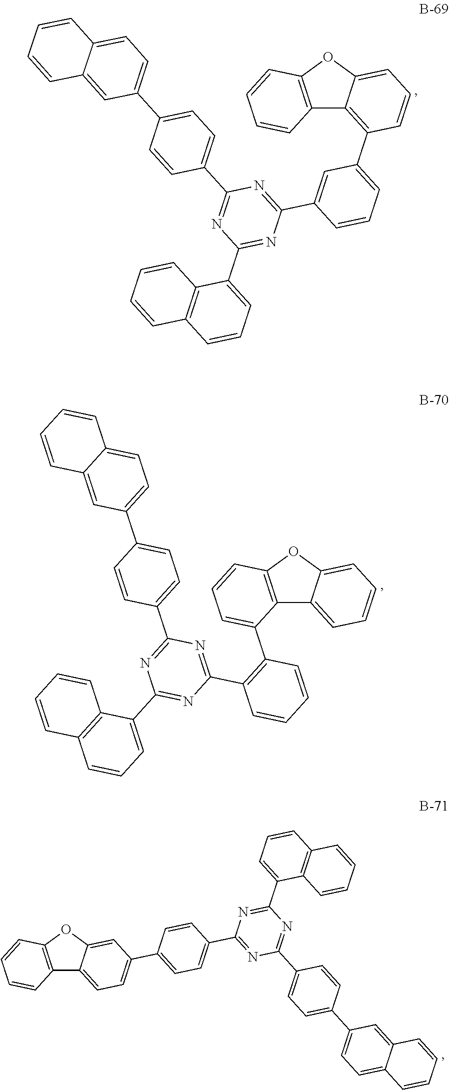 [SMILES: c1cc(-c2nc(-c3ccc(-c4ccc5ccccc5c4)cc3)nc(-c3cccc4ccccc34)n2)cc(-c2cccc3oc4ccccc4c23)c1 ; c1ccc(-c2cccc3oc4ccccc4c23)c(-c2nc(-c3ccc(-c4ccc5ccccc5c4)cc3)nc(-c3cccc4ccccc34)n2)c1 ; c1ccc2cc(-c3ccc(-c4nc(-c5ccc(-c6ccc7c(c6)oc6ccccc67)cc5)nc(-c5cccc6ccccc56)n4)cc3)ccc2c1] 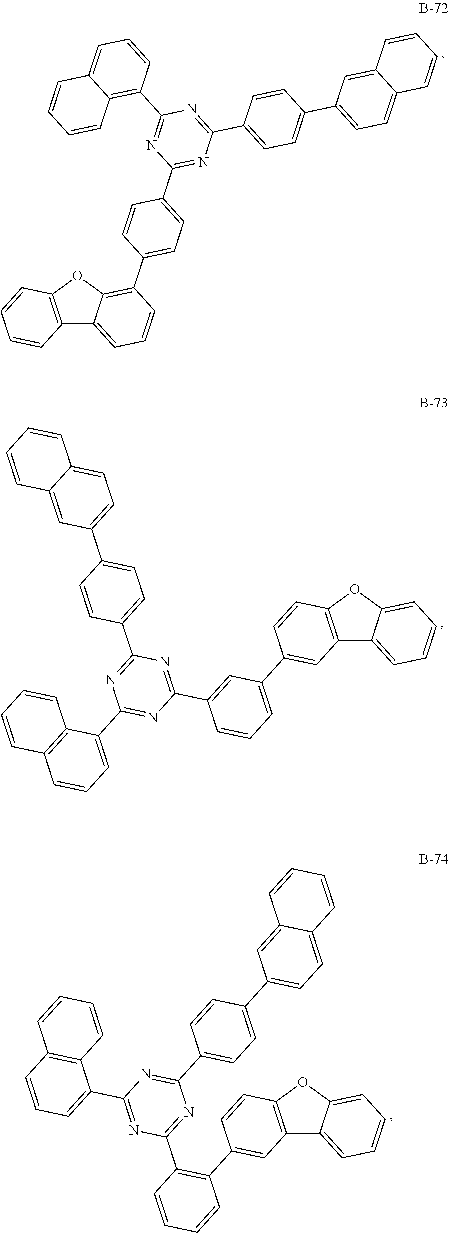 [SMILES: c1ccc2cc(-c3ccc(-c4nc(-c5ccc(-c6cccc7c6oc6ccccc67)cc5)nc(-c5cccc6ccccc56)n4)cc3)ccc2c1 ; c1cc(-c2ccc3oc4ccccc4c3c2)cc(-c2nc(-c3ccc(-c4ccc5ccccc5c4)cc3)nc(-c3cccc4ccccc34)n2)c1 ; c1ccc(-c2nc(-c3ccc(-c4ccc5ccccc5c4)cc3)nc(-c3cccc4ccccc34)n2)c(-c2ccc3oc4ccccc4c3c2)c1] 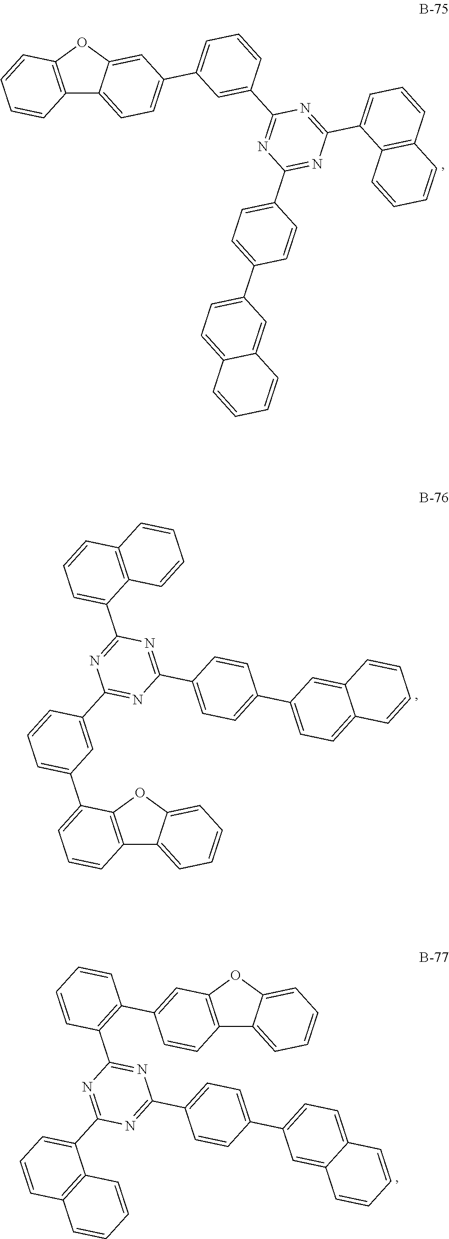 [SMILES: c1cc(-c2ccc3c(c2)oc2ccccc23)cc(-c2nc(-c3ccc(-c4ccc5ccccc5c4)cc3)nc(-c3cccc4ccccc34)n2)c1 ; c1cc(-c2nc(-c3ccc(-c4ccc5ccccc5c4)cc3)nc(-c3cccc4ccccc34)n2)cc(-c2cccc3c2oc2ccccc23)c1 ; c1ccc(-c2nc(-c3ccc(-c4ccc5ccccc5c4)cc3)nc(-c3cccc4ccccc34)n2)c(-c2ccc3c(c2)oc2ccccc23)c1] 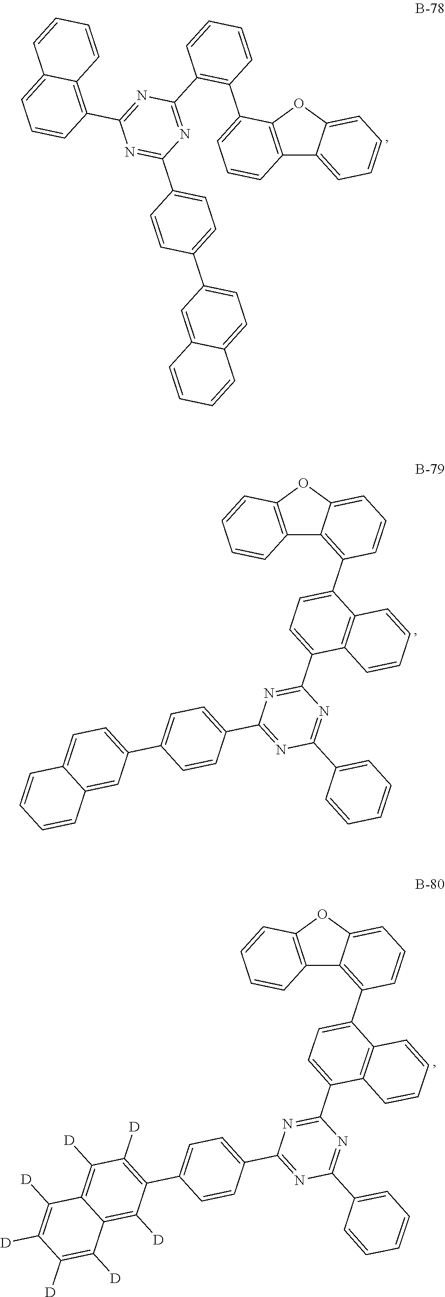 [SMILES: c1ccc(-c2cccc3c2oc2ccccc23)c(-c2nc(-c3ccc(-c4ccc5ccccc5c4)cc3)nc(-c3cccc4ccccc34)n2)c1 ; c1ccc(-c2nc(-c3ccc(-c4ccc5ccccc5c4)cc3)nc(-c3ccc(-c4cccc5oc6ccccc6c45)c4ccccc34)n2)cc1 ; [2H]c1c([2H])c([2H])c2c([2H])c(-c3ccc(-c4nc(-c5ccccc5)nc(-c5ccc(-c6cccc7oc8ccccc8c67)c6ccccc56)n4)cc3)c([2H])c([2H])c2c1[2H]] 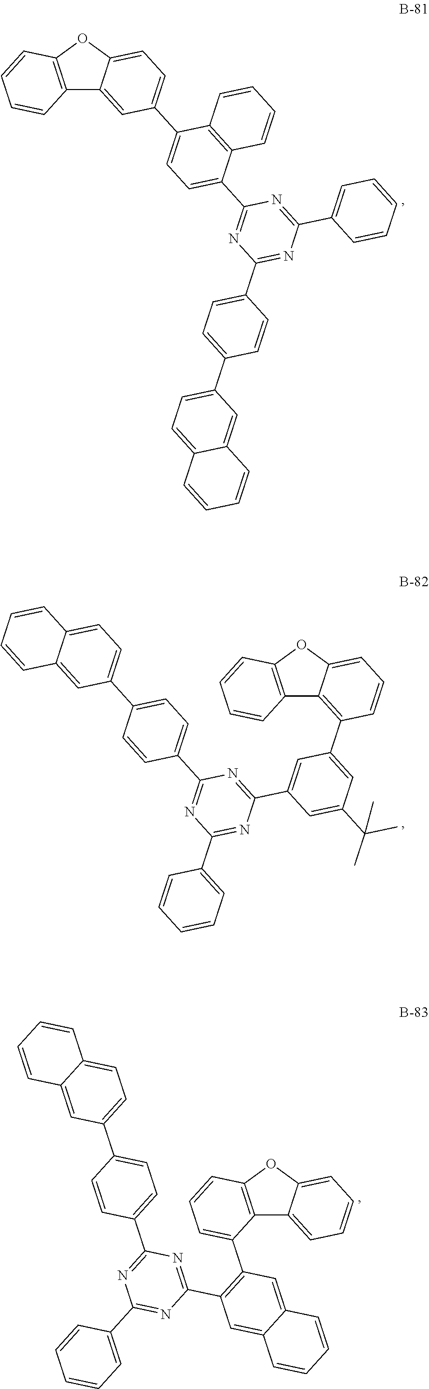 [SMILES: c1ccc(-c2nc(-c3ccc(-c4ccc5ccccc5c4)cc3)nc(-c3ccc(-c4ccc5oc6ccccc6c5c4)c4ccccc34)n2)cc1 ; CC(C)(C)c1cc(-c2nc(-c3ccccc3)nc(-c3ccc(-c4ccc5ccccc5c4)cc3)n2)cc(-c2cccc3oc4ccccc4c23)c1 ; c1ccc(-c2nc(-c3ccc(-c4ccc5ccccc5c4)cc3)nc(-c3cc4ccccc4cc3-c3cccc4oc5ccccc5c34)n2)cc1] 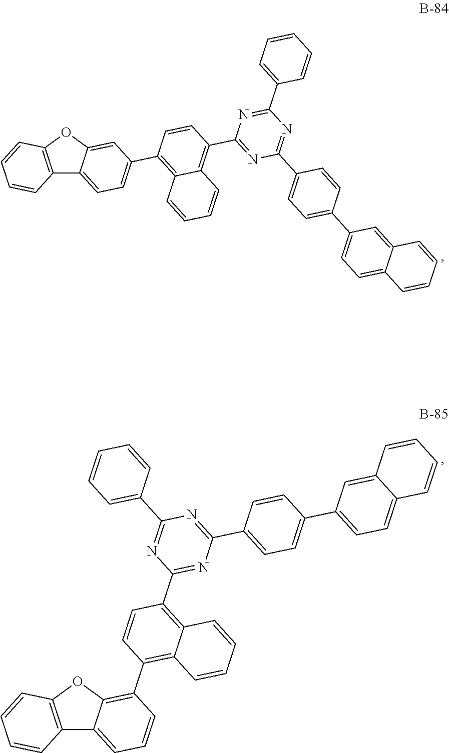 [SMILES: c1ccc(-c2nc(-c3ccc(-c4ccc5ccccc5c4)cc3)nc(-c3ccc(-c4ccc5c(c4)oc4ccccc45)c4ccccc34)n2)cc1 ; c1ccc(-c2nc(-c3ccc(-c4ccc5ccccc5c4)cc3)nc(-c3ccc(-c4cccc5c4oc4ccccc45)c4ccccc34)n2)cc1] 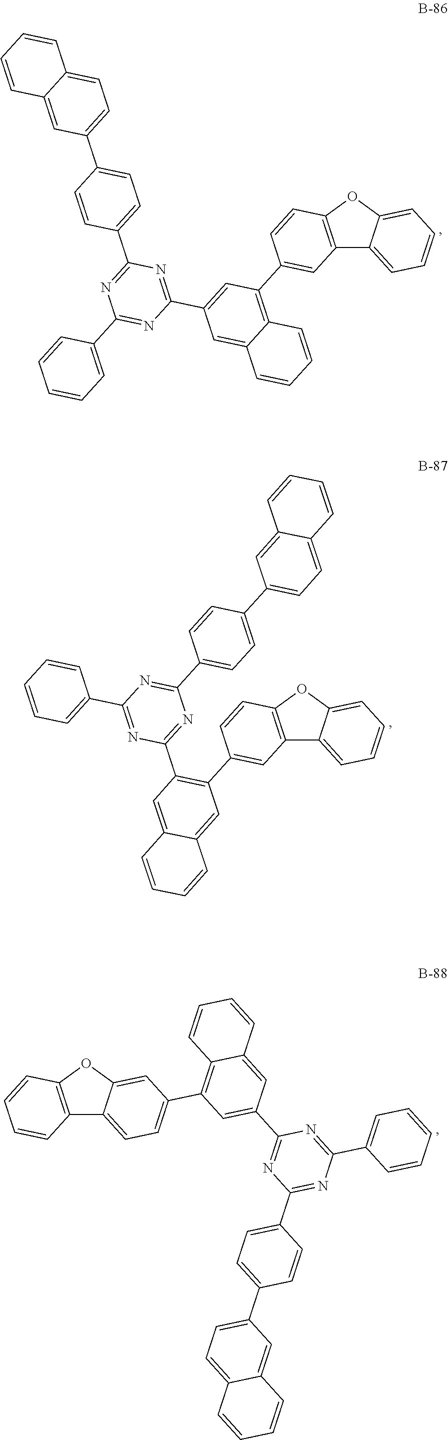 [SMILES: c1ccc(-c2nc(-c3ccc(-c4ccc5ccccc5c4)cc3)nc(-c3cc(-c4ccc5oc6ccccc6c5c4)c4ccccc4c3)n2)cc1 ; c1ccc(-c2nc(-c3ccc(-c4ccc5ccccc5c4)cc3)nc(-c3cc4ccccc4cc3-c3ccc4oc5ccccc5c4c3)n2)cc1 ; c1ccc(-c2nc(-c3ccc(-c4ccc5ccccc5c4)cc3)nc(-c3cc(-c4ccc5c(c4)oc4ccccc45)c4ccccc4c3)n2)cc1] 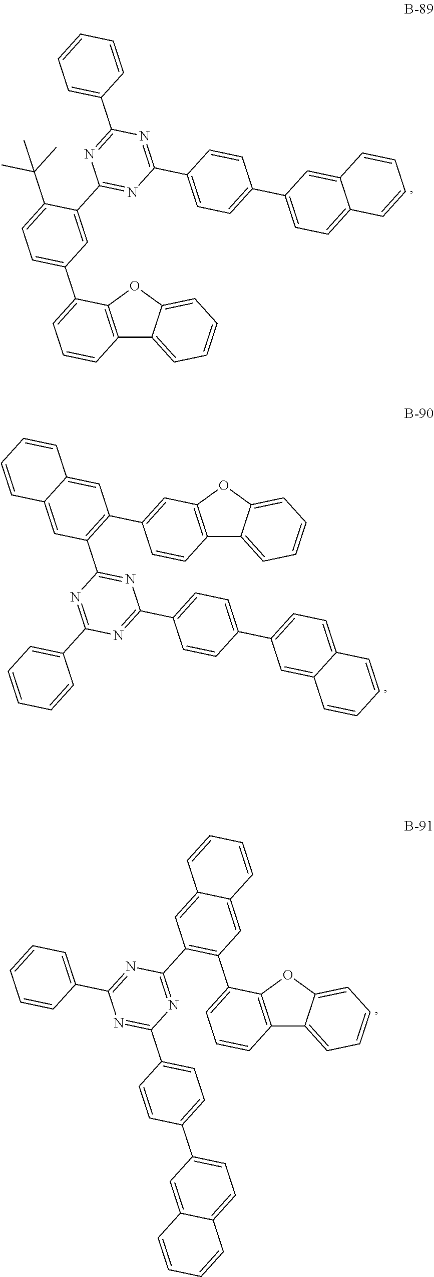 [SMILES: CC(C)(C)c1ccc(-c2cccc3c2oc2ccccc23)cc1-c1nc(-c2ccccc2)nc(-c2ccc(-c3ccc4ccccc4c3)cc2)n1 ; c1ccc(-c2nc(-c3ccc(-c4ccc5ccccc5c4)cc3)nc(-c3cc4ccccc4cc3-c3ccc4c(c3)oc3ccccc34)n2)cc1 ; c1ccc(-c2nc(-c3ccc(-c4ccc5ccccc5c4)cc3)nc(-c3cc4ccccc4cc3-c3cccc4c3oc3ccccc34)n2)cc1] 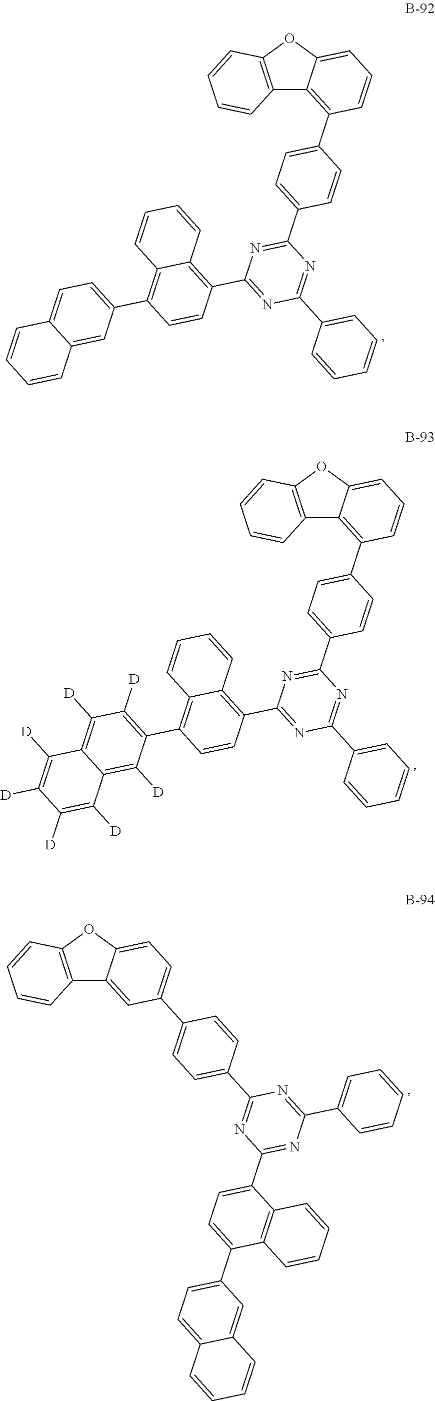 [SMILES: c1ccc(-c2nc(-c3ccc(-c4cccc5oc6ccccc6c45)cc3)nc(-c3ccc(-c4ccc5ccccc5c4)c4ccccc34)n2)cc1 ; [2H]c1c([2H])c([2H])c2c([2H])c(-c3ccc(-c4nc(-c5ccccc5)nc(-c5ccc(-c6cccc7oc8ccccc8c67)cc5)n4)c4ccccc34)c([2H])c([2H])c2c1[2H] ; c1ccc(-c2nc(-c3ccc(-c4ccc5oc6ccccc6c5c4)cc3)nc(-c3ccc(-c4ccc5ccccc5c4)c4ccccc34)n2)cc1] 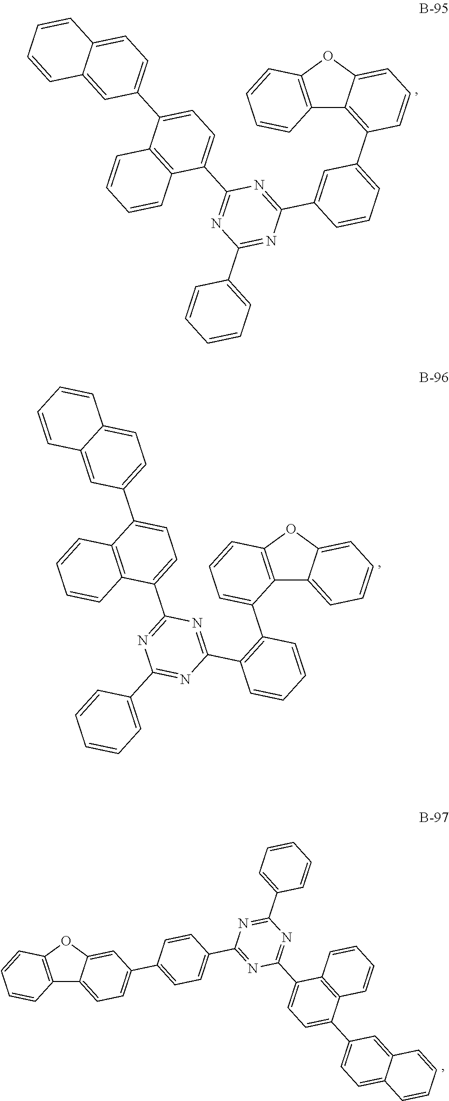 [SMILES: c1ccc(-c2nc(-c3cccc(-c4cccc5oc6ccccc6c45)c3)nc(-c3ccc(-c4ccc5ccccc5c4)c4ccccc34)n2)cc1 ; c1ccc(-c2nc(-c3ccccc3-c3cccc4oc5ccccc5c34)nc(-c3ccc(-c4ccc5ccccc5c4)c4ccccc34)n2)cc1 ; c1ccc(-c2nc(-c3ccc(-c4ccc5c(c4)oc4ccccc45)cc3)nc(-c3ccc(-c4ccc5ccccc5c4)c4ccccc34)n2)cc1] 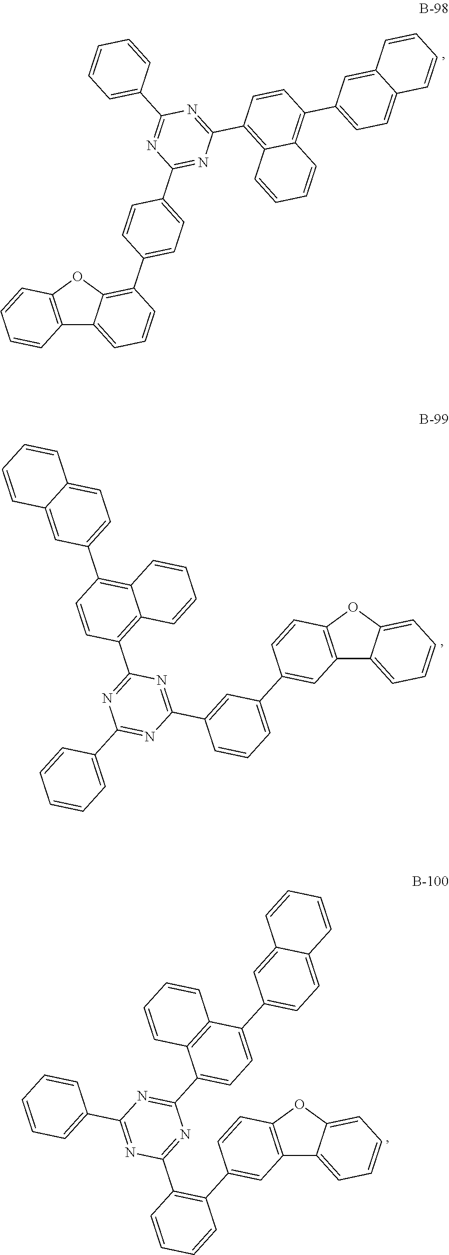 [SMILES: c1ccc(-c2nc(-c3ccc(-c4cccc5c4oc4ccccc45)cc3)nc(-c3ccc(-c4ccc5ccccc5c4)c4ccccc34)n2)cc1 ; c1ccc(-c2nc(-c3cccc(-c4ccc5oc6ccccc6c5c4)c3)nc(-c3ccc(-c4ccc5ccccc5c4)c4ccccc34)n2)cc1 ; c1ccc(-c2nc(-c3ccccc3-c3ccc4oc5ccccc5c4c3)nc(-c3ccc(-c4ccc5ccccc5c4)c4ccccc34)n2)cc1] 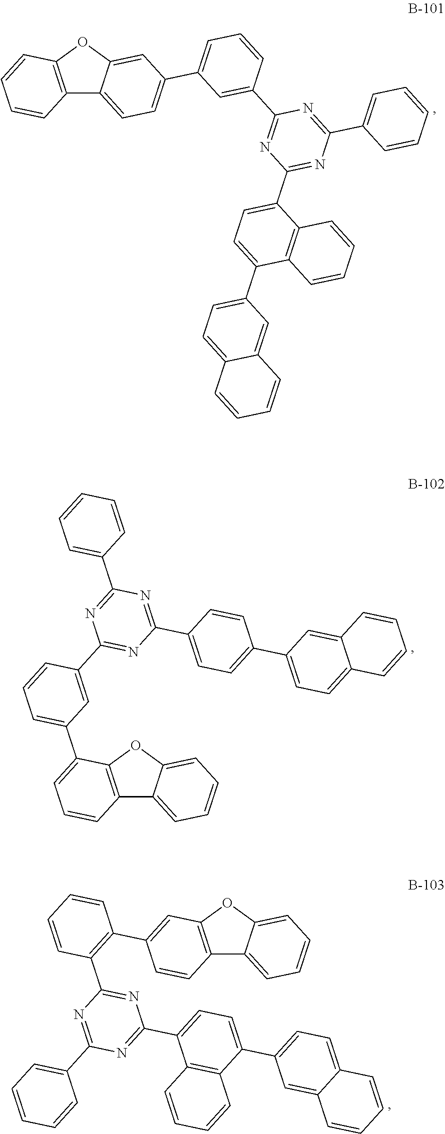 [SMILES: c1ccc(-c2nc(-c3cccc(-c4ccc5c(c4)oc4ccccc45)c3)nc(-c3ccc(-c4ccc5ccccc5c4)c4ccccc34)n2)cc1 ; c1ccc(-c2nc(-c3ccc(-c4ccc5ccccc5c4)cc3)nc(-c3cccc(-c4cccc5c4oc4ccccc45)c3)n2)cc1 ; c1ccc(-c2nc(-c3ccccc3-c3ccc4c(c3)oc3ccccc34)nc(-c3ccc(-c4ccc5ccccc5c4)c4ccccc34)n2)cc1] 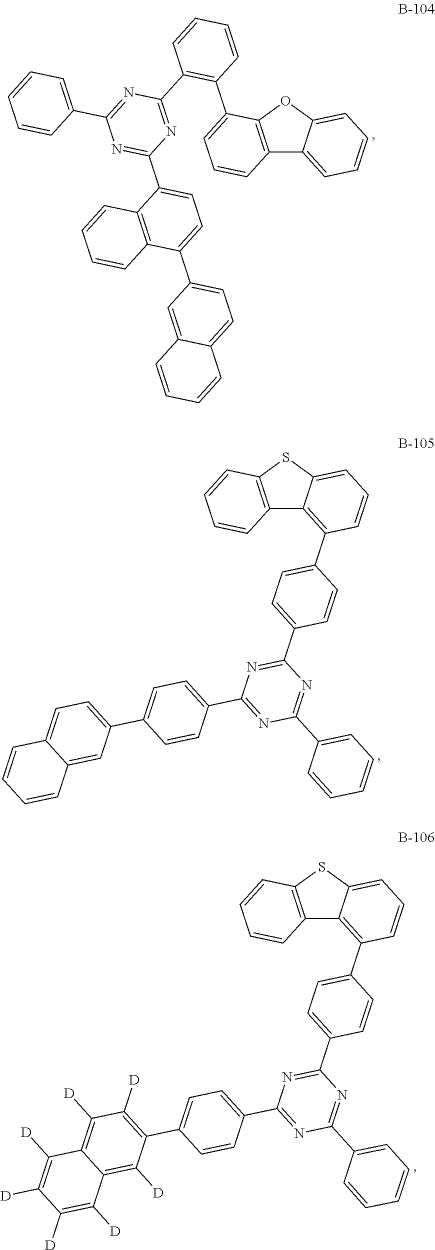 [SMILES: c1ccc(-c2nc(-c3ccccc3-c3cccc4c3oc3ccccc34)nc(-c3ccc(-c4ccc5ccccc5c4)c4ccccc34)n2)cc1 ; c1ccc(-c2nc(-c3ccc(-c4ccc5ccccc5c4)cc3)nc(-c3ccc(-c4cccc5sc6ccccc6c45)cc3)n2)cc1 ; [2H]c1c([2H])c([2H])c2c([2H])c(-c3ccc(-c4nc(-c5ccccc5)nc(-c5ccc(-c6cccc7sc8ccccc8c67)cc5)n4)cc3)c([2H])c([2H])c2c1[2H]] 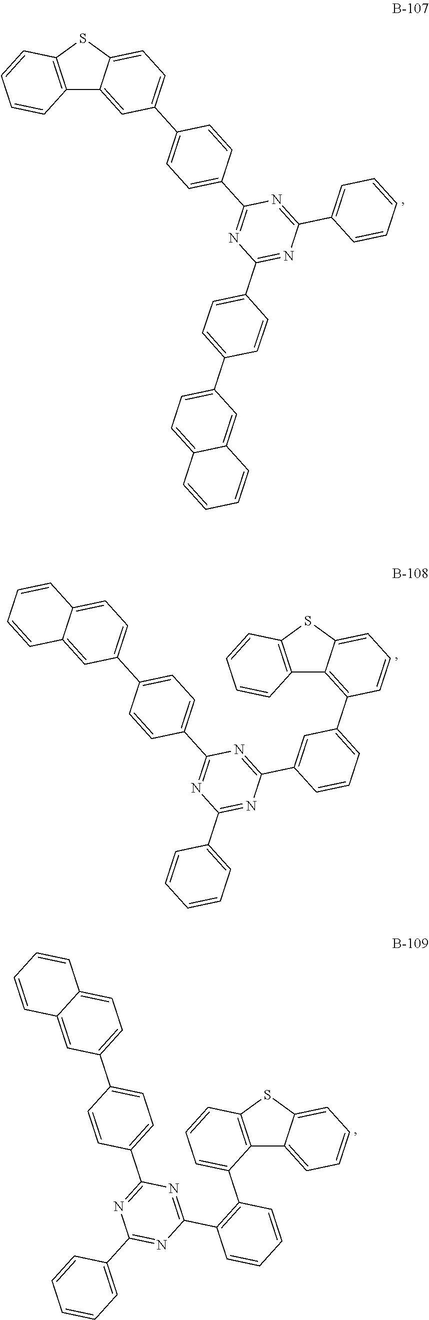 [SMILES: c1ccc(-c2nc(-c3ccc(-c4ccc5ccccc5c4)cc3)nc(-c3ccc(-c4ccc5sc6ccccc6c5c4)cc3)n2)cc1 ; c1ccc(-c2nc(-c3ccc(-c4ccc5ccccc5c4)cc3)nc(-c3cccc(-c4cccc5sc6ccccc6c45)c3)n2)cc1 ; c1ccc(-c2nc(-c3ccc(-c4ccc5ccccc5c4)cc3)nc(-c3ccccc3-c3cccc4sc5ccccc5c34)n2)cc1] 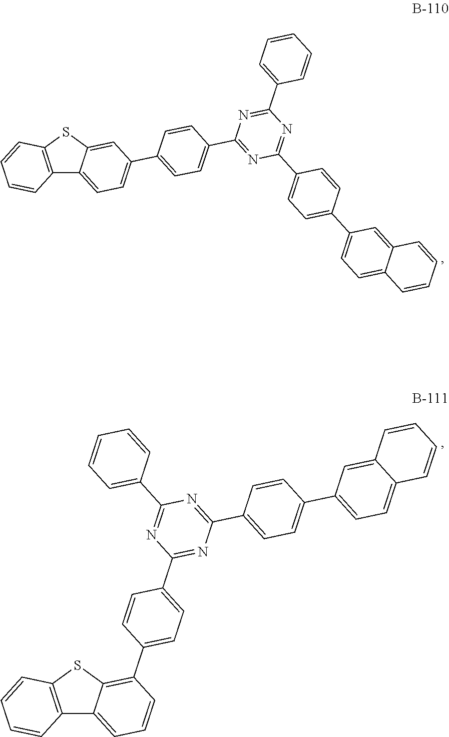 [SMILES: c1ccc(-c2nc(-c3ccc(-c4ccc5ccccc5c4)cc3)nc(-c3ccc(-c4ccc5c(c4)sc4ccccc45)cc3)n2)cc1 ; c1ccc(-c2nc(-c3ccc(-c4ccc5ccccc5c4)cc3)nc(-c3ccc(-c4cccc5c4sc4ccccc45)cc3)n2)cc1] 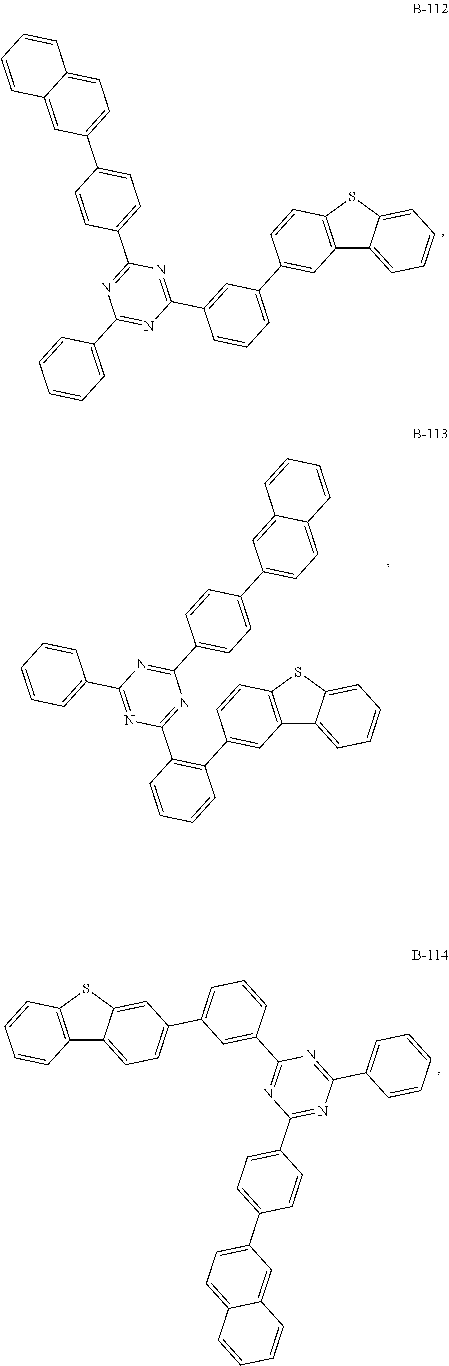 [SMILES: c1ccc(-c2nc(-c3ccc(-c4ccc5ccccc5c4)cc3)nc(-c3cccc(-c4ccc5sc6ccccc6c5c4)c3)n2)cc1 ; c1ccc(-c2nc(-c3ccc(-c4ccc5ccccc5c4)cc3)nc(-c3ccccc3-c3ccc4sc5ccccc5c4c3)n2)cc1 ; c1ccc(-c2nc(-c3ccc(-c4ccc5ccccc5c4)cc3)nc(-c3cccc(-c4ccc5c(c4)sc4ccccc45)c3)n2)cc1] 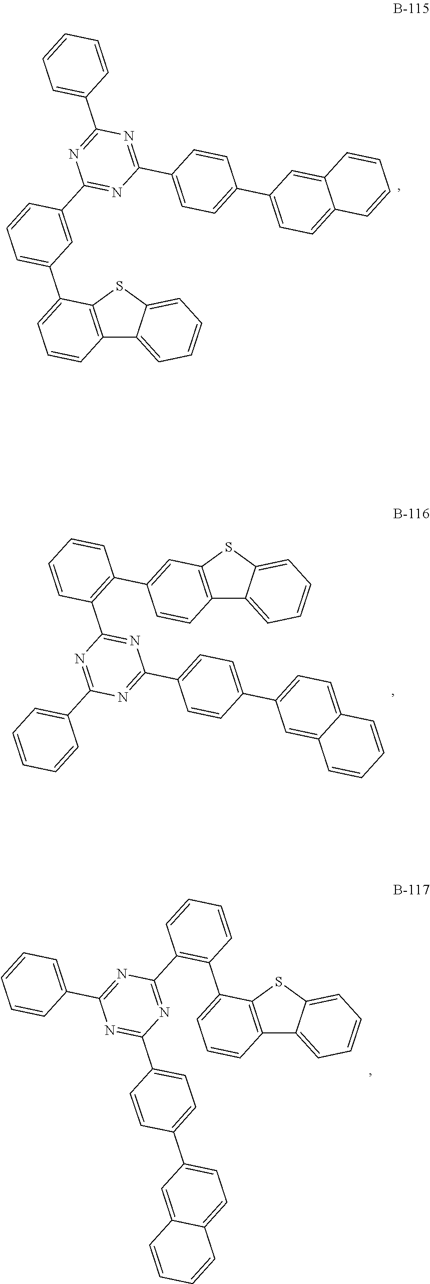 [SMILES: c1ccc(-c2nc(-c3ccc(-c4ccc5ccccc5c4)cc3)nc(-c3cccc(-c4cccc5c4sc4ccccc45)c3)n2)cc1 ; c1ccc(-c2nc(-c3ccc(-c4ccc5ccccc5c4)cc3)nc(-c3ccccc3-c3ccc4c(c3)sc3ccccc34)n2)cc1 ; c1ccc(-c2nc(-c3ccc(-c4ccc5ccccc5c4)cc3)nc(-c3ccccc3-c3cccc4c3sc3ccccc34)n2)cc1] 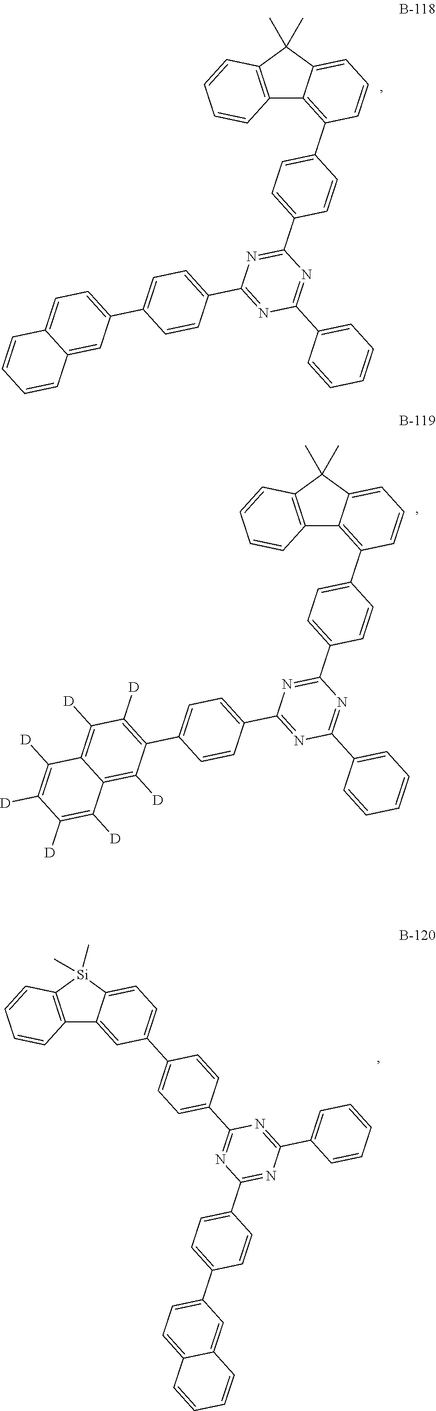 [SMILES: CC1(C)c2ccccc2-c2c(-c3ccc(-c4nc(-c5ccccc5)nc(-c5ccc(-c6ccc7ccccc7c6)cc5)n4)cc3)cccc21 ; [2H]c1c([2H])c([2H])c2c([2H])c(-c3ccc(-c4nc(-c5ccccc5)nc(-c5ccc(-c6cccc7c6-c6ccccc6C7(C)C)cc5)n4)cc3)c([2H])c([2H])c2c1[2H] ; C[Si]1(C)c2ccccc2-c2cc(-c3ccc(-c4nc(-c5ccccc5)nc(-c5ccc(-c6ccc7ccccc7c6)cc5)n4)cc3)ccc21] 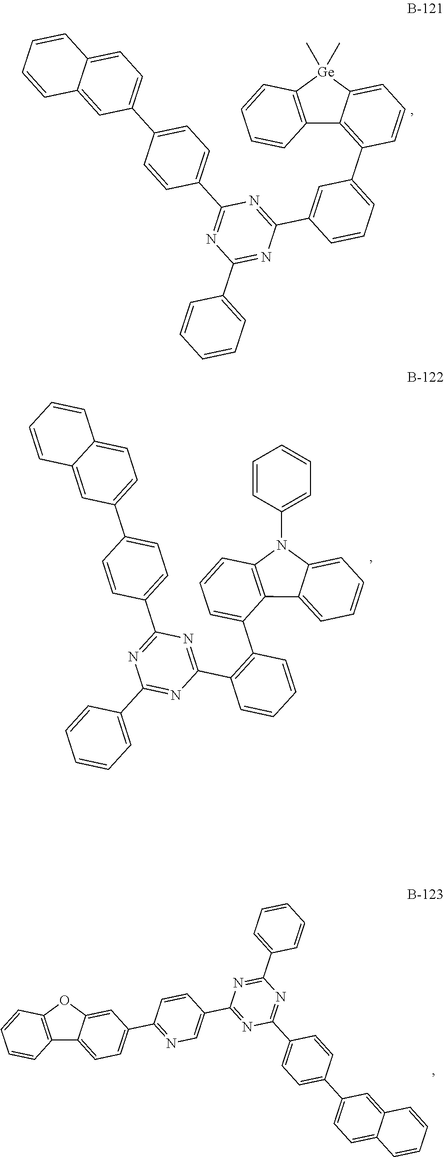 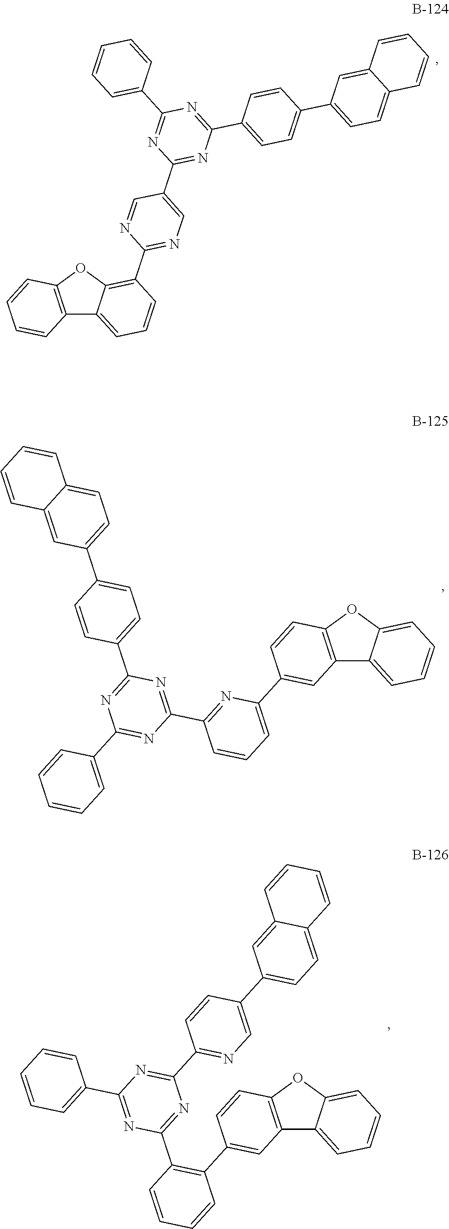 [SMILES: c1ccc(-c2nc(-c3ccc(-c4ccc5ccccc5c4)cc3)nc(-c3cnc(-c4cccc5c4oc4ccccc45)nc3)n2)cc1 ; c1ccc(-c2nc(-c3ccc(-c4ccc5ccccc5c4)cc3)nc(-c3cccc(-c4ccc5oc6ccccc6c5c4)n3)n2)cc1 ; c1ccc(-c2nc(-c3ccc(-c4ccc5ccccc5c4)cn3)nc(-c3ccccc3-c3ccc4oc5ccccc5c4c3)n2)cc1] 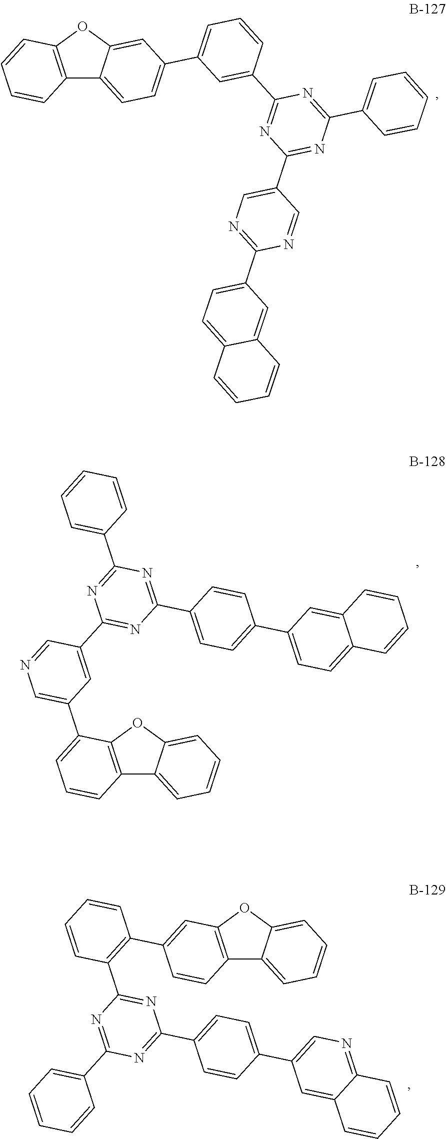 [SMILES: c1ccc(-c2nc(-c3cnc(-c4ccc5ccccc5c4)nc3)nc(-c3cccc(-c4ccc5c(c4)oc4ccccc45)c3)n2)cc1 ; c1ccc(-c2nc(-c3ccc(-c4ccc5ccccc5c4)cc3)nc(-c3cncc(-c4cccc5c4oc4ccccc45)c3)n2)cc1 ; c1ccc(-c2nc(-c3ccc(-c4cnc5ccccc5c4)cc3)nc(-c3ccccc3-c3ccc4c(c3)oc3ccccc34)n2)cc1] 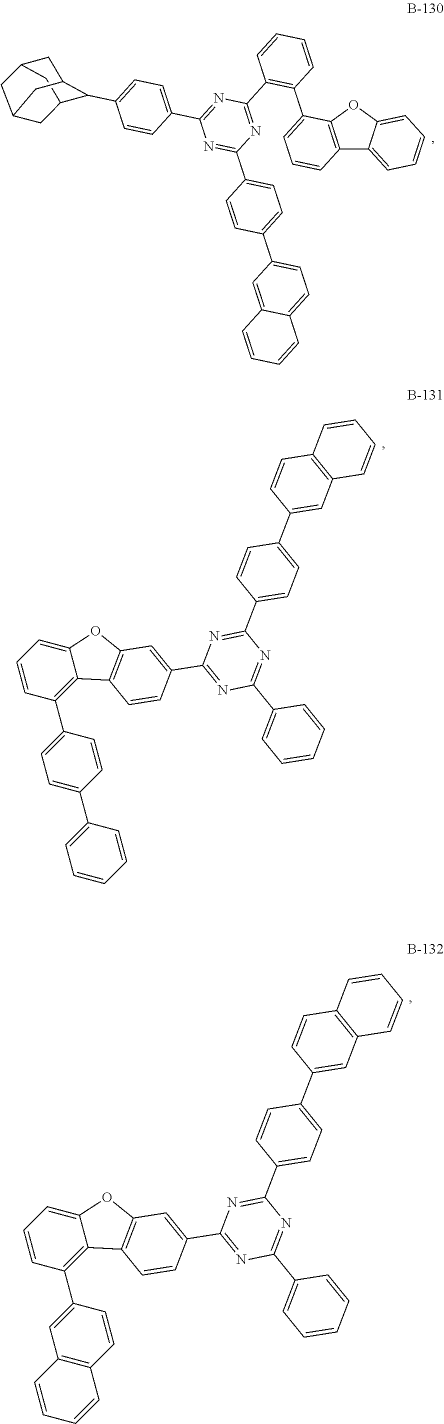 [SMILES: c1ccc(-c2cccc3c2oc2ccccc23)c(-c2nc(-c3ccc(-c4ccc5ccccc5c4)cc3)nc(-c3ccc(C4C5CC6CC(C5)CC4C6)cc3)n2)c1 ; c1ccc(-c2ccc(-c3cccc4oc5cc(-c6nc(-c7ccccc7)nc(-c7ccc(-c8ccc9ccccc9c8)cc7)n6)ccc5c34)cc2)cc1 ; c1ccc(-c2nc(-c3ccc(-c4ccc5ccccc5c4)cc3)nc(-c3ccc4c(c3)oc3cccc(-c5ccc6ccccc6c5)c34)n2)cc1] 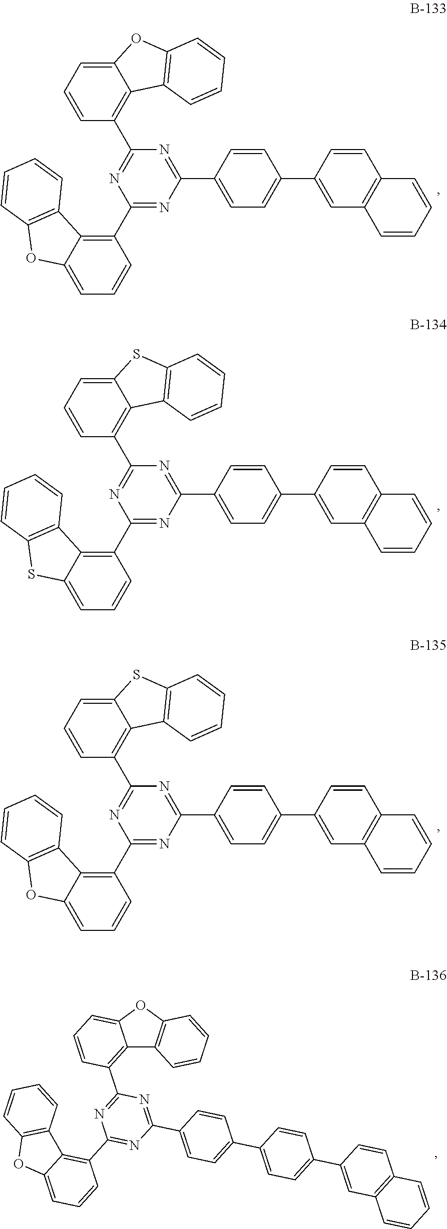 [SMILES: c1ccc2cc(-c3ccc(-c4nc(-c5cccc6oc7ccccc7c56)nc(-c5cccc6oc7ccccc7c56)n4)cc3)ccc2c1 ; c1ccc2cc(-c3ccc(-c4nc(-c5cccc6sc7ccccc7c56)nc(-c5cccc6sc7ccccc7c56)n4)cc3)ccc2c1 ; c1ccc2cc(-c3ccc(-c4nc(-c5cccc6oc7ccccc7c56)nc(-c5cccc6sc7ccccc7c56)n4)cc3)ccc2c1 ; c1ccc2cc(-c3ccc(-c4ccc(-c5nc(-c6cccc7oc8ccccc8c67)nc(-c6cccc7oc8ccccc8c67)n5)cc4)cc3)ccc2c1] 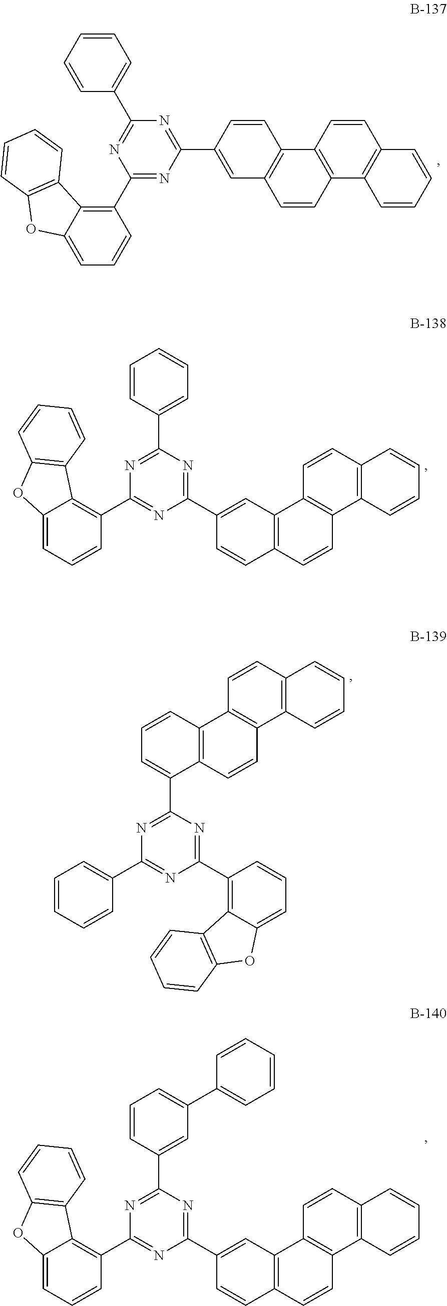 [SMILES: c1ccc(-c2nc(-c3ccc4c(ccc5c6ccccc6ccc45)c3)nc(-c3cccc4oc5ccccc5c34)n2)cc1 ; c1ccc(-c2nc(-c3ccc4ccc5c6ccccc6ccc5c4c3)nc(-c3cccc4oc5ccccc5c34)n2)cc1 ; c1ccc(-c2nc(-c3cccc4c3ccc3c5ccccc5ccc43)nc(-c3cccc4oc5ccccc5c34)n2)cc1 ; c1ccc(-c2cccc(-c3nc(-c4ccc5ccc6c7ccccc7ccc6c5c4)nc(-c4cccc5oc6ccccc6c45)n3)c2)cc1] 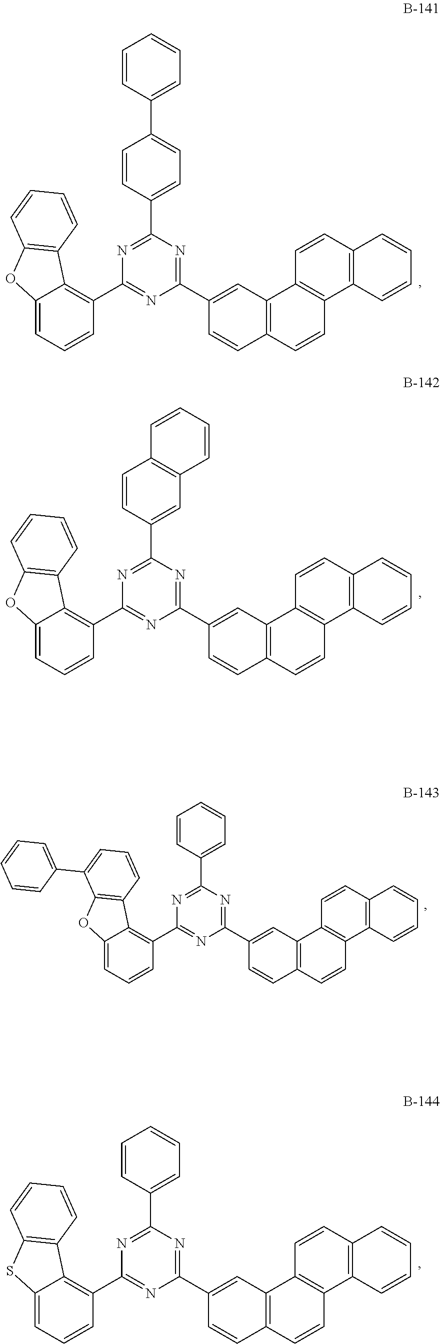 [SMILES: c1ccc(-c2ccc(-c3nc(-c4ccc5ccc6c7ccccc7ccc6c5c4)nc(-c4cccc5oc6ccccc6c45)n3)cc2)cc1 ; c1ccc2cc(-c3nc(-c4ccc5ccc6c7ccccc7ccc6c5c4)nc(-c4cccc5oc6ccccc6c45)n3)ccc2c1 ; c1ccc(-c2nc(-c3ccc4ccc5c6ccccc6ccc5c4c3)nc(-c3cccc4oc5c(-c6ccccc6)cccc5c34)n2)cc1 ; c1ccc(-c2nc(-c3ccc4ccc5c6ccccc6ccc5c4c3)nc(-c3cccc4sc5ccccc5c34)n2)cc1] 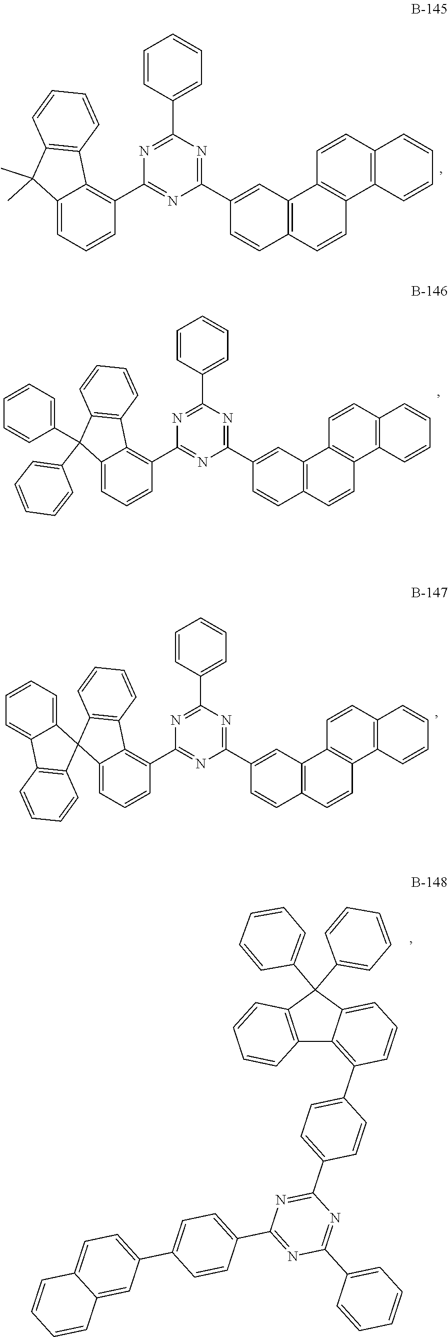 [SMILES: CC1(C)c2ccccc2-c2c(-c3nc(-c4ccccc4)nc(-c4ccc5ccc6c7ccccc7ccc6c5c4)n3)cccc21 ; c1ccc(-c2nc(-c3ccc4ccc5c6ccccc6ccc5c4c3)nc(-c3cccc4c3-c3ccccc3C4(c3ccccc3)c3ccccc3)n2)cc1 ; c1ccc(-c2nc(-c3ccc4ccc5c6ccccc6ccc5c4c3)nc(-c3cccc4c3-c3ccccc3C43c4ccccc4-c4ccccc43)n2)cc1 ; c1ccc(-c2nc(-c3ccc(-c4ccc5ccccc5c4)cc3)nc(-c3ccc(-c4cccc5c4-c4ccccc4C5(c4ccccc4)c4ccccc4)cc3)n2)cc1] 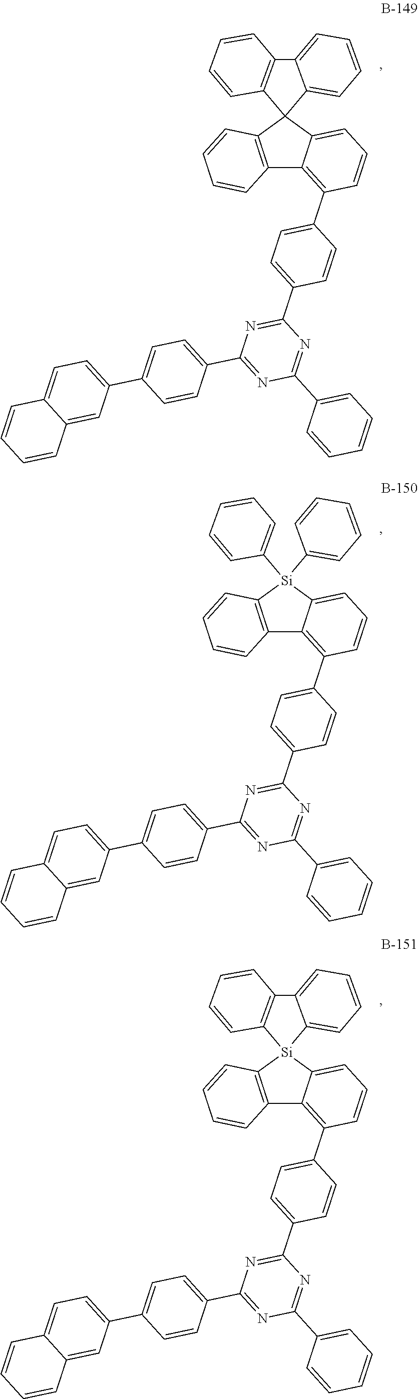 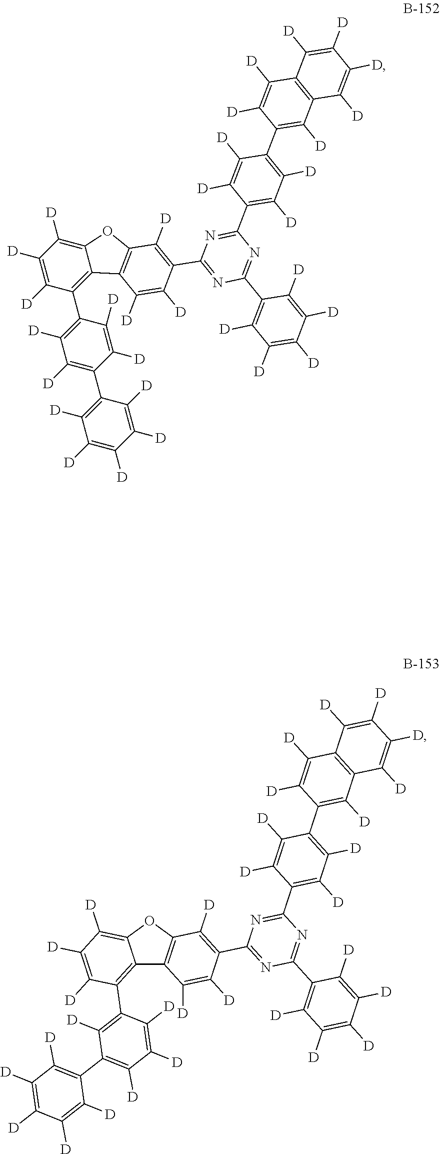 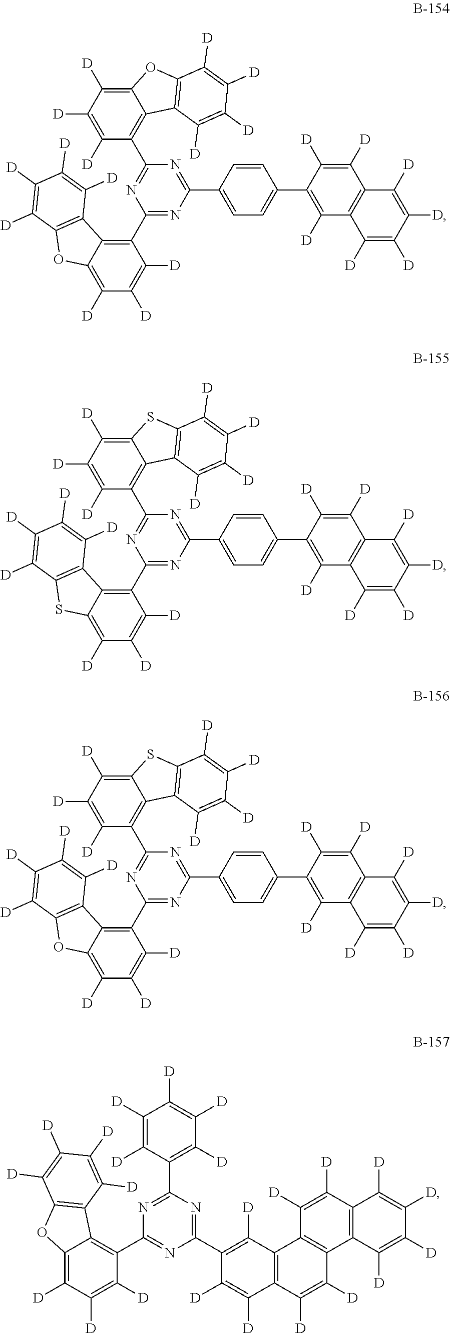 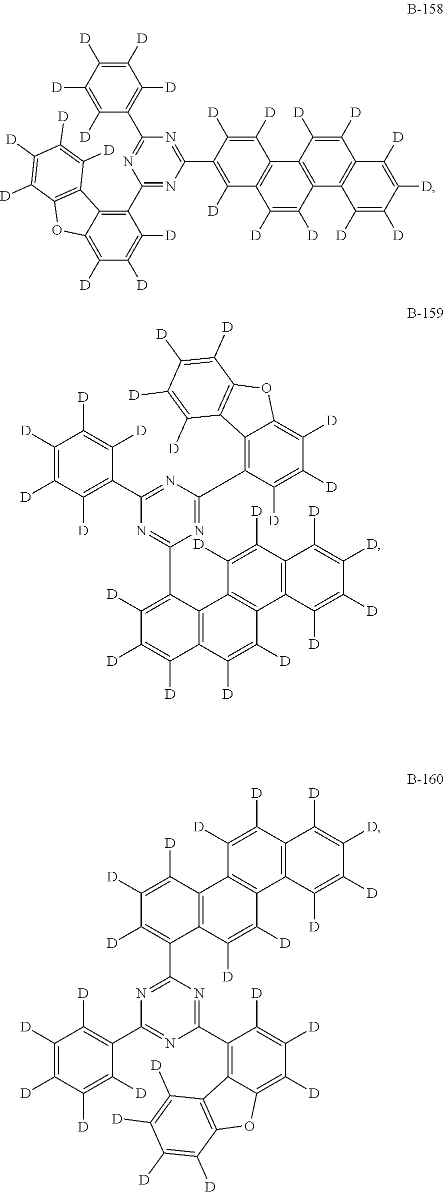 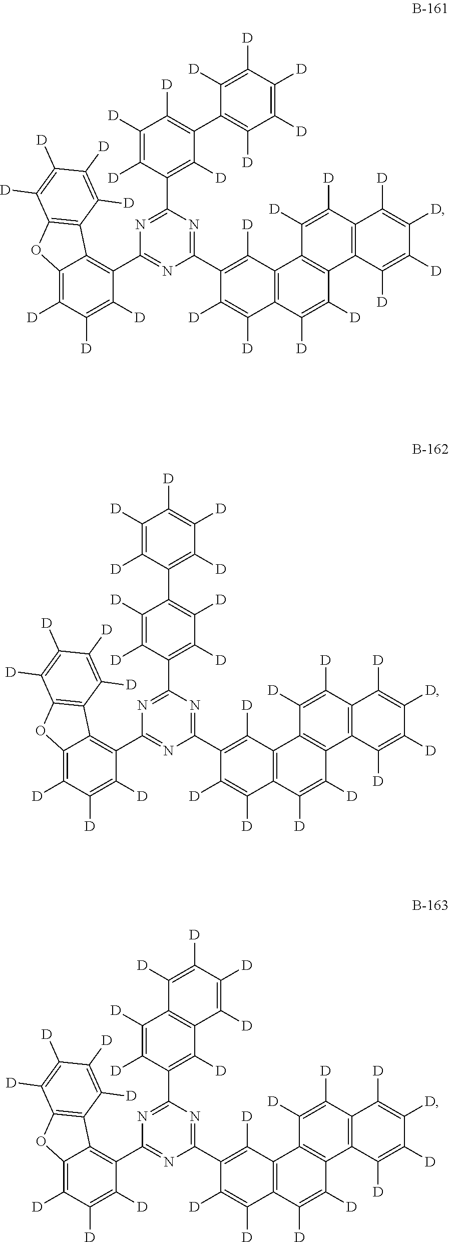 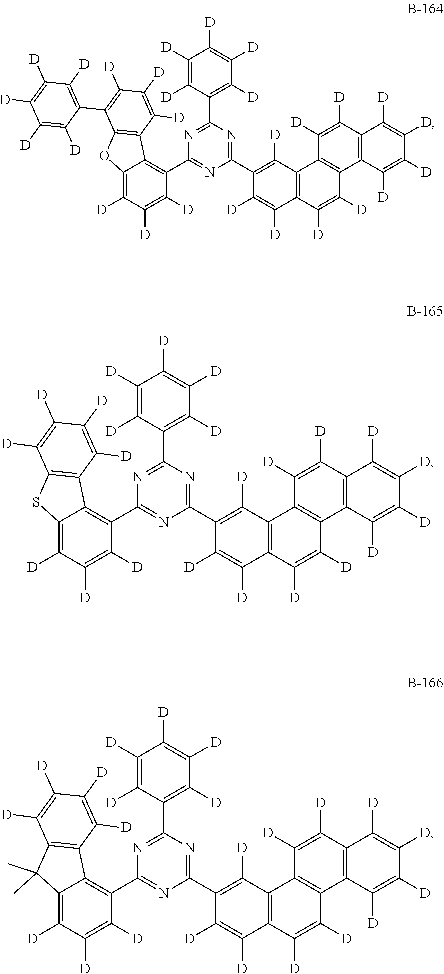 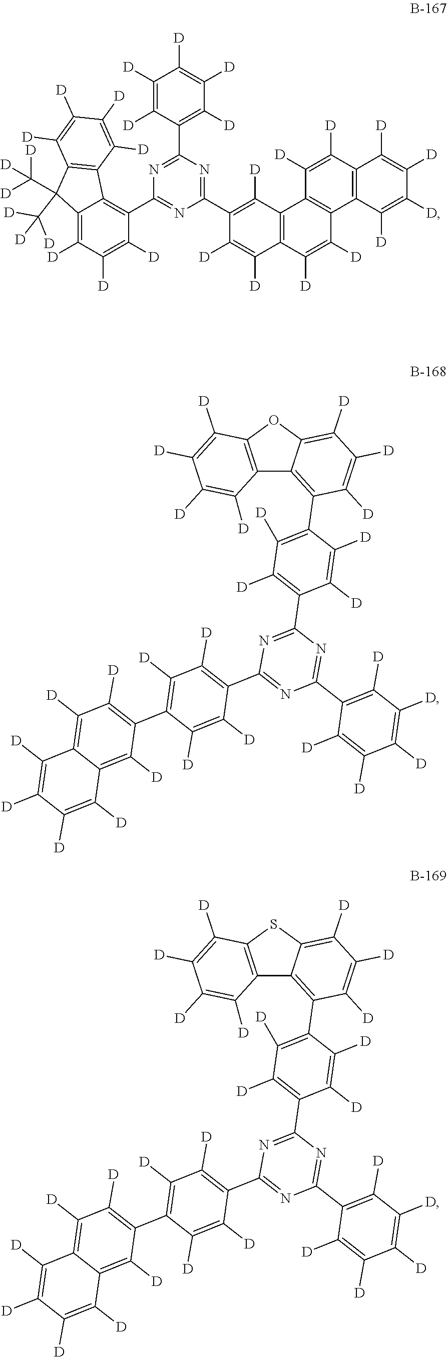 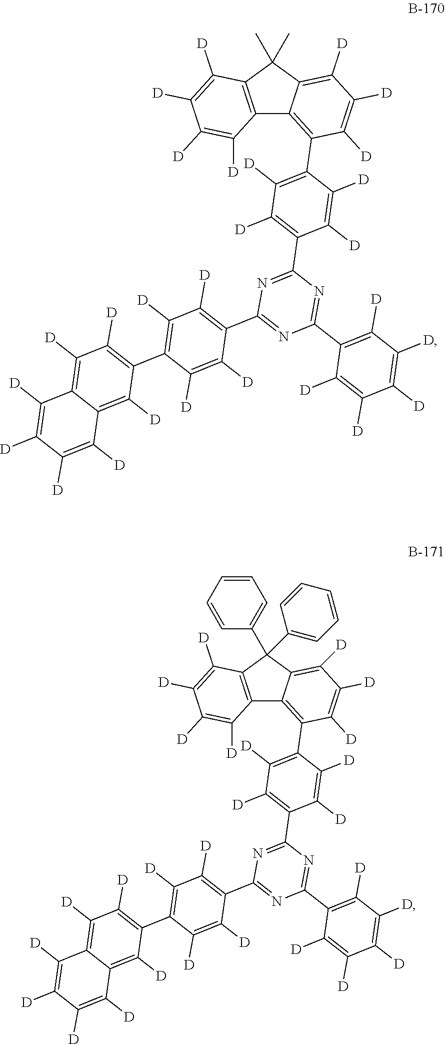 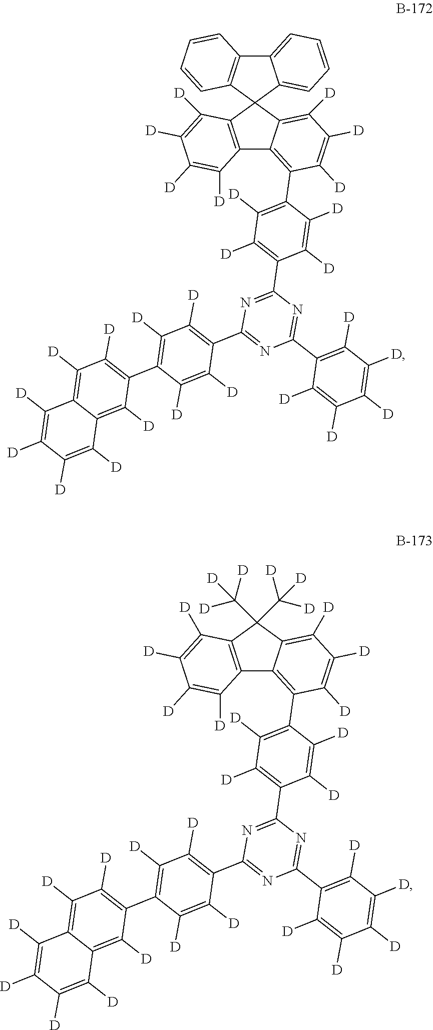 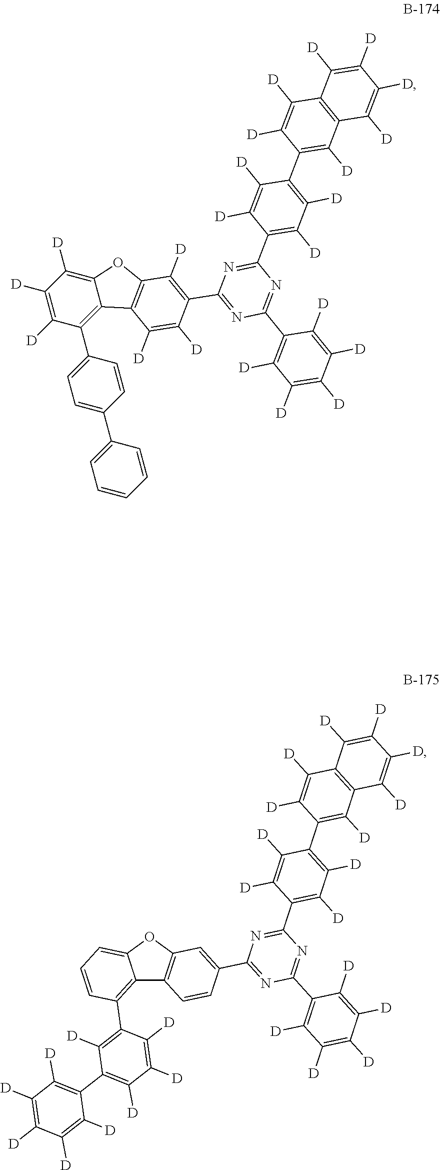 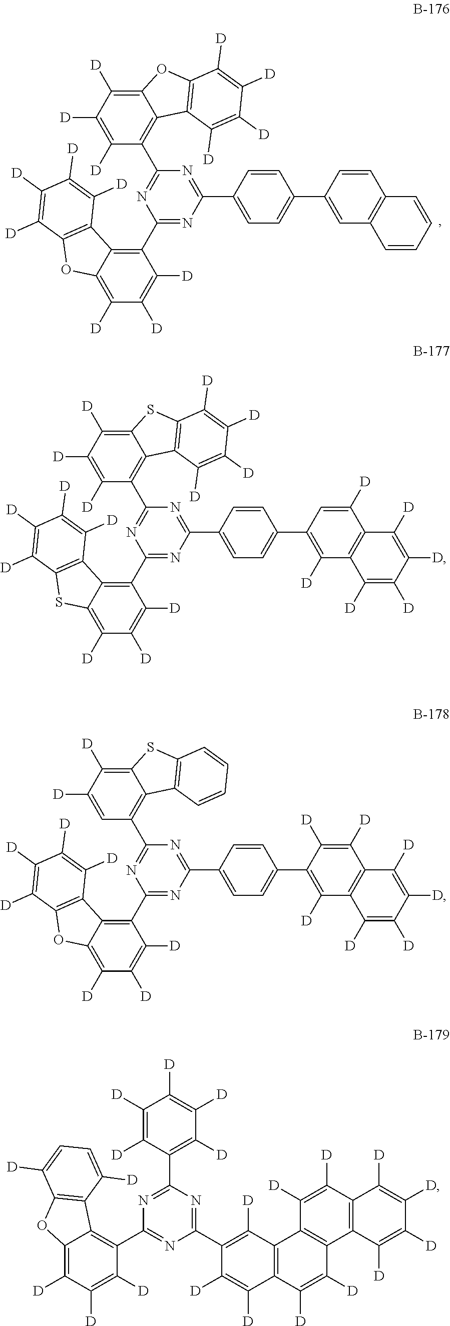 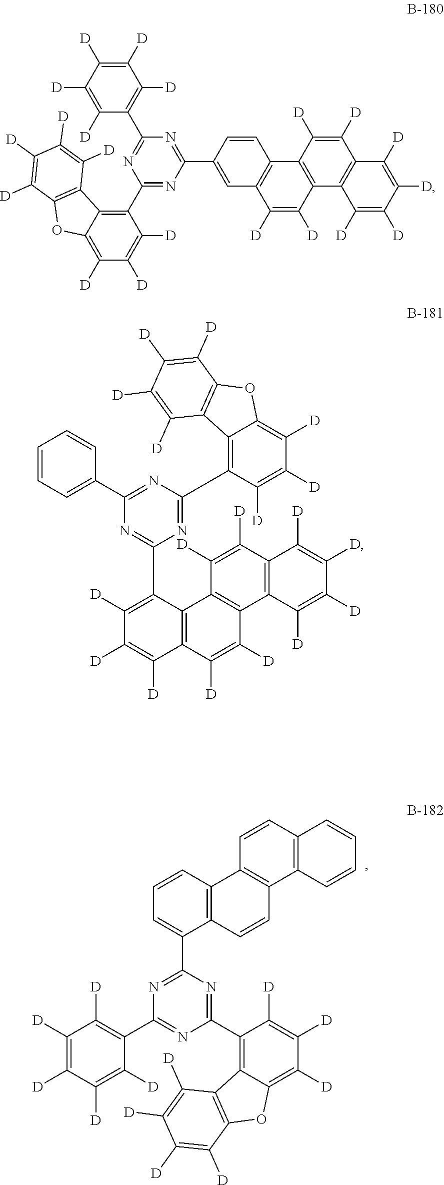 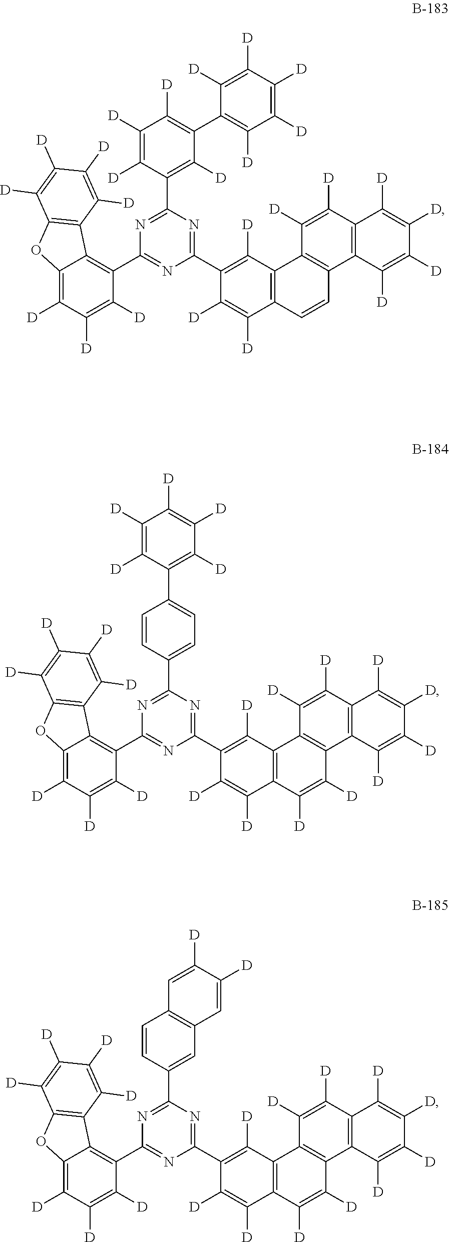 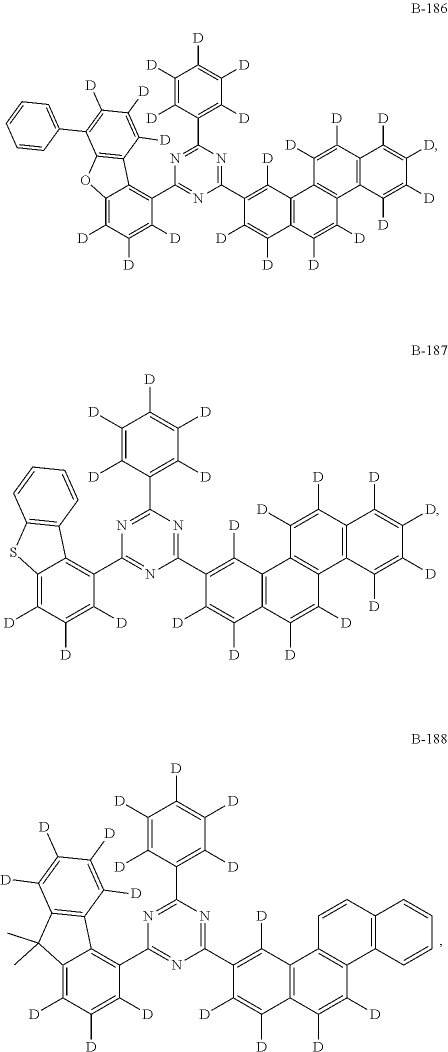 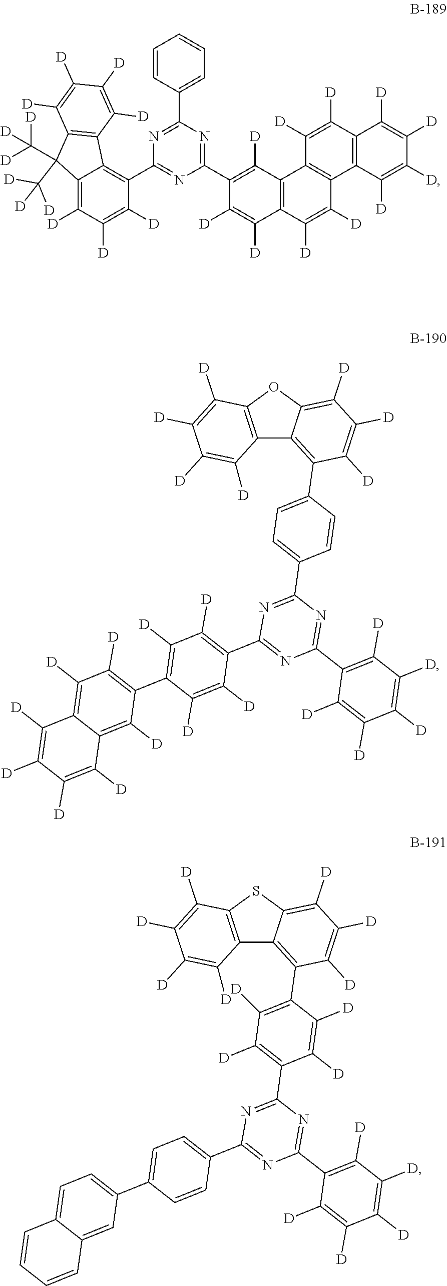 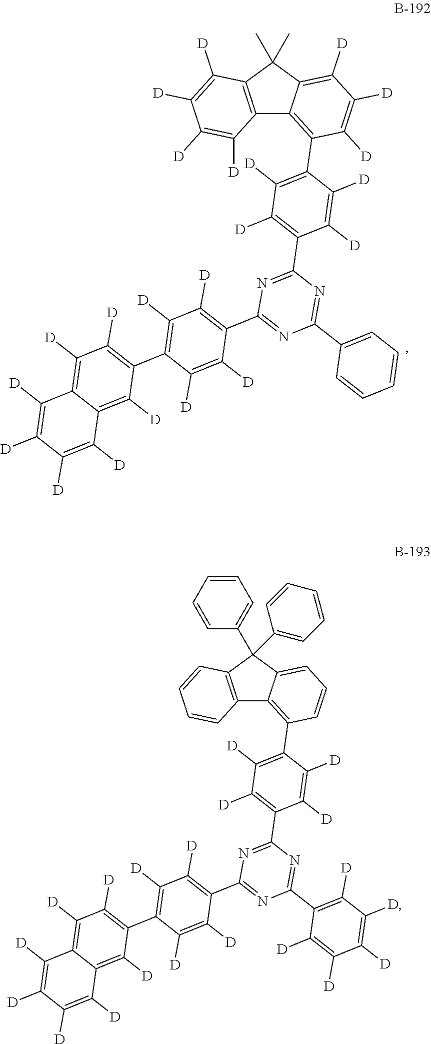 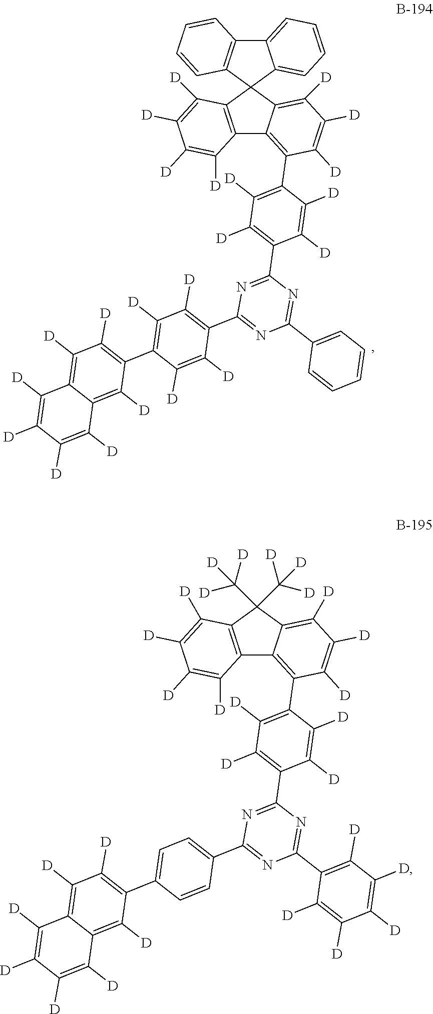 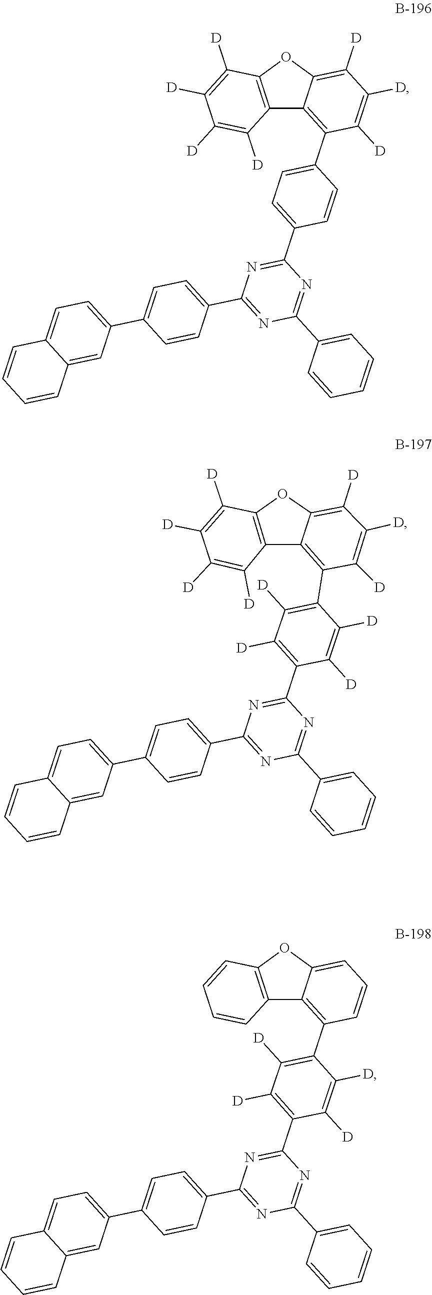 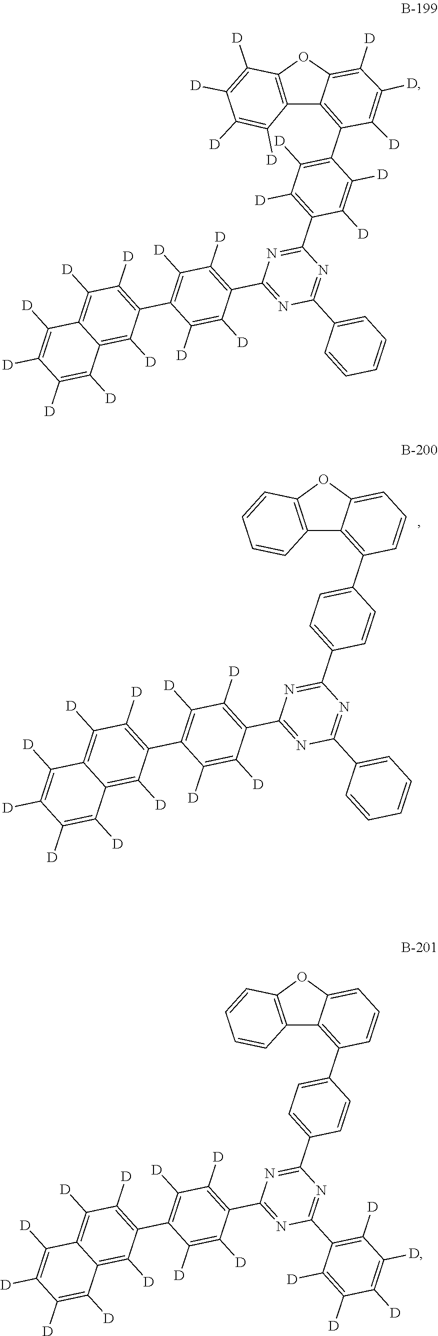 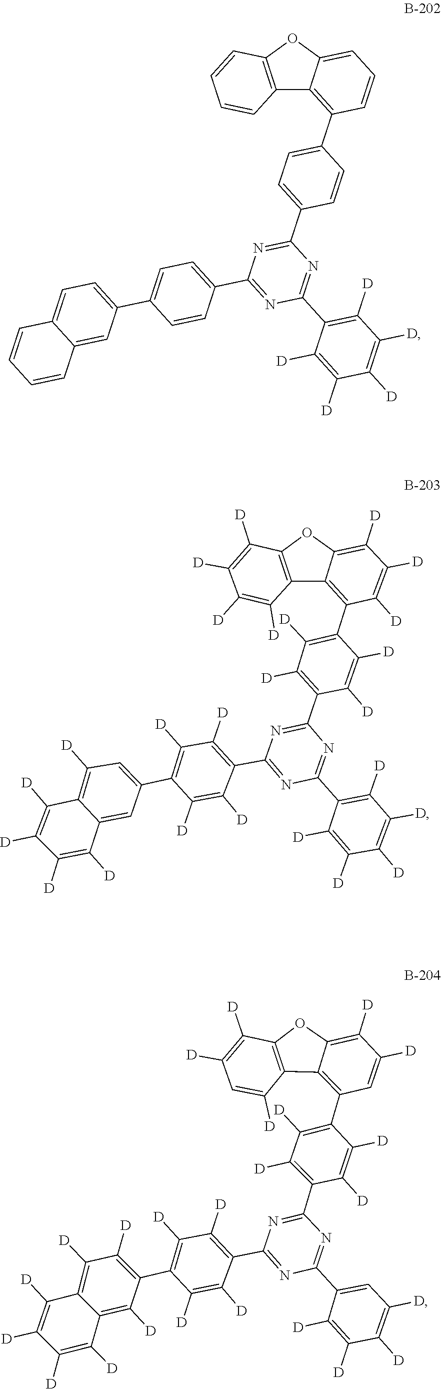 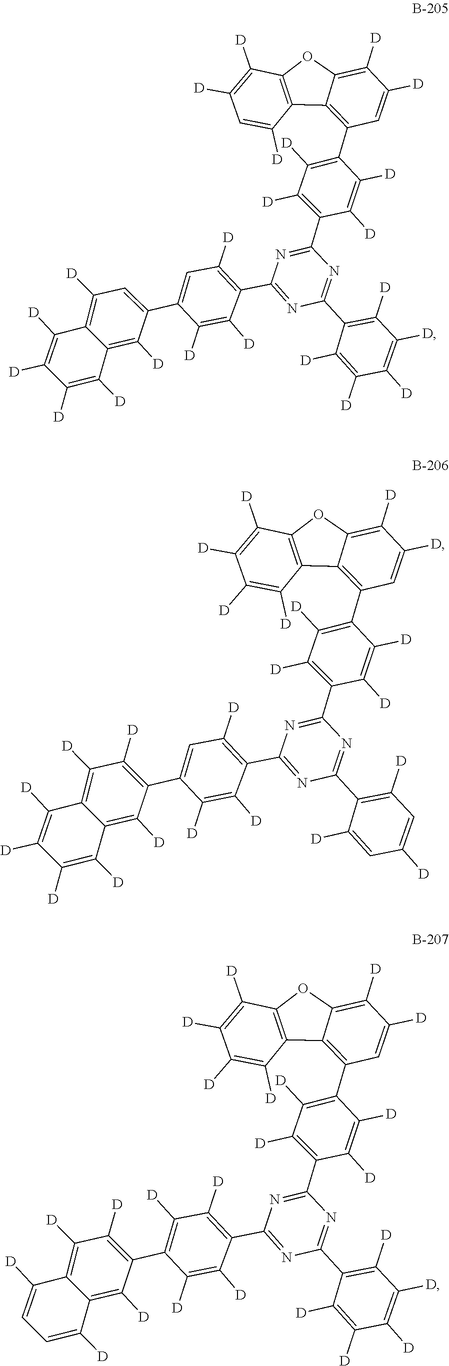 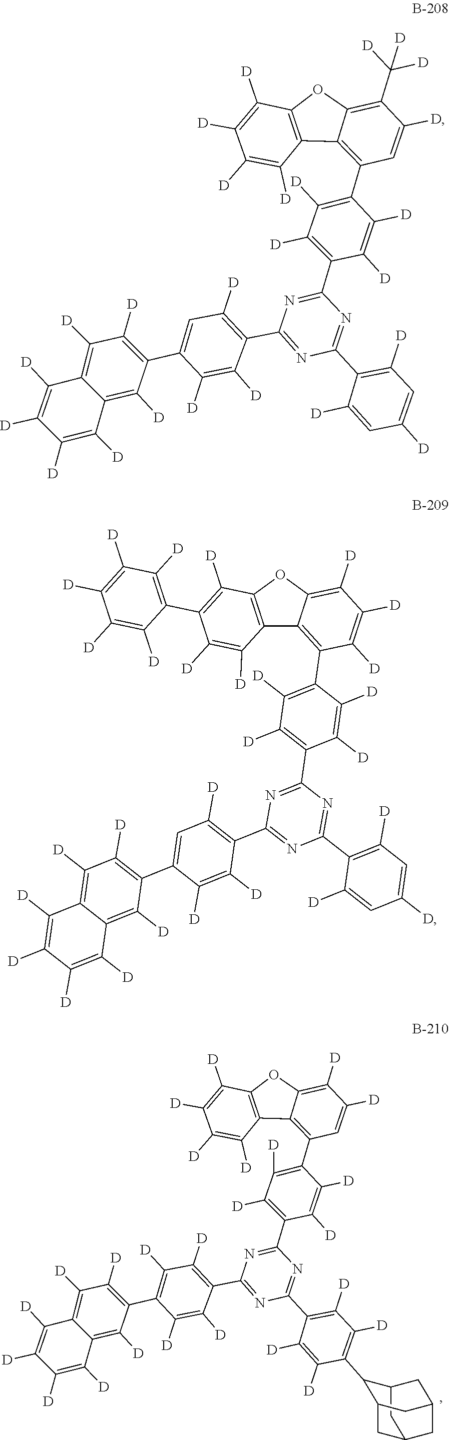 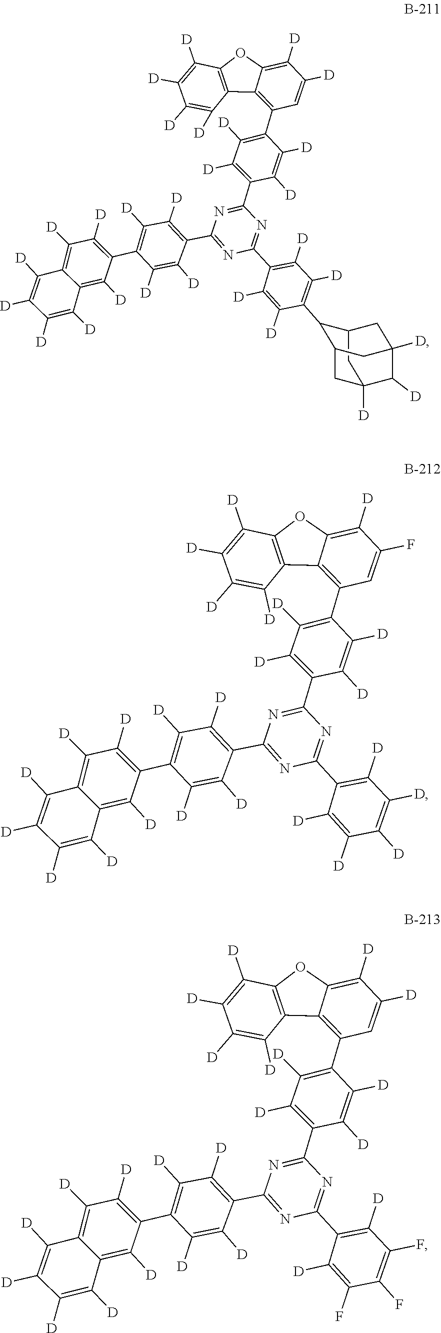 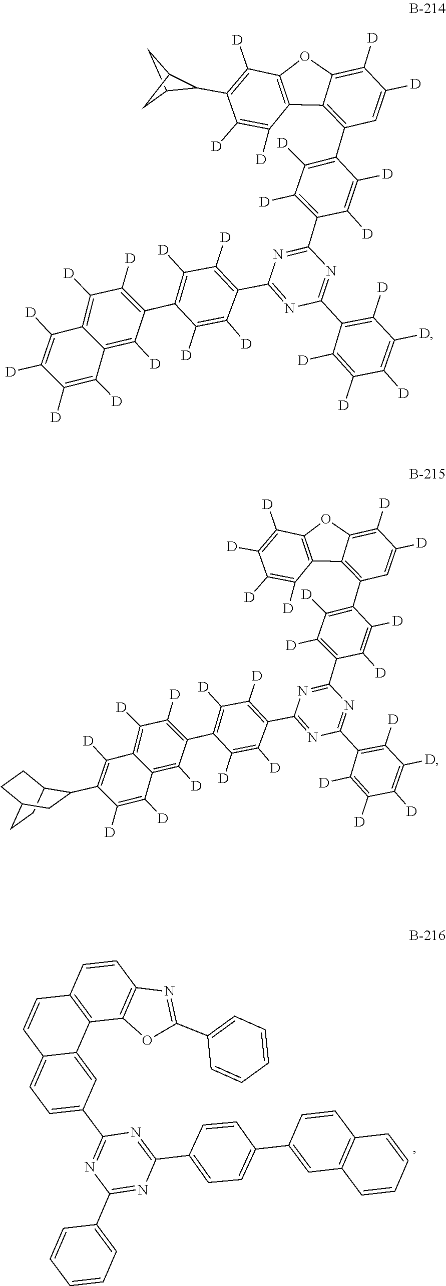 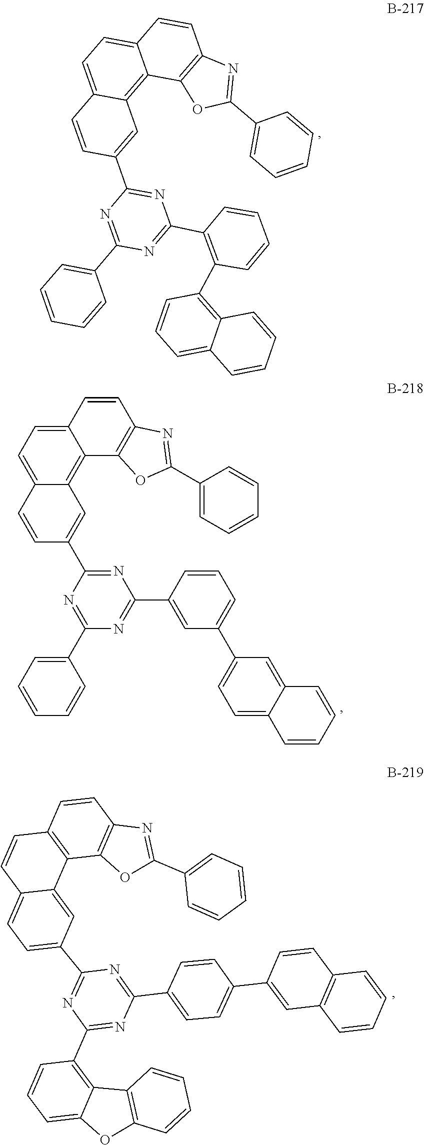 [SMILES: c1ccc(-c2nc(-c3ccc4ccc5ccc6nc(-c7ccccc7)oc6c5c4c3)nc(-c3ccccc3-c3cccc4ccccc34)n2)cc1 ; c1ccc(-c2nc(-c3cccc(-c4ccc5ccccc5c4)c3)nc(-c3ccc4ccc5ccc6nc(-c7ccccc7)oc6c5c4c3)n2)cc1 ; c1ccc(-c2nc3ccc4ccc5ccc(-c6nc(-c7ccc(-c8ccc9ccccc9c8)cc7)nc(-c7cccc8oc9ccccc9c78)n6)cc5c4c3o2)cc1] 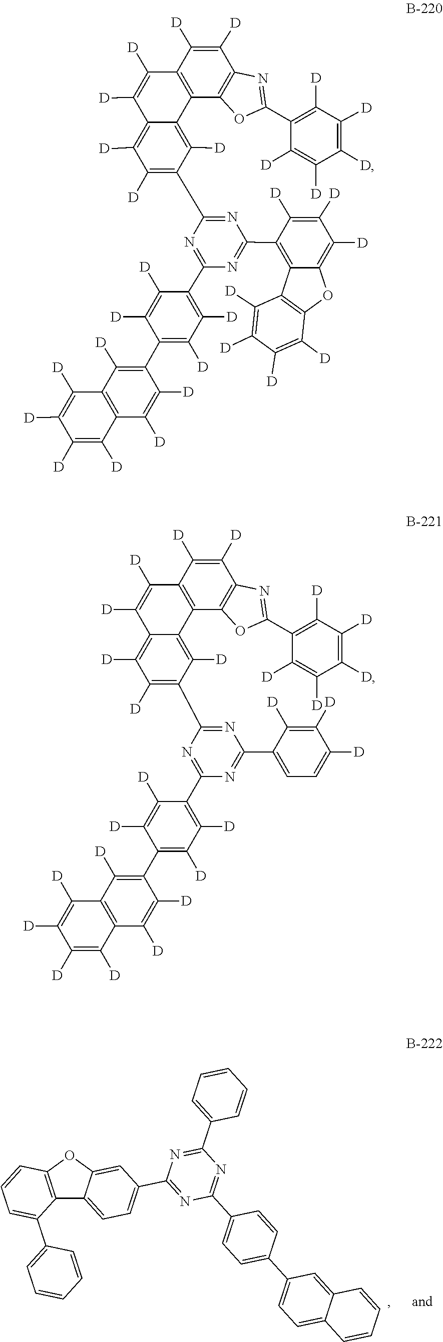 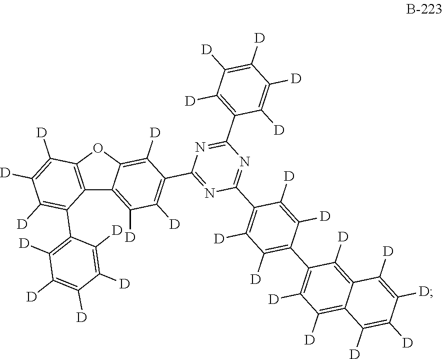
wherein hydrogen in Compound B-1 to Compound B-223 can be partially or fully substituted with deuterium.

14. A compound composition, comprising a first compound represented by Formula 1, a second compound represented by Formula 2 and a third compound represented by Formula 1 or Formula 2, wherein the first compound, the second compound and the third compound are different from each other in structure; 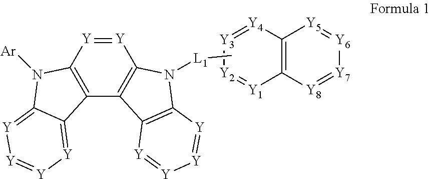
wherein
Ar is selected from substituted or unsubstituted aryl having 6 to 30 carbon atoms, substituted or unsubstituted heteroaryl having 3 to 30 carbon atoms or a combination thereof;
L₁ is selected from a single bond, substituted or unsubstituted arylene having 6 to 30 carbon atoms, substituted or unsubstituted heteroarylene having 3 to 30 carbon atoms or a combination thereof;
Y is, at each occurrence identically or differently, selected from CR_{y} or N;
Y₁ to Y₄ are, at each occurrence identically or differently, selected from C, CR_{y} or N, and at least one of Y₁ to Y₄ is selected from C and joined to L₁;
Y₅ to Y₈ are, at each occurrence identically or differently, selected from CR_{y} or N;
adjacent substituents R_{y} can be optionally joined to form a ring; 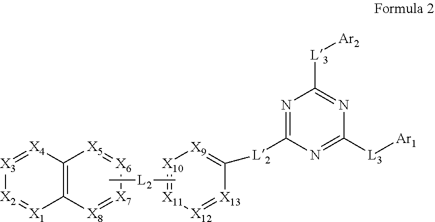
wherein
X₁ to X₄ are, at each occurrence identically or differently, selected from CRₓ or N;
X₅ to X₁₃ are, at each occurrence identically or differently, selected from C, CRₓ or N, at least one of X₅ to X₈ is selected from C and joined to L₂, and at least one of X₉ to X₁₃ is selected from C and joined to L₂;
L₂, L′₂, L₃ and L′₃ are, at each occurrence identically or differently, selected from a single bond, substituted or unsubstituted arylene having 6 to 30 carbon atoms, substituted or unsubstituted heteroarylene having 3 to 30 carbon atoms or a combination thereof;
Ar₁ and Ar₂ are, at each occurrence identically or differently, selected from substituted or unsubstituted aryl having 6 to 30 carbon atoms, substituted or unsubstituted heteroaryl having 3 to 30 carbon atoms or a combination thereof;
adjacent substituents Rₓ can be optionally joined to form a ring;
Rₓ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

15. The compound composition of claim 14, wherein the first compound has a structure represented by Formula 1-1: 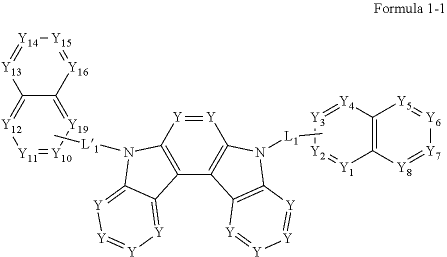
wherein
L₁ and L′₁ are, at each occurrence identically or differently, selected from a single bond, substituted or unsubstituted arylene having 6 to 30 carbon atoms, substituted or unsubstituted heteroarylene having 3 to 30 carbon atoms or a combination thereof;
Y is, at each occurrence identically or differently, selected from CR_{y} or N;
Y₁ to Y₄ and Y₉ to Y₁₂ are, at each occurrence identically or differently, selected from C, CR_{y} or N, at least one of Y₁ to Y₄ is selected from C and joined to L₁, and at least one of Y₉ to Y₁₂ is selected from C and joined to L′₁;
Y₅ to Y₈ and Y₁₃ to Y₁₆ are, at each occurrence identically or differently, selected from CR_{y} or N;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{y} can be optionally joined to form a ring.

16. The compound composition of claim 15, wherein L₁ and L′₁ are, at each occurrence identically or differently, selected from a single bond, substituted or unsubstituted arylene having 6 to 20 carbon atoms, substituted or unsubstituted heteroarylene having 3 to 20 carbon atoms or a combination thereof;
preferably, L₁ and L′₁ are, at each occurrence identically or differently, selected from a single bond, substituted or unsubstituted arylene having 6 to 20 carbon atoms or a combination thereof;
more preferably, L₁ and L′₁ are, at each occurrence identically or differently, selected from a single bond, substituted or unsubstituted phenylene, substituted or unsubstituted naphthylene, substituted or unsubstituted biphenylylene, substituted or unsubstituted phenanthrylene, substituted or unsubstituted triphenylenylene, substituted or unsubstituted 9,9-dimethylfluorenylene, substituted or unsubstituted terphenylene, substituted or unsubstituted chrysenylene or a combination thereof.

17. The compound composition of claim 15, wherein Y₁ to Y₄ and Y₉ to Y₁₂ are, at each occurrence identically or differently, selected from C or CR_{y}, at least one of Y₁ to Y₄ is selected from C and joined to L₁, and at least one of Y₉ to Y₁₂ is selected from C and joined to L′₁;
preferably, Y₁ to Y₄ and Y₉ to Y₁₂ are, at each occurrence identically or differently, selected from C or CR_{y}, Y₁ or Y₂ is selected from C and joined to L₁, and Y₁₀ or Y₁₁ is selected from C and joined to L′₁.

18. The compound composition of claim 14, wherein Y is selected from CR_{y};
preferably, R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, and combinations thereof;
more preferably, R_{y} is, at each occurrence identically or differently, selected from the group consisting of: hydrogen, deuterium, halogen, phenyl, pyridyl, vinyl, adamantyl, methyl, ethyl, isopropyl, cyclopropyl, naphthyl, biphenyl, phenanthryl, triphenylenyl, tert-butyl, trifluoromethyl, 9,9-dimethylfluorenyl, terphenyl, dibenzothienyl, dibenzofuryl, benzothiazolyl, benzoxazolyl, phenanthroxazolyl, phenanthrothiazolyl, carbazolyl, chrysenyl, and combinations thereof.

19. The compound composition of claim 14, wherein L₂, L′₂, L₃ and L′₃ are, at each occurrence identically or differently, selected from a single bond, substituted or unsubstituted arylene having 6 to 20 carbon atoms, substituted or unsubstituted heteroarylene having 3 to 20 carbon atoms or a combination thereof;
preferably, L₂, L′₂, L₃ and L′₃ are, at each occurrence identically or differently, selected from a single bond, substituted or unsubstituted arylene having 6 to 20 carbon atoms or a combination thereof;
more preferably, L₂, L′₂, L₃ and L′₃ are, at each occurrence identically or differently, selected from a single bond, substituted or unsubstituted phenylene, substituted or unsubstituted naphthylene, substituted or unsubstituted biphenylylene, substituted or unsubstituted phenanthrylene, substituted or unsubstituted triphenylenylene, substituted or unsubstituted 9,9-dimethylfluorenylene, substituted or unsubstituted terphenylene, substituted or unsubstituted chrysenylene or a combination thereof.

20. The compound composition of claim 14, wherein X₁ to X₄ are, at each occurrence identically or differently, selected from CRₓ, X₈ to X₁₃ are, at each occurrence identically or differently, selected from C or CRₓ, X₆ or X₇ is selected from C and joined to L₂, and X₁₁ is selected from C and joined to L₂;
preferably, Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, and combinations thereof;
more preferably, Rₓ is, at each occurrence identically or differently, selected from the group consisting of: hydrogen, deuterium, halogen, phenyl, pyridyl, vinyl, adamantyl, methyl, ethyl, isopropyl, cyclopropyl, naphthyl, biphenyl, phenanthryl, triphenylenyl, tert-butyl, trifluoromethyl, 9,9-dimethylfluorenyl, terphenyl, dibenzothienyl, dibenzofuryl, benzothiazolyl, benzoxazolyl, phenanthroxazolyl, phenanthrothiazolyl, carbazolyl, chrysenyl, and combinations thereof.

21. The compound composition of claim 14, wherein Ar₁ and Ar₂ have, at each occurrence identically or differently, a structure represented by any one of Formula Ar-1 to Formula Ar-6 or a combination thereof: 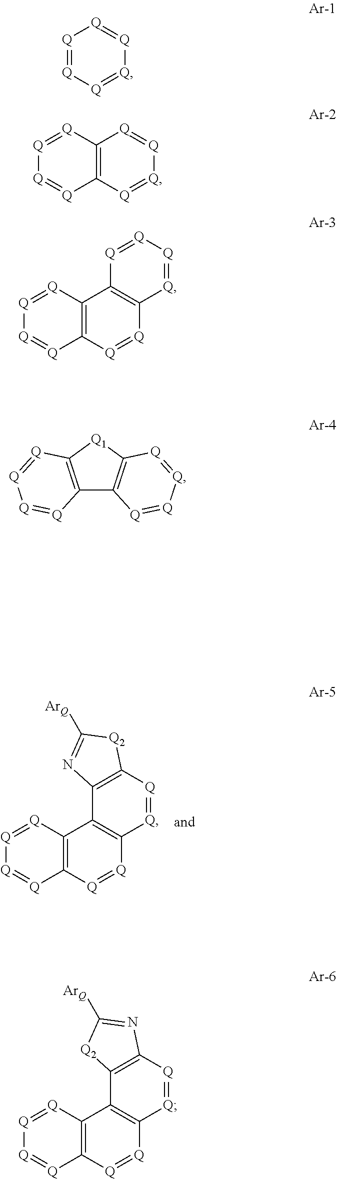
wherein
Ar_{Q} is, at each occurrence identically or differently, selected from substituted or unsubstituted aryl having 6 to 30 carbon atoms, substituted or unsubstituted heteroaryl having 3 to 30 carbon atoms or a combination thereof;
Q is, at each occurrence identically or differently, selected from C, CR_{Q} or N, and at least one Q is selected from C and joined to L₃ or L′₃;
Q₁ is selected from O, S, Se, NR_{Q} or CR_{Q}R_{Q};
Q₂ is selected from O, S or Se;
R_{Q} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{Q} can be optionally joined to form a ring;
preferably, Ar₁ and Ar₂ have, at each occurrence identically or differently, a structure represented by any one of Formula Ar-1, Formula Ar-2 or Formula Ar-4.

22. The compound composition of claim 21, wherein in Formula Ar-1 to Formula Ar-6, Q is, at each occurrence identically or differently, selected from CR_{Q}, Q₁ is selected from O, S or CR_{Q}R_{Q}, and Q₂ is selected from O or S;
preferably, R_{Q} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, and combinations thereof;
more preferably, R_{Q} is, at each occurrence identically or differently, selected from the group consisting of: hydrogen, deuterium, halogen, phenyl, pyridyl, vinyl, adamantyl, methyl, ethyl, isopropyl, cyclopropyl, naphthyl, biphenyl, phenanthryl, triphenylenyl, tert-butyl, trifluoromethyl, 9,9-dimethylfluorenyl, terphenyl, dibenzothienyl, dibenzofuryl, benzothiazolyl, benzoxazolyl, phenanthroxazolyl, phenanthrothiazolyl, carbazolyl, chrysenyl, and combinations thereof.

23. The compound composition of claim 14, wherein at least two compounds among the first compound, the second compound and the third compound are isomers of each other or isotopologues of each other;
preferably, the third compound has a structure represented by Formula 1, and the third compound and the first compound are isomers of each other or isotopologues of each other; or the third compound has a structure represented by Formula 2, and the third compound and the second compound are isomers of each other or isotopologues of each other.

24. The compound composition of claim 14, wherein a deuterated rate of at least one compound among the first compound, the second compound and the third compound is 5%-100%; preferably, the deuterated rate is 30%-100%; more preferably, the deuterated rate is 50%-90%.

25. An electroluminescent device, comprising a first electrode, a second electrode and an organic layer disposed between the first electrode and the second electrode, wherein the organic layer at least comprises the mixture of claim 1.

26. An electroluminescent device, comprising a first electrode, a second electrode and an organic layer disposed between the first electrode and the second electrode, wherein the organic layer at least comprises the compound composition of claim 14.

27. The electroluminescent device of claim 25, wherein the organic layer is an emissive layer, and the mixture is a host material.

28. The electroluminescent device of claim 25, wherein the organic layer is an emissive layer, and the emissive layer further comprises at least one phosphorescent material; preferably, a maximum emission wavelength of the phosphorescent material is 580 nm or above.

29. The electroluminescent device of claim 28, wherein the phosphorescent material is a metal complex, and the metal complex has a general formula of M(Lₐ)ₘ(L_{b})ₙ(L_{c})_{q};
wherein M is selected from a metal with a relative atomic mass greater than 40;
Lₐ, L_{b} and L_{c} are a first ligand, a second ligand and a third ligand coordinated to M, respectively; Lₐ, L_{b} and L_{c} can be optionally joined to form a multidentate ligand;
Lₐ, L_{b} and L_{c} may be the same or different; m is 1, 2 or 3; n is 0, 1 or 2; q is 0, 1 or 2; a sum of m, n and q equals an oxidation state of M; when m is greater than or equal to 2, a plurality of Lₐ may be the same or different; when n is 2, two L_{b} may be the same or different; when q is 2, two L_{c} may be the same or different;
Lₐ has a structure represented by Formula 3: 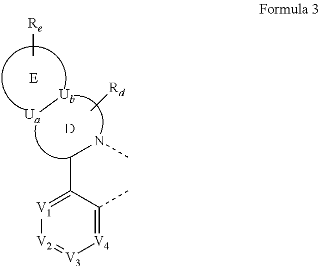
wherein
the ring D is selected from a 5-membered heteroaromatic ring or a 6-membered heteroaromatic ring;
the ring E is selected from a 5-membered unsaturated carbocyclic ring, a benzene ring, a 5-membered heteroaromatic ring or a 6-membered heteroaromatic ring;
the ring D and the ring E are fused via Uₐ and U_{b};
Uₐ and U_{b} are, at each occurrence identically or differently, selected from C or N;
R_{d} and Rₑ represent, at each occurrence identically or differently, mono-substitution, multiple substitutions or no substitution;
V₁ to V₄ are, at each occurrence identically or differently, selected from CRᵥ or N;
R_{d}, Rₑ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{d}, Rₑ and Rᵥ can be optionally joined to form a ring;
L_{b} and L_{c} are, at each occurrence identically or differently, selected from any one of the following structures: 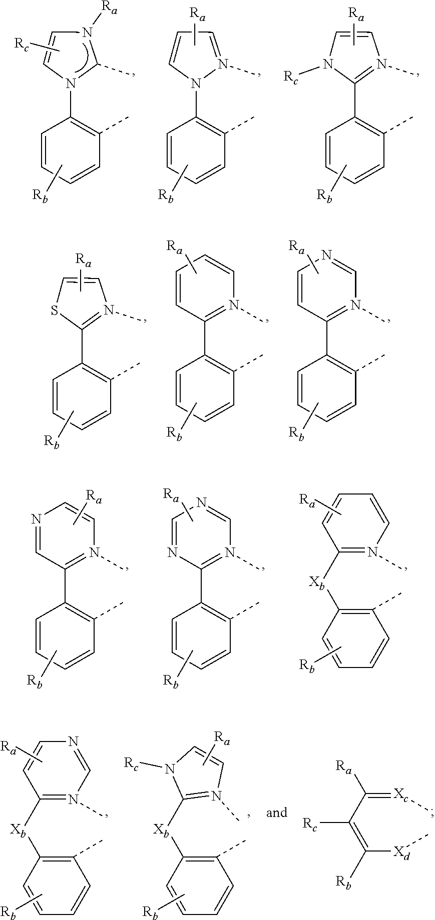
wherein
Rₐ, R_{b} and R_{c} represent, at each occurrence identically or differently, mono-substitution, multiple substitutions or non-substitution;
X_{b} is, at each occurrence identically or differently, selected from the group consisting of: O, S, Se, NR_{N1} and CR_{C1}R_{C2};
X_{c} and X_{d} are, at each occurrence identically or differently, selected from the group consisting of: O, S, Se, and NR_{N2};
Rₐ, R_{b}, R_{c}, R_{N1}, R_{N2}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
in the structures of the ligands L_{b} and L_{c}, adjacent substituents Rₐ, R_{b}, R_{c}, R_{N1}, R_{N2}, R_{C1} and R_{C2} can be optionally joined to form a ring.

30. The electroluminescent device of claim 28, wherein the phosphorescent material is a metal complex, and the metal complex has a general formula of M(Lₐ)ₘ(L_{b})ₙ;
wherein M is selected from a metal with a relative atomic mass greater than 40;
Lₐ and L_{b} are a first ligand and a second ligand coordinated to M, respectively; Lₐ and L_{b} can be optionally joined to form a multidentate ligand;
m is 1, 2 or 3; n is 0, 1 or 2; a sum of m and n equals an oxidation state of M; when m is greater than or equal to 2, a plurality of Lₐ may be the same or different; when n is 2, two L_{b} may be the same or different;
Lₐ has a structure represented by Formula 3: 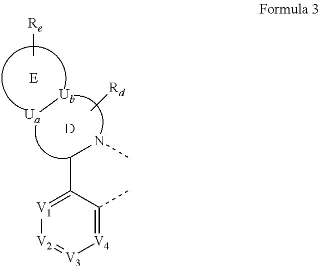
wherein
the ring D is selected from a 5-membered heteroaromatic ring or a 6-membered heteroaromatic ring;
the ring E is selected from a 5-membered unsaturated carbocyclic ring, a benzene ring, a 5-membered heteroaromatic ring or a 6-membered heteroaromatic ring;
the ring D and the ring E are fused via Uₐ and U_{b};
Uₐ and U_{b} are, at each occurrence identically or differently, selected from C or N;
R_{d} and Rₑ represent, at each occurrence identically or differently, mono-substitution, multiple substitutions or no substitution;
V₁ to V₄ are, at each occurrence identically or differently, selected from CRᵥ or N;
R_{d}, Rₑ and Rᵥ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{d}, Rₑ and Rᵥ can be optionally joined to form a ring;
the ligand L_{b} has the following structure: 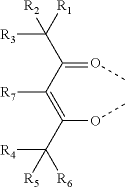
wherein R₁ to R₇ are each independently selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclyl having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
preferably, at least one or two of R₁ to R₃ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof, and/or at least one or two of R₄ to R₆ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof;
more preferably, at least two of R₁ to R₃ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof, and/or at least two of R₄ to R₆ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof.

31. A method for preparing an electroluminescent device, wherein the electroluminescent device comprises the mixture of claim 1 and the method comprises the following steps:
step 1: providing a substrate, and setting a first electrode on the substrate;
step 2: per-mixing the first compound, the second compound and the third compound to form the mixture, loading the mixture into a container of a high-vacuum deposition tool, and when the mixture in the container is evaporated at a rate of 0.01-5 Å/s using the high-vacuum deposition tool with a vacuum degree of 10⁻⁶ torr or below, evaporating the mixture on a surface positioned at a certain distance from the mixture to be evaporated to form an organic layer; wherein a mass ratio of at least one compound among the first compound, the second compound and the third compound in the mixture is C₀, and a mass ratio of the at least one compound in the organic layer to a total mass of the first compound, the second compound and the third compound is C′;
wherein |C′−C₀|≤2%; and
step 3: depositing a second electrode on the organic layer.

32. A method for preparing an electroluminescent device, wherein the electroluminescent device comprises the compound composition of claim 14 and the method comprises the following steps:
step 1: providing a substrate, and setting a first electrode on the substrate;
step 2: loading a first compound, a second compound and a third compound into different containers of a high-vacuum deposition tool, respectively, and when the compounds in the containers are evaporated simultaneously at a rate of 0.01-5 Å/s using the high-vacuum deposition tool with a vacuum degree of 10⁻⁶ torr or below, evaporating the first compound, the second compound and the third compound on a surface positioned at a certain distance from the compounds to be evaporated such that the three compounds are co-deposited to form an organic layer; and
step 3: depositing a second electrode on the organic layer.

33. An electronic device, comprising the electroluminescent device of claim 25.
